SEQUENCE LISTING (53 sequences)

SEQ ID NO: 1 - DNA, 30 bp, Artificial Sequence
  Primer OVINs2
taggcagagcaataggactctcaacctcgt

SEQ ID NO: 2 - DNA, 30 bp, Artificial Sequence
  Primer OVMa2
aagcttctgcagcactctgggagttactca

SEQ ID NO: 3 - DNA, 20 bp, Artificial Sequence
  Primer OVINs1
gggaaacaatctgccttgca

SEQ ID NO: 4 - DNA, 21 bp, Artificial Sequence
  Primer OVMUa1
aagccacaaagcacgaaagag

SEQ ID NO: 5 - DNA, 20 bp, Artificial Sequence
  Primer T3
taatacgactcactataggg

SEQ ID NO: 6 - DNA, 20 bp, Artificial Sequence
  Primer T7
attaaccctcactaaaggga

SEQ ID NO: 7 - DNA, 20 bp, Artificial Sequence
  Primer OVINs4
agatgaggtggatggtttac

SEQ ID NO: 8 - DNA, 20 bp, Artificial Sequence
  Primer OVINs5
cagcttctgctagcgtaggt

SEQ ID NO: 9 - DNA, 20 bp, Artificial Sequence
  Primer OVINs6
acgtgaactcaaagaggcac

SEQ ID NO: 10 - DNA, 20 bp, Artificial Sequence
  Primer OVINs7
atctcctgagctcggtgctt

SEQ ID NO: 11 - DNA, 20 bp, Artificial Sequence
  Primer OVINs8
acgaggttccatgtctttca

SEQ ID NO: 12 - DNA, 31 bp, Artificial Sequence
  Primer OVMUa3
taaatagcacagaacgctgaggggagtaagg

SEQ ID NO: 13 - DNA, 20 bp, Artificial Sequence
  Primer OVMUa4
gaagagcttggtagaagact

SEQ ID NO: 14 - DNA, 21 bp, Artificial Sequence
  Primer OVMUa5
atggaaatatgggtttccttc

SEQ ID NO: 15 - DNA, 20 bp, Artificial Sequence
  Primer OVMUa6
gcagcttatggctaatcgct

SEQ ID NO: 16 - DNA, 20 bp, Artificial Sequence
  Primer OVMUa7
agtgaccactatctgacctg

SEQ ID NO: 17 - DNA, 20 bp, Artificial Sequence
  Primer OVMUa8
taatcaggaaggcacacagc

SEQ ID NO: 18 - DNA, 20 bp, Artificial Sequence
  Primer OVMUP4. 7. 1
agatctggagcagcacttgt

SEQ ID NO: 19 - DNA, 20 bp, Artificial Sequence
  Primer OVMUP4. 7. 2
agcatgaagttcctcaccca

SEQ ID NO: 20 - DNA, 20 bp, Artificial Sequence
  Primer OVMUP4. 7. 3
atggagaggaatattccctt

SEQ ID NO: 21 - DNA, 18 bp, Artificial Sequence
  Primer OVMUP4. 7. 4
atttctccaggcgtgtgg

SEQ ID NO: 22 - DNA, 18 bp, Artificial Sequence
  Primer OVMUP5. 5. 1
atttctccaggcgtgtgg

SEQ ID NO: 23 - DNA, 20 bp, Artificial Sequence
  Primer VMUP5. 5. 2
atgcgagtgaaggagagttc

SEQ ID NO: 24 - DNA, 20 bp, Artificial Sequence
  Primer OVMUP5. 5. 3
gcagcacgtgtaagcttgta

SEQ ID NO: 25 - DNA, 20 bp, Artificial Sequence
  Primer OVMUP5. 5. 4
caaggcaaattatcagcaga

SEQ ID NO: 26 - DNA, 9980 bp, Gallus gallus
  3′UTR (1)..(255): 3′ untranslated region of ovoinhibitor
taggcagagcaataggactctcaacctcgtgagtatggcagcatgttaactctgcactggagtccagcgtgggaaacaatctgccttgcacatgagtcttcgtgggccaatattccccaacggttttccttcagcttgtcttgtctcctaagctctcaaaacacctttttggtgaataaactcacttggcaacgtttatctgtcttaccttagtgtcacgtttcatccctattcccctttctcctcctccgtgtggtacacagtggtgcacactggttcttctgttgatgttctgctctgacagccaatgtgggtaaagttcttcctgccacgtgtctgtgttgttttcacttcaaaaagggccctgggctccccttggagctctcaggcatttccttaatcatcacagtcacgctggcaggattagtccctcctaaaccttagaatgacctgaacgtgtgctccctctttgtagtcagtgcagggagacgtttgcctcaagatcagggtccatctcacccacagggccattcccaagatgaggtggatggtttactctcacaaaaagttttcttatgtttggctagaaaggagaactcactgcctacctgtgaattcccctagtcctggttctgctgccactgctgcctgtgcagcctgtcccatggagggggcagcaactgctgtcacaaaggtgatcccaccctgtctccactgaaatgacctcagtgccacgtgttgtatagggtataaagtacgggagggggatgcccggctcccttcagggttgcagagcagaagtgtctgtgtatagagtgtgtcttaatctattaatgtaacagaacaacttcagtcctagtgttttgtgggctggaattgcccatgtggtagggacaggcctgctaaatcactgcaatcgcctatgttctgaaggtatttgggaaagaaagggatttgggggattgcctgtgattggctttaattgaatggcaaatcacaggaaagcagttctgctcaacagttggttgtttcagccaattcttgcagccaaagagccgggtgcccagcgatataatagttgtcacttgtgtctgtatggatgacagggaggtagggtgacctgaggaccaccctccagcttctgctagcgtaggtacagtcaccacctccagctccacacgagtcccatcgtggtttaccaaagaaacacaattatttggaccagtttggaaagtcacccgctgaattgtgaggctagattaatagagctgaagagcaaatgttcccaacttggagatactagttggtattagtatcagaggaacagggccatagcacctccatgctattagattccggctggcatgtacttttcaagatgatttgtaactaacaatggcttattgtgcttgtcttaagtctgtgtcctaatgtaaatgttcctttggtttatataaccttcttgccatttgctcttcaggtgttcttgcagaacactggctgctttaatctagtttaactgttgcttgattattcttagggataagatctgaataaactttttgtggctttggcagactttagcttgggcttagctcccacattagcttttgctgccttttctgtgaagctatcaagatcctactcaatgacattagctgggtgcaggtgtaccaaatcctgctctgtggaacacattgtctgatgataccgaaggcaaacgtgaactcaaagaggcacagagttaagaagaagtctgtgcaattcagaggaaaagccaaagtggccattagacacactttccatgcagcatttgccagtaggtttcatataaaactacaaaatggaataaaccactacaaatgggaaaagcctgatactagaatttaaatattcacccaggctcaaggggtgtttcatggagtaatatcactctataaaagtagggcagccaattattcacagacaaagcttttttttttctgtgctgcagtgctgtttttcggctgatccagggttacttattgtgggtctgagagctgaatgatttctccttgtgtcatgttggtgaaggagatatggccagggggagatgagcatgttcaagaggaaacgttgcattttggtggcttgggagaaaggtagaacgatatcaggtccatagtgtcactaagagatctgaaggatggttttacagaacagttgacttggctgggtgcaggcttggctgtaaatggatggaaggatggacagatgggtggacagagatttctgtgcaggagatcatctcctgagctcggtgcttgacagactgcagatccatcccataaccttctccagcatgagagcgcggggagctttggtactgttcagtctgctgcttgttgcttcctgggtgcacagtggtgattttcttactcacacagggcaaaaacctgagcagcttcaaagtgaacaggttgctctcataggccattcagttgtcaagatgaggtttttggtttcttgttttgtaaggtgggaagaagcactgaaggatcagttgcgagggcaggggtttagcactgttcagagaagtcttattttaactcctctcatgaacaaaaagagatgcaggtgcagattctggcaagcatgcagtgaaggagaaagccctgaatttctgatatatgtgcaatgttgggcacctaacattccccgctgaagcacagcagctccagctccatgcagtactcacagctggtgcagccctcggctccagggtctgagcagtgctgggactcacgaggttccatgtctttcacactgataatggtccaatttctggaatgggtgcccatccttggaggtccccaaggccaggctggctgcgtctccgagcagcccgatctggtggtgagtagccagcccatggcaggagttagagcctgatggtctttaaggtcccttccaacctaagccatcctacgattctaggaatcatgacttgtgagtgtgtattgcagaggcaatattttaaagttataaatgttttctccccttccttgtttgtcaaagttatcttgatcgccttatcaatgcttttggagtctccagtcatttttcttacamcaaaaagaggaggaagaatgaagagaatcatttaatttcttgattgaatagtaggattcagaaagctgtacgtaatgccgtctctttgtatcgagctgtaaggtttctcatcatttatcagcgtggtacatatcagcacttttccatctgatgtggaaaaaaaaatccttatcatctacagtctctgtacctaaacatcgctcagactctttaccaaaaaagctataggttttaaaactacatctgctgataatttgccttgttttagctcttcttccatatgctgcgtttgtgagaggtgcgtggatgggcctaaactctcagctgctgagcttgatgggtgcttaagaatgaagcactcactgctgaaactgttttcatttcacaggaatgttttagtggcattgtttttataactacatattcctcagataaatgaaatccagaaataattatgcaaactcactgcatccgttgcacaggtctttatctgctagcaaaggaaataatttggggatggcaaaaacattccttcagacatctatatttaaaggaatataatcctggtacccacccacttcatccctcattatgttcacactcagagatactcattctcttgttgttatcatttgatagcgttttctttggttctttgccacgctctgggctatggctgcacgctctgcactgatcagcaagtagatgcgagggaagcagcagtgagaggggctgccctcagctggcacccagccgctcagcctaggaggggaccttgcctttccaccagctgaggtgcagccctacaagcttacacgtgctgcgagcaggtgagcaaagggagtcttcatggtgtgtttcttgctgcccggaagcaaaactttactttcattcattccccttgaagaatgaggaatgtttggaaacggactgctttacgttcaatttctctcttccctttaaggctcagccaggggccattgctgaggacggcatcggggccccctggaccaaatctgtggcacagatggtttcacttacatcagtggatgtgggatctgcgcctgtaatgtgtccttctgaaggaaggaacgtgccttccaagtgccagccccacagcccccagcccctccctgtgctgctccaattcatctcctcttcctccttctccctttgctgtttgtgctcgggtagaaatcatgaagatttagaagagaaaacaaaataactggagtggaaacccaggtgatgcagttcattcagctgtcataggtttgtcgttgctataggtctgtatcagagatgctarcaccactttgctgtcggtgcttaactcgggtgaactctccttcactcgcatcatttgcgggccttatttacatccccagcatccatcaccctctgggaaaatgggcgcactggatctctaatggaagactttccctctttcagagcctgtgggatgtgcagtgacaagaaacgtggaggggctgagcagcagcactgcccccagggagcaggagcggatgccatcggtggcagcatcccaaatgatgtcagcggatgctgagcaggcagcggacgaacggacagaagcgatgcgtacaccttctgttgacatggtatttggcagcgatttaacactcgcttcctagtcctgctattctccacaggctgcattcaaatgaacgaagggaagggaggcaaaaagatgcaaaatccgagacaagcagcagaaatatttcttcgctacggaagcgtgcgcaaacaaccttctccaacagcaccagaagagcacagcgtaacctttttcaagaccagaaaaggaaattcacaaagcctctgtggataccagcgcgttcagctctcctgatagcagatttcttgtcaggttgcgaatggggtatggtgccaggaggtgcagggaccatatgatcatatacagcacagcagtcattgtgcatgtattaatatatattgagtagcagtgttactttgccaaagcaatagttcagagatgagtcctgctgcatacctctatcttaaaactaacttataaatagtaaaaccttctcagttcagccacgtgctcctctctgtcagcaccaatggtgcttcgcctgcacccagctgcaaggaatcagcccgtgatctcattaacactcagctctgcaggataaattagattgttccactctcttttgttgttaattacgacggaacaattgttcagtgctgatggtcctaattgtcagctacagaaaacgtctccatgcagttccttctgcgccagcaaactgtccaggctatagcaccgtgatgcatgctacctctcactccatccttcttctctttcccaccagggagagctgtgtgttttcactctcagccactctgaacaataccaaactgctacgcactgcctccctcggaaagagaatccccttgttgcttttttatttacaggatccttcttaaaaagcagaccatcattcactgcaaacccagagcttcatgcctctccttccacaaccgaaaacagccggcttcatttgtcttttttaaatgctgttttccaggtgaattttggccagcgtgttggctgagatccaggagcacgtgtcagctttctgctctcattgctcctgttctgcattgcctctttctggggtttccaagagggggggagactttgcgcggggatgagataatgccccttttcttagggtggctgctgggcagcagagtggctctgggtcactgtggcaccaatgggaggcaccagtgggggtgtgttttgtgcaggggggaagcattcacagaatggggctgatcctgaagcttgcagtccaaggctttgtctgtgtacccagtgaaatccttcctctgttacataaagcccagataggactcagaaatgtagtcattccagcccccctcttcctcagatctggagcagcacttgtttgcagccagtcctccccaaaatgcacagacctcgccgagtggagggagatgtaaacagcgaaggttaattacctccttgtcaaaaacactttgtggtccatagatgtttctgtcaatcttacaaaacagaaccgagaggcagcgagcactgaagagcgtgttcccatgctgagttaatgagacttggcagctcgctgtgcagagatgatccctgtgcttcatgggaggctgtaacctgtctccccatcgccttcacaccgcagtgctgtcctggacacctcaccctccataagctgtaggatgcagctgcccagggatcaagagacttttcctaaggctcttaggactcatctttgccgctcagtagcgtgcagcaattactcatcccaactatactgaatgggtttctgccagctctgcttgtttgtcaataagcatttcttcattttgcctctaagtttctctcagcagcaccgctctgggtgacctgagtggccacctggaacccgaggggcacagccaccacctccctgttgctgctgctccagggactcatgtgctgctggatggggggaagcatgaagttcctcacccagacacctgggttgcaatggctgcagcgtgctcttcttggtatgcagattgtttccagccattacttgtagaaatgtgctgtggaagccctttgtatctctttctgtggcccttcagcaaaagctgtgggaaagctctgaggctgctttcttgggtcgtggaggaattgtatgttccttctttaacaaaaattatccttaggagagagcactgtgcaagcattgtgcacataaaacaattcaggttgaaagggctctctggaggtttccagcctgactactgctcgaagcaaggccaggttcaaagatggctcaggatgctgtgtgccttcctgattatctgtgccaccaatggaggagattcacagccactctgcttcccgtgccactcatggagaggaatattcccttatattcagatagaatgttatcctttagctcagccttccctataaccccatgagggagctgcagatccccatactctccccttctctggggtgaaggccgtgtcccccagccccccttcccaccctgtgccctaagcagcccgctggcctctgctggatgtgtgcctatatgtcaatgcctgtccttgcagtccagcctgggacatttaattcatcaccagggtaatgtggaactgtgtcatcttcccctgcagggtacaaagttctgcacggggtcctttcggttcaggaaaaccttcactggtgctacctgaatcaagctctatttaataagttcataagcacatggatgtgttttcctagagatacgttttaatggtatcagtgatttttatttgctttgttgcttacttcaaacagtgcctttgggcaggaggtgagggacgggtctgccgttggctctgcagtgatttctccaggcgtgtggctcaggtcagatagtggtcactctgtggccagaagaaggacaaagatggaaattgcagattgagtcacgttaagcaggcatcttggagtgatttgaggcagtttcatgaaagagctacgaccacttattgttgttttccccttttacaacagaagttttcatcaaaataacgtggcaaagcccaggaatgtttgggaaaagtgtagttaaatgttttgtaattcatttgtcggagtgctaccagctaagaaaaaagtcctacctttggtatggtagtcctgcagagaatacaacatcaatattagtttggaaaaaaacaccaccaccaccagaaactgtaatggaaaatgtaaaccaagaaattccttgggtaagagagaaaggatgtcgtatactggccaagtcctgcccagctgtcagcctgctgaccctctgcagttcaggaccatgaaacgtggcactgtaagacgtgtcccctgcctttgcttgcccacagatctctgcccttgtgctgactcctgcacacaagagcatttccctgtagccaaacagcgattagccataagctgcacctgactttgaggattaagagtttgcaattaagtggattgcagcaggagatcagtggcagggttgcagatgaaatccttttctaggggtagctaagggctgagcaacctgtcctacagcacaagccaaaccagccaagggttttcctgtgctgttcacagaggcagggccagctggagctggaggaggttgtgctgggacccttctccctgtgctgagaatggagtgatttctgggtgctgttcctgtggcttgcactgagcagctcaagggagatcggtgctcctcatgcagtgccaaaactcgtgtttgatgcagaaagatggatgtgcacctccctcctgctaatgcagccgtgagcttatgaaggcaatgagccctcagtgcagcaggagctgtagtgcactcctgtaggtgctagggaaaatctctggttcccagggatgcattcataagggcaatatatcttgaggctgcgccaaatctttctgaaatattcatgcgtgttcccttaatttatagaaacaaacacagcagaataattattccaatgcctcccctcgaaggaaacccatatttccatgtagaaatgtaacctatatacacacagccatgctgcatccttcagaacgtgccagtgctcatctcccatggcaaaatactacaggtattctcactatgttggacctgtgaaaggaaccatggtaagaaacttcggttaaaggtatggctgcaaaactactcataccaaaacagcagagctccagacctcctcttaggaaagagccacttggagagggatggtgtgaaggctggaggtgagagacagagcctgtcccagttttcctgtctctattttctgaaacgtttgcaggaggaaaggacaactgtactttcaggcatagctggtgccctcacgtaaataagttccccgaacttctgtgtcatttgttcttaagatgctttggcagaacactttgagtcaattcgcttaactgtgactaggtctgtaaataagtgctccctgctgataaggttcaagtgacatttttagtggtatttgacagcatttaccttgctttcaagtcttctaccaagctcttctatacttaagcagtgaaaccgccaagaaacccttccttttatcaagctagtgctaaataccattaacttcataggttagatacggtgctgccagcttcacctggcagtggttggtcagttctgctggtgacaaagcctccctggcctgtgcttttacctagaggtgaatatccaagaatgcagaactgcatggaaagcagagctgcaggcacgatggtgctgagccttagctgcttcctgctgggagatgtggatgcagagacgaatgaaggacctgtcccttactcccctcagcattctgtgctatttagggttctaccagagtccttaagaggttttttttttttttggtccaaaagtctgtttgtttggttttgaccactgagagcatgtgacacttgtctcaagctattaaccaagtgtccagccaaaatcaattgcctgggagacgcagaccattacctggaggtcaggacctcaataaatattaccagcctcattgtgccgctgacagattcagctggctgctccgtgttccagtccaacagttcggacgccacgtttgtatatatttgcaggcagcctcggggggaccatctcaggagcagagcaccggcagccgcctgcagagccgggcagtactctcaccatggccatggcaggtgtcttcgtgctgttctctttcgtgctttgtggcttcctcccaggtgagtaactcccagagtgctgcagaagctt

SEQ ID NO: 27 - DNA, 20 bp, Artificial Sequence
  Primer OVMUa9
aaatgaagccggctgttttc

SEQ ID NO: 28 - DNA, 20 bp, Artificial Sequence
  Primer OVINs9
ctctcagccactctgaacaa

SEQ ID NO: 29 - DNA, 40 bp, Artificial Sequence
  Primer
gcgcggccgcccgggacatgtccatggtgagagtactgcc

SEQ ID NO: 30 - DNA, 29 bp, Artificial Sequence
  Primer
ggcccgggattcgcttaactgtgactagg

SEQ ID NO: 31 - DNA, 802 bp, Artificial Sequence
  primer
gcgcggccgcccgggacatgtccatggtgagagtactgcccggctctgcaggcggctgccggtgctctgctcctgagatggtccccccgaggctgcctgcaaatatatacaaacgtggcgtccgaactgttggactggaacacggagcagccagctgaatctgtcagcggcacaatgaggctggtaatatttattgaggtcctgacctccaggtaatggtctgcgtctcccaggcaattgattttggctggacacttggttaatagcttgagacaagtgtcacatgctctcagtggtcaaaaccaaacaaacagacttttggaccaaaaaaaaaaaaaacctcttaaggactctggtagaaccctaaatagcacagaatgctgaggggagtaagggacaggtccttcattcgtctctgcatccacatctcccagcaggaagcagctaaggctcagcaccatcgtgcctgcagctctgctttccatgcagttctgcattcttggatattcacctctaggtaaaagcacaggccagggaggctttgtcaccagcagaactgaccaaccactgccaggtgaagctggcagcaccgtatctaacctatgaagttaatggtatttagcactagcttgataaaaggaagggtttcttggcggtttcactgcttaagtatagaagagcttggtagaagacttgaaagcaaggtaaatgctgtcaaataccactaaaaatgtcacttgaaccttatcagcagggagcacttatttacagacctagtcacagttaagcgaattcccgggcc

SEQ ID NO: 32 - DNA, 17 bp, Artificial Sequence
  Primer
ctccacatggccatggc

SEQ ID NO: 33 - DNA, 17 bp, Artificial Sequence
  Primer
gagtggtaccggtaccg

SEQ ID NO: 34 - DNA, 17 bp, Artificial Sequence
  Primer
ctcaccatggacatgga

SEQ ID NO: 35 - DNA, 17 bp, Artificial Sequence
  Primer
gagtggtaccggtaccg

SEQ ID NO: 36 - DNA, 75815 bp, chicken
aagctttgtgctttctgcctgaataaaagaaacctgaactctgttcacccagtccctgtcaggcaattactgacagagcacctatggtctgtgtttggccagaacataggctaaggaagatacctcctgtttataaagcacgcctttggcatctggcaagtaattagtgatggcgcatgagagctctgactagggcagggtgtgggacaggctggctctaattgtgccctgtttatcttgttgatgcacacggctggtttctttcacccacagctgtctctctagacaacatacctttatggagaggaacgtgtcttttccaatcttgggttttcattcagaattggagtgaactggtctccatcagatagcattggctgcggtgatttattcttttacacttcctagttaagcaggataactctctggctctgctgtgtctaggcaatttaaatgatttataaagcatagctgttttaaggaaatctttttttaaacatttgacttgccaatgtgtggtcctaaaggcagaaggactgttccagagtgtcaggcagagacctaccctggatttcgttgttcagctacccattcagtgtggcttttggcaaggaattctctggacctgacttccctacctgcagagctgggataagctatcaaaccatctcctccacacactgtgagggtgggaaaaaaacccaaacccttaaaagtgctgtataaaggcgccttaaggctcagtatagcatgtgtgctgctgatgccccagacctgtttgcgggtcctgaaggtcataggagaactgctcagaagagacagaaatgcttaagaaggttttactacaaaagtcttgtgatgttaacacataatatcacattgtgcagaaggtacaaatgccccctcctatccctgcacacctggaagctcaaggtatggaagggtttgttgtctgcagcctcttcgctgccctctgctttttaagatcctgggtagtgtgctcagtgtgtgccctcagcagtttgggaaacggacatcttcatgcaaaattaagcaaggaagtgttgcttttatactcagagtagaatctaagttcttcaggcaggctcttgtgtgccgcctctattagaaataaaactcccccggatcagaagatgaatgtgctcagctaagaacacagatttatttgctttacaatgcgtgctatggtttaagaaaaacacatcaggcaaacaatttatggtttgccactgagttgtgcctgaaggaaacacaactgttagagatgtaattgattgggcggtgacgctgtgtggattcatgggagatgcatcttggtcagcatgtctgtgtgaaaccacatttctggtgctgctgcaggacgagtgccgggagttccgggatctgttcaagaatgggaagctttcctgcacgagggagaatgatcccgtccgggattcctcggggaagcagcacagcaataagtgcatcatgtgtgcggagaagttgtgagtagaggaagccaatgtttgttatcgagagtggcaatggggccggggtgggctcctacagcaatgttctcctcactttctcatccttctctttcagcaaaagggagaatgagcagaaggcgacctcaaccagagggaaacaaaaggtgaggttaaagtattgggttcatatacaagtctataggattcttacccaatattaccacacttgatttctttgtcactctggggatccatgtggcttttcctgcttgtatctcgttgatgctctttcatgccctgagagaatagtttgtctgaacgctgcagtctatcccactgaccgcagtgacatgggagcaaaccccatcgcaataagaagctgagcagaactgccctgacatctggcacaagggcaagaaggcactgctgctgagagcgctaatgaggttgaaaagaaaatctgggtgagaagctttaaatgtgagctctgagatgctcaaaagttcattatgtcgtgggaggagagttcagccctgtgctgtccctggggtggctcggtttcagctttccctgattggaaacctcactctcatgatgcagctgctgtgcccttgtgcaccgatacttctctggtgagagcaattcagcaaggggaaggaaaaagaagcactaagtaaatcttgccatttctgtcttgcgaggaactggtacggtccccttaagcctcattcttggggataatcctgtttcagtgcttttcctaatgacagtggcacaaaaaaaatggaagcgttaatgaaacttgctgatggcaaagctgggagggaggatcagcagatcactcaggactaattggatagcactgaggcctggagtaatagaaacaagataaaatgtaataacagagagtgcaagatcacacaggcagtgattaacgagaattcctgctcatcaattagaaatgacaaaggataagaaagctctgcatttattagtgggtcacggatgcggcaggcctgagaaggaggcaaatgcacatctcagcaaggtctgtgcagcagaggtcgggctggcagcaaatctccagaaatactgctttgaagagagagggtttgagagacgctgttagggagaagcagctctgccacagcaggtctggggttcacctggggtttggctcattgcctccctgtgtccctcctccacgctgccagtgctgcactgggaaggtgtgggtaagaagcaatggctaagggatctggttatacacctcctgtatctgctatttgggattggctactgcagggcctcaggtccctgacttaaaagtggggacttcgaagcatgtttgcattgtgctgtcgtgccttagatgttgctgctgggtcctcaaagtcctgttggttgtggggtgggggggacttcttgcttcctatgtgaagttttctgagctgcaacttcagcaacagctgtaagagtgcattaagggcagtgggagaagtgggagggaccccattacctcatcgggtatcgctggcatgctttggatagccccacgtggagcgtgacaattagagcacggcagagagctcccaacacgtgccatgcaggcagaggcacccgccgctcttctgactcactctgtttgtagccatgaggctgtgccacgtgccctcttctctctctcacacctgggctctcctggggcgcgtttgggaagcctctggaggatcggagggatgtggcagggtgccctgactgctgctccttccgcaggatgactgcagtgagtaccgctcccagtttgaggctggcggacgcctgtcctgcacgcgggagaacgaccccgtcagggattcctctggcaagcagcacaccaacaagtgcctcatgtgtgccgagaagctgtgagtacagttcctggcaacagcaaagagggaaacctcacattgcgaaactgcagcttctgcctgtgtggctgcgcctgggggagtcccgagtcccagcggccccccaggagctgctcctgctgtagggctgtggctactgcccctcttcccacctcccccctaacccctcagggagcagaggagaagcagggttgatagagagcagccctttccttggggcagctcccaaggaaagtttcccacgcgtgtactttgccttccagatgctctctctactcccatagagcatatgcagaagcagccctgatatgaaagcagccacctggagccgggatgtagcatacagtgggaatggtgaggagaagggagaaggcttaggggtgggaattaggtgcagggccaccagggatggggaggctggtgcctaatgacatgatgctggcttgcagggcagccccaggtcctggcagcgttcgcactgccatagtgctcctttctttctcctctcccttttttccagcaaaaaagaagctcaaagaggaggtcagtctggtggaactgcccagcgcaacaagcagtccactgcagagtgtgcaaaccaggtgagactgagctcagagcctcaccaggcttgggaaaaggggttggtggatctggggaccccgatggtcaagggctgcctgtggtcctggtgtttggggtgcaggagcctgctggtgatggcagagaggcaggttgcattgcaagccctgctagttcatgggatgggtttgtgtatgagcgtgcatagtgggcagttctggactcctctatggggcacgcatcagagctatttcttcagaaagagccccatggttcctagggtccagggggatgagagggaaggacaggagctgctttaatctcactgctttactgcttggttgtcaaacacgatcctgccccttttccagaagagctgcagtggctcagggttacagcggggtgtaaatgagagacggccgttctccacaaacagagggtgagtacagcagcactgggatcccagcctggccccacaagtcctggggtcttgacactgagaagaaacacataaaatagggcatatacaaccctttctcctttccaaagacattcttgcttcccctgcacacgaagcactggtgactgctacactcaaaatccctccccagccttgccccctgaatcctgcctcctggcaggcacacacttgtcctgctgcctggtccagcgcatcctcatctgctgacctgaggcagtgctgtgtgtgcaccatgtgctgtctgggcactgagcgactcctctgggtttttagggctgccaggctctggcagggtgcagatgctgtgttatctaagccttgaggaactctcttagtcttcctgtttttgttggtgaggcccattcatctgcccccagtcagcactgccagcagacaaacagtgcacagctctccatggcagcaatggctgtagcatatgtaggggccaggtttctgggatcatctctgtgacggacatctcttgctgaccgcccataaggactcaaaagtcccgttgcagggagtgcctccatcccatggcaagccaagtgccctgttgaaaaaacaaggtgcagaataatggcaatggaccttagtgcagtttaattccaccctggggtgatgatgtggctgagtgggtctgcatacccttggctgtgccatgagctctgtgctttctctccctgccagcccacaaggagacttggctcaggactgcagcccggcacctggccgccagggacagagcggaggcaccaacacctaccagccggtatgcccagctcatgtgggtcaggcacagcctttcccagcagctgccccagtttccattgtcaacctaaagcctcacaatgggacctgtatccttggaggggtttaaatgggtggtagagtccgtaccctgatgctgtcccctggcctcaaagaggagtgaggctgcacacgtccaaacgggagtcactgaagccagtgctgctgctggtgttggctcactgtagaagtatgtcaggtatgagagagcatcctccaggaggtgatggtggtgtcccttcctgcatgctgagatgttgggttgaagactgtggccagagcagggtgctggggctgagcgggggataaggacaaggctgataagaggaggggagagggagtagtgggggaggacacggtgagcaatagataacgactgtttgtggaatcatgtgggagggagaagagggtgtatgctctctccatctccacaaaaagaaaatttgttattttcaaccaagctaaagcagaaattatgaaactaataggagaaaataagttactataaaaaggatgactaacctgtggatcttgctgtcacggggtgttgccaagagctacagtgattaaaaaaaatgacttgccacttatagtccatacagcaatttaggtaacattttggaagggataggaaatgcctttctgtggggctggagggacctgagtgcagactgccttaactctctctgaagtctctgtcactgactgcccttagaaaaatgatattagaatagaaaaaccagggaggcggttcaggtatggcagttttaatgcattccagaggaagcattaggcataataatgccagtctgcttcagggcttagtggtatttcctggtagctccggtgaaggagtggatgctgatcagcctgactgacgaggggtgattcagagagcagatctgtgtctctcctcgctgcagggccacccgtgggctctgtcccagggagatgctgtcctgaaggagaggtggcagtcactgtgaggactgtgggggactgttggtgtggcggcggttgcacacgcgtgggtcacaccgtgggcagtggtgtctggtgtgtgggaaggcatctggcagggaactgcaaaggtcagcgctgtctgtctttgtgtcatcgttaattacccaggtgagggaggaagcagcacattaatgaaattagcaagtgatgtttaaacagagggtgttactgcagcaacctgtgccactgaaccccctgcattgcccagctgggaaacctttcttctccatggtgctttcaaccccatagtgctgctgaccccagcaaagcaatgagccattgcttagtgctgaatggggttttttttctccaagtgggacaggaggtgagatgtccttcctgcagctcttctccaattgcaccatttgcagtcattgcaacattttttataggacctggagaaggggatgggaacagagaattcactccttttgtctctgcatctttttttttttggcctttggtgcagaggtgggcagtgaggctgaggaagagagggggctgtaggatctctgacctctgctgtctgaaacttgccatgattctgcaggcacctgtgccagaatgctcatgggctgataatctaatcatgaggagtcttgttcctcctgctccgagctctttctagctgtgccacgtctgctttgtaggaaattcgatgcctagatgctcctgctgttatgctggagaataaaacgagagggcacgcttaattagtcagagcttttcatacatgtttgcatctcttcattccgtgggtgtcaagttgtgctgtgtgtcgggctgcccttgggcagctggactcaattgtcaaggttttccctttgtttctgccaagtggcttgcagaagcaacaggtgtgaaagctctgataaaggacaaaggacaggtagcagaagtttattgtattctcgtggatttgcagggagaagtaaaagtgccctggactgagatgtcagggtggatcagatgagtgtatccatgcctggcaatggggtcagggcagctttgtccccacatcgtggctggttggcccaataggaggcgttacctctttgctgaaggtgtgatggagctcagggcaacgcctggtttgtgagtgctttgagcggtgcgcaggagggtcttgcaagagaaccagcaccaaatgtgatttctttctctcttcagctggactgtgatcgaattctgcacggggtaaagggtggaaggattttctgcagcgaatcctcacaacccgtctgtggcactgatgggaaaacatacagaaatgaatgtgacttgtgttcagctgccatgtgagtaggcggagagatttcagtaatacagggccatccaccattcccgagtgtcttttgcagcacagtgtttgttttgatataccatgactcactatcaagtgtgtccttggtgcctcgctgttaagcaaacatagatcaaatgtctgagattaatatgatgacagctaattaagatacacaactttccagagtcccttattccctttctgctcaatcataggattgtttggggagtaataaatgccatcaaattggaagtagcatcaaaggtttaaggagcccacagaggaccaccgtgacgatgtcagggagctgtggcactggaagtgaataagcaatgtcttgttctccctttgcaggagagcatcagtttacatcacggtaaactaccgaggtgaatgccgaaagactgtccctgaaatggtaagtgcctccctgctgtggcatcccatttcttgttctgggtgtgtgctggagacccagcctggatcccgtatctgtggtgggatcatcagagccctgttagcagggtgcttgtggttcacatgcgtaaatacacttcaggcttggatttaaggcattttgaggcataatctccacgttttttccaggctgtgtggtaggggagtgacatgtctgggaaaacatgtggctttcctcctgggattttggtgaggccaagaaaagattgcaatcgcacaaaccataagggcctaatttcccaaatgatatccaggcagttggttgggaaggaaatatattccctaagtggtatccttttgggaaaggtcttgaatcttgtgtgattgccttgtagtagatgagtcaaagatttgttagtggtgctttgtcttcccgctcgtggcagctcagcggcattcagagctttggtttggagccagggtgtcccagtttgtgtgtcttgagtgtatgggactgaccttagtgttggcatggactgttggaaagctgagtattcatttccccagggaaacaccgacatctatccccattccaaacttggaatgaatcaaaatatcaaatcagccaaatggagaagttgtgcaagttttttttgcaatgagagagatggcttctgaatatgaatttgctgacagtttgtaggtaaaacagtattgcccgttgaaaagctttagagcaaaattaccatcatagggcttttactctcctctgcttattgacaggatgcccacccatccccacaacattagaaatgaggcatccccattcctcttcctctcttctgtgaagtaccagagtgctctcaacgctgtttaaagctgaagaaaaaatgcagagaaagagttttgcttgtgatcgtgctggaggtctttgtgtctcgccctttggtgcgatggagccattgctggtttgtgtatgctgggagtggaggcactatgcatacctgctggtggctgtgctaatgatgctggagacagacaaggttgggtgtaccacggcaactgaaaaccagagaggactccctcagagttgtgcctggctgggattcctcaccattttgtgttttaccaagacgttttaccagctctccagtctttgcagttagaggaatatgccatacactaaaagtcagacaatttgtagctattccaaggagagctggaagcaattaaagggaaagtgataaggtttttccactggggaaaatcccccacaaaaaacacccctccaaacaaagacttattatttcgttctttatgtatattgtgtcacctgaagaatcagattggaaatttatggaagcccatttccttagcaaaccccttgtgtccatcaaagacttcccttttttttctcagttggaagcttatgaacaatgtactgaccagtgttattttatgcctctgaaattcatgctaacattcagcttaatgcatccttctgaaggcccaggcactcgctgtgtgaaggagatcacagtgcctttggcgtcagaaatgatttcaggctgttgcaatacgcagcacgaagatgcaaaggcccaaagacttgagccttggaaaaagataggagattgctgcccgaaaatgtagtttgtccttgagttgtgttttgaaattagccacggtaatgctgtgttgcctgccaaaatgtgtgtccaagctcagagcctgcagccattcctgctagcaaagcccctcctggatttccagcagtttgtggcagtccttccctagcagtggctggattgccatcagggagggatggctgtaggaagggacaggagaaatgtggttggagagagatctgacattaaagggtgcatccggacagcctgcactgatgtggtggaaaaccttcctgcagagagagccctggggctggctggcagctgggcccctgctgcctgtgtgagctctgtgccacaaccagcctcctctgatcctgttctgctttactgcagatgaatgtagctgagtctagggtttagatttctatgtttatttttaacaaggcagctggcctctgcgtcctccatgctgtgacatacagctgtattaatggtgggtctttccagaatgtttcactttcaatgctgtatttttttttattttgcagtttctctttttgttcagatgctttttcacacatctcccatgtgacagataccagtctgtccatgttagttgacaggtcaggcaaaaaaaaaaaagggatatccagtttctcctttttaatctgttttctaaagaacaaagaactcccagctttctaatgggcaaggccattttcttacagtgctctttttgtcatacctttcttaagaatgtagtagaagggaaaagaaacaaacaaaaaacccaggaccttttccagcttgatattggttttggaaagcacacagatccaggctgaaatctgtttgttttctgagtctggcagtgacccatccactgccccatcccacctggttcctgtggccactgagctgcccaaaggggctgtcatgtagcccctaatgctctgccagcgtaacagcagtggatgtacttgtggatccacttatattttgctctttctttccagaaataatggagttcagactgccagcaaataccagggatcagctgtgaccaaaggtacagtggtgcggtgatttgctccctcttggacaacttgtccgcatttcacaagggtttgggtgtcagaccttgcctgggcaggctgctgggtatgtctggggcaaagggctctgcaacacacccttccctattgccacagcacaagaatgaggcgtgtgtcttttgcagaagtagcaaggtgatgggaagcccctgccaagggggctgagccctttggggtgtgcaaacttcatgaggacctcctcatctctcaggggtgggccttgcccgttccttttccctcagatatccctgcagagggggaaggatgctggcagagcagagtactgcagtccctcctcacaaggaggtggaggtggcccaaagcaacctggctttgagctttccttgtggttcttctgtgtcccttgccttttggagccatagtaataaacccgtctgccccctgtttctctaggacaagtaaaggaagatctgatgtcaggcaccagggaagctgctgagttccccagtgctgttggatccaccttcatctccttctgcagccaacgggcctgtccttgctcaggtggagggtgaagggctgtggggacccagtggtggcttcccacgttggccccacgcatgttgttgtagtcgctgctcggctcgggctctgccgcctcgctgtgtcttagcatgtttctacaataaagataactccacagcgtcctgtcgcttttcttcactgagcctcacgggagggacgtgtgagtccccgctccggctgctcgccacgcgtcccttgagctctaaagcaccaaacccaagcggagatgtcagacgcagagaagaagaacgtggtctgggttctgttagcagggaccagcagttgggttctctgactcgctgtgtagggctttgggtgtatctctttgtctcccttcagcccttttctcttgcctgtaaaaacggacattaaaggatgcttacctacctcagagggttgtttggagattttaattggtttacgttagagagcccacgggtggaattctgttcctatgtgccaatgctggtgtgcaggaggtttaactgttgcagtcatggcctcttccagccaacacccgatgggccgtatgtatttcctgttctttcgtttatggctgttacttaaagcaaatatgttcttatttgtataaactttattgcaggacatttccagaagaccttgagtgaacgtacagtgtttgagtccactttagctgtgacctgatctgcaaatacactctgctgtagataaggctggagtaactttcagattttggcagggtttcgctcaatgccaattaatttggctccctccacagatattgatttttttttttcttttcaattaagttatcgagatctttttttcttaatgcagctaatgaaaatcgatttttactctcataaagtacttccgcatgtgtcacattgatctgtctatggcttgattatcggcaggctttgacatgaggttaatattttgtgtgctggttttttttcaccgtgtgcaaacactgtggtttagaaatatgttaccgctgcttatttctacgtggaaaatcccacggcgtggttatgcatggcagaagtcaccagtttgatccaatttagctgtttctagggatgcaagattcctctgcctttgagcgggtgaatcctcgggtgttatttatacattctgagaaggatgaacagaagacggtaaaaacgtttgctaatgatgtctgctggctgattccggctaaaatcgtgtgcagggacctcgacgtgatttttataaaggcagctcacaatttgaggcttaaagtaagttcttgcaaatgaaaatgggcgcacttgagcgcgctattataacttgtagtgatttcaagcacttagattttgaaataatcgcccataaaaacctgcattaattgtgctccaaaaccaatgagctgatgaggagggtgccctggtagcctcttttgctggatttgagcaccttctgaatttctcctgccaccagcagaaattagccacagaaatcatagctgctataagggtttattaatcagattacgaaactgctaagaaggcacacaacagtgacttgctgaagctgcctgtgctgctgttagcgagcctcccgtaggtagcaatgctaactccttccttttagcagtttacccactgcttccttccatcactccttccttttgtagggcctacttttgcagtttgatccagtggcttgcaggcaatatctgtccccagcggtgctctatgcagctgacctccaggtagggctccatgtgagcgatgcaatgtgttatttccatggggttcctaagaaggaggaagcaaaaagctcaggaggtgctccaaatatattatcctgtcctctgttttgctctttgtggtgccctttaacactgtaaagagaccataggagtcctctatgaacctggaaaggtaccagcactatgggaggtcttcagtttgctgtaaattatgctttattagaggtatttcttctgccaagacccactgaccccatgcggctcacagtgttttctaaggctttgcaggactggtgttacgaattggcaccctccaggcctctcacaaatctcctgcttctcacagcgtttcttcaagttctcccaagcacagctgagttttgagctcaactgctccctgcaggggccttgagcctcctgcctttttgcataaaaggtgtcaggtacttatgcaatccttagaggcatgcaaatgctgctctggttatatactgaggactgttgattctggcagaaccctttgcagaccttgtactcccttgctatttcccaatccctgcagcctagcagctctgcctaacaactgccatagccaacacagcagcaggctgtgcatggtgcaaggtgatgtggaaagggatgattgtatgaaagcgtgatgctgtggtactgcctctgcaggagactcgcactatttgtgtaagaggaccttatttgtctgctgcagagctgtttcaaggctgtccatacacccctgtgatgctgagcccctccaagcaatgcactgggaaaaggaggctggggggagaccttattgctctcctccaatatttgaaaggtgcttacagcgagagcagggttggtctcttctcactggtgacaggatgaggggaaatggcctcaagttgcaccagggtatgtttagattggatatcaggaaacacttatttactaaaaggttgttaagcactggaatcagctccccagggaggtggttgagtcaccatccctggatgtgtttaaaaactgtttggatatggtgctcagggacatgatttagcggagggttgttagttagggtagtgtggttaggttgtggttcactcgatggtctttaaggtcttttccaacctgagcaattctatgatatggatccctggggctttcagtcttatctccctggattatcacaggttcagctctatggcccatttgatttataccggggtctgatgaacaggtttttctcttggctcttcagggatcctatttagcactttttggtacattcccctgccctacaagtctccctgatacacagagctcttatccaagacttgggaccttccctactccagccctctgcaggaggtttcttgctaaccagtcctccaaccaggactgcagtacacgacaaagagctggaagaggtctgcaatacttccccagcatgaaggtatgagcactccttttgagtaggttactgaaagtagtaagatgtcaatacaaccaactgcaagatacaaaaccgcatgaaaattcagtttactttgatgctgaagggctgaaaagaaatgctgtggtgttagcacagatgcactgctggcaaagtgaaaatgagcaaagaggatgagatggatggacagctgatggaaaaactcttcctaattgctccacagagcagcttgctcgcctgcagggctgcagcatggagctgcttgtgcataatgcagacaccccaagaccagtgctgtttgtcttagccaagacacagttgcagctgcagcaattttttctagatgtcagttccttccctatgttgctgacaggtgtttgctgttctgtccctttaatctgtatcctacagcaaacattccttgaatttaataacttagctggaagacaattgctgtgatcttgatagaacatgctgagccaatctattttaactgcagatttagtttgcaaatactgtctccttgccgataagattcaggtgtcatctttgtggacattggcaggaattttcttgaccgtgacaggttttacagagtctggcaattaagctgtcaagacacattttcctctgccaggaagcattaattgatgatagtcttggctgcaataggcacagagagatggatattgtaatcagaatgaatagaggtccttgtagttgagagctacgttggtccaaagttttgtagtcgttgacgtttggtgatactgagataaggaacaaggcacgagatattagagctaaatatcaggcacagcatgagaataaagacctctctagctggaactgttggtatctggggagattttaactttctggatgcatactgcaaagtactaatattagtagagctactggatgcgagagcaaatagttttccattaagtaatcccaaaaatcatgttgttgttggtttgcttttcaagtgcgaggggtgttggagatgtatttccctcagaaaataaacctgatatgattcaacctgagctctctctgtttaaatcacactgaaaatagatctgcaaatggggattttgattaccgagtacagaatatgaaagattaaaacttgggaaagttagggttctgattgagaaaacttttgtttttgtggccgacccttgcagcttacaaaaatctgcctaaataaaggagaaaaccacatttagaacccatccaagctatgctacttcagtactgggcaaaacttcaggagacgtttgaagaaaactgaagacgtgaagtataaaggaatgattgatgtgcacagtaaactttcttggaaggtaatcacgcatgggctaatatcaatctttacaaagttggctgacttcctagataaaggaagtacagtagatctagtctacccaggcagcaaaaatgtttgacctgttgccctgtggggtggtgtcacctgggcttggggaggggggtcaggatgaggttacaggggatgtggaagcatactgtggaggagcaggtggggcacccacaggagttagcagtgagcagacagaaaggtggatctgaggaccgaacttcgtatttttgttccttgcattaatacacaaaaagcagacacacacacagagcagattgctgctggtttttgttttcttttttaaacagcagaagagcaggatttttcccacagagaatggggtgaccttctaggctgtgattgcctgggctcaagctgagatgaaacgcagtgatgaggagcacaaaaccgtgctctgaggttaaataatgagggcttcggctatcagttcagagctcagtaaaaactgcagaggaggaggaagacctaattgcatgtagccagccacagggcaaatgagagctgcagcgtgctggggcagatccgggagcagaggggccgtggcacgctccctgttcactggctcccctggagccacacaaaaggccccttcctggcaattgtgcccacatcaatcattagctagaaacccagagctgggtaaatacgttttggcttcccgtcttgatgacagattgggtgttacatcacaaggtgggaccacttgatatgacaacacgctatatattcccgctgctacctctgcccttcctcccccactctgagagcaagcgggctgtgtgtgcaccgaggtgctctgccatgaggactgccaggcagtttgtacaggtggctctggccctctgctgctttgcaggtgagtgtttcctgctataccccgtaggtgactatagctagaccagagactaggctatctgtgagagtatctgggtattgtaatgtgttagagagccttgttccatgaaggaatgctctttctgacagtgtagcaaaacaccagactgcaagatccaggtttcagcaaacctcatacagacgactgttttcgtcgtggtttataggagcaaattgctgagggagcagtgctagtgcagggcaggagcttgcacgtgcaagcactgagtataacggcaaagcaaagctatgtgaaatggctcctgtgtccatgtaagcaatacaaacactgcatcttgtatcatctataaattttctgtgctgttcctggcagctgagaagtttgttgtgggaagaacagtgctagtggtcaacagccacctgaaacgtgcatgtctgagctcctgcaagtcaaatacagagtcttgcagaagagtttaaactcagtgcaggcttgaaaatacctacatttcttccctggggcatcttaggaactggctaacacatgtggcctcctactgaaagtgcagtgaaacttcatttaataacctctgattcattttatggacgtacatcactggcataatgtaaaattgcattttcctaaacccaataagccaatcaacaacggtatctaaatgtaactgtttcatcgaaagatttgcatatgtcatctctgcatattaataatatgtatttattttctgtctctacttttcttttagatattgcctttggaattgaggtgagttacagattttttttcccatttattcttttctattccaggcttctggtcaaataagagcagtatataattacctgatgagcaagtggattaatctaatgaaagcctggttgctcaaataatacttgccagtgcatgattgaatgatattgccaagtcacgaaaaagtaaaacacaccccgtttatactattttccattcatgcaataaaatgaagaaaggaagaattgtacgatcctattatgttaacttttggatataactgcgttagtccaagtcaaggggtggtagttacctcctcgagaggaaagctgtcttaagatgataagctccaaagcatcaaagacagtgattctggtatctttttctatacagtaagacacacactacagtgttcctgcctatacccatatcaaagcgaggaaagcagcagggtctgtgcagtgcatttgtctgcaggttcttcccacgcagttatgagattcctgcaaatcaccagagactgcagcgtgattggaaacgatcagattttgagttgagcggctgtggagcatggccaggctcccaattaccagctgccttcgttaggcgctgtctcacccacagctctccttcctccatgtcatgcttcccccagtcccccgcaggaaagcgtgatcagaagaagattcccacctcctgactgcctgagcagattccaaatgatacctcaggtgtttgtcccggctggagctgtgggtggcaggaggtttccatactgtcttttgttgtggaaactgaccccagggctgatgttgtgctgcttccataggttaattgcagcctgtatgccagcggcatcggcaaggatgggacgagttgggtagcctgcccgaggaacttgaagcctgtctgtggcacagatggctccacatacagcaatgagtgcgggatctgcctctacaacaggtgagcttatgtggaagcccaggggagctgcagggcaggagactcgaggtgagggcggcagctctgtccccaaaatatggtctgtgtggaggagtatgtgagttagtaccaggatgctgacctccagcctgggggtggtggctgctctctgccatctctgacacagatctgcgttcttccagggagcacggggcaaacgtggagaaggaatatgatggagagtgcaggccaaagcacgttacggtaagtccaacagtaagatgaagtcttgctctgttggtgcccataaagacttatttttatttcatagaatcattgaacagcttaggttggaagggaccttaaagatcattgggctctaacccccctggcctggccgggctgccttcaaccaaatcagtttgcccagtcaaatgggccttgggcacctccagggatggggcacctgctctgctcagcctgttacttatttacttgtttttttcccattcctgctatccttacagattgattgctctccgtacctccaagttgtaagagatggtaacaccatggtagcctgcccaaggattctgaaaccagtctgtggctcagatagcttcacttatgacaacgaatgtgggatttgcgcctacaacgcgtaagtcttttctgtggagcatccttctgggtaattagagatggctaagtcccttggaaacgcttacataaaacactttctaagcctttcttagggtagatgtttctgtgggactctttgaagctggctacttgtgattctccagccagctgcagatttcttccccatcctctgtctgtgctcatgaagggaatcacaaaaaagacagaggacaacccacagcagaggcatgaatagatcaaagtgttgctcagtgctgtgtgatatggaaataccatgcattttctgctcacaagtggttgctaccacctgtgggctgcatccagaccactcagcagttccttacgtgaagggtgggaccttgctttcttgccccagtatctaaggcttttcacgaggctctctaactaaaacagctctttctttcagagaacatcacaccaacatttccaaactgcacgatggagaatgcaagctggagatcggctcggtaagtgtaacagaaataaaaatccatctcctagggctgttaacggagagaatcccattgattttcctaagaaaatgtatgaccgggctgatcgggggtcccggtccacgctctgcttcctgcctggtgagggtggcttctgaaacaaagcggtaaaggaagaggccccagattttccttgcattgtgctgtgcagattggcaggtttctctctggaggcgacaagcatttccaccctttgtaacaagcattcaaaattctagtgctggtagcttggttagatatagtgagattcataagagcaccaagcatacatatttatagggtatagcttattgtatatttatactggggtaagagtccagtgcctcaggaagaaaagcttatatatttcagcacaaaaattctgggatgcagggagtccgttctccaacagacggattcctcctttatcacttcaactcccgtgcttaactgcagggaatctgaattattaagcaatcacagcactggggaaggaaggagaaaaaccaacacaaaccaaaacaatgttaatcagatttccagctgttggaaaatatttcccacttaattcaaggctgttgtgtcgatgagaagagggctgaaaaggctgttttcagttcctctgcctgaaggtttcattctctaagagaggtcccttttcttgtctcctagagaatgagggtagtgttctgaaagcctatttctgatagacagtttagttaagtgtagcagggctttgtcctgtcacaaaaactaggaagccgggaatacaggatgaaaaggtgttacattgacttctcccgtgtagcacaggctccgggagggcttattctccttattttggcaggttgactgcagtaagtacccatccacagtctctaaggatggcaggactttggtagcctgcccaaggatcctgagcccggtttgcggcaccgatggtttcacctatgacaacgaatgcgggatctgcgcccacaatgcgtaagtgctgctcatctcccactcctccaaagtagccagcaatgctttgccgtgctgggagccttccttctacgttgctgcttatgcctgtttcttcaagcctcttagaaactgcattttttttgttgttgttcttactgagttttcttctgatgccttctttgtgatcacgaggggaaatctgcaagactcagaacacagctccttggattagtctgtgggctgggcagtgactgagcagagaaaggaatagttcagaatcttgctttaaataacacgagaagacgtgatgagcttgttaacgagcagagtaatgtagctatatcaatacaatcgtgcagagaggctgaagccctactttgttaggtacctgctttaggctacgtctggttcattctgcatgcaagtgtttaaaccaagagttaaagcatctccttactcactttgtctccctctttcagagagcagaggacccatgtcagcaagaagcatgatggaaaatgcaggcaggagattcctgaagtgagtatacaacgtaaggtgtatttctccccttgcctctgcccactgagctatttgctgaggccacgtctactctgaaagtgagctggcttgaagcctggctctctgcacgtgtcctttgggatgtgccaacgtgtatccaacacacaaacagtgtggaagttgggcagggggaacttaggtcttttaaggatgatcactaaatgcattgccagcaaagtccttttgtgccagtgaagtcctattatgtttgccttcttttgtttcattctatagtgcagagagaaaaggagatgatatatctttgttggttttttttttgtttgtttgttttgcttttctgccatatctagcaaactgtttcagtaggttgtgacccctttggatcacaagtgaagctcagtggcatttgggattgactgagctgtctgccctggtgatttggcatctcacagattacacagcgccatgtagctcctcctgggcatgagagagtttctgcagagctgactcaggctggctttgagagaactgaagtgtagcaccagcgttgtttcagcatcccagcgtaaaagacatggattgcagcaggaggcaatgctagggtttgtctttgagagcaagggctttttcagggctgacgctcctactttttgcagattgactgtgatcaatacccaacaagaaaaaccactggtggcaaactcctggtgcgctgcccaaggattctgctcccagtctgtggcacagacggatttacttatgacaacgagtgtggcatttgtgcccataatgcgtaagtactgcaaacaggacttccttttgtagcgactagccacgttagtactgcagatggcttcccctccacccttcatcttcttctttctttctttttttttgatagcagtatgtctatatgtctcctgttcttccttcaacctcctgaagctctgtcgcctcggtttcctttcctgatgtgctcctcagggagctgtgggagagccagctaacagctgagtgtcctatgagggctgtggcatttgtgcagaggaaaaagagaatgggtctgctacaagtagacctgagaagcctgtaacttcttaggatcatgatccctaatggcagcctttccctttcagacaacatgggactgaggttaagaagagccacgatggaagatgcaaggagcggagcaccccggtaagtggggatggatgtcagatgagcgccagctcctgtacgtgccttgtggctgcagaggttgctaaccagggtctgtccattcaggcagcagagaaggggaatgggccaggatttaggtaacaaaatgtcccaatactgcaggtctctggagggaaacatcagaggcagcccagaacagcacagcctgttttagcacagtaggagaggaagagcagaagctgtgttagatgcctgtgtagtcattcagtgctaggatttccattgcagcagacaggttaaaaaatctctgtaccgtggtcagccaagaaaaggctgcttgcaggaatgcacgcagaaatagctctataaacatgcacggtaacaatatgtgctgataatatctcagcacatttattctgcttatgcagagcagctctaaaacactgaaaataactttgtgcatctcaagggattgctgtatcttttctgtagtaaagacacactgttatggtgctgtctttgctataatttgctcttggactgtgtggggaaatatgggtaataagagctactacacaggggaaggtatgcaaaacgattgtgaagtgtcagaagcttagccagtgtagactgacttccagtgccatcagtagatacttgcttatttatcctcaaatattggaactgtttttaagtactgtgaggatttctgcagcagcagctgatgagctgatggaacagtttcttcttgccgttttgaaaacgtggaaacaaaatctaaggcttagctaagtcaggcatgacctaatgtcaaactggacataacatcaaactccttatatcaaattcctttgaataatgcttgttttgaaacttggacatacgctgcataaggaagatgatctttctggtctgctattcctttgcgttccctttgttagtgagcaatatcaaacccaaccacaattagttcatttataatgggagactaaactgaaatcaaccctgatttttcctatggctcgaggcagtctgtcccccagctcccagcacctgactcagcatccttactgttttctccccagcttgactgcacccaatacctgagcaatacccaaaacggtgaagccattaccgcctgccccttcatcctgcaggaggtctgtggcactgacggcgtcacctacagcaacgactgttctctgtgtgcccacaacatgtaagccctgcaggtcacccactcgtgtgtcaccgcagctgcttgttgagctttgtcaactctgttttctctctcttccagtgaattgggaaccagcgttgccaaaaagcacgatgggaggtgcagagaggaggttcctgaggtaagcgataaagaaaacaagagcttgaggtggtgcttattgcctaacaagtacaacgctggctggttttggtgatgctgggtcatgccctcctgctgccatccttcctgcaggtaaacatcaaccctggcagcagggatgctgtgcattttctgcatgtagtcagggaaagaaagagaagaggacgggtgaggaatgagttatgatgcaggtagcataaatgatttaaggcgttacgaagaaatctctttcccacagcagtctatcatacctgccgtgggagtgtagctgtctgttctggcaatatgggaaagggacacagagcacccgcaggtacctggtgccttctggatacctgtgctgtgcaaaaggatgttgtgcaaagatcagaaaactacctgcattttgaatgcttttacctaatgtaccagaggattcaaacacctctctcttcctattgtaaatgcgatataatgtaatgtataccaacaatgaatcttgtaaaaataccagataaactatatttggccagctctaaactatttacgctcactggggaatagaaaaacaaagccatctcattatcttgtgtttgaaagagtcaacgtcgtgagtcagatatttcatttctatgcaaacagactatgaaatgtcattgctttgtttcctgcgtatgctctgtgctcagaccaagtcagatgcataaatcagtgaggaagagctcacactggagaaactgggatagctgaaactcaaggccagttcttcaaatggcataaatcattttgaactgctgttggtccttctgtccgattgcaacacacagaaccagcccctcgcaacaaaaggcatgtcagcacatctcctcagttcttgtgggccgtgacacactccttggccacactgagcttctcttgcaggaattgcataaatcacgccagtttgatttgcagattatttatgagctgcgttttgcagcgtcccagcaagtggttcagcaagctctaagggcatcgtgataaatgcagggctgaatgagtgatacgcgccttcaagctttgattcagtcttctccagtataaggctgtgacagaaaattgatagttttcaatgaagaatgagtcaatgcataaccataatccatcctgtggcagatcttgaaaggcagaggcgtaaggaagggggttgtgtctgagcacccttacacagagcatttgctgcctttgtttcctagcttgactgcagcaagtacaaaacctccacgctgaaggatggcagacaggtggtggcctgcaccatgatctacgatcccgtctgtgctaccaatggtgtcacctatgccagcgaatgcacgctgtgcgctcacaacctgtaagtactcattcatctccagggggacccaccgtggctgtgactggacacatctttgagtgctgaataacatgcaagggctctgtctaaaatctcgtgctgcatgggtcctgtctgcctatccccgtttccctggttgccatggttggtgtttgagatgggcatttagcaaggcccactgcccccagtgacccagaaaaagggttcactgcctgggaaagcattattccaaaagacacatccctagtccttaagggcatgttcttgctaatgcttctcaggcaatgcttagctaatttatctgaaattgtcctgtgtaccacatgggaacgaggttgtgctcttgtactacggttgtaaatgggaagggtttctgctaatatccatctctccttcctccagggagcagcggaccaatcttggcaagagaaagaatggaagatgtgaagaggatataacaaaggtgagtgtgaaaggatgggcacaaagagttacagtcgtaggggaccgtcctctgctccacatcaaaaactgggggagcggtgtgcagccctggcgaggtcgcttgggaatgtcatactggttatagaatagctgccatccatcccatgggaatggacatggcagtgaacaggaacagtgtgaggtcacatccctcaccaggaggaactgagctgattactgccgtaattttccagtttcactctttgtgctgggggaatactgtttgctcccaggcagagactcacatcttccttgtgtgtgcaggaacattgccgtgagttccagaaagtctctcccatctgcaccatggaatacgtaccccactgtggctctgatggcgtaacatacagcaacagatgtttcttctgcaacgcatatgtgtaagtataggagtgaaacccttcctgtaactgctacaaacgcagagttgattttataaggagttctttactaacactttatgggtgtgtgctagacatttcggatgcaccgtgacgtgcaaggaggtgcttttttgctttttaagaaaaaatgcaaagcacccacatctgcccatgtgtatgtggcttcctgttttatttagtttcaaagacattttgctaattttcaccagcatagtttgtcccacaagctcatcagggtatggggaaagtacttcaccaaactacctggagcgtttcaagtgtgtgaaacctgtcatctttcctttaattttcataatgaaaggaagtggttggccttctgagactgttctttatcttctgccaacattatcaacatttgggctggtaaggagaggaacaaggctgcagcacaaattctattgtgtttaatcctttcttctcttttcattaggcagagcaataggactctcaacctcgtgagtatggcagcgtgttaactctgcactggagtccatcgtgggaaacaatctgccttgcacatgagtcttcgtgggccaatattccccaacggttttccttcagcttgtcttgtctcccaagctctcaaaacacctttttggtgaataaactcacttggcaacgtttatctgtcttaccttagtgtcacgtttcatccctattcccctttctcctcctccgtgtggtacacagtggtgcacactggttcttctgttgatgttctgctctgacagccaatgtgggtaaagttcttcctgccatgtgtctgtgttgttttcacttcaaaaagggccctgggctccccttggagctctcaggcatttccttaatcatcacagtcacgctggcaggattagtctctcctaaaccttagaatgacctgaacgtgtgctccctctttgtagtcagtgcagggagacgtttgcctcaagatcagggtccatctcacccacagggcaattcccaagatgaggtggatggtttactctcacaaaaagttttcttacgttttgctagaaaggagagctcactgcctacctgtgaattcccctagtcctggttctgctgccaccgctgcctgtgcagcctgtcccatggagggggcagcaactgctgtcacaaaggtgatcccaccctgtctccactgaaatgacctcagtgccacgtgttgtataggatataaagtacgggaggggaatgcccggctcccttcagggttgcagggcagaagtgtctgtgtatagagtgtgtgtcttaatctattaatgcaacagaacaacttcagtcctggtgttttgtgggctggaattgcccatgtggtagggacaggcctgctaaatcactgcaatcgcctatgttctgaaggtatttgggaaagaaagggatttgggggattgcctgtgattggctttaattgaatggcaaatcacaggaaagcagttctgctcaacagttggttgtttcagccaattcttgcagccaaagagccgggtgcccagcgatataatagttgtcacttgtgtctgtatggatgacagggaggtagggtgacctgaggaccaccctccagcttctgccagcgtaggtacagtcaccacctccagctccacacgagtcccatcgtggtttaccaaagaaacacaattatttggaccagtttggaaagtcacccggtgtattgtgaggctagattaataggctgaaggcaaatgttcccaacttggagatactgttggtattgtatcagggaacagggccatagcacctccatgctattagattccggctggcatgtacttttcaagatgatttgtaactaacaatggcttattgtgcttgtcttaagtctgtgtcctaatgtaaatgttcctttggtttatataaccttcttgccgtttgctcttcaggtgttcttgcagaacactggctgctttaatctagtttaactgttgcttgattattcttagggataagatctgaataaactttttgtggctttggcagactttagcttgggcttagctcccacattagcttttgcagccttttctgtgaagctatcaagatcctactcagtgacattagctgggtgcaggtgtaccaaatcctgctctgtggaacacattgtctgatgataccgaaggcaaacgtgaactcaaagaggcacagagttaagaagaagtctgtgcaattcagaggaaaagccaaagtggccattagacacactttccatgcagtatttgccagtaggtttcatataaaactacaaaatggaataaaccactacaaatgggaaaaacctgatactggaatttaaatattcacccaggctcaaggggtgtttcatggagtaacatcactctataaaagtagggcagccaattattcacagacaaagcttttttttttttctgtgctgcagtgctgtttttcggctgatccagggttacttattgtgggtctgagagctgaatgatttctccttgtgtcatgttggtgaaggagatatggccagggggagatgagcatgttcgagaggaaacgttgcattttggtggcttgggagaaaggtagaacgatatcaggtctacagtgtcactaagggatctgaaggatggttttacagaacagttgacttggctgggtgcaggcttggctgtaaatggatggaaggatggacagatgggtggacagagatttctgtgcaggagatcatctcctgagctcggtgcttgacagactgcagatccatcccataaccttctccagcatgagagcgcggggagctttggtactgttcagtctgctgcttgttgcttcctgggtgcacagtggtgattttcttactcacacagggcaaaaacctgagcagcttcaaagtgaacaggttgctctcataggccattcagttgtcaagatgaggtttttggtttcttgttttgtaaggtgggaagaagcactgaaggatcggttgcgagggcaggggtttagcactgttcagagaagtcttattttaactcctctcatgaacaaaaagagatgcaggtgcagattctggcaaggatgcagtgaaggagaaagccctgaatttctgatatatgtgcaatgttgggcacctaacattccctgctgaagcacagcagctccagctccatgcagtactcacagctggtgcagccctcggctccagggtctgagcagtgctgggactcatgaggttccatgtctttcacactgataatggtccaatttctggaatgggtgcccatccttggaggtccccaaggccaggctggctgcgtctccgagcagcccgatctggtggtgagtagccagcccatggcaggagttagagcctgatggtctttaaggtcccttccaacctaagccatcctacgattctaggaatcatgacttgtgagtgtgtattgcagaggcaatattttaaagttataaatgttttctccccttccttgtttgtcaaagttatcttgatcgccttatcaatgcttttggagtctccagtcatttttcttacaacaaaaagaggaggaagaatgaagagaatcatttaatttcttgattgaatagtaggattcagaaagctgtacgtaatgccgtctctttgtatcgagctgtaaggtttctcatcatttatcagcgtggtacatatcagcacttttccatctgatgtggaaaaaaaaatccttatcatctacagtctctgtacctaaacatcgctcagactctttaccaaaaaagctataggttttaaaactacatctgctgataatttgccttgttttagctcttcttccatatgctgcgtttgtgagaggtgcgtggatgggcctaaactctcagttgctgagcttgatgggtgcttaagaatgaagcactcactgctgaaactgttttcatttcacaggaatgttttagtggcattgtttttataactacatattcctcagataaatgaaatccagaaataattatgcaaactcactgcatccgttgcacaggtctttatctgctagcaaaggaaataatttggggatggcaaaaacattccttcagacatctatatttaaaggaatataatcctggtacccacccacttcatccctcattatgttcacactcagagatactcattctcttgttgttatcatttgatagcgttttctttggttctttgccacgctctgggctatggctgcacgctctgcactgatcagcaagtagatgcgagggaagcagcagtgagaggggctgccctcagctggcacccagccgctcagcctaggaggggaccttgcctttccaccagctgaggtgcagccctacaagcttacacgtgctgcgagcaggtgagcaaagggagtcctcatggtgtgtttcttgctgcccggaagcaaaactttactttcattcattccccttgaagaatgaggaatgtttggaaacggactgctttacgttcaatttctctcttccctttaaggctcagccaggggccattgctgaggacggcatcggggccccctggaccaaatctgtggcacagatggtttcacttacatcagtggatgtgggatctgcgcctgtaatgtgtccttctgaaggaaggaacgtgccttccaagtgccagccccacagcccccagcccctccctgtgctgctccaattcatctcctcttcctccttctccctttgctgtttgtgctcgggtagaaatcatgaagatttagaagagaaaacaaaataactggagtggaaacccaggtgatgcagttcattcagctgtcataggtttgtcattgctataggtctgtatcagagatgctaacaccactttgctgtcggtgcttaactcgggtgaactctccttcactcgcatcatttgcgggccttatttacatccccagcatccatcaccctctgggaaaatgggcacactggatctctaatggaagactttccctctttcagagcctgtgggatgtgcagtgacaagaaacgtggaggggctgagcagcagcactgcccccagggagcaggagcggatgccatcggtggcagcatcccaaatgatgtcagcggatgctgagcaggcagcggacgaacagacagaagcgatgcgtacaccttctgttgacatggcatttggcagcgatttaacactcgcttcctagtcctgctattctccacaggctgcattcaaatgaacgaagggaagggaggcaaaaagatgcaaaatccgagacaagcagcagaaatatttcttcgctacggaagcgtgcgcaaacaaccttctccaacagcaccagaagagcacagcgtaacctttttcaagaccagaaaaggaaattcacaaagcctctgtggataccagcgcgttcagctctcctgatagcagatttcttgtcaggttgcaaatggggtatggtgccaggaggtgcagggaccatatgatcatatacagcacagcagtcattgtgcatgtattaatatatattgagtagcagtgttactttgccaaagcaatagttcagagatgagtcctgctgcatacctctatcttaaaactaacttataaatagtaaaaccttctcagttcagccacgtgctcctctctgtcagcaccaatggtgcttcgcctgcacccagctgcaaggaatcagcccgtgatctcattaacactcagctctgcaggataaattagattgttccactctcttttgttgttaattacgacggaacaattgttcagtgctgatggtcctaattgtcagctacagaaaacgtctccatgcagttccttctgctccagcaaactgtccaggctatagcaccgtgatgcatgctacctctcactccatccttcttctctttcccaccagggagagctgtgtgttttcactctcagccgctctgaacaataccaaactgctacgcactgcctccctcggaaagagaatccccttgttgcttttttatttacaggatccttcttaaaaagcagaccatcattcactgcaaacccagagcttcctgcctctccttccacaaccgaaaacagccggcttcatttgtcttttttaaatgctgttttccaggtgaattttggccagcgtgttggctgagatccaggagcacgtgtcagctttctgctctcattgctcctgttctgcattgcctctttctggggcttccaagagggggggagactttgcacggggatgagataatgccccttttcttagggtggctgctgggcagcagagtggctctgggtcactgtggcaccaatgggaggcaccagtgggggtgtgttttgtgcagggaggaagcattcacagaatggggctgatcctgaagcttgcagtccaaggctttgtctgtgtacccagtgaaatccttcctctgttacataaagcccagataggactcagaaatgtagtcattccagcccccctcttcctcagatctggagcagcacttgtttgcagccagtcctccccaaaatgcacagacctcgccgagtggagggagatgtaaacagcgaaggttaattacctccttgtcaaaaacactttgtggtccatagatgtttctgtcaatcttacaaaacagaaccgagggcagcgagcactgaaggcgtgttcccatgctgagttaatgagacttggcagctcgctgtgcagagatgatccctgtgcttcatgggaggctgtaacctgtctccccatcgccttcacaccgcagtgctgtcctggacacctcaccctccataagctgtaggatgcagctgcccagggatcaagagacttttcctaaggctcttaggactcatctttgccgctcagtagcgtgcagcaattactcatcccaactatactgaatgggtttctgccagctctgcttgtttgtcaataagcattttttcattttgcctctaagtttctctcagcagcaccgctttgggtgacttcagtggccgcctggaacccgaggggcacagccaccacctccctgttgctgctgctccggggactcacgtgctgctggatggggggaagcatgaagttcctcacccagacacctgggttgcaatggttgcagtgtgctcttcttggtatgcagattgtttctagccattacttgtagaaatgtgctgtggaagccctttgtatctctttctgtggcccttcagcaaaagctgtgggaaagctctgaggctgctttcttgggtcgtggaggaattgtatgttccttctttaacaaaaattatccttaggagagagcactgtgcaagcattgtgcacataaaacaattcaggttgaaagggctctctggaggtttccagcctgactactgctcgaagcaaggccaggttcaaagatggctcaggatgctgtgtgccttcctgattatctgtgccaccaatggaggagattcacagccactctgcttcccgtgccactcatggagaggaatattcccttatattcagatagaatgtcatcctttagctcagccttccctataaccccatgagggagctgcagatccccatactctcctcttctctggggtgaaggccgtgtcctccagccccccttcccaccctgtgccctgagcagcccgctggcctctgctggatgtgtgcccatatgtcaatgcctgtccttgcagtccagcctggaacatttaattcatcaccagggtaatgtggaactgtgtcatcttcccctgcagggtacaaagttctgcacggggtcctttcggttcaggaaaaccttcgctggtgctacctgaatcaagctctatttaataagttcataagcacatggatgtgttttcctagagatacgttttaatggtatcagtgatttttatttgctttgttgcttacttcaaacagtgcctttgggcaggaggtgagggacgggtctgccgttggctctgcagtgatttctccaggcgtgtggctcaggtcagatagtggtcactctgtggccagaagaaggacaaagatggaaattgcagattgagtcatgttaagcaggcatcttggagtgatttgaggcagtttcatgaaagagctacgaccacttattgttgttttccccttttacaacagaagttttcatcaaaataacgtggcaaagcccaggaatgtttgggaaaagtgtagttaaatgttttgtaattcatttgtcggagtgttaccagctaagaaaaaagtcctacctttggtatggtagtcctgcagagaatacgacatcaatattagtttggaaaaaaacaccaccaccaccagaaactgtaatggaaaatgtaaaccaagaaattccttgggtaagagagaaaggatgtcgtatactggccaagtcctgcccagctgtcagcctgctgaccctctgcagctcaggaccatgaaacgtggcactgtaagacgtgtccctgcctttgcttgctcacagatctctgccctcgtgctgactcctgcacacaagagcatttccctgtagccaaacagcgattagccataagctgcacctgactttgaggattaagagtttgcaattaagtggattgcagcaggagatcagtggcagggttgcagatgaaatcctttctaggggtagctaagggctgagcaacctgtcctacagcacaagccaaaccagccaagggttttcctgtgctgttcacagaggcagggccagctggagctggaggaggttgtgctgggactcttctccctgtgctgagaatggagtgatttctgggtgctgttcctgtggcttgcactgagcagctcaagggagatcggtgctcctcatgcagtgccaaaactcgtgtttgatgcagaaagatggatgtgcacctccctcctgctaatgcagccgtgagcttatgaaggcaatgagccctcagtgcagcaggagctgtagtgcactcctgtaggtgctagggaaaatctctggttcccagggatgcattcataaggacaatatatcttgaggctgtgccaaatctttctgaaatattcatgcatgttcccttaatttatagaaacaaacacagcagaataattattccaatgcctcccctcgaaggaaacccatatttccatgtagaaatgtaacctatatacacacagccatgctgcatccttcagaacatgccagtgctcatctcccatggcaaaatactacaggtattctcactatgttggacctgtgaaaggaaccatggtaagaaactcaggttaaaggtatggctgcaaaactactcataccaaaacagcagagctccagacctcctcttaggaaagagccacttggagagggatggtgtgaaggctggaggtgagagacagagcctgtcccagttttcctgtctctattttctgaaatgtctgcaggaggaaaggacaactgtactttcaggcatagctggtgccctcacgtaaataagttccccgaacttctgtgtcatttgttcttaagatgctttggcagaacactttgagtcaattcgcttaactgtgactaggtctgtaaataagtgctccctgctgataaggttcaagtgacatttttagtggtatttgacagcatttaccttgctttcaagtcttctaccaagctcttctatacttaagcagtgaaaccgccaagaaacccttccttttatcaagctagtgctaaataccattaacttcataggttagatacggtgctgccagcttcacctggcagtggttggtcagttctgctggtgacaaagcctccctggcctgtgcttttacctagaggtgaatatccaagaatgcagaactgcatggaaagcagagctgcaggcacgatggtgctgagccttagctgcttcctgctgggagatgtggatgcagagacgaatgaaggacctgtcccttactcccctcagcgttctgtgctatttagggttctaccagagtccttaagaggtttttttttttttttggtccaaaagtctgtttgtttggttttgaccactgagagcatgtgacacttgtctcaagctattaaccaagtgtccagccaaaatcaattgcctgggagacgcagaccattacctggaggtcaggacctcaataaatattaccagcctcattgtgccgctgacagattcagctggctgctctgtgttccagtccaacagttcggacgccacgtttgtatatatttgcaggcagcctcggggggaccatctcaggagcagagcaccggcagccgcctgcagagccgggcagtacctcaccatggccatggcaggcgtcttcgtgctgttctctttcgtgctttgtggcttcctcccaggtgagtaactcccagagtgctgcagaagctttgtgcctgccagtcctggctctccttagcagaacatggtggtgaccatcagagagagactcccctacaaagtgcctgcaaaggctgcctcagtacatcagtattaaacggattactgttgtgctgggtgtctgttgggttctgtgctcccaacacatttcttacgctctcagctctgttacactgcttgcatttgctgcacagttgcatagaatggataaatgcttgaaacaaggccataacgaggtggtcagacctccaggaactagttagggaaatattgtcatggcccaagcaagctctgtgcaggaacctggcagctttcctgcaatgcttttgctgctaatggagaaacaagagatgcaaacaagccaggatctgatgttctccttctgtatttacatctcatgaaattacaaagtcaaagacaagcgtggtttatttcttacactcagcttctttaaaatgtatatccctgacaacagatgctgtgtatgtttgcttatcctgtatgtgactatttgcatttgcatttatctctattgactcaggtttcttttcagatatgtgatagatgttttctagggacaaaacggatgtgtgaatagataaggaaggaaaagatattcatttttcaattaataaatctacctatctcttaacttttttttttttttaagaacagagctattcaagaactcgtttcatcagccagcaataagaagctaaattatgtttatcagcattaaacaaaaatcatatatagtttgcttagttcaagaatcgaatcggtggaaatcactcagtttggttctctgtgctggagttttgcacacacatttcagctagctgtggtctcactgatcagactgcctttgtttcccatttttgtcccctttttttccccagatgctgcctttggggctgaggtgagtaagagagttcttcttgtccacttttctcttttctcttttctctctctctctttttttccccccgtcttaattagtatcactataatcagatcccagagtgtaaaatgttaaattatgcagttctgagctctacatctatgctgcatgtaagtaatgtagcagtgatataaaactgttagatgaattaatttctgaccaactctgaactggtctaagctttaagttgatcatatgttctactaaataatacagtggtttgggttggaagggtcctttaagatcatctacttccaacccctctgctataggcagggacaactcccactagacaagattgctcaaagctccatccatatgatcagctgtagactgatggctgtagactatagcattaaaaactaccccaaagcagcctactgaaagaagaaagtactgtgaggtgctacagcttccaaatcccatgttgttagacctgttcttttgaataaacgtgtttgtacgttgagaatgaatgagtaacaatggcagaacactggaggggccaactctcaggctttgcaaaatggtgcctggggggcatgatagatccctgctggtttatcacatggggagctgcatggctataaccccattgcccagttctctcccactgcatggagagaaggctggatctggtcgctgccctgctgaaaatggcagatgtaactacaaaatgtcactttgtcctgttactgtgtgtttctttgtcaggtggactgcagtaggtttcccaacgctacagacaaggaaggcaaagatgtattggtttgcaacaaggacctccgccccatctgtggtaccgatggagtcacttacaccaacgattgcttgctgtgtgcctacagcatgtgtgtactgcagagagagctcatactgcaagcaagcagctgtgcttagggctcctgacagcacccctttccaacaaacagtgatctgtcacatgtcacttatgtcaactctttcagggaaagcttgagtatcactgcgtgacactcggttgcctagacatcactttggttactgtgtcttttttgttgatgtaatttattcaggtttttctcctccatctcggggatgaggcagatgacagcccctagggcatatttcatcccagcaaaaaaggagcaaaaggatggagaggtgctccagtctgaatggtccaaaacagtcctaaagatttcagagtctttagatccctgccagccactcagtatggcactaccctctccaatacaaatatatatatatacaaagatgacttagccagactcagcctcattgcattaggtacatattcccaataacgagaagctgagcttcctaatacctgttttccctcttcagagaatttggaaccaatatcagcaaagagcacgatggagaatgcaaggaaactgttcctgtaagtgaaaccaagttcatcctttgtgcagccaaaactgcttattgacttgcccaataaataatgtaaatgctgactaagaggccatgtgagatgtcagaatcttgtattgatcatcttcaggtgaagtttcatcacaataacacaaaaaaagactttatttcctgctgaggtggcattttaggagacccaacgcacgcgctccgctggtctacgtggtccctgtaagccctcaccagcgctttgctgtgtgctccttccacagatgaactgcagtagttatgccaacacgacaagcgaggacggaaaagtgatggtcctctgcaacagggccttcaaccccgtctgtggtactgatggagtcacctacgacaatgagtgtctgctgtgtgcccacaaagtgtaagtaccgagctgtgctcccttggcaggaatgggtcctgcgctcctggcagccactctttgagcactgggatttccaatgaggctttttctgtatggctcttggactccgtccctcctctccctgataacctcatgctgttttcctttgtgattagaaagagaactgtggctttgatcttgagagagaagcagagagctgggtggggacttaagagaagcactctgttctgtgttaactaagttaaaagggtctgtgtggcacacactgccttgcagaggacagcagtgaacctctgctgcacctatattgtaaaacaacctagctcctaggccatgacagcctgtcacctctcctcctttgcatcatgcaatactgcaacactgtggcacatagtaccacctcccataaggactgatatgttgaaccagtgtgtcagagaccagtagcatctctgtcttcaggatcatcaggtagcattctatatacagggtgttgcccaggactccgagtcccatgaagtatggcaggggttttggaactggatgaccttcgaggtcacttccaacccaagccattctattattctgtgaaagccagggaggtgggggtgcttgcagggctggtatcttgagcagtgtgggcacaaactaggctgggcatctgcagcccatcagcactgcggggatgtggagttcagcacagcaggatgcaggcacagctccctaacatggatttttttcctttcagagagcagggggccagcgttgacaagaggcatgatggtggatgtaggaaggaacttgctgctgtgagtgtgagtagcacaatgaaggagcaggttctggtcccactgatgtcaagggaaacatggccagcatctttagtagcctcaggagcatcagttgtgcttcagcacagagaagattttactttctacacacgtaatacacattatccacagtaatgtcaggaagggaagaggatgactgcacaggcagggatcagtaaaagaccataagcagaaataacccatgagggcagaactgagaataagaactgagactagatccagggggtcagaccaatgggccatcaaacccatgatggtttgatgcagagtccactctttcagcattcataagaattgagtaggggggagtaagggtggggtgagtacgtacggatcttcccaaacacccttccaacctacagctatgcacctcagccaggtgtgatttctgtgtagttcacaagcctcagtggatttctctcccatgggattctccagcctctttctggacctgtatacacggtagttgggttggttttttttttctgtctctctttttttccccccactacaatgtccctcagcaaacatagtcctcatctctcaaacaaacaaatctcattctctaagtacccagataagagctgatttttgctttaagcctgtgggggagatgctggactattataaaggtatcagtgctgcctcttctccagacaccaatgttttttccatttaatttcctgaacaggtcaggaacacggtgcaacatgattgtaagcacagcacgttcatggagcgagctgctgctgcagctcagaaatgcagcagtcagattgtgatatgcatctcttacacaggaaattatgctctatttttatattattaaatctagcatacgagaaaggacatccagtttatatcagatcgtgcaaggaagttaattatttttagtttgatcattatcatcggcactgcagctgtagctagggaggggttgaagctcttcagctatcgactccttcatatcctccacgttacaattgtgtttttgcaggttgactgcagcgagtaccctaagcctgactgcacggcagaagacagacctctctgtggctccgacaacaaaacatatggcaacaagtgcaacttctgcaatgcagtcgtgtacgtacagccctgattgcattcacgttgtcggctgcctcctacaggcaccagcttgcacagttcctgctttcgttgctgattgctgaccaggatctgggggcagaaaagaacaccgggcatcacgccagccattcatttgatttttcaccagagcttgtctggtttgttaggatggatgttttgaacgccattaaccttaagggaagttttccttgctgcgaagaaaatcagatttggtgtttcattatagttttcagaaggggttaaacgatttcactcatctcctaataatcaggtagctgaggagatgctgagtctgccagttcttgggctctgggcaggatcccatctcctgccttctctaggacagagctcagcaggcagggctctgtggctctgtgtctaacccacttcttcctctcctcgctttcagggaaagcaacgggactctcactttaagccattttggaaaatgctgaatatcagagctgagagaattcaccacaggatccccactggcgaatcccagcgagaggtctcacctcggttcatctcgcactctggggagctcagctcactcccgattttctttctcaataaactaaatcagcaacactcctttgtcttgtttaatgctctgcctcatgcaatgttttcttctgatttgttggacggtgataccagactcaatatgttccatgctcgtggctctggggtataacaagaacaacatcttgctcccatccctgtcataaaaggcagaaaattaaatacagatgcataaacctcggctgtgtgactttgcgcataaatgacagtcagcctccattagtgttcagacccttttagacagctgaaatactgctacgaactgctgatgctggctgagctccccatggtacgtgtggtgcactttccctgcgcagcattagcagtgaaagcagctcagggtgcggtggtggccaaacccagggccgatcccacggcctcctgtacctggtcatacccacgggcacagctgctagtgaggtgcgtgcttttcagacacgtcatataagtgtgccctgcctacatgtctgggtcctccaaatgacgttgcaaggtttatctcatcttggaattgtcccttactgaccaccaagtgttttgagatgaatgccctcctaggtctggttctgctcttgcctgctggtcttttctcatagtagtccttgccagcccaagtatctgagcagtgttttgcaatccaaggacaaagtacccctctgcctttgagagtgtgacctctgtcattggcacattgtccgtgaaatatattttgcttttgtcctttgttggtgtattgaactgatgttttcttgatccacatgagagaaactttaataaaaattataaaaaataatgcctcccttaagcatttcttttccctgatggaatgaggccattcaaaagaaggatgctttggcggtaaaacagaggatttatgttgagatgggcagatgaatcaagcagtgatttccagtttggattgaacttttctgggatccaggctgtgggcctcatgtcattctgtcatcatcaggctatcagtctgctgctgcaaatcctccccacaacgctaatggcttttagggaaaatcgcaattgttagttctttgctaatgcccataaaacttcttccatcacttgtccagctccaggactcccttcagccccaggtttccctcttgctctctctcccagttcagtttttctggatttgctatgatttgatgatgcattattgacaggacaaggggaaatggtttcaaaccagaggagaggagatttagactggacataagcaagacattttttacaatggtggtgaggcactgacagaggttgcccagagaggtggtggtgccccatccatggagacagccaaggtcaggaggggctctgagcactgatggagctgtgggtgcccctgttcattgcagggggttggaccagatggcctttaaagatcccttccaactcaaatgcttcaatgattctgtgattctattgggttgaagcatgccaactaagactttccactctggaaaacattcaattcagttcaacaacattttccagcaacagtgagaaagcactgcatataggtaagcactgataacatgcacatggaggaaatcctgcagcattctctcttcaggtttgtacagttgcccttttgcccacaggaattttccatggtccttcagcaggcacctgtcacacacttcactggaaataatgaagccgagggcgtacttcacatatttaaacctgcaattgctgttgataaagaagcattctttgtggctcacttgtgtaagtgccatcaagatttacaaccctgacaccagagctggaacgctggttatttcaaagtagggggtggctaaaccaaacgtgaatgcacacagccacgcacacacagatcaggtggccatccaagggcagaagggccgcattccatgagcacgatgcacttctgccctttgctgctgcccaggtgagtggctgtgctcctgctccgtgcttcgtcgagtgctggctgtaaaaacacaacaaacatcctcagactggaaagagctgtgttctacaaggacttatttactcctagagggatggtgttgaaaagacttgacatcaaagactatcacttatggggtaatattttagcaacagaactgagtgggtaagaacaactgtgggaacagctccgcgctcggtgctagtttatgcataatgaaagcagtgacacgtacgtggtaccacgacatccaccattgaacctccgaaacgctgcagaatcacaaattcttttactgaatggaagcgagcgtttcccgcagtcatcctgaactgagatgcaattggaggggctgagcggctgcagcagcgttaggggagtttcacctcgctgagccctcccgttatttcagtgctgttgtggagctgcacgcaggagctgccgccagtccgtgccagctctgcggccctgcttccccggcaccttgcttatctctgagcacctgtccttgctcatcctgtgaatcacggagaattgctttctcttcctccctttcatttcgcgcgtccttctccacccgggctgtaaccctcctgagaaaaaacgtagtacggaatcgatgttgtaaacactcagcgtggcacaacgttttgcctgaaatcccttttgtctgagagtcacacactgaattgcaagttgtttattcaggacatgcactcacggattttaacactaacgaaggagatgaattgcatttgtgtcacacttcctattcccttctttactccagaccccactgcactgaaggtaagggacagatctttcaggtttttttttttttttctccatcatttctttcctcaaagcagtttccgtataaatcattactaatcgcattgtgatcgagcgtttgaaagccctgagtcatcccacagcctgagcaatatttgctacagatattaccgagtgaaatggccattttcatctgatggtttcaaaaaaaaaaaaaagataataataataataataataataaataaatagcgcagcattcagttggtgtccaagttattgtcacggttactgcagcagcactgaggatgtttacatgggatttacatcactggaggctgaaagggcactgcaggcgtgtaccgcgctattcgctgccccatccttaagctcttctttgacatctgctgatggtcggtgctgggggaagcccggggctgtgggggtctcctggcatctgccctgctgatagctgtgctgctgagggtatttctgtgagcacaaggctgcatcgatccacagggcgactgcagtgcctgcgccgtaccccgcaatttctgctctcgggagcgcatcccacactgcgggtctgatggcgtaacatatgccagcgagtgtttattccgcaatgcatttctgggtgtatgaaaataaatctcttcgctcactgagtggtgaacttcaactgtcttatcaacctcagggactgcctggagatggaaggtggttgtgtttggcgctctcctcttctcttgctagcaagggcagcacttttttttttaaactgggaggatttaccagggactcctttctttcaggtaaaaagaagtcacatttagcagagatcttcatctccacgttgggtaatttgctgaagagctcgcttccagcaaatacagtctatttcctacagcctatttgttcttcttttaaattaagtctttatcgtgcctttgaatgttagtaataagaggaagtagctggaatagctttccgaatgttctgttttggttaagttcctctgtgatgtatccttaagcagagggagggatgcacagcagaagcgcagaggttcaatctctgaggccctgagctctttctctccagaactcattgagttctcaccttgctgtgccctgcgcagcgctcacatcacagcccaccgggctccagctcagacaggaggaccctctctggctgtgttccttacaggggatgctgcccaaagcctcgtcctgaactttgagtgctcctgataaagcctgaagctatgctcaataaaaaaaaaaaaccttcagcattttggtcttgctttcatactacgtatcatgctgttgtttttttttcttaagatgctgtgtgattgcatcactgcaacagtcctggggtgtgggtcttaatgggaaaattacagggagaaagaacgggttgtctgatttatgaagaaatcaacccctccaaaaggccatgagcttctgctttcttccagatttccaaaagaaagccactgctggggatgagatccagtgcagtgttcagggcatcctgtgcagacattgactccttaggagctgaaaataaagtagtggtgggtacccgtaggtgtgggaagcctttctgcagccacctggtctgcctcccaaagcagaggatgggatgttttcccctccgggcagcaccaacagaggggtggcagcagggtgaggaagatgattggcccctctgctctgctcttgtggggaccacatgcagtattgcatccaggcctggggccccagcatgagaaagacgtggaactgttggagtgggtccataggaggccatgaagacaatcacagggctggagcacctctcttatgaagaaaggctgagggagctgggcttgttcagcatcaagaagggaaagctgagaggacacctcattggagtcttccagtacttgaagggagcttgcaagcaggaaggggaacaaacttctacatggtctgacagagatagaacaagggggagtggctttaagctaaaagagggaagatttgggtgagatgttgggaagaaatactttactcagaggttggtgtgacactggcactgctgcccagagctgtgggtgccccatccctgtacatgagctgaaggccagattggatggggctctgtgcagcctgatctggtggggggcagccagcccatggcaggggttggggtagatgggttgtatggcccttttcaacccaaaccattcaatgattctatgattctcagataagcctgcctgcccacatctgagctcacggtgctcgctgggggtggggtatggtacactaaatgatgctcagaggactgcacgcaggacctgccgcagacgtttatcacctcacccaccacttagctgctgcttgtagttaattacgtcagctgtcacttgtagagaatcctttgagatccttgggcctccggaaatcttggctgatgaaaggaagggctcagagtcatagcgttaatttattattcattaacaccaaagtgtcggctgtacgggcagtgggctcacagtcaaatagttaatgatcttaagtgacaatgtgtcactttgcagacagcagagagaacagctctcctaagggagacagcatctttccaattctgcagccattcagtgccaagctcctctttgggacgaaagtgaagatgaggaaggcaatgaggatgaggaggggcctcaaggaacctggctggcttggagacaagtgatgatcccagctgctctcagggtcccagcggtcttcaaagggcatcttgcaggggctgtgtcctctgaacagcaaaacccaggtcatagaggggaaagtgtgagcagagatgggacaaatctcccatcctgccacggagctgcactgctaagggggtgatggggagcagcatgggaccccagcgttccccccatccctgcaccaggcccagctctgcgggatggcgaggaggacaaggctctgtcacaagcatcgctggcaattattattttgttgttgctgctcaataaaatcctgacacagtacaacacaatatcctctcatcattactaatctaactctccctccaggaaatttcaggcaggaaacgttgtctgcctgccgaggtgctttatggcactgttctttagtggtacctcagcacttcgtgtcattatctggtgtcagtgaatttaggaaatgccattcaattaccccgcaaactgattaacgcattgcgtgcagttattttgttctgctctattttatatcagttcctctgttttatgtatttctctacttgttgctggccagaacacacctcgggccagtctagaccttgctgttgatgcagcttttccccagggcttcatcagcacaaatggtttgtcaacgtggggaaaaataaaattatgctttaaaataaaaccacctggagatgctgttctggggtctggctgtgtcacagctattgcagcgatggagctgagggattgggatgtgctgggccggatcctcagcgctttgctataagccaaataattccagacacccttcttccctcagatatcatctgtgcttaagcagcaggagatatgcaggcagcgatcagatagctgagctgcaaggagaaatatcacaagagcgcggcttagagcaggggctttgctcgctctaaattgaattcccatcctcataggagatccagtcctgcccccgtgtgcatcgctccggtaacagcaatgtgttttgctccatcttgcagagggtccagaagctggggaaaggaaatgtgtcgtgcgttcgtccctgcagcagctcggcccataaaattaatgaaaatcttttttaggtcatggtagattacagatttctttgagatagagaatctcaagagcagaggagaagattctcagaaaatagcagtgatatgagatggcataacgctgagttggaaactggggaggatttccagggttactggaaatttacttaagcacgagagaatgcatcgtgtgactgccagtgcttccccactcacatggctataaccttcttgcatacaattaccatcttggaacttgaaatagctgaaagagttttatttgatcttttcaatggatcttacatctgcagaaaaaaaaaaaaaaggctagaaataatcctgcactcaaactcactttactgaaccaccatcatgaaactccagcaacacacagggatttgggcaggcgtgttcatcttcctcttcccatttgcaacatgtgtatggcatttcctgaagctcactcctccaaatgcattgagacagttgtttttcattcttcctaatgcctgcatccacccatctgctgatcggcaattatttctatcccattcccttctgtttcttattaatcaagctctttatgcaatcccacgtaacactttgcccagctgccctgccctaaccactaccaattatctcatcctgttttatagaccctgtagcaagactctggccttgctcctcttcctctccctgatagagcttttggtgcagggctggctggctcctcaggtgttcagaggatcagaggtctcccagaaggatcttgttaatcaaggacaggtgctggctatatgggaggatggcaccgtatcctaaagctctacaagaaggagacggagctcagcctgggaggacagagagaagcagcagcacaggtttcaggatccagggatggcagacctgggtgtgggctcataggattgaagaagggataggctgtgctcctgtagcctcactgcagaagcagcactgctatctccccagcgaagctgtgtgtgccccatccctggaggtgctcaggaccaggtgggatggggccctgggcagtctgagccggagggagcagccggcccacagcaggggttggaatggggtgggttttaagttcccctccaaccaaagccatttcttgatctctgttggtggctggtgcaagttctgaggaaacctcattttcagctcaggcgttcttgtccctggggaaaaatcaatattaatgcttcagtgattactgctcgccttccaaatgtgcttctgatcagttcaagaaatctgacagtcacgtcgctcaggatgctaagaatacaacagaaacagctttgaaaggaacccttcaactcttgatatttgtgaatgagctccaaagaacattactcatttatttttcaggaaaatgatttcattgacatgaacaggccaaagcctacaagctctgttttgtgactgcagctccttacactttcagctgcattttcatgatttatgtgcccatgatgagacttgaacacctcccaggataatgggaaaagcagttctgatttcccatttaaaacgtaggctgcctttaagccatgtgtgtggctcaggctccttctgaagcacaaaggtgttccacccctcgctcctttttcattacaactttcaatcaaaaatgtgttttatgagatatttgttttgccatgtatctgtgacggagttgaaccccttagtgaaacctctgttcttcacttagctgagaggtatttcttagggaatgtgatgccctaaatttattgtggtgtaatagaaggggggatgtgtggactcaccttctgtttgttgtggctgcagtggttttatgcactacctgagtattaagcaagcccttttcatctgcacggaacacctcctgcttgccagtgggatgaaacaacaacaacaaagatttaaggtttgctattctcaatgtttcttaatcgggttcacattgattgccaacagatgaataattcctccttctccatggatgtacctcttaaacttgtgaagtcttaggtaacgcttttctgctgtgatgactgtttcagtcccctcagtgagaaatcaggcgcaccagtaagacacaaaggagaccgtggagatgttcattgtgccctcagcatctccaaaaggcactgctgcctgccgagccccagacttcgctcctgtaaaagcaaagcatgtccaattctgctgtgccataagagtcctgtggagcccagacacggcgtagcgtgtgtaacatagcgtgcacgagctcaaacgctttcaacaaatcagcttttttgctttgccaacttccatatgtaatttcacaacatctagtattgagacagtgctgttgtttgggcagcataaatcactcattgtacagcagggcgcctctcttaacaagttgggtgtagttcatgtttttgtctaattcctctgcgcatctctctaacaaacaactattctttagggctcgactcaataatcaatacatttttttcagtttacagagcaaataattacttgacctgatgacttcacaaggttagggagatgggtgtataaagtctgcagtgtgaaggcagagcaacatctctgcagaccttgagagcaacaggtctgcaagtaacaggctgcacagccacctctgccatggaggcaatgagagctgctgccctccttggattggtgcttctcagctcctttcctggtaagttgtttttgttacattctctgcttatatctctactcctactgaactaaatgtggttcaggatgcctttagaatcctaaaagagagctcagcctgccggagaagtgatggtttggtaaaacatgagctctcttctaatgatctttatccttgtgcaaatatttacgtaactctagcaggatgcctctgtctgacataaactcattatcctcagtaagtctcatagcactcgagagagaaaatgtataccctatttcttccttagtgagtcaaagtttatattttcacccaaaatggctattttttttaatcataggatatagcttgcttataggaactggataaaatatttaggaaacaagtaattctcagtgataaaaaagaagtatgtgatgactctgtagggaaattgataattccagaggaattgtaaccaaggacgccgtaacattctgtattttataacctctgttttttccagatattgtttctggtcatcaacgggtgagtagcagatctgcatcatttagttgtggtttctatgaatagatgaataattcatactcacaccatatcctacgggagcctagagggagaaaaaaaaaaaagaaaagaaaataacaagggaaggagaaaaagggcccccaggaattatgtgacatttttcccccagcaaataagaaaacatctttgtcagagaaagataacgtaccacgttggtgataagagttggcaattaataatgcagagtgggagccggcgtggcacagcgtgccagcagaaaatctgcacagcttttccctaactgcctccatatctcccctgcctgattccctgaggacccatcagtcagtcgtgtgtctgccatgccaaaagcctcagtagtgacactgtgctcaggcatactgtaaggaacgctgtaatttgctcccacttcttcaccgtggaggagtgacagagaataaaatgaccgcctgcagcacggctatgcgtggaaaacacaagcagacccttccgtgccctgcagagctgtcccacttgtgctcttcccaggcctcctgcggtgagtaccggctgttaggcagcaggaacctcgcctgttccaggatcttccagcccgtctgtggcaccaataacatcacctaccccaatgagtgctcgctctgcagagaaatcctgtgagtagcgatcgcccgattacccatcgtgatggctcaggtggcagacagaagccttttgaattgtgactaatcacgggtggattcgattttttttccccctgtttctgtcttcccagagtgcaggctgtgtttcttccttgtcaaaactcctgagtctaattaattagtggggctgggcgtggagaggcttgatgagtgaggtgactgcatggcaccaccaggttaacccttcccctccttctctcctagccggagtgggacggttgacaagaagcacgatgggaggtgtgtgaaggtatggttccagctcagccactgtgtggagcgatggcagaatcccttcccagcactgattgtacatttagaatggacagctccaaacccattggaaatgtaacagaaaggaagaatttcaggtcttttatatatatatatatatatatatatatgtatgtattaatttcattttgaacagtgcaaatctgtttcaacggtgagttttgagatgttatcttgtgtagcacagctgacttaaaaacagaatcctctcatttcaataatcctttggtgttgttgaaatagttccctttagacttagacagaagtctgttgaaattaagaagttccccaaggaagtctggattttgactaaatcataattttgtaacagggaaaaagaaaaaaaaaaaggattccatcagaacatctaccctgaggtttgtttatcaatacacggagctgccacgaagtggagaagtgtctctatttttagattagagagataatgtaaagaaacactccggctgtgcaattgaacataatgctacaattttcacttcagtacactcagagtaatggcaggaacaccgaggtgagcatcagctccattttcaagtggagcagacatttcacagcagcagttgctgccatgtagggcatgttaggcacagatcctatgtggtggcatttggggtggaaagccctaagatgacaccaacaaaacccattctgtgaacccatttcctccaggattctgctgggctcatgtcctcaaaggcaggacttcacctgcctgtgctcccttgcccgcactgtgctgggttggaagctcacatctccatacagccccactcaccgtgagtctgggggtgggagacacctctcacaccatgcaccattacacagggctgacggaagtgttgttctgtggctgtttcaggttgattgcactggctacatgagaacaactgatgggcttggaacagcctgcatccagcagtacagcccgctctatgccaccaacgggctcgtctacagcaacaagtgcaccttctgctcggcagtggcgtgagtggtgggtcacaccctgggtgctggggtctgggtggtggtgtttgcagcatattgaggcttctggagtggctgtgctgtgctcattcattctcaacttgctttcttccccaaggaatggagaggacatagatctgctcgctgttggaaaagagcccgaggtaaagctcgaaagtctgcgctatgaactgttgttataatatattatacagcacaaattcagtgagtcagaactacgcaatagcaatgtcttcactgtgctggtgtatttgtcctggaaaaagggtttgaggaaaatgactcaagtatgccagggtcagaggacgatgaacaaaactcctggctcctgtgtcagtatcacctgcacagcccctgacaggggttgatgctcagagcattgttcagatggtggctgtgccagaggtgctcaccgctcctggtgagcgtggggctcatgcagcaccagctgtcattacttgggtgggtggacttcatagtgtgctgttggagacacactgcttcctggcagcccctctctgctggctgctgaaccagagcagagcaggtagcgggccgccagccggggagcactgctttggctgtgtcgctgcttctgagggtatttagtagatttttccctctgacttctccttttgtgctctgctgggcaagagcattagaatttgcagagttgctagaacaacaggagcctgcatctgaaaaaatgttttttttgctttgccatgacataaatgtaaagcgcccatgtaggaaaatacaccaaacaaaggcttctcaatacgttcttgctccattacctacagattgactgcagtgaattcaagagcactgatgcctactgcactgaagagtacatgcccctttgcggctctgacggcgtaacgtatgggaacaaatgccacttctgcattgcagttttgtaagtacagtgctccccatgcagccatgaaaccactgctgtgccggagtatgaaggcagaagctgccaggaagcctttgtgctcccgttatccccttggtaaatccgtccccatccccaacctgatcccagctctacctctgctgtgccttccccaagcactgcagatcttgaacacaggtgagtcttctccctccctcaccattaaattcagattctcatttgcgggctcatagcgctcctgatccatccctgcgagagtaatttgagtggtaactgtagaaggagtatccaaaattacagggtttgtcccagatctctctaacatgacaaaacgtgtaacctggggaatcaggagacgggtgaaggtgcaactgggacagcatggagcattggcttgcccatgcaaagtcagcagtggcaccatcagggctataaaaccaccttccatgtcagtgattttggcctcctcctttctctgcaggaagagtcatggatctctgtctctgcagcaccgtggagaatgctgaatgctggatcgtaacctttaccctcatccatctttcacttccaaagcctgcaattccaacacgctcttccccgctccctgctgtacattgctttctgccttgacccgccagtaaatcacagacagcaactctcttcgccatgggctggtgtgttatttatttatttatttatttattgttgttattattttttccagggcagaggtaaaagtcttcaggctttcaggcacttatctgtcaggcaggagaagttttgaaataaaccacaataaaggccaaagtgcaacacccatcacacaaaagccataagccctcacgaaagtgcgtcaccccattccaaaccatcagaagaggaaatgttgctataaaacacatgctgctctccccagttctgtgtcttacagcacataaatggatttgctttaagagtcaggatgtggctttgtagaagcacggagccctggaggaagcagtccttttgggagccttggtatggaggaaagatggctttgatacacctgagcaaggggcaagtctggcggcacgttacaaggaggcttatggcaaagggaggagactatctcacagggaagaaaattaggaactgttgcttccttgaagggtgtgtcccttgagagtgtggtgatcagcagaaaattgcagccagctgggcaaggctgtaatgagcctaatgaggaccagaggagaaaccagattgggctcaggcttcttggaaaagagatctgaaaagctgcactgggagcgtttgaggcagaggaaagagaaaggactcttcaggaaaaggtttgggagtcttcatgcctagaaaagaaaggacagaaggagtgcttggtagctccaaggtcgtttctgtctgcagtgaaaggtgatgtgtggatgatgcgtgtgagcgttcacagtgatgtgccatctctttgggcgagtcaaggaatgagtatgcaaacaacaggtgaaaagtcccaagtgcctccactcatgccaccttccccttcctttctccacctcccatcctctcattacgtaggaagacattcagctgttcaggctgatattgaggacaaaatctgtgacttccaagcttttctctggctttatttcctgaaataggctgtatcttgacctagaaatcttatgggtgcttcctgccagaagatgggaagctgtcctttaatagcgtgtcagggcagtgctccgtcctaggaagacagatggaactttgaaatgtttattctattagcacaggcagtataaagcacagtgtgcctctgtgcctgctggtgagaaaaggcaagctgcagagccgtgagggtgctccctgctaatctgcctagaagggaaaagagtagacaagaaatagcatatgctactactgaatgtgagcagaagacctttagtgaaggacacagctcagctgtaatgtcctgttggccaggaggtttgttgagttatcgcagagcggtagagttctggtcagagcaggaaggtgccttcaacagcaagatcccatggtaggcctcttctgcagtgtgctggcacaagcctggtacctgctcaggagcaaaaaaaggctttggaaaagctcaaagaagggctgatgtcttacagggaaagggagggcaaaaggcaagtgcagagcatatggctgtacagacaaaaacccttcagaaaatggaaaaggtttttatcaagtaagcccagaagttggcccagtgcaggtaaacacttggctaggtaacagtgaggctctgcccagccatacccattcctctgtaaggcaaatcccaggtgcctttgtcttgtctggtcctgttctgttcctatttttctgagaaatcagacagaacttccccacctacagcatcaagcagctactttataggtgaagaagtgcaaagagaagcaataaggataatcaccacttggctaatttagtctcttcctctcagcccacaaaggactggtccctgtggtacattttctaaggcttttcccagtcagctgtgctgtagcaaatgaaatgtttggctagataaagagctgaggtattagtgctggggcggcgagcagtgtctggagcaagaaaaggcaaacgagggattctgcgagtggcagaactaagcctgattttgaatggcgttgtggctggcggacttgtaaattatatgagaggctgtgctgtgagctcaccctaatagacatctgagaactcacctgtcaatcgcggttcctctgctgtgtgggttttatggtgtctagtgagctgcaagctctaatgctttcccaggtgcagggcagttgtggcattgctctcctacagaaactctcacttgctggctgaggatgtttaggaagtccttggttgctagaaaaaatatattgaagtgctttttttgtttgtttgttttccattcttgtgtgaaattttgttggaatcacagaatcatagaggttgaaagagaaactctggaaattatcaagttcaaccccttgctaaagcaggcttcatacagtaggttgcagttacaacatttgctggggaaatgaatatgaagatctgtctataaagagtgttcccatagcacttgtttctttaggaaagcatgctgaaattctaaaggctgtgcctatctgaagagatactttgcaagtggtgcaactaaatgctgctcttggtggagagatggctggagatggatcgatggttgggtgatcttcgtggtcttttccaactttaatgattctatgattctatactctttacacagaatcagctgggaatagagtgagagtctcctgattccccaccaaattcctttgattgatgcttggtgtggaagcagagctctgggacacgttggtgagtgtgaaaactggaaaacattgacagctatagtttaaatagttcagggaggagaggcagccatcctatgtgggactctgcacacggctatgagagcatcagtgcgcttctccaccccaacccaacaaatttagagccatcctccaaaatagccagggaacaacgcataattggtttcacagacaacacattctcatgctgtgatttatttcgtaatgtctggtgagtgtcatcacgccgtgctcaaagcctggagctggcattcagcgaggacccagagaatgaaaattaccagcttccccgatgaatcaccactttgaaaattcacccttgtgagaatcctgtgactattcagaaaaaaaaaaaaaaaagaagaagaagaagaagaagatattacaggcccaagtctatcagtcatgtaattagccctttctaggtttgatgtggacagggcggcattcctaaagcaccataaacacggccgggaccaataatggctctagaatcgaagcggagaagttctcacaattaaggtgaggaatgaggccagcagcggataggtacataaatacacggaggcagggccgtgagcacgctgtgggcttgtggctgagacaacacctcccaaaccggtcgcttgccggggactaaaagagcagcatgaaggcaacaggcacctcggtgctcctcagcctgctgctgctgctgtcgttcttctcgggtaagttatatttctgtagcctagaaagaaactttatgacgagagcaacttcagagagccttgatcaacggatgacaggcttgaagagaaagctgagcaagtagaaaatatctgcgggactcgcttgcttgtgtcacatctttccattcctcgtgtgcctccgcagtgaataacactgtggaggtgtcactgggagacagaatgagcaaattgtaagcagctcgttcagcagaggcaccaaagcagagcgtaattatgagttttggtggaaatgtttgctggagagctttgctgaaccagttagagaagaaactcatacctcagggtcatcagctcctgttctgatgctaagcacttgggggttggtgttctcctcagagatgtggcagcgtaattagatgaaagtttcagcttccaaatacgttgcagaggagggctcgaaaattaaattcagatgtcctcgaggaacccgaacaaagagggcaaattgaaagggtccagcgtttatttatcttgaggtttacacgtctctctgttggtctggggaggctggctgatggtttgggggtgtgtagggcacaccggggtgctcaaatgctcgcgtgcggccgatgcgaatgtggaagcgttgcggtggccattactgaagactgcagaccaaggattatttatacttgtttttctgtgaataatttgaataaagaattcgcttgagaaaatcgcaggctgtgcatggagagaagaggtgaattactttgtacacatcattaattatgaaatattcatctgtctttaattgagtcttaattggggctgggttccgtcagagtgctaaagcttctttccaaggccaggcagaatagcagcaaactctgtgatctcaaataagataaacagatgccaagagacgttctcacaaagtcttgtgtagctgcatgtaatatttataaaaattatctaatgagctgttttgtaaataatatgcagatagccctaacggcggcttccctgtccagcctagctgaggatgtgacagatacagcagtggcaaggatcaaacactgaaaggcatcgcagcaggcagaagctgggtggggtgatggatggtcccgctgagcgtgatgctgcaatgctcccagcctgcaccctaaccaaagggatgccccattgcaatgcgccccagcccctgcagcgctgtgtgcagcccactccctgtccccgacaccacaggatccatcccgtggctgtgacctggccccatgcaaagtttgcaggcaggaaatagcaaagaggatggactgattgtctccaggcccagagcctgtgcctgcagcaggtatttttgctctgctgctgtctggcactgcctgttctgccccagatcacgccaggctatccctttgtatctcatccggatgaggctgttctgggagcctcggctgtgctgtactgcagacggctctgatgctgactgcggggtctcctccatctcccctgtgtgcttttgttaccgtactggccagttttgtaattcagaggtgcaagagcctaaaagccataagactcaatgaagctttaaaatctctgctgagagaggctcagctcttacatagctccccgcttccccggcggtggctgcctgccagggagatgggtttatgtgtctgtggtgcagttagcagctgaatgactgattacatggtattttagtaacatttttcaaatagcaaaatactgaaaagcaattccgataatgtatttcctacccctcctccaccacacagaacggcagaggagggaaaacctggtgtgtgctgtgctgcagtttgcaaagggatttgtgacttcggttcagtcctctcagaaaataatgctaatgtggataaaatctttttttttgttgcaattctaggtgtagcagctcaagacattgaagaggttagtgcagctctttctgctttctgaatctgcattttctcctggctctggaagaatgcttttctaacagatcttggtgcattggtgcatgctgaactgctttgggttttgctgggatcaggtgggtcctgccaaggtgccccaatgcttcggagtgctcacacagtacaggggtgttagctatggccacagtagcaaacaagttggggatgatttagctggtttagcacatgctccccatggtctgatccagcacagggctgtctgcagtatcgcttctgtctgctttgctcctccacgaaacaaatgtgatatcaggagtgatatactcctttaaaccatatccataactggggcttgtccaaaagcctgttcacttcatagaatcattaaggttggaaagaccactatggtcatcgagtgcaaccactccatgcccagatccctgtgtatggcagccccaggccacgtggtggtgtgagctgcatggtaccgggcactgatatggggctgcatcagtgctgatgctctcctgttgaacccactcatgttcttggaacaccagagctgctccctggtggtgacagcttccctcctctgccacagggcagaaattcccccatttcagccagttctgacaggcctttgtttttcaagtaagcaggccgtgcctcgttgctgcttttggcctctgggtgggaagaagatcacattagagatcttctttcctgtttggaaagcgaaacccgacggtttattgctgttattatttttgatttcttttgcagatctgcaaagagttcttaaacaggagcgtgttctgcaccagggagtccaaccctcactgcggcacggatggcgtgacgtacggcaacaagtgtgccttctgcaaggccgtgctgtaagtgggggcggtgggatacggacccacacagggatggtccacttccaaccccgcgctgctgctcccctcacacagagcaatccctggccatagaatcatagaactagagaatggttaaggttggaaaagaccaataagtgcatctagttcaaatggcagctcctcaccgccacgcttgggaatatttcagcttaatgttgattcatttctaggcttagtgtgatgctcatagccgtacagagatggcacagagcctgggaggccattgtacctgcctgtaccttctgcgtgggctaaattgatgcacattttcctctgtgtgccacaggctgaagctctccctgtccacacctctggatgctgaagtgtgtggaggaacgcaggcttatgcatgccaaattattagaggaaagtcatagactcgtagaatcatagattcgtttgagtcgaatgggacctttgaaggtcatctggtccagcatccctgcaacgagcagggaaagtgctgaaatgaaagtctgaatggacttagtggaaaagtacacaaaatctcagaggaagggctgcagtttctcctctcctgtctcctctaaaggagctgtaataggagccaacacctctggactgaaggcctgcaaaaattgatttatccttatcaatcctgcactctggaggctgccttatcctaagggaaattagagaagagggaaagatggcttgatgctccctgtgaggcaccagagtgaggcaaatgatcgtgctcggagggacaagctccctgtcccagccgctgtgtctgtgctggatgccatacactgctttgtttccataccgctccttttacaggaggagtggagggaagatacgattgaagcacatggggaagtgctgagcctgagcaccaagcactgatcttcgtcggtcacaggtgcaggagcctgggcacggcagcagctgtcctcatctctgccatatctgctcaataaagtaaagctcagcacacctccttgactggattcctttttccataacacccggataagccttccatgcagccgtgctagcagctaaaatgtttgccgcactgtgctgttacatcttagaatcacagaatcaggcaccatgctgcctgagcaggagcaatgattcccacagctcttccatgccatgccatgccatgccatgccatgccatgccatgccatgccatgccatgccatgccatgccatgccatgccatgccatcccatcccatcccatcccatcccatcccactgacaaatggacacatggccacccagcttgactgtcccatgggtgggtgacagcatgcaacgttgcctctcagcagcctccccatatgtgtccctctcgctgaggtgtgagcatgaaggtggcagagagctatgagtggtgtggctgtggatgcctcatctgcttgggaagccagaagcaaacaggctgaggctgaggagtgttgctgcatgtaagcctgcaccgggaaggtggcaggggaagctggctttaggcagaaacacaaaggctttgctttccttgtgtgtcctaagagaggactttgcctcaaagactgtcaactcgccagcatcaggttgcagttgcacacaaacttgatttctttctttagttttcacactgctgctctctctctccttgatgctggctggaaaatccttctttgcgccagcgagggaaaataaagcctatagtctctccccattcgctgtacaaaatatacacagggaaatgcttgtggcatcccctcgttaaaacgttggcagcacatcaatgggactctactcacttaatgttgaacacttaagtttcaaagggagctttagattttatcgtgaggtcagccaactcattttgcaaacacctctatgctgagcatctcagctcctggatggtgtttggacagagctgagtgtttgcctgtggtgccacgctgcaggctttgaagtgaattgggacattatattttgtagccaaggagagttgcagtttgctttgttccaattcagatgtttctttagtaaacacaacagctagacctccagaacatggataagcttgaggggaggaaaaagcacctcctgcacgaggacagctgatcacaaaggaccccagtgggcagtgggagaaccttcatcatcctctctaccgcctggatcaggatgagccctgcataccctttccaactggagttaccctgtgagccaacttgtggctctggagtagtgctgtatctcaatacagtttctcagatgggaagaggcatttcaatgagaggggggatatgggacatttctatgcctgagatggctctcggagactccaaaagcctcacggcgtatccccatgcctaatcctttttaatctggaggctgaaataacaaggacagatcacaagagaacagaagcggcgagacttctctgctttataatcagcctgcattttgctctttcagtgcaaacagcaaatagaaccgcctctgtacccctccagacccaaccaccatccccagcaacactgtggcaggctggagaagggtggctctgcccctccttgcctcaactggttgtgtcagcacgaccataaccagagctctccttggccccagctgggcttatccatgtaaacctctcagtgccccaggagctggctggtggtcctgtccatttcactttcctccagcaggtgttccctttaacaagcatccaagtgcctggagcaggagcaggcactgcagaagatgagctcaggcaaggacatggcatgtggggatccatgctgttgtgcaatgcagatgacgttagatacgtgcaaagcagatctcagcaatcacccaacgactcataactgcaatcatggaacgcaattgcatctggaagtataaaagcacagtgataccaggaagctcttgttaatggcacagccattttggagcaatttgcccaggtggggagagccctcacagcgccttcagtcacagggagtggtgtgagtgcccccatggctgctcccagcccccagccctgggtgatgggggtcacttggctgtaaccctctgaacacagggacagtgagacagccctctggcctggctgagctcttggctacgtccagctgcagtcctgggcacatactgaaccagaaagcaagcattcagctggtatttttcctttaatttccttcctccacattttaagttgtgggattttttttttttttttttgacagctttgagagatgagtgagtcacgaagcactcgagatctctattagataacagagcatctctgcagctcttcctggggagggagttccttggaccaagggccaaggctgggtgagaattgtcccagcatcacagtggctgctccatcacctgacacagcccctctgcagtgaaacaagggaagcattacatctttgcacggctgctttcactgaacaaaaagcgctgcttcacagctgagcaccatgatgaaggggaaggagcatctccatgatgaaggggaaggagcatctccacatctccatcacgagctctgctctgctggtgatgcggctgacaccatggtgtgccctgactcctggcccatttaactgctgtgcaccagtgcctcctccccagcatagccctgtgtccctgccacaactcattgcaatcctttgtcctacttcttcccttgacattcacagctcttgataaggctttttgagccactcctggctgatgtgggctggtggttcctgctgcagggttcccaccacccagctgggcagcattcggttgttgttccagttcccaggggattgggacagattggaagggtctttgggactgtggaagagtatctcctgaagtcagggcagactgctcagcgctttgtcccatccagacttgaaaacatccaagggtggagaacacacagactccctgggctgccagtcccagagtttgactgtcatcacgttgaagactttttgccttgtctccatttgcaacctctttcctttcagctgccccatctctcagccatgcaccactggggagcccagctctgtctggtcaggaacagagcccttacagagccacagcatcctcctgaagtgtccatctcaccactcagcctcagcaagtgctccagccctcaactcccattttccattatctttctatcactggatatgggagggaaggcagagctgtggggccaagagaaacgattgctcaggaggcagttgggagaactttattgcaaagcactgaagagatataaagtgacatttgcaggaaaaagtagaagggtatctgtgtgtgttggttcctttaaggattagagagcagctgagctttgggatgagagggctcccagatgctgtgaatcagctaacagatccctccaccccgtcattggtggtgaagttaaataggggcccaggggaaacatcagggttgtttttctttttacggactccagagcaaggagaaggtgagggggttgtgctttggaatgggagtgaaagagtttgttggtgttttcctctccccagaataagtagtgtggtgtaggagcgtctcataggagtagctgcgttaattgtggctggtgttagcatcctataatgttgctccagaaatgctggagcaggcttataatgatgtgtatgtattaccataatacatgaagggagaatggggggggggggggtagatttaagatgtatgcccttagaaaggcgggtgtcacttaaagaagtacttgctttatagctccagtgatagaattcattgagatactctgaacctatggggcatgaagtgaccagatcttcagtttggtcagctctgggggtttctggggggagcggggatagagcctcaatccaggtctgaaagacaaggctgagatgtgctgggcctggggtgctgccctgagcaacgtggggctggccctagagagcagcattagtgcctgcagcagggctggcccttgtgcccagtgtgtggggtaaggtggggaacgtaggtgctgcataatgtggtgcttctgatctaaaactgctctgttaattgggagtgaccagagatggccctatggctttcttcccaaagagctctgtgtccttctctgcagggtaatctgtgataaaaacatcgcctatgctctgccctgcagatgcaggggtttttgtcatcctccttctcgagacatactctaatccttacgcaagcagggagctccaagcttttggtgataacctctcaaggaggagctggaagggcagctctgccgagcagtgactgcgctgcacggggcgcatcctgcaggaggcggtggtgtaagcgggactccgctcgttcccggctatggggctccccctgctgaccgccgggcggtggccaggagacctcggggccgctgctgcccctcggtggtgcttttcgggacagctttcaggatggggcagcccagctgctctcgcggggaattaagcggctcggtgcagggcggcacggcgctgagctgccccagcaaagcgccgctcgtcccgcggcaccttcggtagatgctctctgcttggcagctccttggtcgttctcttggccggtggccaccccagcatcgctcggggctcggtgccatcccccccagggcctgcggaggtgccggtgcccgtcccgggggtggcggacgggcggtgcagtaccgatgctgggcgctgggtgctgccgcagaccgagcggcgctgcgcggctccggggcgctcctggagtgcgagctgagcaacctggtagaaaaataagtgttgtcccgtgataaacgtcatcgtgctgagctctcagactctgccagaggcctgaatgaagctgcgtcaggggagaatcaggttggggctaaggaaaggtcctgccccagagggcggtgggtatagaaggggtgcccagggcagtgggtgcagtgctgggctcccagagctggaggagcgtctggacagtgctcaggtttggatgttgggtggttttctgaagggacggattctgggctcgtttatcctgagggtcccttccaacttgggttgttctattcaatgaatattgtttatgttcattctattctatgatcttgttcaggctctcactgctgcctccaagggttcagctcccccagagctggcagggcttcagccacttgcttacagtgctcatttcatgcctggcccatggcttctgcctgagccttgtgggagatcagctgctgccagaaacccagccctcagcactccacttgcccagcttgctgccttagtagtctaacttggcagtggtctgacatgacttgaggttgttttttatttccaaggtgccactgacttttttccttccatagtttctggaagcatttccttcctacttgactgagtcgtgctctgtggatctgtaattatccaccttggctatgtgtcctttacgggattttatatgttaacctcccaagatcattttgctgctctcatcttagtggctgctgtgagctccaccagcaccacactggatgagctgcaggctgaggccgggcacctctcctgactctgctcttctctgaccccagagctgtgcagttgggatcctaacaccatgcagatgctccaggacctgcaccgagccccagcactggcactcatctcttctttccacccctctgagagcaacaagtggctctgcaatggcaatgtaagtgaaaccgggcgggtatcttagagcacctggaagcttgcatgcctgcaggtcgactctagaggatccccgggtaccgagctcgaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgaaccccttgcggccgcatcgaatataacttcgtataatgtatgctatacgaagttattagcgatgagctcggacttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccgagctcgcgagctcgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcgactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttacatctggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcccgggcggccgcaaggggttcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactc

SEQ ID NO: 37 - DNA, 26 bp, chicken
cgggcagtacctcaccatggacatgt

SEQ ID NO: 38 - DNA, 21 bp, chicken
attcgcttaactgtgactagg

SEQ ID NO: 39 - DNA, 20 bp, chicken
cgaggaacttgaagcctgtc

SEQ ID NO: 40 - DNA, 20 bp, chicken
ggcctgcactctccatcata

SEQ ID NO: 41 - DNA, 1680 bp, chicken
  misc_feature (823)..(1203): n is a, c, g, or t
gatttcactcatctcctaataatcaggtagctgaggagatgctgagtctgccagttcttgggctctgggcaggatcccatctcctgccttctctaggacagagctcagcaggcagggctctgtggctctgtgtctaacccacttcttcctctcctcgctttcagggaaagcaacgggactctcactttaagccattttggaaaatgctgaatatcagagctgagagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataagcttgccacaaccatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagggatccactagtccagtgtggtggaattcaccacaggatccccactggcgaatcccagcgagaggtctcacctcggttcatctcgcactctggggagctcagctcactcccgattttctttctcaataaactaaatcagcaacactcctttgtctt

SEQ ID NO: 42 - DNA, 2340 bp, chicken
  misc_feature (823)..(1224): n is a, c, g, or t
gatttcactcatctcctaataatcaggtagctgaggagatgctgagtctgccagttcttgggctctgggcaggatcccatctcctgccttctctaggacagagctcagcaggcagggctctgtggctctgtgtctaacccacttcttcctctcctcgctttcagggaaagcaacgggactctcactttaagccattttggaaaatgctgaatatcagagctgagagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataagcttgccacaaccatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatagggatccactagtccagtgtggtggaattcaccacaggatccccactggcgaatcccagcgagaggtctcacctcggttcatctcgcactctggggagctcagctcactcccgattttctt

SEQ ID NO: 43 - protein, 11 aa, SV40
CGGPKKKRKVG

SEQ ID NO: 44 - DNA, 77872 bp, chicken
attcaccacaggatccccactggcgaatcccagcgagaggtctcacctcggttcatctcgcactctggggagctcagctcactcccgattttctttctcaataaactaaatcagcaacactcctttgtcttgtttaatgctctgcctcatgcaatgttttcttctgatttgttggacggtgataccagactcaatatgttccatgctcgtggctctggggtataacaagaacaacatcttgctcccatccctgtcataaaaggcagaaaattaaatacagatgcataaacctcggctgtgtgactttgcgcataaatgacagtcagcctccattagtgttcagacccttttagacagctgaaatactgctacgaactgctgatgctggctgagctccccatggtacgtgtggtgcactttccctgcgcagcattagcagtgaaagcagctcagggtgcggtggtggccaaacccagggccgatcccacggcctcctgtacctggtcatacccacgggcacagctgctagtgaggtgcgtgcttttcagacacgtcatataagtgtgccctgcctacatgtctgggtcctccaaatgacgttgcaaggtttatctcatcttggaattgtcccttactgaccaccaagtgttttgagatgaatgccctcctaggtctggttctgctcttgcctgctggtcttttctcatagtagtccttgccagcccaagtatctgagcagtgttttgcaatccaaggacaaagtacccctctgcctttgagagtgtgacctctgtcattggcacattgtccgtgaaatatattttgcttttgtcctttgttggtgtattgaactgatgttttcttgatccacatgagagaaactttaataaaaattataaaaaataatgcctcccttaagcatttcttttccctgatggaatgaggccattcaaaagaaggatgctttggcggtaaaacagaggatttatgttgagatgggcagatgaatcaagcagtgatttccagtttggattgaacttttctgggatccaggctgtgggcctcatgtcattctgtcatcatcaggctatcagtctgctgctgcaaatcctccccacaacgctaatggcttttagggaaaatcgcaattgttagttctttgctaatgcccataaaacttcttccatcacttgtccagctccaggactcccttcagccccaggtttccctcttgctctctctcccagttcagtttttctggatttgctatgatttgatgatgcattattgacaggacaaggggaaatggtttcaaaccagaggagaggagatttagactggacataagcaagacattttttacaatggtggtgaggcactgacagaggttgcccagagaggtggtggtgccccatccatggagacagccaaggtcaggaggggctctgagcactgatggagctgtgggtgcccctgttcattgcagggggttggaccagatggcctttaaagatcccttccaactcaaatgcttcaatgattctgtgattctattgggttgaagcatgccaactaagactttccactctggaaaacattcaattcagttcaacaacattttccagcaacagtgagaaagcactgcatataggtaagcactgataacatgcacatggaggaaatcctgcagcattctctcttcaggtttgtacagttgcccttttgcccacaggaattttccatggtccttcagcaggcacctgtcacacacttcactggaaataatgaagccgagggcgtacttcacatatttaaacctgcaattgctgttgataaagaagcattctttgtggctcacttgtgtaagtgccatcaagatttacaaccctgacaccagagctggaacgctggttatttcaaagtagggggtggctaaaccaaacgtgaatgcacacagccacgcacacacagatcaggtggccatccaagggcagaagggccgcattccatgagcacgatgcacttctgccctttgctgctgcccaggtgagtggctgtgctcctgctccgtgcttcgtcgagtgctggctgtaaaaacacaacaaacatcctcagactggaaagagctgtgttctacaaggacttatttactcctagagggatggtgttgaaaagacttgacatcaaagactatcacttatggggtaatattttagcaacagaactgagtgggtaagaacaactgtgggaacagctccgcgctcggtgctagtttatgcataatgaaagcagtgacacgtacgtggtaccacgacatccaccattgaacctccgaaacgctgcagaatcacaaattcttttactgaatggaagcgagcgtttcccgcagtcatcctgaactgagatgcaattggaggggctgagcggctgcagcagcgttaggggagtttcacctcgctgagccctcccgttatttcagtgctgttgtggagctgcacgcaggagctgccgccagtccgtgccagctctgcggccctgcttccccggcaccttgcttatctctgagcacctgtccttgctcatcctgtgaatcacggagaattgctttctcttcctccctttcatttcgcgcgtccttctccacccgggctgtaaccctcctgagaaaaaacgtagtacggaatcgatgttgtaaacactcagcgtggcacaacgttttgcctgaaatcccttttgtctgagagtcacacactgaattgcaagttgtttattcaggacatgcactcacggattttaacactaacgaaggagatgaattgcatttgtgtcacacttcctattcccttctttactccagaccccactgcactgaaggtaagggacagatctttcaggtttttttttttttttctccatcatttctttcctcaaagcagtttccgtataaatcattactaatcgcattgtgatcgagcgtttgaaagccctgagtcatcccacagcctgagcaatatttgctacagatattaccgagtgaaatggccattttcatctgatggtttcaaaaaaaaaaaaaagataataataataataataataataaataaatagcgcagcattcagttggtgtccaagttattgtcacggttactgcagcagcactgaggatgtttacatgggatttacatcactggaggctgaaagggcactgcaggcgtgtaccgcgctattcgctgccccatccttaagctcttctttgacatctgctgatggtcggtgctgggggaagcccggggctgtgggggtctcctggcatctgccctgctgatagctgtgctgctgagggtatttctgtgagcacaaggctgcatcgatccacagggcgactgcagtgcctgcgccgtaccccgcaatttctgctctcgggagcgcatcccacactgcgggtctgatggcgtaacatatgccagcgagtgtttattccgcaatgcatttctgggtgtatgaaaataaatctcttcgctcactgagtggtgaacttcaactgtcttatcaacctcagggactgcctggagatggaaggtggttgtgtttggcgctctcctcttctcttgctagcaagggcagcacttttttttttaaactgggaggatttaccagggactcctttctttcaggtaaaaagaagtcacatttagcagagatcttcatctccacgttgggtaatttgctgaagagctcgcttccagcaaatacagtctatttcctacagcctatttgttcttcttttaaattaagtctttatcgtgcctttgaatgttagtaataagaggaagtagctggaatagctttccgaatgttctgttttggttaagttcctctgtgatgtatccttaagcagagggagggatgcacagcagaagcgcagaggttcaatctctgaggccctgagctctttctctccagaactcattgagttctcaccttgctgtgccctgcgcagcgctcacatcacagcccaccgggctccagctcagacaggaggaccctctctggctgtgttccttacaggggatgctgcccaaagcctcgtcctgaactttgagtgctcctgataaagcctgaagctatgctcaataaaaaaaaaaaaccttcagcattttggtcttgctttcatactacgtatcatgctgttgtttttttttcttaagatgctgtgtgattgcatcactgcaacagtcctggggtgtgggtcttaatgggaaaattacagggagaaagaacgggttgtctgatttatgaagaaatcaacccctccaaaaggccatgagcttctgctttcttccagatttccaaaagaaagccactgctggggatgagatccagtgcagtgttcagggcatcctgtgcagacattgactccttaggagctgaaaataaagtagtggtgggtacccgtaggtgtgggaagcctttctgcagccacctggtctgcctcccaaagcagaggatgggatgttttcccctccgggcagcaccaacagaggggtggcagcagggtgaggaagatgattggcccctctgctctgctcttgtggggaccacatgcagtattgcatccaggcctggggccccagcatgagaaagacgtggaactgttggagtgggtccataggaggccatgaagacaatcacagggctggagcacctctcttatgaagaaaggctgagggagctgggcttgttcagcatcaagaagggaaagctgagaggacacctcattggagtcttccagtacttgaagggagcttgcaagcaggaaggggaacaaacttctacatggtctgacagagatagaacaagggggagtggctttaagctaaaagagggaagatttgggtgagatgttgggaagaaatactttactcagaggttggtgtgacactggcactgctgcccagagctgtgggtgccccatccctgtacatgagctgaaggccagattggatggggctctgtgcagcctgatctggtggggggcagccagcccatggcaggggttggggtagatgggttgtatggcccttttcaacccaaaccattcaatgattctatgattctcagataagcctgcctgcccacatctgagctcacggtgctcgctgggggtggggtatggtacactaaatgatgctcagaggactgcacgcaggacctgccgcagacgtttatcacctcacccaccacttagctgctgcttgtagttaattacgtcagctgtcacttgtagagaatcctttgagatccttgggcctccggaaatcttggctgatgaaaggaagggctcagagtcatagcgttaatttattattcattaacaccaaagtgtcggctgtacgggcagtgggctcacagtcaaatagttaatgatcttaagtgacaatgtgtcactttgcagacagcagagagaacagctctcctaagggagacagcatctttccaattctgcagccattcagtgccaagctcctctttgggacgaaagtgaagatgaggaaggcaatgaggatgaggaggggcctcaaggaacctggctggcttggagacaagtgatgatcccagctgctctcagggtcccagcggtcttcaaagggcatcttgcaggggctgtgtcctctgaacagcaaaacccaggtcatagaggggaaagtgtgagcagagatgggacaaatctcccatcctgccacggagctgcactgctaagggggtgatggggagcagcatgggaccccagcgttccccccatccctgcaccaggcccagctctgcgggatggcgaggaggacaaggctctgtcacaagcatcgctggcaattattattttgttgttgctgctcaataaaatcctgacacagtacaacacaatatcctctcatcattactaatctaactctccctccaggaaatttcaggcaggaaacgttgtctgcctgccgaggtgctttatggcactgttctttagtggtacctcagcacttcgtgtcattatctggtgtcagtgaatttaggaaatgccattcaattaccccgcaaactgattaacgcattgcgtgcagttattttgttctgctctattttatatcagttcctctgttttatgtatttctctacttgttgctggccagaacacacctcgggccagtctagaccttgctgttgatgcagcttttccccagggcttcatcagcacaaatggtttgtcaacgtggggaaaaataaaattatgctttaaaataaaaccacctggagatgctgttctggggtctggctgtgtcacagctattgcagcgatggagctgagggattgggatgtgctgggccggatcctcagcgctttgctataagccaaataattccagacacccttcttccctcagatatcatctgtgcttaagcagcaggagatatgcaggcagcgatcagatagctgagctgcaaggagaaatatcacaagagcgcggcttagagcaggggctttgctcgctctaaattgaattcccatcctcataggagatccagtcctgcccccgtgtgcatcgctccggtaacagcaatgtgttttgctccatcttgcagagggtccagaagctggggaaaggaaatgtgtcgtgcgttcgtccctgcagcagctcggcccataaaattaatgaaaatcttttttaggtcatggtagattacagatttctttgagatagagaatctcaagagcagaggagaagattctcagaaaatagcagtgatatgagatggcataacgctgagttggaaactggggaggatttccagggttactggaaatttacttaagcacgagagaatgcatcgtgtgactgccagtgcttccccactcacatggctataaccttcttgcatacaattaccatcttggaacttgaaatagctgaaagagttttatttgatcttttcaatggatcttacatctgcagaaaaaaaaaaaaaaggctagaaataatcctgcactcaaactcactttactgaaccaccatcatgaaactccagcaacacacagggatttgggcaggcgtgttcatcttcctcttcccatttgcaacatgtgtatggcatttcctgaagctcactcctccaaatgcattgagacagttgtttttcattcttcctaatgcctgcatccacccatctgctgatcggcaattatttctatcccattcccttctgtttcttattaatcaagctctttatgcaatcccacgtaacactttgcccagctgccctgccctaaccactaccaattatctcatcctgttttatagaccctgtagcaagactctggccttgctcctcttcctctccctgatagagcttttggtgcagggctggctggctcctcaggtgttcagaggatcagaggtctcccagaaggatcttgttaatcaaggacaggtgctggctatatgggaggatggcaccgtatcctaaagctctacaagaaggagacggagctcagcctgggaggacagagagaagcagcagcacaggtttcaggatccagggatggcagacctgggtgtgggctcataggattgaagaagggataggctgtgctcctgtagcctcactgcagaagcagcactgctatctccccagcgaagctgtgtgtgccccatccctggaggtgctcaggaccaggtgggatggggccctgggcagtctgagccggagggagcagccggcccacagcaggggttggaatggggtgggttttaagttcccctccaaccaaagccatttcttgatctctgttggtggctggtgcaagttctgaggaaacctcattttcagctcaggcgttcttgtccctggggaaaaatcaatattaatgcttcagtgattactgctcgccttccaaatgtgcttctgatcagttcaagaaatctgacagtcacgtcgctcaggatgctaagaatacaacagaaacagctttgaaaggaacccttcaactcttgatatttgtgaatgagctccaaagaacattactcatttatttttcaggaaaatgatttcattgacatgaacaggccaaagcctacaagctctgttttgtgactgcagctccttacactttcagctgcattttcatgatttatgtgcccatgatgagacttgaacacctcccaggataatgggaaaagcagttctgatttcccatttaaaacgtaggctgcctttaagccatgtgtgtggctcaggctccttctgaagcacaaaggtgttccacccctcgctcctttttcattacaactttcaatcaaaaatgtgttttatgagatatttgttttgccatgtatctgtgacggagttgaaccccttagtgaaacctctgttcttcacttagctgagaggtatttcttagggaatgtgatgccctaaatttattgtggtgtaatagaaggggggatgtgtggactcaccttctgtttgttgtggctgcagtggttttatgcactacctgagtattaagcaagcccttttcatctgcacggaacacctcctgcttgccagtgggatgaaacaacaacaacaaagatttaaggtttgctattctcaatgtttcttaatcgggttcacattgattgccaacagatgaataattcctccttctccatggatgtacctcttaaacttgtgaagtcttaggtaacgcttttctgctgtgatgactgtttcagtcccctcagtgagaaatcaggcgcaccagtaagacacaaaggagaccgtggagatgttcattgtgccctcagcatctccaaaaggcactgctgcctgccgagccccagacttcgctcctgtaaaagcaaagcatgtccaattctgctgtgccataagagtcctgtggagcccagacacggcgtagcgtgtgtaacatagcgtgcacgagctcaaacgctttcaacaaatcagcttttttgctttgccaacttccatatgtaatttcacaacatctagtattgagacagtgctgttgtttgggcagcataaatcactcattgtacagcagggcgcctctcttaacaagttgggtgtagttcatgtttttgtctaattcctctgcgcatctctctaacaaacaactattctttagggctcgactcaataatcaatacatttttttcagtttacagagcaaataattacttgacctgatgacttcacaaggttagggagatgggtgtataaagtctgcagtgtgaaggcagagcaacatctctgcagaccttgagagcaacaggtctgcaagtaacaggctgcacagccacctctgccatggaggcaatgagagctgctgccctccttggattggtgcttctcagctcctttcctggtaagttgtttttgttacattctctgcttatatctctactcctactgaactaaatgtggttcaggatgcctttagaatcctaaaagagagctcagcctgccggagaagtgatggtttggtaaaacatgagctctcttctaatgatctttatccttgtgcaaatatttacgtaactctagcaggatgcctctgtctgacataaactcattatcctcagtaagtctcatagcactcgagagagaaaatgtataccctatttcttccttagtgagtcaaagtttatattttcacccaaaatggctattttttttaatcataggatatagcttgcttataggaactggataaaatatttaggaaacaagtaattctcagtgataaaaaagaagtatgtgatgactctgtagggaaattgataattccagaggaattgtaaccaaggacgccgtaacattctgtattttataacctctgttttttccagatattgtttctggtcatcaacgggtgagtagcagatctgcatcatttagttgtggtttctatgaatagatgaataattcatactcacaccatatcctacgggagcctagagggagaaaaaaaaaaaagaaaagaaaataacaagggaaggagaaaaagggcccccaggaattatgtgacatttttcccccagcaaataagaaaacatctttgtcagagaaagataacgtaccacgttggtgataagagttggcaattaataatgcagagtgggagccggcgtggcacagcgtgccagcagaaaatctgcacagcttttccctaactgcctccatatctcccctgcctgattccctgaggacccatcagtcagtcgtgtgtctgccatgccaaaagcctcagtagtgacactgtgctcaggcatactgtaaggaacgctgtaatttgctcccacttcttcaccgtggaggagtgacagagaataaaatgaccgcctgcagcacggctatgcgtggaaaacacaagcagacccttccgtgccctgcagagctgtcccacttgtgctcttcccaggcctcctgcggtgagtaccggctgttaggcagcaggaacctcgcctgttccaggatcttccagcccgtctgtggcaccaataacatcacctaccccaatgagtgctcgctctgcagagaaatcctgtgagtagcgatcgcccgattacccatcgtgatggctcaggtggcagacagaagccttttgaattgtgactaatcacgggtggattcgattttttttccccctgtttctgtcttcccagagtgcaggctgtgtttcttccttgtcaaaactcctgagtctaattaattagtggggctgggcgtggagaggcttgatgagtgaggtgactgcatggcaccaccaggttaacccttcccctccttctctcctagccggagtgggacggttgacaagaagcacgatgggaggtgtgtgaaggtatggttccagctcagccactgtgtggagcgatggcagaatcccttcccagcactgattgtacatttagaatggacagctccaaacccattggaaatgtaacagaaaggaagaatttcaggtcttttatatatatatatatatatatatatatgtatgtattaatttcattttgaacagtgcaaatctgtttcaacggtgagttttgagatgttatcttgtgtagcacagctgacttaaaaacagaatcctctcatttcaataatcctttggtgttgttgaaatagttccctttagacttagacagaagtctgttgaaattaagaagttccccaaggaagtctggattttgactaaatcataattttgtaacagggaaaaagaaaaaaaaaaaggattccatcagaacatctaccctgaggtttgtttatcaatacacggagctgccacgaagtggagaagtgtctctatttttagattagagagataatgtaaagaaacactccggctgtgcaattgaacataatgctacaattttcacttcagtacactcagagtaatggcaggaacaccgaggtgagcatcagctccattttcaagtggagcagacatttcacagcagcagttgctgccatgtagggcatgttaggcacagatcctatgtggtggcatttggggtggaaagccctaagatgacaccaacaaaacccattctgtgaacccatttcctccaggattctgctgggctcatgtcctcaaaggcaggacttcacctgcctgtgctcccttgcccgcactgtgctgggttggaagctcacatctccatacagccccactcaccgtgagtctgggggtgggagacacctctcacaccatgcaccattacacagggctgacggaagtgttgttctgtggctgtttcaggttgattgcactggctacatgagaacaactgatgggcttggaacagcctgcatccagcagtacagcccgctctatgccaccaacgggctcgtctacagcaacaagtgcaccttctgctcggcagtggcgtgagtggtgggtcacaccctgggtgctggggtctgggtggtggtgtttgcagcatattgaggcttctggagtggctgtgctgtgctcattcattctcaacttgctttcttccccaaggaatggagaggacatagatctgctcgctgttggaaaagagcccgaggtaaagctcgaaagtctgcgctatgaactgttgttataatatattatacagcacaaattcagtgagtcagaactacgcaatagcaatgtcttcactgtgctggtgtatttgtcctggaaaaagggtttgaggaaaatgactcaagtatgccagggtcagaggacgatgaacaaaactcctggctcctgtgtcagtatcacctgcacagcccctgacaggggttgatgctcagagcattgttcagatggtggctgtgccagaggtgctcaccgctcctggtgagcgtggggctcatgcagcaccagctgtcattacttgggtgggtggacttcatagtgtgctgttggagacacactgcttcctggcagcccctctctgctggctgctgaaccagagcagagcaggtagcgggccgccagccggggagcactgctttggctgtgtcgctgcttctgagggtatttagtagatttttccctctgacttctccttttgtgctctgctgggcaagagcattagaatttgcagagttgctagaacaacaggagcctgcatctgaaaaaatgttttttttgctttgccatgacataaatgtaaagcgcccatgtaggaaaatacaccaaacaaaggcttctcaatacgttcttgctccattacctacagattgactgcagtgaattcaagagcactgatgcctactgcactgaagagtacatgcccctttgcggctctgacggcgtaacgtatgggaacaaatgccacttctgcattgcagttttgtaagtacagtgctccccatgcagccatgaaaccactgctgtgccggagtatgaaggcagaagctgccaggaagcctttgtgctcccgttatccccttggtaaatccgtccccatccccaacctgatcccagctctacctctgctgtgccttccccaagcactgcagatcttgaacacaggtgagtcttctccctccctcaccattaaattcagattctcatttgcgggctcatagcgctcctgatccatccctgcgagagtaatttgagtggtaactgtagaaggagtatccaaaattacagggtttgtcccagatctctctaacatgacaaaacgtgtaacctggggaatcaggagacgggtgaaggtgcaactgggacagcatggagcattggcttgcccatgcaaagtcagcagtggcaccatcagggctataaaaccaccttccatgtcagtgattttggcctcctcctttctctgcaggaagagtcatggatctctgtctctgcagcaccgtggagaatgctgaatgctggatcgtaacctttaccctcatccatctttcacttccaaagcctgcaattccaacacgctcttccccgctccctgctgtacattgctttctgccttgacccgccagtaaatcacagacagcaactctcttcgccatgggctggtgtgttatttatttatttatttatttattgttgttattattttttccagggcagaggtaaaagtcttcaggctttcaggcacttatctgtcaggcaggagaagttttgaaataaaccacaataaaggccaaagtgcaacacccatcacacaaaagccataagccctcacgaaagtgcgtcaccccattccaaaccatcagaagaggaaatgttgctataaaacacatgctgctctccccagttctgtgtcttacagcacataaatggatttgctttaagagtcaggatgtggctttgtagaagcacggagccctggaggaagcagtccttttgggagccttggtatggaggaaagatggctttgatacacctgagcaaggggcaagtctggcggcacgttacaaggaggcttatggcaaagggaggagactatctcacagggaagaaaattaggaactgttgcttccttgaagggtgtgtcccttgagagtgtggtgatcagcagaaaattgcagccagctgggcaaggctgtaatgagcctaatgaggaccagaggagaaaccagattgggctcaggcttcttggaaaagagatctgaaaagctgcactgggagcgtttgaggcagaggaaagagaaaggactcttcaggaaaaggtttgggagtcttcatgcctagaaaagaaaggacagaaggagtgcttggtagctccaaggtcgtttctgtctgcagtgaaaggtgatgtgtggatgatgcgtgtgagcgttcacagtgatgtgccatctctttgggcgagtcaaggaatgagtatgcaaacaacaggtgaaaagtcccaagtgcctccactcatgccaccttccccttcctttctccacctcccatcctctcattacgtaggaagacattcagctgttcaggctgatattgaggacaaaatctgtgacttccaagcttttctctggctttatttcctgaaataggctgtatcttgacctagaaatcttatgggtgcttcctgccagaagatgggaagctgtcctttaatagcgtgtcagggcagtgctccgtcctaggaagacagatggaactttgaaatgtttattctattagcacaggcagtataaagcacagtgtgcctctgtgcctgctggtgagaaaaggcaagctgcagagccgtgagggtgctccctgctaatctgcctagaagggaaaagagtagacaagaaatagcatatgctactactgaatgtgagcagaagacctttagtgaaggacacagctcagctgtaatgtcctgttggccaggaggtttgttgagttatcgcagagcggtagagttctggtcagagcaggaaggtgccttcaacagcaagatcccatggtaggcctcttctgcagtgtgctggcacaagcctggtacctgctcaggagcaaaaaaaggctttggaaaagctcaaagaagggctgatgtcttacagggaaagggagggcaaaaggcaagtgcagagcatatggctgtacagacaaaaacccttcagaaaatggaaaaggtttttatcaagtaagcccagaagttggcccagtgcaggtaaacacttggctaggtaacagtgaggctctgcccagccatacccattcctctgtaaggcaaatcccaggtgcctttgtcttgtctggtcctgttctgttcctatttttctgagaaatcagacagaacttccccacctacagcatcaagcagctactttataggtgaagaagtgcaaagagaagcaataaggataatcaccacttggctaatttagtctcttcctctcagcccacaaaggactggtccctgtggtacattttctaaggcttttcccagtcagctgtgctgtagcaaatgaaatgtttggctagataaagagctgaggtattagtgctggggcggcgagcagtgtctggagcaagaaaaggcaaacgagggattctgcgagtggcagaactaagcctgattttgaatggcgttgtggctggcggacttgtaaattatatgagaggctgtgctgtgagctcaccctaatagacatctgagaactcacctgtcaatcgcggttcctctgctgtgtgggttttatggtgtctagtgagctgcaagctctaatgctttcccaggtgcagggcagttgtggcattgctctcctacagaaactctcacttgctggctgaggatgtttaggaagtccttggttgctagaaaaaatatattgaagtgctttttttgtttgtttgttttccattcttgtgtgaaattttgttggaatcacagaatcatagaggttgaaagagaaactctggaaattatcaagttcaaccccttgctaaagcaggcttcatacagtaggttgcagttacaacatttgctggggaaatgaatatgaagatctgtctataaagagtgttcccatagcacttgtttctttaggaaagcatgctgaaattctaaaggctgtgcctatctgaagagatactttgcaagtggtgcaactaaatgctgctcttggtggagagatggctggagatggatcgatggttgggtgatcttcgtggtcttttccaactttaatgattctatgattctatactctttacacagaatcagctgggaatagagtgagagtctcctgattccccaccaaattcctttgattgatgcttggtgtggaagcagagctctgggacacgttggtgagtgtgaaaactggaaaacattgacagctatagtttaaatagttcagggaggagaggcagccatcctatgtgggactctgcacacggctatgagagcatcagtgcgcttctccaccccaacccaacaaatttagagccatcctccaaaatagccagggaacaacgcataattggtttcacagacaacacattctcatgctgtgatttatttcgtaatgtctggtgagtgtcatcacgccgtgctcaaagcctggagctggcattcagcgaggacccagagaatgaaaattaccagcttccccgatgaatcaccactttgaaaattcacccttgtgagaatcctgtgactattcagaaaaaaaaaaaaaaaagaagaagaagaagaagaagatattacaggcccaagtctatcagtcatgtaattagccctttctaggtttgatgtggacagggcggcattcctaaagcaccataaacacggccgggaccaataatggctctagaatcgaagcggagaagttctcacaattaaggtgaggaatgaggccagcagcggataggtacataaatacacggaggcagggccgtgagcacgctgtgggcttgtggctgagacaacacctcccaaaccggtcgcttgccggggactaaaagagcagcatgaaggcaacaggcacctcggtgctcctcagcctgctgctgctgctgtcgttcttctcgggtaagttatatttctgtagcctagaaagaaactttatgacgagagcaacttcagagagccttgatcaacggatgacaggcttgaagagaaagctgagcaagtagaaaatatctgcgggactcgcttgcttgtgtcacatctttccattcctcgtgtgcctccgcagtgaataacactgtggaggtgtcactgggagacagaatgagcaaattgtaagcagctcgttcagcagaggcaccaaagcagagcgtaattatgagttttggtggaaatgtttgctggagagctttgctgaaccagttagagaagaaactcatacctcagggtcatcagctcctgttctgatgctaagcacttgggggttggtgttctcctcagagatgtggcagcgtaattagatgaaagtttcagcttccaaatacgttgcagaggagggctcgaaaattaaattcagatgtcctcgaggaacccgaacaaagagggcaaattgaaagggtccagcgtttatttatcttgaggtttacacgtctctctgttggtctggggaggctggctgatggtttgggggtgtgtagggcacaccggggtgctcaaatgctcgcgtgcggccgatgcgaatgtggaagcgttgcggtggccattactgaagactgcagaccaaggattatttatacttgtttttctgtgaataatttgaataaagaattcgcttgagaaaatcgcaggctgtgcatggagagaagaggtgaattactttgtacacatcattaattatgaaatattcatctgtctttaattgagtcttaattggggctgggttccgtcagagtgctaaagcttctttccaaggccaggcagaatagcagcaaactctgtgatctcaaataagataaacagatgccaagagacgttctcacaaagtcttgtgtagctgcatgtaatatttataaaaattatctaatgagctgttttgtaaataatatgcagatagccctaacggcggcttccctgtccagcctagctgaggatgtgacagatacagcagtggcaaggatcaaacactgaaaggcatcgcagcaggcagaagctgggtggggtgatggatggtcccgctgagcgtgatgctgcaatgctcccagcctgcaccctaaccaaagggatgccccattgcaatgcgccccagcccctgcagcgctgtgtgcagcccactccctgtccccgacaccacaggatccatcccgtggctgtgacctggccccatgcaaagtttgcaggcaggaaatagcaaagaggatggactgattgtctccaggcccagagcctgtgcctgcagcaggtatttttgctctgctgctgtctggcactgcctgttctgccccagatcacgccaggctatccctttgtatctcatccggatgaggctgttctgggagcctcggctgtgctgtactgcagacggctctgatgctgactgcggggtctcctccatctcccctgtgtgcttttgttaccgtactggccagttttgtaattcagaggtgcaagagcctaaaagccataagactcaatgaagctttaaaatctctgctgagagaggctcagctcttacatagctccccgcttccccggcggtggctgcctgccagggagatgggtttatgtgtctgtggtgcagttagcagctgaatgactgattacatggtattttagtaacatttttcaaatagcaaaatactgaaaagcaattccgataatgtatttcctacccctcctccaccacacagaacggcagaggagggaaaacctggtgtgtgctgtgctgcagtttgcaaagggatttgtgacttcggttcagtcctctcagaaaataatgctaatgtggataaaatctttttttttgttgcaattctaggtgtagcagctcaagacattgaagaggttagtgcagctctttctgctttctgaatctgcattttctcctggctctggaagaatgcttttctaacagatcttggtgcattggtgcatgctgaactgctttgggttttgctgggatcaggtgggtcctgccaaggtgccccaatgcttcggagtgctcacacagtacaggggtgttagctatggccacagtagcaaacaagttggggatgatttagctggtttagcacatgctccccatggtctgatccagcacagggctgtctgcagtatcgcttctgtctgctttgctcctccacgaaacaaatgtgatatcaggagtgatatactcctttaaaccatatccataactggggcttgtccaaaagcctgttcacttcatagaatcattaaggttggaaagaccactatggtcatcgagtgcaaccactccatgcccagatccctgtgtatggcagccccaggccacgtggtggtgtgagctgcatggtaccgggcactgatatggggctgcatcagtgctgatgctctcctgttgaacccactcatgttcttggaacaccagagctgctccctggtggtgacagcttccctcctctgccacagggcagaaattcccccatttcagccagttctgacaggcctttgtttttcaagtaagcaggccgtgcctcgttgctgcttttggcctctgggtgggaagaagatcacattagagatcttctttcctgtttggaaagcgaaacccgacggtttattgctgttattatttttgatttcttttgcagatctgcaaagagttcttaaacaggagcgtgttctgcaccagggagtccaaccctcactgcggcacggatggcgtgacgtacggcaacaagtgtgccttctgcaaggccgtgctgtaagtgggggcggtgggatacggacccacacagggatggtccacttccaaccccgcgctgctgctcccctcacacagagcaatccctggccatagaatcatagaactagagaatggttaaggttggaaaagaccaataagtgcatctagttcaaatggcagctcctcaccgccacgcttgggaatatttcagcttaatgttgattcatttctaggcttagtgtgatgctcatagccgtacagagatggcacagagcctgggaggccattgtacctgcctgtaccttctgcgtgggctaaattgatgcacattttcctctgtgtgccacaggctgaagctctccctgtccacacctctggatgctgaagtgtgtggaggaacgcaggcttatgcatgccaaattattagaggaaagtcatagactcgtagaatcatagattcgtttgagtcgaatgggacctttgaaggtcatctggtccagcatccctgcaacgagcagggaaagtgctgaaatgaaagtctgaatggacttagtggaaaagtacacaaaatctcagaggaagggctgcagtttctcctctcctgtctcctctaaaggagctgtaataggagccaacacctctggactgaaggcctgcaaaaattgatttatccttatcaatcctgcactctggaggctgccttatcctaagggaaattagagaagagggaaagatggcttgatgctccctgtgaggcaccagagtgaggcaaatgatcgtgctcggagggacaagctccctgtcccagccgctgtgtctgtgctggatgccatacactgctttgtttccataccgctccttttacaggaggagtggagggaagatacgattgaagcacatggggaagtgctgagcctgagcaccaagcactgatcttcgtcggtcacaggtgcaggagcctgggcacggcagcagctgtcctcatctctgccatatctgctcaataaagtaaagctcagcacacctccttgactggattcctttttccataacacccggataagccttccatgcagccgtgctagcagctaaaatgtttgccgcactgtgctgttacatcttagaatcacagaatcaggcaccatgctgcctgagcaggagcaatgattcccacagctcttccatgccatgccatgccatgccatgccatgccatgccatgccatgccatgccatgccatgccatgccatgccatgccatgccatcccatcccatcccatcccatcccatcccactgacaaatggacacatggccacccagcttgactgtcccatgggtgggtgacagcatgcaacgttgcctctcagcagcctccccatatgtgtccctctcgctgaggtgtgagcatgaaggtggcagagagctatgagtggtgtggctgtggatgcctcatctgcttgggaagccagaagcaaacaggctgaggctgaggagtgttgctgcatgtaagcctgcaccgggaaggtggcaggggaagctggctttaggcagaaacacaaaggctttgctttccttgtgtgtcctaagagaggactttgcctcaaagactgtcaactcgccagcatcaggttgcagttgcacacaaacttgatttctttctttagttttcacactgctgctctctctctccttgatgctggctggaaaatccttctttgcgccagcgagggaaaataaagcctatagtctctccccattcgctgtacaaaatatacacagggaaatgcttgtggcatcccctcgttaaaacgttggcagcacatcaatgggactctactcacttaatgttgaacacttaagtttcaaagggagctttagattttatcgtgaggtcagccaactcattttgcaaacacctctatgctgagcatctcagctcctggatggtgtttggacagagctgagtgtttgcctgtggtgccacgctgcaggctttgaagtgaattgggacattatattttgtagccaaggagagttgcagtttgctttgttccaattcagatgtttctttagtaaacacaacagctagacctccagaacatggataagcttgaggggaggaaaaagcacctcctgcacgaggacagctgatcacaaaggaccccagtgggcagtgggagaaccttcatcatcctctctaccgcctggatcaggatgagccctgcataccctttccaactggagttaccctgtgagccaacttgtggctctggagtagtgctgtatctcaatacagtttctcagatgggaagaggcatttcaatgagaggggggatatgggacatttctatgcctgagatggctctcggagactccaaaagcctcacggcgtatccccatgcctaatcctttttaatctggaggctgaaataacaaggacagatcacaagagaacagaagcggcgagacttctctgctttataatcagcctgcattttgctctttcagtgcaaacagcaaatagaaccgcctctgtacccctccagacccaaccaccatccccagcaacactgtggcaggctggagaagggtggctctgcccctccttgcctcaactggttgtgtcagcacgaccataaccagagctctccttggccccagctgggcttatccatgtaaacctctcagtgccccaggagctggctggtggtcctgtccatttcactttcctccagcaggtgttccctttaacaagcatccaagtgcctggagcaggagcaggcactgcagaagatgagctcaggcaaggacatggcatgtggggatccatgctgttgtgcaatgcagatgacgttagatacgtgcaaagcagatctcagcaatcacccaacgactcataactgcaatcatggaacgcaattgcatctggaagtataaaagcacagtgataccaggaagctcttgttaatggcacagccattttggagcaatttgcccaggtggggagagccctcacagcgccttcagtcacagggagtggtgtgagtgcccccatggctgctcccagcccccagccctgggtgatgggggtcacttggctgtaaccctctgaacacagggacagtgagacagccctctggcctggctgagctcttggctacgtccagctgcagtcctgggcacatactgaaccagaaagcaagcattcagctggtatttttcctttaatttccttcctccacattttaagttgtgggattttttttttttttttttgacagctttgagagatgagtgagtcacgaagcactcgagatctctattagataacagagcatctctgcagctcttcctggggagggagttccttggaccaagggccaaggctgggtgagaattgtcccagcatcacagtggctgctccatcacctgacacagcccctctgcagtgaaacaagggaagcattacatctttgcacggctgctttcactgaacaaaaagcgctgcttcacagctgagcaccatgatgaaggggaaggagcatctccatgatgaaggggaaggagcatctccacatctccatcacgagctctgctctgctggtgatgcggctgacaccatggtgtgccctgactcctggcccatttaactgctgtgcaccagtgcctcctccccagcatagccctgtgtccctgccacaactcattgcaatcctttgtcctacttcttcccttgacattcacagctcttgataaggctttttgagccactcctggctgatgtgggctggtggttcctgctgcagggttcccaccacccagctgggcagcattcggttgttgttccagttcccaggggattgggacagattggaagggtctttgggactgtggaagagtatctcctgaagtcagggcagactgctcagcgctttgtcccatccagacttgaaaacatccaagggtggagaacacacagactccctgggctgccagtcccagagtttgactgtcatcacgttgaagactttttgccttgtctccatttgcaacctctttcctttcagctgccccatctctcagccatgcaccactggggagcccagctctgtctggtcaggaacagagcccttacagagccacagcatcctcctgaagtgtccatctcaccactcagcctcagcaagtgctccagccctcaactcccattttccattatctttctatcactggatatgggagggaaggcagagctgtggggccaagagaaacgattgctcaggaggcagttgggagaactttattgcaaagcactgaagagatataaagtgacatttgcaggaaaaagtagaagggtatctgtgtgtgttggttcctttaaggattagagagcagctgagctttgggatgagagggctcccagatgctgtgaatcagctaacagatccctccaccccgtcattggtggtgaagttaaataggggcccaggggaaacatcagggttgtttttctttttacggactccagagcaaggagaaggtgagggggttgtgctttggaatgggagtgaaagagtttgttggtgttttcctctccccagaataagtagtgtggtgtaggagcgtctcataggagtagctgcgttaattgtggctggtgttagcatcctataatgttgctccagaaatgctggagcaggcttataatgatgtgtatgtattaccataatacatgaagggagaatggggggggggggggtagatttaagatgtatgcccttagaaaggcgggtgtcacttaaagaagtacttgctttatagctccagtgatagaattcattgagatactctgaacctatggggcatgaagtgaccagatcttcagtttggtcagctctgggggtttctggggggagcggggatagagcctcaatccaggtctgaaagacaaggctgagatgtgctgggcctggggtgctgccctgagcaacgtggggctggccctagagagcagcattagtgcctgcagcagggctggcccttgtgcccagtgtgtggggtaaggtggggaacgtaggtgctgcataatgtggtgcttctgatctaaaactgctctgttaattgggagtgaccagagatggccctatggctttcttcccaaagagctctgtgtccttctctgcagggtaatctgtgataaaaacatcgcctatgctctgccctgcagatgcaggggtttttgtcatcctccttctcgagacatactctaatccttacgcaagcagggagctccaagcttttggtgataacctctcaaggaggagctggaagggcagctctgccgagcagtgactgcgctgcacggggcgcatcctgcaggaggcggtggtgtaagcgggactccgctcgttcccggctatggggctccccctgctgaccgccgggcggtggccaggagacctcggggccgctgctgcccctcggtggtgcttttcgggacagctttcaggatggggcagcccagctgctctcgcggggaattaagcggctcggtgcagggcggcacggcgctgagctgccccagcaaagcgccgctcgtcccgcggcaccttcggtagatgctctctgcttggcagctccttggtcgttctcttggccggtggccaccccagcatcgctcggggctcggtgccatcccccccagggcctgcggaggtgccggtgcccgtcccgggggtggcggacgggcggtgcagtaccgatgctgggcgctgggtgctgccgcagaccgagcggcgctgcgcggctccggggcgctcctggagtgcgagctgagcaacctggtagaaaaataagtgttgtcccgtgataaacgtcatcgtgctgagctctcagactctgccagaggcctgaatgaagctgcgtcaggggagaatcaggttggggctaaggaaaggtcctgccccagagggcggtgggtatagaaggggtgcccagggcagtgggtgcagtgctgggctcccagagctggaggagcgtctggacagtgctcaggtttggatgttgggtggttttctgaagggacggattctgggctcgtttatcctgagggtcccttccaacttgggttgttctattcaatgaatattgtttatgttcattctattctatgatcttgttcaggctctcactgctgcctccaagggttcagctcccccagagctggcagggcttcagccacttgcttacagtgctcatttcatgcctggcccatggcttctgcctgagccttgtgggagatcagctgctgccagaaacccagccctcagcactccacttgcccagcttgctgccttagtagtctaacttggcagtggtctgacatgacttgaggttgttttttatttccaaggtgccactgacttttttccttccatagtttctggaagcatttccttcctacttgactgagtcgtgctctgtggatctgtaattatccaccttggctatgtgtcctttacgggattttatatgttaacctcccaagatcattttgctgctctcatcttagtggctgctgtgagctccaccagcaccacactggatgagctgcaggctgaggccgggcacctctcctgactctgctcttctctgaccccagagctgtgcagttgggatcctaacaccatgcagatgctccaggacctgcaccgagccccagcactggcactcatctcttctttccacccctctgagagcaacaagtggctctgcaatggcaatgtaagtgaaaccgggcgggtatcttagagcacctggaagcttgcatgcctgcaggtcgactctagaggatccccgggtaccgagctcgaattccaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccaagccgaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgaaccccttgcggccgcatcgaatataacttcgtataatgtatgctatacgaagttattagcgatgagctcggacttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccgagctcgcgagctcgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcgactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttacatctggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcccgggcggccgcaaggggttcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctttgtgctttctgcctgaataaaagaaacctgaactctgttcacccagtccctgtcaggcaattactgacagagcacctatggtctgtgtttggccagaacataggctaaggaagatacctcctgtttataaagcacgcctttggcatctggcaagtaattagtgatggcgcatgagagctctgactagggcagggtgtgggacaggctggctctaattgtgccctgtttatcttgttgatgcacacggctggtttctttcacccacagctgtctctctagacaacatacctttatggagaggaacgtgtcttttccaatcttgggttttcattcagaattggagtgaactggtctccatcagatagcattggctgcggtgatttattcttttacacttcctagttaagcaggataactctctggctctgctgtgtctaggcaatttaaatgatttataaagcatagctgttttaaggaaatctttttttaaacatttgacttgccaatgtgtggtcctaaaggcagaaggactgttccagagtgtcaggcagagacctaccctggatttcgttgttcagctacccattcagtgtggcttttggcaaggaattctctggacctgacttccctacctgcagagctgggataagctatcaaaccatctcctccacacactgtgagggtgggaaaaaaacccaaacccttaaaagtgctgtataaaggcgccttaaggctcagtatagcatgtgtgctgctgatgccccagacctgtttgcgggtcctgaaggtcataggagaactgctcagaagagacagaaatgcttaagaaggttttactacaaaagtcttgtgatgttaacacataatatcacattgtgcagaaggtacaaatgccccctcctatccctgcacacctggaagctcaaggtatggaagggtttgttgtctgcagcctcttcgctgccctctgctttttaagatcctgggtagtgtgctcagtgtgtgccctcagcagtttgggaaacggacatcttcatgcaaaattaagcaaggaagtgttgcttttatactcagagtagaatctaagttcttcaggcaggctcttgtgtgccgcctctattagaaataaaactcccccggatcagaagatgaatgtgctcagctaagaacacagatttatttgctttacaatgcgtgctatggtttaagaaaaacacatcaggcaaacaatttatggtttgccactgagttgtgcctgaaggaaacacaactgttagagatgtaattgattgggcggtgacgctgtgtggattcatgggagatgcatcttggtcagcatgtctgtgtgaaaccacatttctggtgctgctgcaggacgagtgccgggagttccgggatctgttcaagaatgggaagctttcctgcacgagggagaatgatcccgtccgggattcctcggggaagcagcacagcaataagtgcatcatgtgtgcggagaagttgtgagtagaggaagccaatgtttgttatcgagagtggcaatggggccggggtgggctcctacagcaatgttctcctcactttctcatccttctctttcagcaaaagggagaatgagcagaaggcgacctcaaccagagggaaacaaaaggtgaggttaaagtattgggttcatatacaagtctataggattcttacccaatattaccacacttgatttctttgtcactctggggatccatgtggcttttcctgcttgtatctcgttgatgctctttcatgccctgagagaatagtttgtctgaacgctgcagtctatcccactgaccgcagtgacatgggagcaaaccccatcgcaataagaagctgagcagaactgccctgacatctggcacaagggcaagaaggcactgctgctgagagcgctaatgaggttgaaaagaaaatctgggtgagaagctttaaatgtgagctctgagatgctcaaaagttcattatgtcgtgggaggagagttcagccctgtgctgtccctggggtggctcggtttcagctttccctgattggaaacctcactctcatgatgcagctgctgtgcccttgtgcaccgatacttctctggtgagagcaattcagcaaggggaaggaaaaagaagcactaagtaaatcttgccatttctgtcttgcgaggaactggtacggtccccttaagcctcattcttggggataatcctgtttcagtgcttttcctaatgacagtggcacaaaaaaaatggaagcgttaatgaaacttgctgatggcaaagctgggagggaggatcagcagatcactcaggactaattggatagcactgaggcctggagtaatagaaacaagataaaatgtaataacagagagtgcaagatcacacaggcagtgattaacgagaattcctgctcatcaattagaaatgacaaaggataagaaagctctgcatttattagtgggtcacggatgcggcaggcctgagaaggaggcaaatgcacatctcagcaaggtctgtgcagcagaggtcgggctggcagcaaatctccagaaatactgctttgaagagagagggtttgagagacgctgttagggagaagcagctctgccacagcaggtctggggttcacctggggtttggctcattgcctccctgtgtccctcctccacgctgccagtgctgcactgggaaggtgtgggtaagaagcaatggctaagggatctggttatacacctcctgtatctgctatttgggattggctactgcagggcctcaggtccctgacttaaaagtggggacttcgaagcatgtttgcattgtgctgtcgtgccttagatgttgctgctgggtcctcaaagtcctgttggttgtggggtgggggggacttcttgcttcctatgtgaagttttctgagctgcaacttcagcaacagctgtaagagtgcattaagggcagtgggagaagtgggagggaccccattacctcatcgggtatcgctggcatgctttggatagccccacgtggagcgtgacaattagagcacggcagagagctcccaacacgtgccatgcaggcagaggcacccgccgctcttctgactcactctgtttgtagccatgaggctgtgccacgtgccctcttctctctctcacacctgggctctcctggggcgcgtttgggaagcctctggaggatcggagggatgtggcagggtgccctgactgctgctccttccgcaggatgactgcagtgagtaccgctcccagtttgaggctggcggacgcctgtcctgcacgcgggagaacgaccccgtcagggattcctctggcaagcagcacaccaacaagtgcctcatgtgtgccgagaagctgtgagtacagttcctggcaacagcaaagagggaaacctcacattgcgaaactgcagcttctgcctgtgtggctgcgcctgggggagtcccgagtcccagcggccccccaggagctgctcctgctgtagggctgtggctactgcccctcttcccacctcccccctaacccctcagggagcagaggagaagcagggttgatagagagcagccctttccttggggcagctcccaaggaaagtttcccacgcgtgtactttgccttccagatgctctctctactcccatagagcatatgcagaagcagccctgatatgaaagcagccacctggagccgggatgtagcatacagtgggaatggtgaggagaagggagaaggcttaggggtgggaattaggtgcagggccaccagggatggggaggctggtgcctaatgacatgatgctggcttgcagggcagccccaggtcctggcagcgttcgcactgccatagtgctcctttctttctcctctcccttttttccagcaaaaaagaagctcaaagaggaggtcagtctggtggaactgcccagcgcaacaagcagtccactgcagagtgtgcaaaccaggtgagactgagctcagagcctcaccaggcttgggaaaaggggttggtggatctggggaccccgatggtcaagggctgcctgtggtcctggtgtttggggtgcaggagcctgctggtgatggcagagaggcaggttgcattgcaagccctgctagttcatgggatgggtttgtgtatgagcgtgcatagtgggcagttctggactcctctatggggcacgcatcagagctatttcttcagaaagagccccatggttcctagggtccagggggatgagagggaaggacaggagctgctttaatctcactgctttactgcttggttgtcaaacacgatcctgccccttttccagaagagctgcagtggctcagggttacagcggggtgtaaatgagagacggccgttctccacaaacagagggtgagtacagcagcactgggatcccagcctggccccacaagtcctggggtcttgacactgagaagaaacacataaaatagggcatatacaaccctttctcctttccaaagacattcttgcttcccctgcacacgaagcactggtgactgctacactcaaaatccctccccagccttgccccctgaatcctgcctcctggcaggcacacacttgtcctgctgcctggtccagcgcatcctcatctgctgacctgaggcagtgctgtgtgtgcaccatgtgctgtctgggcactgagcgactcctctgggtttttagggctgccaggctctggcagggtgcagatgctgtgttatctaagccttgaggaactctcttagtcttcctgtttttgttggtgaggcccattcatctgcccccagtcagcactgccagcagacaaacagtgcacagctctccatggcagcaatggctgtagcatatgtaggggccaggtttctgggatcatctctgtgacggacatctcttgctgaccgcccataaggactcaaaagtcccgttgcagggagtgcctccatcccatggcaagccaagtgccctgttgaaaaaacaaggtgcagaataatggcaatggaccttagtgcagtttaattccaccctggggtgatgatgtggctgagtgggtctgcatacccttggctgtgccatgagctctgtgctttctctccctgccagcccacaaggagacttggctcaggactgcagcccggcacctggccgccagggacagagcggaggcaccaacacctaccagccggtatgcccagctcatgtgggtcaggcacagcctttcccagcagctgccccagtttccattgtcaacctaaagcctcacaatgggacctgtatccttggaggggtttaaatgggtggtagagtccgtaccctgatgctgtcccctggcctcaaagaggagtgaggctgcacacgtccaaacgggagtcactgaagccagtgctgctgctggtgttggctcactgtagaagtatgtcaggtatgagagagcatcctccaggaggtgatggtggtgtcccttcctgcatgctgagatgttgggttgaagactgtggccagagcagggtgctggggctgagcgggggataaggacaaggctgataagaggaggggagagggagtagtgggggaggacacggtgagcaatagataacgactgtttgtggaatcatgtgggagggagaagagggtgtatgctctctccatctccacaaaaagaaaatttgttattttcaaccaagctaaagcagaaattatgaaactaataggagaaaataagttactataaaaaggatgactaacctgtggatcttgctgtcacggggtgttgccaagagctacagtgattaaaaaaaatgacttgccacttatagtccatacagcaatttaggtaacattttggaagggataggaaatgcctttctgtggggctggagggacctgagtgcagactgccttaactctctctgaagtctctgtcactgactgcccttagaaaaatgatattagaatagaaaaaccagggaggcggttcaggtatggcagttttaatgcattccagaggaagcattaggcataataatgccagtctgcttcagggcttagtggtatttcctggtagctccggtgaaggagtggatgctgatcagcctgactgacgaggggtgattcagagagcagatctgtgtctctcctcgctgcagggccacccgtgggctctgtcccagggagatgctgtcctgaaggagaggtggcagtcactgtgaggactgtgggggactgttggtgtggcggcggttgcacacgcgtgggtcacaccgtgggcagtggtgtctggtgtgtgggaaggcatctggcagggaactgcaaaggtcagcgctgtctgtctttgtgtcatcgttaattacccaggtgagggaggaagcagcacattaatgaaattagcaagtgatgtttaaacagagggtgttactgcagcaacctgtgccactgaaccccctgcattgcccagctgggaaacctttcttctccatggtgctttcaaccccatagtgctgctgaccccagcaaagcaatgagccattgcttagtgctgaatggggttttttttctccaagtgggacaggaggtgagatgtccttcctgcagctcttctccaattgcaccatttgcagtcattgcaacattttttataggacctggagaaggggatgggaacagagaattcactccttttgtctctgcatctttttttttttggcctttggtgcagaggtgggcagtgaggctgaggaagagagggggctgtaggatctctgacctctgctgtctgaaacttgccatgattctgcaggcacctgtgccagaatgctcatgggctgataatctaatcatgaggagtcttgttcctcctgctccgagctctttctagctgtgccacgtctgctttgtaggaaattcgatgcctagatgctcctgctgttatgctggagaataaaacgagagggcacgcttaattagtcagagcttttcatacatgtttgcatctcttcattccgtgggtgtcaagttgtgctgtgtgtcgggctgcccttgggcagctggactcaattgtcaaggttttccctttgtttctgccaagtggcttgcagaagcaacaggtgtgaaagctctgataaaggacaaaggacaggtagcagaagtttattgtattctcgtggatttgcagggagaagtaaaagtgccctggactgagatgtcagggtggatcagatgagtgtatccatgcctggcaatggggtcagggcagctttgtccccacatcgtggctggttggcccaataggaggcgttacctctttgctgaaggtgtgatggagctcagggcaacgcctggtttgtgagtgctttgagcggtgcgcaggagggtcttgcaagagaaccagcaccaaatgtgatttctttctctcttcagctggactgtgatcgaattctgcacggggtaaagggtggaaggattttctgcagcgaatcctcacaacccgtctgtggcactgatgggaaaacatacagaaatgaatgtgacttgtgttcagctgccatgtgagtaggcggagagatttcagtaatacagggccatccaccattcccgagtgtcttttgcagcacagtgtttgttttgatataccatgactcactatcaagtgtgtccttggtgcctcgctgttaagcaaacatagatcaaatgtctgagattaatatgatgacagctaattaagatacacaactttccagagtcccttattccctttctgctcaatcataggattgtttggggagtaataaatgccatcaaattggaagtagcatcaaaggtttaaggagcccacagaggaccaccgtgacgatgtcagggagctgtggcactggaagtgaataagcaatgtcttgttctccctttgcaggagagcatcagtttacatcacggtaaactaccgaggtgaatgccgaaagactgtccctgaaatggtaagtgcctccctgctgtggcatcccatttcttgttctgggtgtgtgctggagacccagcctggatcccgtatctgtggtgggatcatcagagccctgttagcagggtgcttgtggttcacatgcgtaaatacacttcaggcttggatttaaggcattttgaggcataatctccacgttttttccaggctgtgtggtaggggagtgacatgtctgggaaaacatgtggctttcctcctgggattttggtgaggccaagaaaagattgcaatcgcacaaaccataagggcctaatttcccaaatgatatccaggcagttggttgggaaggaaatatattccctaagtggtatccttttgggaaaggtcttgaatcttgtgtgattgccttgtagtagatgagtcaaagatttgttagtggtgctttgtcttcccgctcgtggcagctcagcggcattcagagctttggtttggagccagggtgtcccagtttgtgtgtcttgagtgtatgggactgaccttagtgttggcatggactgttggaaagctgagtattcatttccccagggaaacaccgacatctatccccattccaaacttggaatgaatcaaaatatcaaatcagccaaatggagaagttgtgcaagttttttttgcaatgagagagatggcttctgaatatgaatttgctgacagtttgtaggtaaaacagtattgcccgttgaaaagctttagagcaaaattaccatcatagggcttttactctcctctgcttattgacaggatgcccacccatccccacaacattagaaatgaggcatccccattcctcttcctctcttctgtgaagtaccagagtgctctcaacgctgtttaaagctgaagaaaaaatgcagagaaagagttttgcttgtgatcgtgctggaggtctttgtgtctcgccctttggtgcgatggagccattgctggtttgtgtatgctgggagtggaggcactatgcatacctgctggtggctgtgctaatgatgctggagacagacaaggttgggtgtaccacggcaactgaaaaccagagaggactccctcagagttgtgcctggctgggattcctcaccattttgtgttttaccaagacgttttaccagctctccagtctttgcagttagaggaatatgccatacactaaaagtcagacaatttgtagctattccaaggagagctggaagcaattaaagggaaagtgataaggtttttccactggggaaaatcccccacaaaaaacacccctccaaacaaagacttattatttcgttctttatgtatattgtgtcacctgaagaatcagattggaaatttatggaagcccatttccttagcaaaccccttgtgtccatcaaagacttcccttttttttctcagttggaagcttatgaacaatgtactgaccagtgttattttatgcctctgaaattcatgctaacattcagcttaatgcatccttctgaaggcccaggcactcgctgtgtgaaggagatcacagtgcctttggcgtcagaaatgatttcaggctgttgcaatacgcagcacgaagatgcaaaggcccaaagacttgagccttggaaaaagataggagattgctgcccgaaaatgtagtttgtccttgagttgtgttttgaaattagccacggtaatgctgtgttgcctgccaaaatgtgtgtccaagctcagagcctgcagccattcctgctagcaaagcccctcctggatttccagcagtttgtggcagtccttccctagcagtggctggattgccatcagggagggatggctgtaggaagggacaggagaaatgtggttggagagagatctgacattaaagggtgcatccggacagcctgcactgatgtggtggaaaaccttcctgcagagagagccctggggctggctggcagctgggcccctgctgcctgtgtgagctctgtgccacaaccagcctcctctgatcctgttctgctttactgcagatgaatgtagctgagtctagggtttagatttctatgtttatttttaacaaggcagctggcctctgcgtcctccatgctgtgacatacagctgtattaatggtgggtctttccagaatgtttcactttcaatgctgtatttttttttattttgcagtttctctttttgttcagatgctttttcacacatctcccatgtgacagataccagtctgtccatgttagttgacaggtcaggcaaaaaaaaaaaagggatatccagtttctcctttttaatctgttttctaaagaacaaagaactcccagctttctaatgggcaaggccattttcttacagtgctctttttgtcatacctttcttaagaatgtagtagaagggaaaagaaacaaacaaaaaacccaggaccttttccagcttgatattggttttggaaagcacacagatccaggctgaaatctgtttgttttctgagtctggcagtgacccatccactgccccatcccacctggttcctgtggccactgagctgcccaaaggggctgtcatgtagcccctaatgctctgccagcgtaacagcagtggatgtacttgtggatccacttatattttgctctttctttccagaaataatggagttcagactgccagcaaataccagggatcagctgtgaccaaaggtacagtggtgcggtgatttgctccctcttggacaacttgtccgcatttcacaagggtttgggtgtcagaccttgcctgggcaggctgctgggtatgtctggggcaaagggctctgcaacacacccttccctattgccacagcacaagaatgaggcgtgtgtcttttgcagaagtagcaaggtgatgggaagcccctgccaagggggctgagccctttggggtgtgcaaacttcatgaggacctcctcatctctcaggggtgggccttgcccgttccttttccctcagatatccctgcagagggggaaggatgctggcagagcagagtactgcagtccctcctcacaaggaggtggaggtggcccaaagcaacctggctttgagctttccttgtggttcttctgtgtcccttgccttttggagccatagtaataaacccgtctgccccctgtttctctaggacaagtaaaggaagatctgatgtcaggcaccagggaagctgctgagttccccagtgctgttggatccaccttcatctccttctgcagccaacgggcctgtccttgctcaggtggagggtgaagggctgtggggacccagtggtggcttcccacgttggccccacgcatgttgttgtagtcgctgctcggctcgggctctgccgcctcgctgtgtcttagcatgtttctacaataaagataactccacagcgtcctgtcgcttttcttcactgagcctcacgggagggacgtgtgagtccccgctccggctgctcgccacgcgtcccttgagctctaaagcaccaaacccaagcggagatgtcagacgcagagaagaagaacgtggtctgggttctgttagcagggaccagcagttgggttctctgactcgctgtgtagggctttgggtgtatctctttgtctcccttcagcccttttctcttgcctgtaaaaacggacattaaaggatgcttacctacctcagagggttgtttggagattttaattggtttacgttagagagcccacgggtggaattctgttcctatgtgccaatgctggtgtgcaggaggtttaactgttgcagtcatggcctcttccagccaacacccgatgggccgtatgtatttcctgttctttcgtttatggctgttacttaaagcaaatatgttcttatttgtataaactttattgcaggacatttccagaagaccttgagtgaacgtacagtgtttgagtccactttagctgtgacctgatctgcaaatacactctgctgtagataaggctggagtaactttcagattttggcagggtttcgctcaatgccaattaatttggctccctccacagatattgatttttttttttcttttcaattaagttatcgagatctttttttcttaatgcagctaatgaaaatcgatttttactctcataaagtacttccgcatgtgtcacattgatctgtctatggcttgattatcggcaggctttgacatgaggttaatattttgtgtgctggttttttttcaccgtgtgcaaacactgtggtttagaaatatgttaccgctgcttatttctacgtggaaaatcccacggcgtggttatgcatggcagaagtcaccagtttgatccaatttagctgtttctagggatgcaagattcctctgcctttgagcgggtgaatcctcgggtgttatttatacattctgagaaggatgaacagaagacggtaaaaacgtttgctaatgatgtctgctggctgattccggctaaaatcgtgtgcagggacctcgacgtgatttttataaaggcagctcacaatttgaggcttaaagtaagttcttgcaaatgaaaatgggcgcacttgagcgcgctattataacttgtagtgatttcaagcacttagattttgaaataatcgcccataaaaacctgcattaattgtgctccaaaaccaatgagctgatgaggagggtgccctggtagcctcttttgctggatttgagcaccttctgaatttctcctgccaccagcagaaattagccacagaaatcatagctgctataagggtttattaatcagattacgaaactgctaagaaggcacacaacagtgacttgctgaagctgcctgtgctgctgttagcgagcctcccgtaggtagcaatgctaactccttccttttagcagtttacccactgcttccttccatcactccttccttttgtagggcctacttttgcagtttgatccagtggcttgcaggcaatatctgtccccagcggtgctctatgcagctgacctccaggtagggctccatgtgagcgatgcaatgtgttatttccatggggttcctaagaaggaggaagcaaaaagctcaggaggtgctccaaatatattatcctgtcctctgttttgctctttgtggtgccctttaacactgtaaagagaccataggagtcctctatgaacctggaaaggtaccagcactatgggaggtcttcagtttgctgtaaattatgctttattagaggtatttcttctgccaagacccactgaccccatgcggctcacagtgttttctaaggctttgcaggactggtgttacgaattggcaccctccaggcctctcacaaatctcctgcttctcacagcgtttcttcaagttctcccaagcacagctgagttttgagctcaactgctccctgcaggggccttgagcctcctgcctttttgcataaaaggtgtcaggtacttatgcaatccttagaggcatgcaaatgctgctctggttatatactgaggactgttgattctggcagaaccctttgcagaccttgtactcccttgctatttcccaatccctgcagcctagcagctctgcctaacaactgccatagccaacacagcagcaggctgtgcatggtgcaaggtgatgtggaaagggatgattgtatgaaagcgtgatgctgtggtactgcctctgcaggagactcgcactatttgtgtaagaggaccttatttgtctgctgcagagctgtttcaaggctgtccatacacccctgtgatgctgagcccctccaagcaatgcactgggaaaaggaggctggggggagaccttattgctctcctccaatatttgaaaggtgcttacagcgagagcagggttggtctcttctcactggtgacaggatgaggggaaatggcctcaagttgcaccagggtatgtttagattggatatcaggaaacacttatttactaaaaggttgttaagcactggaatcagctccccagggaggtggttgagtcaccatccctggatgtgtttaaaaactgtttggatatggtgctcagggacatgatttagcggagggttgttagttagggtagtgtggttaggttgtggttcactcgatggtctttaaggtcttttccaacctgagcaattctatgatatggatccctggggctttcagtcttatctccctggattatcacaggttcagctctatggcccatttgatttataccggggtctgatgaacaggtttttctcttggctcttcagggatcctatttagcactttttggtacattcccctgccctacaagtctccctgatacacagagctcttatccaagacttgggaccttccctactccagccctctgcaggaggtttcttgctaaccagtcctccaaccaggactgcagtacacgacaaagagctggaagaggtctgcaatacttccccagcatgaaggtatgagcactccttttgagtaggttactgaaagtagtaagatgtcaatacaaccaactgcaagatacaaaaccgcatgaaaattcagtttactttgatgctgaagggctgaaaagaaatgctgtggtgttagcacagatgcactgctggcaaagtgaaaatgagcaaagaggatgagatggatggacagctgatggaaaaactcttcctaattgctccacagagcagcttgctcgcctgcagggctgcagcatggagctgcttgtgcataatgcagacaccccaagaccagtgctgtttgtcttagccaagacacagttgcagctgcagcaattttttctagatgtcagttccttccctatgttgctgacaggtgtttgctgttctgtccctttaatctgtatcctacagcaaacattccttgaatttaataacttagctggaagacaattgctgtgatcttgatagaacatgctgagccaatctattttaactgcagatttagtttgcaaatactgtctccttgccgataagattcaggtgtcatctttgtggacattggcaggaattttcttgaccgtgacaggttttacagagtctggcaattaagctgtcaagacacattttcctctgccaggaagcattaattgatgatagtcttggctgcaataggcacagagagatggatattgtaatcagaatgaatagaggtccttgtagttgagagctacgttggtccaaagttttgtagtcgttgacgtttggtgatactgagataaggaacaaggcacgagatattagagctaaatatcaggcacagcatgagaataaagacctctctagctggaactgttggtatctggggagattttaactttctggatgcatactgcaaagtactaatattagtagagctactggatgcgagagcaaatagttttccattaagtaatcccaaaaatcatgttgttgttggtttgcttttcaagtgcgaggggtgttggagatgtatttccctcagaaaataaacctgatatgattcaacctgagctctctctgtttaaatcacactgaaaatagatctgcaaatggggattttgattaccgagtacagaatatgaaagattaaaacttgggaaagttagggttctgattgagaaaacttttgtttttgtggccgacccttgcagcttacaaaaatctgcctaaataaaggagaaaaccacatttagaacccatccaagctatgctacttcagtactgggcaaaacttcaggagacgtttgaagaaaactgaagacgtgaagtataaaggaatgattgatgtgcacagtaaactttcttggaaggtaatcacgcatgggctaatatcaatctttacaaagttggctgacttcctagataaaggaagtacagtagatctagtctacccaggcagcaaaaatgtttgacctgttgccctgtggggtggtgtcacctgggcttggggaggggggtcaggatgaggttacaggggatgtggaagcatactgtggaggagcaggtggggcacccacaggagttagcagtgagcagacagaaaggtggatctgaggaccgaacttcgtatttttgttccttgcattaatacacaaaaagcagacacacacacagagcagattgctgctggtttttgttttcttttttaaacagcagaagagcaggatttttcccacagagaatggggtgaccttctaggctgtgattgcctgggctcaagctgagatgaaacgcagtgatgaggagcacaaaaccgtgctctgaggttaaataatgagggcttcggctatcagttcagagctcagtaaaaactgcagaggaggaggaagacctaattgcatgtagccagccacagggcaaatgagagctgcagcgtgctggggcagatccgggagcagaggggccgtggcacgctccctgttcactggctcccctggagccacacaaaaggccccttcctggcaattgtgcccacatcaatcattagctagaaacccagagctgggtaaatacgttttggcttcccgtcttgatgacagattgggtgttacatcacaaggtgggaccacttgatatgacaacacgctatatattcccgctgctacctctgcccttcctcccccactctgagagcaagcgggctgtgtgtgcaccgaggtgctctgccatgaggactgccaggcagtttgtacaggtggctctggccctctgctgctttgcaggtgagtgtttcctgctataccccgtaggtgactatagctagaccagagactaggctatctgtgagagtatctgggtattgtaatgtgttagagagccttgttccatgaaggaatgctctttctgacagtgtagcaaaacaccagactgcaagatccaggtttcagcaaacctcatacagacgactgttttcgtcgtggtttataggagcaaattgctgagggagcagtgctagtgcagggcaggagcttgcacgtgcaagcactgagtataacggcaaagcaaagctatgtgaaatggctcctgtgtccatgtaagcaatacaaacactgcatcttgtatcatctataaattttctgtgctgttcctggcagctgagaagtttgttgtgggaagaacagtgctagtggtcaacagccacctgaaacgtgcatgtctgagctcctgcaagtcaaatacagagtcttgcagaagagtttaaactcagtgcaggcttgaaaatacctacatttcttccctggggcatcttaggaactggctaacacatgtggcctcctactgaaagtgcagtgaaacttcatttaataacctctgattcattttatggacgtacatcactggcataatgtaaaattgcattttcctaaacccaataagccaatcaacaacggtatctaaatgtaactgtttcatcgaaagatttgcatatgtcatctctgcatattaataatatgtatttattttctgtctctacttttcttttagatattgcctttggaattgaggtgagttacagattttttttcccatttattcttttctattccaggcttctggtcaaataagagcagtatataattacctgatgagcaagtggattaatctaatgaaagcctggttgctcaaataatacttgccagtgcatgattgaatgatattgccaagtcacgaaaaagtaaaacacaccccgtttatactattttccattcatgcaataaaatgaagaaaggaagaattgtacgatcctattatgttaacttttggatataactgcgttagtccaagtcaaggggtggtagttacctcctcgagaggaaagctgtcttaagatgataagctccaaagcatcaaagacagtgattctggtatctttttctatacagtaagacacacactacagtgttcctgcctatacccatatcaaagcgaggaaagcagcagggtctgtgcagtgcatttgtctgcaggttcttcccacgcagttatgagattcctgcaaatcaccagagactgcagcgtgattggaaacgatcagattttgagttgagcggctgtggagcatggccaggctcccaattaccagctgccttcgttaggcgctgtctcacccacagctctccttcctccatgtcatgcttcccccagtcccccgcaggaaagcgtgatcagaagaagattcccacctcctgactgcctgagcagattccaaatgatacctcaggtgtttgtcccggctggagctgtgggtggcaggaggtttccatactgtcttttgttgtggaaactgaccccagggctgatgttgtgctgcttccataggttaattgcagcctgtatgccagcggcatcggcaaggatgggacgagttgggtagcctgcccgaggaacttgaagcctgtctgtggcacagatggctccacatacagcaatgagtgcgggatctgcctctacaacaggtgagcttatgtggaagcccaggggagctgcagggcaggagactcgaggtgagggcggcagctctgtccccaaaatatggtctgtgtggaggagtatgtgagttagtaccaggatgctgacctccagcctgggggtggtggctgctctctgccatctctgacacagatctgcgttcttccagggagcacggggcaaacgtggagaaggaatatgatggagagtgcaggccaaagcacgttacggtaagtccaacagtaagatgaagtcttgctctgttggtgcccataaagacttatttttatttcatagaatcattgaacagcttaggttggaagggaccttaaagatcattgggctctaacccccctggcctggccgggctgccttcaaccaaatcagtttgcccagtcaaatgggccttgggcacctccagggatggggcacctgctctgctcagcctgttacttatttacttgtttttttcccattcctgctatccttacagattgattgctctccgtacctccaagttgtaagagatggtaacaccatggtagcctgcccaaggattctgaaaccagtctgtggctcagatagcttcacttatgacaacgaatgtgggatttgcgcctacaacgcgtaagtcttttctgtggagcatccttctgggtaattagagatggctaagtcccttggaaacgcttacataaaacactttctaagcctttcttagggtagatgtttctgtgggactctttgaagctggctacttgtgattctccagccagctgcagatttcttccccatcctctgtctgtgctcatgaagggaatcacaaaaaagacagaggacaacccacagcagaggcatgaatagatcaaagtgttgctcagtgctgtgtgatatggaaataccatgcattttctgctcacaagtggttgctaccacctgtgggctgcatccagaccactcagcagttccttacgtgaagggtgggaccttgctttcttgccccagtatctaaggcttttcacgaggctctctaactaaaacagctctttctttcagagaacatcacaccaacatttccaaactgcacgatggagaatgcaagctggagatcggctcggtaagtgtaacagaaataaaaatccatctcctagggctgttaacggagagaatcccattgattttcctaagaaaatgtatgaccgggctgatcgggggtcccggtccacgctctgcttcctgcctggtgagggtggcttctgaaacaaagcggtaaaggaagaggccccagattttccttgcattgtgctgtgcagattggcaggtttctctctggaggcgacaagcatttccaccctttgtaacaagcattcaaaattctagtgctggtagcttggttagatatagtgagattcataagagcaccaagcatacatatttatagggtatagcttattgtatatttatactggggtaagagtccagtgcctcaggaagaaaagcttatatatttcagcacaaaaattctgggatgcagggagtccgttctccaacagacggattcctcctttatcacttcaactcccgtgcttaactgcagggaatctgaattattaagcaatcacagcactggggaaggaaggagaaaaaccaacacaaaccaaaacaatgttaatcagatttccagctgttggaaaatatttcccacttaattcaaggctgttgtgtcgatgagaagagggctgaaaaggctgttttcagttcctctgcctgaaggtttcattctctaagagaggtcccttttcttgtctcctagagaatgagggtagtgttctgaaagcctatttctgatagacagtttagttaagtgtagcagggctttgtcctgtcacaaaaactaggaagccgggaatacaggatgaaaaggtgttacattgacttctcccgtgtagcacaggctccgggagggcttattctccttattttggcaggttgactgcagtaagtacccatccacagtctctaaggatggcaggactttggtagcctgcccaaggatcctgagcccggtttgcggcaccgatggtttcacctatgacaacgaatgcgggatctgcgcccacaatgcgtaagtgctgctcatctcccactcctccaaagtagccagcaatgctttgccgtgctgggagccttccttctacgttgctgcttatgcctgtttcttcaagcctcttagaaactgcattttttttgttgttgttcttactgagttttcttctgatgccttctttgtgatcacgaggggaaatctgcaagactcagaacacagctccttggattagtctgtgggctgggcagtgactgagcagagaaaggaatagttcagaatcttgctttaaataacacgagaagacgtgatgagcttgttaacgagcagagtaatgtagctatatcaatacaatcgtgcagagaggctgaagccctactttgttaggtacctgctttaggctacgtctggttcattctgcatgcaagtgtttaaaccaagagttaaagcatctccttactcactttgtctccctctttcagagagcagaggacccatgtcagcaagaagcatgatggaaaatgcaggcaggagattcctgaagtgagtatacaacgtaaggtgtatttctccccttgcctctgcccactgagctatttgctgaggccacgtctactctgaaagtgagctggcttgaagcctggctctctgcacgtgtcctttgggatgtgccaacgtgtatccaacacacaaacagtgtggaagttgggcagggggaacttaggtcttttaaggatgatcactaaatgcattgccagcaaagtccttttgtgccagtgaagtcctattatgtttgccttcttttgtttcattctatagtgcagagagaaaaggagatgatatatctttgttggttttttttttgtttgtttgttttgcttttctgccatatctagcaaactgtttcagtaggttgtgacccctttggatcacaagtgaagctcagtggcatttgggattgactgagctgtctgccctggtgatttggcatctcacagattacacagcgccatgtagctcctcctgggcatgagagagtttctgcagagctgactcaggctggctttgagagaactgaagtgtagcaccagcgttgtttcagcatcccagcgtaaaagacatggattgcagcaggaggcaatgctagggtttgtctttgagagcaagggctttttcagggctgacgctcctactttttgcagattgactgtgatcaatacccaacaagaaaaaccactggtggcaaactcctggtgcgctgcccaaggattctgctcccagtctgtggcacagacggatttacttatgacaacgagtgtggcatttgtgcccataatgcgtaagtactgcaaacaggacttccttttgtagcgactagccacgttagtactgcagatggcttcccctccacccttcatcttcttctttctttctttttttttgatagcagtatgtctatatgtctcctgttcttccttcaacctcctgaagctctgtcgcctcggtttcctttcctgatgtgctcctcagggagctgtgggagagccagctaacagctgagtgtcctatgagggctgtggcatttgtgcagaggaaaaagagaatgggtctgctacaagtagacctgagaagcctgtaacttcttaggatcatgatccctaatggcagcctttccctttcagacaacatgggactgaggttaagaagagccacgatggaagatgcaaggagcggagcaccccggtaagtggggatggatgtcagatgagcgccagctcctgtacgtgccttgtggctgcagaggttgctaaccagggtctgtccattcaggcagcagagaaggggaatgggccaggatttaggtaacaaaatgtcccaatactgcaggtctctggagggaaacatcagaggcagcccagaacagcacagcctgttttagcacagtaggagaggaagagcagaagctgtgttagatgcctgtgtagtcattcagtgctaggatttccattgcagcagacaggttaaaaaatctctgtaccgtggtcagccaagaaaaggctgcttgcaggaatgcacgcagaaatagctctataaacatgcacggtaacaatatgtgctgataatatctcagcacatttattctgcttatgcagagcagctctaaaacactgaaaataactttgtgcatctcaagggattgctgtatcttttctgtagtaaagacacactgttatggtgctgtctttgctataatttgctcttggactgtgtggggaaatatgggtaataagagctactacacaggggaaggtatgcaaaacgattgtgaagtgtcagaagcttagccagtgtagactgacttccagtgccatcagtagatacttgcttatttatcctcaaatattggaactgtttttaagtactgtgaggatttctgcagcagcagctgatgagctgatggaacagtttcttcttgccgttttgaaaacgtggaaacaaaatctaaggcttagctaagtcaggcatgacctaatgtcaaactggacataacatcaaactccttatatcaaattcctttgaataatgcttgttttgaaacttggacatacgctgcataaggaagatgatctttctggtctgctattcctttgcgttccctttgttagtgagcaatatcaaacccaaccacaattagttcatttataatgggagactaaactgaaatcaaccctgatttttcctatggctcgaggcagtctgtcccccagctcccagcacctgactcagcatccttactgttttctccccagcttgactgcacccaatacctgagcaatacccaaaacggtgaagccattaccgcctgccccttcatcctgcaggaggtctgtggcactgacggcgtcacctacagcaacgactgttctctgtgtgcccacaacatgtaagccctgcaggtcacccactcgtgtgtcaccgcagctgcttgttgagctttgtcaactctgttttctctctcttccagtgaattgggaaccagcgttgccaaaaagcacgatgggaggtgcagagaggaggttcctgaggtaagcgataaagaaaacaagagcttgaggtggtgcttattgcctaacaagtacaacgctggctggttttggtgatgctgggtcatgccctcctgctgccatccttcctgcaggtaaacatcaaccctggcagcagggatgctgtgcattttctgcatgtagtcagggaaagaaagagaagaggacgggtgaggaatgagttatgatgcaggtagcataaatgatttaaggcgttacgaagaaatctctttcccacagcagtctatcatacctgccgtgggagtgtagctgtctgttctggcaatatgggaaagggacacagagcacccgcaggtacctggtgccttctggatacctgtgctgtgcaaaaggatgttgtgcaaagatcagaaaactacctgcattttgaatgcttttacctaatgtaccagaggattcaaacacctctctcttcctattgtaaatgcgatataatgtaatgtataccaacaatgaatcttgtaaaaataccagataaactatatttggccagctctaaactatttacgctcactggggaatagaaaaacaaagccatctcattatcttgtgtttgaaagagtcaacgtcgtgagtcagatatttcatttctatgcaaacagactatgaaatgtcattgctttgtttcctgcgtatgctctgtgctcagaccaagtcagatgcataaatcagtgaggaagagctcacactggagaaactgggatagctgaaactcaaggccagttcttcaaatggcataaatcattttgaactgctgttggtccttctgtccgattgcaacacacagaaccagcccctcgcaacaaaaggcatgtcagcacatctcctcagttcttgtgggccgtgacacactccttggccacactgagcttctcttgcaggaattgcataaatcacgccagtttgatttgcagattatttatgagctgcgttttgcagcgtcccagcaagtggttcagcaagctctaagggcatcgtgataaatgcagggctgaatgagtgatacgcgccttcaagctttgattcagtcttctccagtataaggctgtgacagaaaattgatagttttcaatgaagaatgagtcaatgcataaccataatccatcctgtggcagatcttgaaaggcagaggcgtaaggaagggggttgtgtctgagcacccttacacagagcatttgctgcctttgtttcctagcttgactgcagcaagtacaaaacctccacgctgaaggatggcagacaggtggtggcctgcaccatgatctacgatcccgtctgtgctaccaatggtgtcacctatgccagcgaatgcacgctgtgcgctcacaacctgtaagtactcattcatctccagggggacccaccgtggctgtgactggacacatctttgagtgctgaataacatgcaagggctctgtctaaaatctcgtgctgcatgggtcctgtctgcctatccccgtttccctggttgccatggttggtgtttgagatgggcatttagcaaggcccactgcccccagtgacccagaaaaagggttcactgcctgggaaagcattattccaaaagacacatccctagtccttaagggcatgttcttgctaatgcttctcaggcaatgcttagctaatttatctgaaattgtcctgtgtaccacatgggaacgaggttgtgctcttgtactacggttgtaaatgggaagggtttctgctaatatccatctctccttcctccagggagcagcggaccaatcttggcaagagaaagaatggaagatgtgaagaggatataacaaaggtgagtgtgaaaggatgggcacaaagagttacagtcgtaggggaccgtcctctgctccacatcaaaaactgggggagcggtgtgcagccctggcgaggtcgcttgggaatgtcatactggttatagaatagctgccatccatcccatgggaatggacatggcagtgaacaggaacagtgtgaggtcacatccctcaccaggaggaactgagctgattactgccgtaattttccagtttcactctttgtgctgggggaatactgtttgctcccaggcagagactcacatcttccttgtgtgtgcaggaacattgccgtgagttccagaaagtctctcccatctgcaccatggaatacgtaccccactgtggctctgatggcgtaacatacagcaacagatgtttcttctgcaacgcatatgtgtaagtataggagtgaaacccttcctgtaactgctacaaacgcagagttgattttataaggagttctttactaacactttatgggtgtgtgctagacatttcggatgcaccgtgacgtgcaaggaggtgcttttttgctttttaagaaaaaatgcaaagcacccacatctgcccatgtgtatgtggcttcctgttttatttagtttcaaagacattttgctaattttcaccagcatagtttgtcccacaagctcatcagggtatggggaaagtacttcaccaaactacctggagcgtttcaagtgtgtgaaacctgtcatctttcctttaattttcataatgaaaggaagtggttggccttctgagactgttctttatcttctgccaacattatcaacatttgggctggtaaggagaggaacaaggctgcagcacaaattctattgtgtttaatcctttcttctcttttcattaggcagagcaataggactctcaacctcgtgagtatggcagcgtgttaactctgcactggagtccatcgtgggaaacaatctgccttgcacatgagtcttcgtgggccaatattccccaacggttttccttcagcttgtcttgtctcccaagctctcaaaacacctttttggtgaataaactcacttggcaacgtttatctgtcttaccttagtgtcacgtttcatccctattcccctttctcctcctccgtgtggtacacagtggtgcacactggttcttctgttgatgttctgctctgacagccaatgtgggtaaagttcttcctgccatgtgtctgtgttgttttcacttcaaaaagggccctgggctccccttggagctctcaggcatttccttaatcatcacagtcacgctggcaggattagtctctcctaaaccttagaatgacctgaacgtgtgctccctctttgtagtcagtgcagggagacgtttgcctcaagatcagggtccatctcacccacagggcaattcccaagatgaggtggatggtttactctcacaaaaagttttcttacgttttgctagaaaggagagctcactgcctacctgtgaattcccctagtcctggttctgctgccaccgctgcctgtgcagcctgtcccatggagggggcagcaactgctgtcacaaaggtgatcccaccctgtctccactgaaatgacctcagtgccacgtgttgtataggatataaagtacgggaggggaatgcccggctcccttcagggttgcagggcagaagtgtctgtgtatagagtgtgtgtcttaatctattaatgcaacagaacaacttcagtcctggtgttttgtgggctggaattgcccatgtggtagggacaggcctgctaaatcactgcaatcgcctatgttctgaaggtatttgggaaagaaagggatttgggggattgcctgtgattggctttaattgaatggcaaatcacaggaaagcagttctgctcaacagttggttgtttcagccaattcttgcagccaaagagccgggtgcccagcgatataatagttgtcacttgtgtctgtatggatgacagggaggtagggtgacctgaggaccaccctccagcttctgccagcgtaggtacagtcaccacctccagctccacacgagtcccatcgtggtttaccaaagaaacacaattatttggaccagtttggaaagtcacccggtgtattgtgaggctagattaataggctgaaggcaaatgttcccaacttggagatactgttggtattgtatcagggaacagggccatagcacctccatgctattagattccggctggcatgtacttttcaagatgatttgtaactaacaatggcttattgtgcttgtcttaagtctgtgtcctaatgtaaatgttcctttggtttatataaccttcttgccgtttgctcttcaggtgttcttgcagaacactggctgctttaatctagtttaactgttgcttgattattcttagggataagatctgaataaactttttgtggctttggcagactttagcttgggcttagctcccacattagcttttgcagccttttctgtgaagctatcaagatcctactcagtgacattagctgggtgcaggtgtaccaaatcctgctctgtggaacacattgtctgatgataccgaaggcaaacgtgaactcaaagaggcacagagttaagaagaagtctgtgcaattcagaggaaaagccaaagtggccattagacacactttccatgcagtatttgccagtaggtttcatataaaactacaaaatggaataaaccactacaaatgggaaaaacctgatactggaatttaaatattcacccaggctcaaggggtgtttcatggagtaacatcactctataaaagtagggcagccaattattcacagacaaagcttttttttttttctgtgctgcagtgctgtttttcggctgatccagggttacttattgtgggtctgagagctgaatgatttctccttgtgtcatgttggtgaaggagatatggccagggggagatgagcatgttcgagaggaaacgttgcattttggtggcttgggagaaaggtagaacgatatcaggtctacagtgtcactaagggatctgaaggatggttttacagaacagttgacttggctgggtgcaggcttggctgtaaatggatggaaggatggacagatgggtggacagagatttctgtgcaggagatcatctcctgagctcggtgcttgacagactgcagatccatcccataaccttctccagcatgagagcgcggggagctttggtactgttcagtctgctgcttgttgcttcctgggtgcacagtggtgattttcttactcacacagggcaaaaacctgagcagcttcaaagtgaacaggttgctctcataggccattcagttgtcaagatgaggtttttggtttcttgttttgtaaggtgggaagaagcactgaaggatcggttgcgagggcaggggtttagcactgttcagagaagtcttattttaactcctctcatgaacaaaaagagatgcaggtgcagattctggcaaggatgcagtgaaggagaaagccctgaatttctgatatatgtgcaatgttgggcacctaacattccctgctgaagcacagcagctccagctccatgcagtactcacagctggtgcagccctcggctccagggtctgagcagtgctgggactcatgaggttccatgtctttcacactgataatggtccaatttctggaatgggtgcccatccttggaggtccccaaggccaggctggctgcgtctccgagcagcccgatctggtggtgagtagccagcccatggcaggagttagagcctgatggtctttaaggtcccttccaacctaagccatcctacgattctaggaatcatgacttgtgagtgtgtattgcagaggcaatattttaaagttataaatgttttctccccttccttgtttgtcaaagttatcttgatcgccttatcaatgcttttggagtctccagtcatttttcttacaacaaaaagaggaggaagaatgaagagaatcatttaatttcttgattgaatagtaggattcagaaagctgtacgtaatgccgtctctttgtatcgagctgtaaggtttctcatcatttatcagcgtggtacatatcagcacttttccatctgatgtggaaaaaaaaatccttatcatctacagtctctgtacctaaacatcgctcagactctttaccaaaaaagctataggttttaaaactacatctgctgataatttgccttgttttagctcttcttccatatgctgcgtttgtgagaggtgcgtggatgggcctaaactctcagttgctgagcttgatgggtgcttaagaatgaagcactcactgctgaaactgttttcatttcacaggaatgttttagtggcattgtttttataactacatattcctcagataaatgaaatccagaaataattatgcaaactcactgcatccgttgcacaggtctttatctgctagcaaaggaaataatttggggatggcaaaaacattccttcagacatctatatttaaaggaatataatcctggtacccacccacttcatccctcattatgttcacactcagagatactcattctcttgttgttatcatttgatagcgttttctttggttctttgccacgctctgggctatggctgcacgctctgcactgatcagcaagtagatgcgagggaagcagcagtgagaggggctgccctcagctggcacccagccgctcagcctaggaggggaccttgcctttccaccagctgaggtgcagccctacaagcttacacgtgctgcgagcaggtgagcaaagggagtcctcatggtgtgtttcttgctgcccggaagcaaaactttactttcattcattccccttgaagaatgaggaatgtttggaaacggactgctttacgttcaatttctctcttccctttaaggctcagccaggggccattgctgaggacggcatcggggccccctggaccaaatctgtggcacagatggtttcacttacatcagtggatgtgggatctgcgcctgtaatgtgtccttctgaaggaaggaacgtgccttccaagtgccagccccacagcccccagcccctccctgtgctgctccaattcatctcctcttcctccttctccctttgctgtttgtgctcgggtagaaatcatgaagatttagaagagaaaacaaaataactggagtggaaacccaggtgatgcagttcattcagctgtcataggtttgtcattgctataggtctgtatcagagatgctaacaccactttgctgtcggtgcttaactcgggtgaactctccttcactcgcatcatttgcgggccttatttacatccccagcatccatcaccctctgggaaaatgggcacactggatctctaatggaagactttccctctttcagagcctgtgggatgtgcagtgacaagaaacgtggaggggctgagcagcagcactgcccccagggagcaggagcggatgccatcggtggcagcatcccaaatgatgtcagcggatgctgagcaggcagcggacgaacagacagaagcgatgcgtacaccttctgttgacatggcatttggcagcgatttaacactcgcttcctagtcctgctattctccacaggctgcattcaaatgaacgaagggaagggaggcaaaaagatgcaaaatccgagacaagcagcagaaatatttcttcgctacggaagcgtgcgcaaacaaccttctccaacagcaccagaagagcacagcgtaacctttttcaagaccagaaaaggaaattcacaaagcctctgtggataccagcgcgttcagctctcctgatagcagatttcttgtcaggttgcaaatggggtatggtgccaggaggtgcagggaccatatgatcatatacagcacagcagtcattgtgcatgtattaatatatattgagtagcagtgttactttgccaaagcaatagttcagagatgagtcctgctgcatacctctatcttaaaactaacttataaatagtaaaaccttctcagttcagccacgtgctcctctctgtcagcaccaatggtgcttcgcctgcacccagctgcaaggaatcagcccgtgatctcattaacactcagctctgcaggataaattagattgttccactctcttttgttgttaattacgacggaacaattgttcagtgctgatggtcctaattgtcagctacagaaaacgtctccatgcagttccttctgctccagcaaactgtccaggctatagcaccgtgatgcatgctacctctcactccatccttcttctctttcccaccagggagagctgtgtgttttcactctcagccgctctgaacaataccaaactgctacgcactgcctccctcggaaagagaatccccttgttgcttttttatttacaggatccttcttaaaaagcagaccatcattcactgcaaacccagagcttcctgcctctccttccacaaccgaaaacagccggcttcatttgtcttttttaaatgctgttttccaggtgaattttggccagcgtgttggctgagatccaggagcacgtgtcagctttctgctctcattgctcctgttctgcattgcctctttctggggcttccaagagggggggagactttgcacggggatgagataatgccccttttcttagggtggctgctgggcagcagagtggctctgggtcactgtggcaccaatgggaggcaccagtgggggtgtgttttgtgcagggaggaagcattcacagaatggggctgatcctgaagcttgcagtccaaggctttgtctgtgtacccagtgaaatccttcctctgttacataaagcccagataggactcagaaatgtagtcattccagcccccctcttcctcagatctggagcagcacttgtttgcagccagtcctccccaaaatgcacagacctcgccgagtggagggagatgtaaacagcgaaggttaattacctccttgtcaaaaacactttgtggtccatagatgtttctgtcaatcttacaaaacagaaccgagggcagcgagcactgaaggcgtgttcccatgctgagttaatgagacttggcagctcgctgtgcagagatgatccctgtgcttcatgggaggctgtaacctgtctccccatcgccttcacaccgcagtgctgtcctggacacctcaccctccataagctgtaggatgcagctgcccagggatcaagagacttttcctaaggctcttaggactcatctttgccgctcagtagcgtgcagcaattactcatcccaactatactgaatgggtttctgccagctctgcttgtttgtcaataagcattttttcattttgcctctaagtttctctcagcagcaccgctttgggtgacttcagtggccgcctggaacccgaggggcacagccaccacctccctgttgctgctgctccggggactcacgtgctgctggatggggggaagcatgaagttcctcacccagacacctgggttgcaatggttgcagtgtgctcttcttggtatgcagattgtttctagccattacttgtagaaatgtgctgtggaagccctttgtatctctttctgtggcccttcagcaaaagctgtgggaaagctctgaggctgctttcttgggtcgtggaggaattgtatgttccttctttaacaaaaattatccttaggagagagcactgtgcaagcattgtgcacataaaacaattcaggttgaaagggctctctggaggtttccagcctgactactgctcgaagcaaggccaggttcaaagatggctcaggatgctgtgtgccttcctgattatctgtgccaccaatggaggagattcacagccactctgcttcccgtgccactcatggagaggaatattcccttatattcagatagaatgtcatcctttagctcagccttccctataaccccatgagggagctgcagatccccatactctcctcttctctggggtgaaggccgtgtcctccagccccccttcccaccctgtgccctgagcagcccgctggcctctgctggatgtgtgcccatatgtcaatgcctgtccttgcagtccagcctggaacatttaattcatcaccagggtaatgtggaactgtgtcatcttcccctgcagggtacaaagttctgcacggggtcctttcggttcaggaaaaccttcgctggtgctacctgaatcaagctctatttaataagttcataagcacatggatgtgttttcctagagatacgttttaatggtatcagtgatttttatttgctttgttgcttacttcaaacagtgcctttgggcaggaggtgagggacgggtctgccgttggctctgcagtgatttctccaggcgtgtggctcaggtcagatagtggtcactctgtggccagaagaaggacaaagatggaaattgcagattgagtcatgttaagcaggcatcttggagtgatttgaggcagtttcatgaaagagctacgaccacttattgttgttttccccttttacaacagaagttttcatcaaaataacgtggcaaagcccaggaatgtttgggaaaagtgtagttaaatgttttgtaattcatttgtcggagtgttaccagctaagaaaaaagtcctacctttggtatggtagtcctgcagagaatacgacatcaatattagtttggaaaaaaacaccaccaccaccagaaactgtaatggaaaatgtaaaccaagaaattccttgggtaagagagaaaggatgtcgtatactggccaagtcctgcccagctgtcagcctgctgaccctctgcagctcaggaccatgaaacgtggcactgtaagacgtgtccctgcctttgcttgctcacagatctctgccctcgtgctgactcctgcacacaagagcatttccctgtagccaaacagcgattagccataagctgcacctgactttgaggattaagagtttgcaattaagtggattgcagcaggagatcagtggcagggttgcagatgaaatcctttctaggggtagctaagggctgagcaacctgtcctacagcacaagccaaaccagccaagggttttcctgtgctgttcacagaggcagggccagctggagctggaggaggttgtgctgggactcttctccctgtgctgagaatggagtgatttctgggtgctgttcctgtggcttgcactgagcagctcaagggagatcggtgctcctcatgcagtgccaaaactcgtgtttgatgcagaaagatggatgtgcacctccctcctgctaatgcagccgtgagcttatgaaggcaatgagccctcagtgcagcaggagctgtagtgcactcctgtaggtgctagggaaaatctctggttcccagggatgcattcataaggacaatatatcttgaggctgtgccaaatctttctgaaatattcatgcatgttcccttaatttatagaaacaaacacagcagaataattattccaatgcctcccctcgaaggaaacccatatttccatgtagaaatgtaacctatatacacacagccatgctgcatccttcagaacatgccagtgctcatctcccatggcaaaatactacaggtattctcactatgttggacctgtgaaaggaaccatggtaagaaactcaggttaaaggtatggctgcaaaactactcataccaaaacagcagagctccagacctcctcttaggaaagagccacttggagagggatggtgtgaaggctggaggtgagagacagagcctgtcccagttttcctgtctctattttctgaaatgtctgcaggaggaaaggacaactgtactttcaggcatagctggtgccctcacgtaaataagttccccgaacttctgtgtcatttgttcttaagatgctttggcagaacactttgagtcaattcgcttaactgtgactaggtctgtaaataagtgctccctgctgataaggttcaagtgacatttttagtggtatttgacagcatttaccttgctttcaagtcttctaccaagctcttctatacttaagcagtgaaaccgccaagaaacccttccttttatcaagctagtgctaaataccattaacttcataggttagatacggtgctgccagcttcacctggcagtggttggtcagttctgctggtgacaaagcctccctggcctgtgcttttacctagaggtgaatatccaagaatgcagaactgcatggaaagcagagctgcaggcacgatggtgctgagccttagctgcttcctgctgggagatgtggatgcagagacgaatgaaggacctgtcccttactcccctcagcgttctgtgctatttagggttctaccagagtccttaagaggtttttttttttttttggtccaaaagtctgtttgtttggttttgaccactgagagcatgtgacacttgtctcaagctattaaccaagtgtccagccaaaatcaattgcctgggagacgcagaccattacctggaggtcaggacctcaataaatattaccagcctcattgtgccgctgacagattcagctggctgctctgtgttccagtccaacagttcggacgccacgtttgtatatatttgcaggcagcctcggggggaccatctcaggagcagagcaccggcagccgcctgcagagccgggcagtacctcaccatggccatggcaggcgtcttcgtgctgttctctttcgtgctttgtggcttcctcccaggtgagtaactcccagagtgctgcagaagctttgtgcctgccagtcctggctctccttagcagaacatggtggtgaccatcagagagagactcccctacaaagtgcctgcaaaggctgcctcagtacatcagtattaaacggattactgttgtgctgggtgtctgttgggttctgtgctcccaacacatttcttacgctctcagctctgttacactgcttgcatttgctgcacagttgcatagaatggataaatgcttgaaacaaggccataacgaggtggtcagacctccaggaactagttagggaaatattgtcatggcccaagcaagctctgtgcaggaacctggcagctttcctgcaatgcttttgctgctaatggagaaacaagagatgcaaacaagccaggatctgatgttctccttctgtatttacatctcatgaaattacaaagtcaaagacaagcgtggtttatttcttacactcagcttctttaaaatgtatatccctgacaacagatgctgtgtatgtttgcttatcctgtatgtgactatttgcatttgcatttatctctattgactcaggtttcttttcagatatgtgatagatgttttctagggacaaaacggatgtgtgaatagataaggaaggaaaagatattcatttttcaattaataaatctacctatctcttaacttttttttttttttaagaacagagctattcaagaactcgtttcatcagccagcaataagaagctaaattatgtttatcagcattaaacaaaaatcatatatagtttgcttagttcaagaatcgaatcggtggaaatcactcagtttggttctctgtgctggagttttgcacacacatttcagctagctgtggtctcactgatcagactgcctttgtttcccatttttgtcccctttttttccccagatgctgcctttggggctgaggtgagtaagagagttcttcttgtccacttttctcttttctcttttctctctctctctttttttccccccgtcttaattagtatcactataatcagatcccagagtgtaaaatgttaaattatgcagttctgagctctacatctatgctgcatgtaagtaatgtagcagtgatataaaactgttagatgaattaatttctgaccaactctgaactggtctaagctttaagttgatcatatgttctactaaataatacagtggtttgggttggaagggtcctttaagatcatctacttccaacccctctgctataggcagggacaactcccactagacaagattgctcaaagctccatccatatgatcagctgtagactgatggctgtagactatagcattaaaaactaccccaaagcagcctactgaaagaagaaagtactgtgaggtgctacagcttccaaatcccatgttgttagacctgttcttttgaataaacgtgtttgtacgttgagaatgaatgagtaacaatggcagaacactggaggggccaactctcaggctttgcaaaatggtgcctggggggcatgatagatccctgctggtttatcacatggggagctgcatggctataaccccattgcccagttctctcccactgcatggagagaaggctggatctggtcgctgccctgctgaaaatggcagatgtaactacaaaatgtcactttgtcctgttactgtgtgtttctttgtcaggtggactgcagtaggtttcccaacgctacagacaaggaaggcaaagatgtattggtttgcaacaaggacctccgccccatctgtggtaccgatggagtcacttacaccaacgattgcttgctgtgtgcctacagcatgtgtgtactgcagagagagctcatactgcaagcaagcagctgtgcttagggctcctgacagcacccctttccaacaaacagtgatctgtcacatgtcacttatgtcaactctttcagggaaagcttgagtatcactgcgtgacactcggttgcctagacatcactttggttactgtgtcttttttgttgatgtaatttattcaggtttttctcctccatctcggggatgaggcagatgacagcccctagggcatatttcatcccagcaaaaaaggagcaaaaggatggagaggtgctccagtctgaatggtccaaaacagtcctaaagatttcagagtctttagatccctgccagccactcagtatggcactaccctctccaatacaaatatatatatatacaaagatgacttagccagactcagcctcattgcattaggtacatattcccaataacgagaagctgagcttcctaatacctgttttccctcttcagagaatttggaaccaatatcagcaaagagcacgatggagaatgcaaggaaactgttcctgtaagtgaaaccaagttcatcctttgtgcagccaaaactgcttattgacttgcccaataaataatgtaaatgctgactaagaggccatgtgagatgtcagaatcttgtattgatcatcttcaggtgaagtttcatcacaataacacaaaaaaagactttatttcctgctgaggtggcattttaggagacccaacgcacgcgctccgctggtctacgtggtccctgtaagccctcaccagcgctttgctgtgtgctccttccacagatgaactgcagtagttatgccaacacgacaagcgaggacggaaaagtgatggtcctctgcaacagggccttcaaccccgtctgtggtactgatggagtcacctacgacaatgagtgtctgctgtgtgcccacaaagtgtaagtaccgagctgtgctcccttggcaggaatgggtcctgcgctcctggcagccactctttgagcactgggatttccaatgaggctttttctgtatggctcttggactccgtccctcctctccctgataacctcatgctgttttcctttgtgattagaaagagaactgtggctttgatcttgagagagaagcagagagctgggtggggacttaagagaagcactctgttctgtgttaactaagttaaaagggtctgtgtggcacacactgccttgcagaggacagcagtgaacctctgctgcacctatattgtaaaacaacctagctcctaggccatgacagcctgtcacctctcctcctttgcatcatgcaatactgcaacactgtggcacatagtaccacctcccataaggactgatatgttgaaccagtgtgtcagagaccagtagcatctctgtcttcaggatcatcaggtagcattctatatacagggtgttgcccaggactccgagtcccatgaagtatggcaggggttttggaactggatgaccttcgaggtcacttccaacccaagccattctattattctgtgaaagccagggaggtgggggtgcttgcagggctggtatcttgagcagtgtgggcacaaactaggctgggcatctgcagcccatcagcactgcggggatgtggagttcagcacagcaggatgcaggcacagctccctaacatggatttttttcctttcagagagcagggggccagcgttgacaagaggcatgatggtggatgtaggaaggaacttgctgctgtgagtgtgagtagcacaatgaaggagcaggttctggtcccactgatgtcaagggaaacatggccagcatctttagtagcctcaggagcatcagttgtgcttcagcacagagaagattttactttctacacacgtaatacacattatccacagtaatgtcaggaagggaagaggatgactgcacaggcagggatcagtaaaagaccataagcagaaataacccatgagggcagaactgagaataagaactgagactagatccagggggtcagaccaatgggccatcaaacccatgatggtttgatgcagagtccactctttcagcattcataagaattgagtaggggggagtaagggtggggtgagtacgtacggatcttcccaaacacccttccaacctacagctatgcacctcagccaggtgtgatttctgtgtagttcacaagcctcagtggatttctctcccatgggattctccagcctctttctggacctgtatacacggtagttgggttggttttttttttctgtctctctttttttccccccactacaatgtccctcagcaaacatagtcctcatctctcaaacaaacaaatctcattctctaagtacccagataagagctgatttttgctttaagcctgtgggggagatgctggactattataaaggtatcagtgctgcctcttctccagacaccaatgttttttccatttaatttcctgaacaggtcaggaacacggtgcaacatgattgtaagcacagcacgttcatggagcgagctgctgctgcagctcagaaatgcagcagtcagattgtgatatgcatctcttacacaggaaattatgctctatttttatattattaaatctagcatacgagaaaggacatccagtttatatcagatcgtgcaaggaagttaattatttttagtttgatcattatcatcggcactgcagctgtagctagggaggggttgaagctcttcagctatcgactccttcatatcctccacgttacaattgtgtttttgcaggttgactgcagcgagtaccctaagcctgactgcacggcagaagacagacctctctgtggctccgacaacaaaacatatggcaacaagtgcaacttctgcaatgcagtcgtgtacgtacagccctgattgcattcacgttgtcggctgcctcctacaggcaccagcttgcacagttcctgctttcgttgctgattgctgaccaggatctgggggcagaaaagaacaccgggcatcacgccagccattcatttgatttttcaccagagcttgtctggtttgttaggatggatgttttgaacgccattaaccttaagggaagttttccttgctgcgaagaaaatcagatttggtgtttcattatagttttcagaaggggttaaacgatttcactcatctcctaataatcaggtagctgaggagatgctgagtctgccagttcttgggctctgggcaggatcccatctcctgccttctctaggacagagctcagcaggcagggctctgtggctctgtgtctaacccacttcttcctctcctcgctttcagggaaagcaacgggactctcactttaagccattttggaaaatgctgaatatcagagctgagagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatgggtgtactgctcacacagaggacgctgctcagtctggtccttgcactcctgtttccaagcatggcgagcatggcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagataccgtgcccagattctgatcaggagcccaaatcttctgacaaaactcacacatccccaccgtccccagcacctgaactcctgggtggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagga

SEQ ID NO: 45 - DNA, 780 bp, SV40
cccagagctgtgcagttgggatcctaacaccatgcagatgctccaggacctgcaccgagccccagcactggcactcatctcttctttccacccctctgagagcaacaagtggctctgcaatggcaatgtaagtgaaaccgggcgggtatcttagagcacctggaagcttgcatgcctgcaggtcgactctagaggatccccgggtaccgagctcgaattccaggtaccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacagccaagccgaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcag

SEQ ID NO: 46 - DNA, 1957 bp, human
ataatcaggtagctgaggagatgctgagtctgccagttcttgggctctgggcaggatcccatctcctgccttctctaggacagagctcagcaggcagggctctgtggctctgtgtctaacccacttcttcctctcctcgctttcagggaaagcaacgggactctcactttaagccattttggaaaatgctgaatatcagagctgagagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatgggtgtactgctcacacagaggacgctgctcagtctggtccttgcactcctgtttccaagcatggcgagcatggcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagataccgtgcccagattctgatcaggagcccaaatcttctgacaaaactcacacatccccaccgtccccagcacctgaactcctgggtggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagg

SEQ ID NO: 47 - DNA, 8565 bp, Artificial Sequence
  pSIN-1.8-OM-I-GCSF-3′UTR
gatctctgcccttgtgctgactcctgcacacaagagcatttccctgtagccaaacagcgattagccataagctgcacctgactttgaggattaagagtttgcaattaagtggattgcagcaggagatcagtggcagggttgcagatgaaatccttttctaggggtagctaagggctgagcaacctgtcctacagcacaagccaaaccagccaagggttttcctgtgctgttcacagaggcagggccagctggagctggaggaggttgtgctgggacccttctccctgtgctgagaatggagtgatttctgggtgctgttcctgtggcttgcactgagcagctcaagggagatcggtgctcctcatgcagtgccaaaactcgtgtttgatgcagaaagatggatgtgcacctccctcctgctaatgcagccgtgagcttatgaaggcaatgagccctcagtgcagcaggagctgtagtgcactcctgtaggtgctagggaaaatctctggttcccagggatgcattcataagggcaatatatcttgaggctgcgccaaatctttctgaaatattcatgcgtgttcccttaatttatagaaacaaacacagcagaataattattccaatgcctcccctcgaaggaaacccatatttccatgtagaaatgtaacctatatacacacagccatgctgcatccttcagaacgtgccagtgctcatctcccatggcaaaatactacaggtattctcactatgttggacctgtgaaaggaaccatggtaagaaacttcggttaaaggtatggctgcaaaactactcataccaaaacagcagagctccagacctcctcttaggaaagagccacttggagagggatggtgtgaaggctggaggtgagagacagagcctgtcccagttttcctgtctctattttctgaaacgtttgcaggaggaaaggacaactgtactttcaggcatagctggtgccctcacgtaaataagttccccgaacttctgtgtcatttgttcttaagatgctttggcagaacactttgagtcaattcgcttaactgtgactaggtctgtaaataagtgctccctgctgataaggttcaagtgacatttttagtggtatttgacagcatttaccttgctttcaagtcttctaccaagctcttctatacttaagcagtgaaaccgccaagaaacccttccttttatcaagctagtgctaaataccattaacttcataggttagatacggtgctgccagcttcacctggcagtggttggtcagttctgctggtgacaaagcctccctggcctgtgcttttacctagaggtgaatatccaagaatgcagaactgcatggaaagcagagctgcaggcacgatggtgctgagccttagctgcttcctgctgggagatgtggatgcagagacgaatgaaggacctgtcccttactcccctcagcattctgtgctatttagggttctaccagagtccttaagaggttttttttttttttggtccaaaagtctgtttgtttggttttgaccactgagagcatgtgacacttgtctcaagctattaaccaagtgtccagccaaaatcaattgcctgggagacgcagaccattacctggaggtcaggacctcaataaatattaccagcctcattgtgccgctgacagattcagctggctgctctgtgttccagtccaacagttcggacgccacgtttgtatatatttgcaggcagcctcggggggaccatctcaggagcagagcaccggcagccgcctgcagagccgggcagtacctcaccattgccttggcaggcgtcttcgtgctgttctctttcgtgctttgtggcttcctcccaggtgagtaactcccagagtgctgcagaagctttgtgcctgccagtcctggctctccttagcagaacatggtggtgaccatcagagagagactcccctacaaagtgcctgcaaaggctgcctcagtacatcagtattaaacggattactgttgtgctgggtgtctgttgggttctgtgctcccaacacatttcttacgctctcagctctgttacactgcttgcatttgctgcacagttgcatagaatggataaatgcttgaaacaaggccataacgaggtggtcagacctccaggaactagttagggaaatattgtcatggcccaagcaagctctgtgcaggaacctggcagctttcctgcaatgcttttgctgctaatggagaaacaagagatgcaaacaagccaggatctgatgttctccttctgtatttacatctcatgaaattacaaagtcaaagacaagcgtggtttatttcttacactcagcttctttaaaatgtatatccctgacaacagatgctgtgtatgtttgcttatcctgtatgtgactatttgcatttgcatttatctctattgactcaggtttcttttcagatatgtgatagatgttttctagggacaaaacggatgtgtgaatagataaggaaggaaaagatattcatttttcaattaataaatctacctatctcttaacttttttttttttttaagaacagagctattcaagaactcgtttcatcagccagcaataagaagctaaattatgtttatcagcattaaacaaaaatcatatatagtttgcttagttcaagaatcgaatcggtggaaatcactcagtttggttctctgtgctggagttttgcacacacatttcagctagctgtggtctcactgatcagactgcctttgtttcccatttttgtcccctttttttccccagattctgccaacatggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctgaaaagtctagtatggggattggtggcgacgactcctggagcccgtcagtatcggcggaattgggtctagaatatcagagctgagagaattcaccacaggatccccactggcgaatcccagcgagaggtctcacctcggttcatctcgcactctggggagctcagctcactcccgattttctttctcaataaactaaatcagcaacactcctttgtcttgtttaatgctctgcctcatgcaatgttttcttctgatttgttggacggtgataccagactcaatatgttccatgctcgtggctctggggtataacaagaacaacatcttgctcccatccctgtcataaaaggcagaaaattaaatacagatgcataaacctcggctgtgtgactttgcgcataaatgacagtcagcctccattagtgttcagacccttttagacagctgaaatactgctacgaactgctgatgctggctgagctccccatggtacgtgtggtgcactttccctgcgcagcattagcagtgaaagcagctcagggtgcggtggtggccaaacccagggccgatcccacggcctcctgtacctggtcatacccacgggcacagctgctagtgaggtgcgtgcttttcagacacgtcatataagtgtgccctgcctacatggcggccgatcccaggaccccggggaatataagtctgagggggacgtaagcaacccttccttttgtaacagggacaacatagcccctatttccttcttagaaggagaggttttcccgcaataggtcttacacgcggacgaaatcacctttatgacggcttccatgcttgatccaccgggcgaccggaatcacgcagagcaaccggaatcacgcctggggtggaccgctcagtcgtcgggcttccttcccgtcttccaacgactctctgagttctcggtagggtatgttggccccctgcagtagggctccctccgacgccactcagcttctgccctcctaagccgcagccccctctactagggtcatcgtccgctccccgaataagcgagacggatgaggacaggatcgccacgccgcctgtggccgaccactattccctaacgatcacgtcggggtcaccaaatgaagccttctgcttcatgcatgtgctcgtagtcgtcagggaatcaacggtccggccatcaacccaggtgcacaccaatgtggtgaatggtcaaatggcgtttattgtatcgagctaggcacttaaatacaatatctctgcaatgcggaattcagtggttcgtccaatccgtgttagacccgtctgttgccttcctaacaaggcacgatcataccacgatcataccaccttactcccaccaatcggcatgcacggtgctttttctctccttataaggcatgttgctaactcatcgttacataagcatgttgcaagactacaagagtattgcataagactacatttccccctccctatgcaaaagcgaaactactatatcctgaggggactcctaaccgcgtacaaccgaagccccgcttttcgcctaaacatgctattgtcccctcagtcaagccttgcccgttacaacccgattcgcaagccttgccctccccacattatccgtagcattatttcctagcagtcatcagagctacagaagatactctatgctgtagccaagtctacaagtttactattcagcgacctcctatattccgcgtgccagccgatcaattaccaatgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgtattccctaacgatcacgtcggggtcaccaaatgaagccttctgcttcatgcatgtgctcgtagtcgtcagggaatcaacggtccggccatcaacccaggtgcacaccaatgtggtgaatggtcaaatggcgtttattgtatcgagctaggcacttaaatacaatatctctgcaatgcggaattcagtggttcgtccaatccgtccccctccctatgcaaaagcgaaactactatatcctgaggggactcctaaccgcgtacaaccgaagccccgcttttcgcctaaacatgctattgtcccctcagtcaagccttgcccgttacaacccgattcgcaagccttgccctccccacattatccgtagcattatttcctagcagtcatcagagctacagaagatactctatgctgtagccaagtctacaagtttactattcagcgacctcctatattccgcgtgccagccgatcaattaccaatccaaccagctatcacacggaatacaagaactcgcctacgctcttctttcgggctgcttataagcctcctgtaatttttttatattcctcg

SEQ ID NO: 48 - DNA, 7313 bp, Artificial Sequence
  Sequence of pOM-5′shuttle
ggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgcatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggcgggccccccctcgaggtcgacggtatcgataagcttgcagtccaaggctttgtctgtgtacccagtgaaatccttcctctgttacataaagcccagataggactcagaaatgtagtcattccagcccccctcttcctcagatctggagcagcacttgtttgcagccagtcctccccaaaatgcacagacctcgccgagtggagggagatgtaaacagcgaaggttaattacctccttgtcaaaaacactttgtggtccatagatgtttctgtcaatcttacaaaacagaaccgagaggcagcgagcactgaagagcgtgttcccatgctgagttaatgagacttggcagctcgctgtgcagagatgatccctgtgcttcatgggaggctgtaacctgtctccccatcgccttcacaccgcagtgctgtcctggacacctcaccctccataagctgtaggatgcagctgcccagggatcaagagacttttcctaaggctcttaggactcatctttgccgctcagtagcgtgcagcaattactcatcccaactatactgaatgggtttctgccagctctgcttgtttgtcaataagcatttcttcattttgcctctaagtttctctcagcagcaccgctctgggtgacctgagtggccacctggaacccgaggggcacagccaccacctccctgttgctgctgctccagggactcatgtgctgctggatggggggaagcatgaagttcctcacccagacacctgggttgcaatggctgcagcgtgctcttcttggtatgcagattgtttccagccattacttgtagaaatgtgctgtggaagccctttgtatctctttctgtggcccttcagcaaaagctgtgggaaagctctgaggctgctttcttgggtcgtggaggaattgtatgttccttctttaacaaaaattatccttaggagagagcactgtgcaagcattgtgcacataaaacaattcaggttgaaagggctctctggaggtttccagcctgactactgctcgaagcaaggccaggttcaaagatggctcaggatgctgtgtgccttcctgattatctgtgccaccaatggaggagattcacagccactctgcttcccgtgccactcatggagaggaatattcccttatattcagatagaatgttatcctttagctcagccttccctataaccccatgagggagctgcagatccccatactctccccttctctggggtgaaggccgtgtcccccagccccccttcccaccctgtgccctaagcagcccgctggcctctgctggatgtgtgcctatatgtcaatgcctgtccttgcagtccagcctgggacatttaattcatcaccagggtaatgtggaactgtgtcatcttcccctgcagggtacaaagttctgcacggggtcctttcggttcaggaaaaccttcactggtgctacctgaatcaagctctatttaataagttcataagcacatggatgtgttttcctagagatacgttttaatggtatcagtgatttttatttgctttgttgcttacttcaaacagtgcctttgggcaggaggtgagggacgggtctgccgttggctctgcagtgatttctccaggcgtgtggctcaggtcagatagtggtcactctgtggccagaagaaggacaaagatggaaattgcagattgagtcacgttaagcaggcatcttggagtgatttgaggcagtttcatgaaagagctacgaccacttattgttgttttccccttttacaacagaagttttcatcaaaataacgtggcaaagcccaggaatgtttgggaaaagtgtagttaaatgttttgtaattcatttgtcggagtgctaccagctaagaaaaaagtcctacctttggtatggtagtcctgcagagaatacaacatcaatattagtttggaaaaaaacaccaccaccaccagaaactgtaatggaaaatgtaaaccaagaaattccttgggtaagagagaaaggatgtcgtatactggccaagtcctgcccagctgtcagcctgctgaccctctgcagttcaggaccatgaaacgtggcactgtaagacgtgtcccctgcctttgcttgcccacagatctctgcccttgtgctgactcctgcacacaagagcatttccctgtagccaaacagcgattagccataagctgcacctgactttgaggattaagagtttgcaattaagtggattgcagcaggagatcagtggcagggttgcagatgaaatccttttctaggggtagctaagggctgagcaacctgtcctacagcacaagccaaaccagccaagggttttcctgtgctgttcacagaggcagggccagctggagctggaggaggttgtgctgggacccttctccctgtgctgagaatggagtgatttctgggtgctgttcctgtggcttgcactgagcagctcaagggagatcggtgctcctcatgcagtgccaaaactcgtgtttgatgcagaaagatggatgtgcacctccctcctgctaatgcagccgtgagcttatgaaggcaatgagccctcagtgcagcaggagctgtagtgcactcctgtaggtgctagggaaaatctctggttcccagggatgcattcataagggcaatatatcttgaggctgcgccaaatctttctgaaatattcatgcgtgttcccttaatttatagaaacaaacacagcagaataattattccaatgcctcccctcgaaggaaacccatatttccatgtagaaatgtaacctatatacacacagccatgctgcatccttcagaacgtgccagtgctcatctcccatggcaaaatactacaggtattctcactatgttggacctgtgaaaggaaccatggtaagaaacttcggttaaaggtatggctgcaaaactactcataccaaaacagcagagctccagacctcctcttaggaaagagccacttggagagggatggtgtgaaggctggaggtgagagacagagcctgtcccagttttcctgtctctattttctgaaacgtttgcaggaggaaaggacaactgtactttcaggcatagctggtgccctcacgtaaataagttccccgaacttctgtgtcatttgttcttaagatgctttggcagaacactttgagtcaattcgcttaactgtgactaggtctgtaaataagtgctccctgctgataaggttcaagtgacatttttagtggtatttgacagcatttaccttgctttcaagtcttctaccaagctcttctatacttaagcagtgaaaccgccaagaaacccttccttttatcaagctagtgctaaataccattaacttcataggttagatacggtgctgccagcttcacctggcagtggttggtcagttctgctggtgacaaagcctccctggcctgtgcttttacctagaggtgaatatccaagaatgcagaactgcatggaaagcagagctgcaggcacgatggtgctgagccttagctgcttcctgctgggagatgtggatgcagagacgaatgaaggacctgtcccttactcccctcagcattctgtgctatttagggttctaccagagtccttaagaggttttttttttttttggtccaaaagtctgtttgtttggttttgaccactgagagcatgtgacacttgtctcaagctattaaccaagtgtccagccaaaatcaattgcctgggagacgcagaccattacctggaggtcaggacctcaataaatattaccagcctcattgtgccgctgacagattcagctggctgctccgtgttccagtccaacagttcggacgccacgtttgtatatatttgcaggcagcctcggggggaccatctcaggagcagagcaccggcagccgcctgcagagccgggcagtacctcaacatgtgaattcggtaccggatcccgggccatggccatggcaggtgtcttcgtgctgttctctttcgtgctttgtggcttcctcccaggtgagtaactcccagagtgctgcagaagctttgtgcctgccagtcctggctctccttagcagaacatggtggtgaccatcagagagagactcccctacaaagtgcctgcaaaggctgcctcagtacatcagtattaaacggattactgttgtgctgggtgtctgttgggttctgtgctcccaacacatttcttacgctctcagctctgttacactgcttgcatttgctgcacagttgcatagaatggataaatgcttgaaacaaggccataacgaggtggtcagacctccaggaactagttagggaaatattgtcatggcccaagcaagctctgtgcaggaacctggcagctttcctgcaatgcttttgctgctaagc

SEQ ID NO: 49 - DNA, 39 bp, Chicken
gcgcacctgcgtattgccttggcaggcgtcttcgtgctg

SEQ ID NO: 50 - DNA, 77 bp, Chicken
ggcgcggccgcgaattcgacatgttggcagaatctggggaaaaaaaggggacaaaaatgggaaacaaaggcagtctg

SEQ ID NO: 51 - DNA, 572 bp, Artificial Sequence
  syn OM Exon1/intron1 junction sequence
caattgcctgggagacgcagaccattacctggaggtcaggacctcaataaatattaccagcctcattgtgccgctgacagattcagctggctgctctgtgttccagtccaacagttcggacgccacgtttgtatatatttgcaggcagcctcggggggaccatctcaggagcagagcaccggcagccgcctgcagagccgggcagtacctcaccattgccttggcaggcgtcttcgtgctgttctctttcgtgctttgtggcttcctcccaggtgagtaactcccagagtgctgcagaagctttgtgcctgccagtcctggctctccttagcagaacatggtggtgaccatcagagagagactcccctacaaagtgcctgcaaaggctgcctcagtacatcagtattaaacggattactgttgtgctgggtgtctgttgggttctgtgctcccaacacatttcttacgctctcagctctgttacactgcttgcatttgctgcacagttgcatagaatggataaatgcttgaaacaaggccataacgaggtggtcagacctccaggaactagt

SEQ ID NO: 52 - DNA, 42 bp, Chicken
ggcccaattgcccgggtctagaatatcagagctgagagaatt

SEQ ID NO: 53 - DNA, 31 bp, Chicken
ggagcggccgccatgtaggcagggcacactt

(19) United States
(12) Patent Application Publication
(10) Pub. No.: US 2010/0333219 A1
(43) Pub. Date: Dec. 30, 2010

(54) Methods of protein production using ovomucoid regulatory regions

(75) Inventor: Alex J. Harvey, Athens, GA (US)

Correspondence Address: Synageva BioPharma Corp., 111 RIVERBEND ROAD, ATHENS, GA, 30605, US

(73) Assignee: Synageva BioPharma Corp.

(21) Appl. No.: 12/799,605

(22) Filed: Apr. 28, 2010

Related U.S. Application Data
(63) Continuation-in-part of application No. 12/313,064, filed on Nov. 17, 2008, now Pat. No. 7,812,215.
Application No. 12/313,064 is a continuation of application No. 11/649,543, filed on Jan. 4, 2007, now Pat. No. 7,507,873.
Application No. 11/649,543 is a continuation of application No. 11/047,184, filed on Jan. 31, 2005, now Pat. No. 7,335,761.
Application No. 11/047,184 is a continuation-in-part of application No. 10/856,218, filed on May 28, 2004, now Pat. No. 7,294,507.
Application No. 10/856,218 is a continuation-in-part of application No. 10/496,731, filed on May 21, 2004, now Pat. No. 7,375,258.
Application No. 10/496,731 is a continuation-in-part of application No. 09/998,716, filed on Nov. 30, 2001, now Pat. No. 6,875,588.
Application No. 11/047,184 is a continuation-in-part of application No. 10/790,455, filed on Mar. 1, 2004, now abandoned.
(60) Provisional application No. 60/476,596, filed on Jun. 6, 2003.
(60) Provisional application No. 60/505,562, filed on Sep. 24, 2003.
(60) Provisional application No. 60/509,122, filed on Oct. 6, 2003.

(51) Int. Cl. C12P 21/00; C12P 21/08; C12P 21/04

(52) U.S. Cl. 800/4

(57) ABSTRACT

Methods of producing protein using a recombinant ovomucoid gene expression controlling region operably linked to one or more useful amino acid coding sequences.

### RELATED APPLICATION INFORMATION

This application is a continuation-in-part of U.S. patent application Ser. No. 12/313,064, filed Nov. 17, 2008, the disclosure of which is incorporated in its entirety herein by reference, which is a continuation of U.S. patent application Ser. No. 11/649,543, filed Jan. 4, 2007, the disclosure of which is incorporated in its entirety herein by reference, which is a continuation of U.S. patent application Ser. No. 11/047,184, now U.S. Pat. No. 7,335,761, issued Feb. 26, 2008, which is a continuation-in-part of U.S. patent application Ser. No. 10/856,218, filed May 28, 2004, now U.S. Pat. No. 7,294,507, issued Nov. 13, 2007, which is a continuation-in-part of U.S. patent application Ser. No. 10/496,731, filed May 21, 2004, now issued U.S. Pat. No. 7,375,258, which is a 371 of PCT/US02/38413, filed Dec. 2, 2002, and is a continuation-in-part of U.S. patent application Ser. No. 09/998,716 filed Nov. 30, 2001, now U.S. Pat. No. 6,875,588, issued Apr. 5, 2005. The disclosure of each of these three continuation-in-part applications and the PCT application is incorporated in its entirety herein by reference. U.S. patent application Ser. No. 11/047,184, now U.S. Pat. No. 7,335,761, issued Feb. 26, 2008, is also a continuation-in-part of U.S. patent application Ser. No. 10/790,455, now abandoned, filed Mar. 1, 2004, which claims the benefit of U.S. provisional patent application No. 60/476,596, filed Jun. 6, 2003, U.S. provisional patent application No. 60/505,562, filed Sep. 24, 2003 and U.S. provisional patent application No. 60/509,122, filed Oct. 6, 2003. The disclosure of the continuation-in-part application is incorporated by reference herein in its entirety.

### GOVERNMENT RIGHTS STATEMENT

This invention was made with government support under a grant from the National Institute of Standards and Technology. Therefore, the U.S. Government may have certain rights in this invention.

### FIELD OF THE INVENTION

The present invention relates generally to avian gene expression controlling regions, for example, from the chicken. The invention includes recombinant nucleic acid molecules and expression vectors, transfected cells and transgenic animals that include an avian gene expression controlling region operably linked to a nucleic acid of interest.

### BACKGROUND

The field of transgenics was initially developed to understand the action of a single gene in the context of the whole animal and the phenomena of gene activation, expression, and interaction. This technology has also been used to produce models for various diseases in humans and other animals and is amongst the most powerful tools available for the study of genetics, and the understanding of genetic mechanisms and function. From an economic perspective, the use of transgenic technology for the production of specific proteins such as substances of pharmaceutical interest (Gordon et al., (1987) Biotechnology 5: 1183-1187; Wilmut et al., (1990) Theriogenology 33: 113-123) offers significant advantages over more conventional methods of protein production by gene expression.

Heterologous nucleic acids have been engineered so that an expressed protein may be joined to a protein or peptide that will allow secretion of the transgenic expression product into milk or urine, from which the protein may then be recovered. These procedures have had limited success and may require maintenance of herds of large species, such as cows, sheep, or goats. Such animals typically have exceedingly long developmental periods and are costly to maintain.

One useful alternative that has shown great promise for heterologous gene expression is the avian reproductive system. The production of an avian egg begins with formation of a large yolk in the ovary of the hen. The unfertilized oocyte or ovum is positioned on top of the yolk sac. After ovulation, the ovum passes into the infundibulum of the oviduct where it is fertilized, if sperm are present, and then moves into the magnum of the oviduct which is lined with tubular gland cells. These cells secrete the egg-white proteins, including ovalbumin, ovomucoid, ovoinhibitor, conalbumin, ovomucin and lysozyme, into the lumen of the magnum where they are deposited onto the avian embryo and yolk.

The hen oviduct offers outstanding potential as a protein bioreactor because of the high levels of protein production, the promise of proper folding and post-translation modification of the target protein, the ease of product recovery, and the shorter developmental period of chickens compared to other animal species used for heterologous gene expression. As a result, efforts have been made to create transgenic chickens expressing heterologous proteins in the oviduct.

Chicken oviduct cells, when stimulated by steroid hormones during egg-laying, secrete three principal amino acid sequences, ovalbumin, ovomucoid and lysozyme (Tsai et al., (1978) Biochemistry 17: 5773-5779). The mRNA transcript encoding ovalbumin constitutes about 50% of the total mRNA of these cells. Ovomucoid and lysozyme mRNAs contribute about 6.6% and 3.4% respectively of the total mRNA of the steroid stimulated cells (Hynes et al. (1977) Cell 11:923-932).

Detailed restriction enzyme analysis of fragments of chicken genomic DNA have shown that the ovomucoid-encoding sequence includes seven intronic sequences (Lindenmaier et al. (1979) Nuc. Acid Res. 7:1221-1232; Catterall et al. (1979) Nature 278:323-327; Lai et al. (1979) Cell 18:829-842). Short stretches of the 5′ flanking region of the ovomucoid gene have been sequenced (Lai et al. (1979) Cell 18:829-842; Genbank Accession No. J00897), but extending only 579 bases upstream of the recognized transcription start site. The 5′ flanking region of the ovomucoid gene has been isolated (Catterall et al. (1979) Nature 278:323-327; Lai et al. (1979) Cell 18: 829-842), but not generally characterized beyond low-resolution restriction site mapping. Scott et al. (1987) Biochemistry 26:6831-6840, identified a CR1-like region within the approximately 10 kb chicken genomic DNA located between the ovoinhibitor-encoding region and the downstream ovomucoid gene. The ovoinhibitor-encoding cDNA and the attached 3′-untranslated region, which extends into the approximately 10 kb ovoinhibitor-ovomucoid region, were also sequenced (Scott et al. (1987) J. Biol. Chem. 262:5899-5907). There is no evidence that any of the previously identified portions of the ovomucoid gene are capable of regulating gene expression. In particular, there is no indication that any of these known portions are functional to assist in the initiation of transcription of the ovomucoid coding sequence. The chicken ovomucoid gene is highly expressed in the tubular glands of the mature hen oviduct and represents a suitable candidate for an efficient promoter for heterologous protein production in transgenic animals, especially avians, such as chickens.

What is needed are functional ovomucoid gene expression controlling nucleic acid sequences, such as ovomucoid promoters.

### SUMMARY OF THE INVENTION

The present invention relates in part to nucleic acids which include an avian ovomucoid gene expression controlling region useful for expression of nucleotide sequences encoding one or more amino acid sequences of interest, such as peptides, polypeptides or proteins.

In one embodiment, an expression vector of the invention contains a nucleotide sequence 75% or more identical to the sequence spanning from about nucleotide 1886 to about nucleotide 2824 of pSIN-1.8-OM-I-GCSF-3′UTR (SEQ ID NO: 47) wherein a heterologous sequence is covalently linked to the 3′ end of the nucleotide sequence. A heterologous sequence in this context refers to a sequence not normally linked to the sequence of about nucleotide 1886 to about nucleotide 2824 of pSIN-1,8-OM-I-GCSF-3′UTR when the sequence is found in nature. In such covalent linkage, a nucleotide sequence may intervene between the heterologous sequence and the 3′ end of the nucleotide sequence, such as a portion of ovomucoid exon 1, for example, as in pSIN-1,8-OM-I-GCSF-3′UTR (SEQ ID NO: 47).

The invention also includes an expression vector containing a nucleotide sequence 75% or more identical to the sequence spanning from about nucleotide 1886 to about nucleotide 2824 of pSIN-1.8-OM-I-GCSF-3′UTR (SEQ ID NO: 47) having a heterologous sequence covalently linked to the 3′ end of the sequence and a second sequence 75% or more identical to the sequence spanning from about nucleotide 3520 to about nucleotide 4117 of pSIN-1,8-OM-I-GCSF-3′UTR (SEQ ID NO: 47) wherein the heterologous sequence is covalently linked to the 5′ end of the second nucleotide sequence. In one embodiment, the heterologous sequence of the expression vector includes a coding sequence such as a therapeutic protein coding sequence.

The invention also includes nucleotide sequences and their use that are 80% identical to the sequence spanning from about nucleotide 1886 to about nucleotide 2824 of SEQ ID NO: 47, a sequence 85% identical to the sequence spanning from about nucleotide 1886 to about nucleotide 2824 of SEQ ID NO: 47, a sequence 90% identical to the sequence spanning from about nucleotide 1886 to about nucleotide 2824 of SEQ ID NO: 47, a sequence 91% identical to the sequence spanning from about nucleotide 1886 to about nucleotide 2824 of SEQ ID NO: 47, a sequence 92% identical to the sequence spanning from about nucleotide 1886 to about nucleotide 2824 of SEQ ID NO: 47, a sequence 93% identical to the sequence spanning from about nucleotide 1886 to about nucleotide 2824 of SEQ ID NO: 47, a sequence 94% identical to the sequence spanning from about nucleotide 1886 to about nucleotide 2824 of SEQ ID NO: 47, a sequence 95% identical to the sequence spanning from about nucleotide 1886 to about nucleotide 2824 of SEQ ID NO: 47, a sequence 96% identical to the sequence spanning from about nucleotide 1886 to about nucleotide 2824 of SEQ ID NO: 47, a sequence 97% identical to the sequence spanning from about nucleotide 1886 to about nucleotide 2824 of SEQ ID NO: 47, a sequence 98% identical to the sequence spanning from about nucleotide 1886 to about nucleotide 2824 of SEQ ID NO: 47, a sequence 99% identical to the sequence spanning from about nucleotide 1886 to about nucleotide 2824 of SEQ ID NO: 47 and a sequence identical to the sequence spanning from about nucleotide 1886 to about nucleotide 2824 of SEQ ID NO: 47. The heterologous nucleotide sequence spanning from about nucleotide 1886 to about nucleotide 2824 of SEQ ID NO: 47 and heterologous sequences that show at least 75% identity thereto are sometimes referred to herein as OM Intron.

The invention includes methods of expressing a protein in an avian that contains a recombinant or heterologous OM Intron in its geneome. In one embodiment, the intron is operably linked to a promoter such as, but not limited to, an ovalbumin promoter, an ovomucin promoter or a lysozyme promoter. In one particular embodiment, the promoter is an ovomucoid promoter, such as, but not limited to the about 1.8 kb ovomucoid promoter. In this particular context, operably linked refers to the OM Intron being in proximity to a promoter such that the activity of the promoter is effected, for example, the activity of the promoter is increased resulting in an expression level greater than the expression level would be without the OM Intron. OM Intron is contemplated for use in conjunction with any useful promoter including, but not limited to, those disclosed herein.

In one useful embodiment, the ovomucoid gene expression controlling region is effective to facilitate expression of certain nucleotide coding sequences in avian cells, for example, oviduct cells. In one embodiment, the amino acid sequence is heterologous, for example, the amino acid sequence is not the native ovomucoid protein product, and may be a mammalian, for example, a human amino acid sequence.

One aspect of the invention provides for a gene expression controlling region which includes nucleotide sequence found upstream of an ovomucoid coding sequence and/or nucleotide sequence found downstream of an ovomucoid coding sequence. In one aspect of the invention, fragments of an ovomucoid promoter gene which are effective to control gene expression of a nucleic acid sequence of interest are provided. For example, the invention provides for a nucleic acid fragment isolated from a region upstream of a transcription start site of an ovomucoid gene effective to control or regulate gene expression. In another example, the nucleic acid fragment is isolated from a region downstream of a transcription start site of an ovomucoid gene effective to control or regulate gene expression. In another embodiment, the fragment is isolated from a region upstream and downstream of a transcription start site of an ovomucoid gene effective to control gene expression.

In one embodiment of the present invention, the ovomucoid gene expression controlling region is isolated from a chicken. In a specific embodiment, the ovomucoid gene expression controlling region has a nucleotide sequence of OMC 70, which is included in the sequence of SEQ ID NO: 36. In one useful aspect, all or substantially all or a functional fragment of OMC 70 is employed to control the expression of a nucleic acid sequence of interest. The sequence of OMC 70 is included in the sequence of SEQ ID NO: 36 which is a BAC clone. A BAC clone which is believed to contain the nucleotide sequence represented by SEQ ID NO: 36 designated OMC24 has been deposited with the ATCC Patent Depository and has been assigned the deposit number of PTA-6234. The avian nucleotide sequence of PTA-6234 is included in the present application as are all functional fragments of the ovomucoid gene expression controlling sequence or region of PTA-6234. In one particularly useful aspect of the invention, the ovomucoid gene expression controlling region is a fragment or portion of OMC 70 which is effective to control gene expression in a cell, for example, an avian cell (e.g., a chicken cell). In a very useful aspect, fragments of the ovomucoid gene expression controlling region are operably linked or attached to a heterologous coding sequence such as a nucleotide sequence encoding a therapeutic protein.

In certain embodiments, the gene expression controlling region of the invention is at least 60% or at least 75% or at least 85% or at least 90% or at least 95% or at least 99% identical or homologous to an ovomucoid gene expression controlling region disclosed herein (e.g., the ovomucoid gene expression controlling region included in SEQ ID NO: 36) or fragments thereof and can regulate or control expression of a nucleotide sequence in a cell, such as an avian cell (e.g., a chicken cell).

In one embodiment, the avian ovomucoid gene expression controlling region of the present invention is useful for directing tissue-specific expression of an amino acid sequence-encoding nucleic acid. The gene expression controlling regions of the invention may be operably linked to a nucleic acid of interest (i.e., a nucleic acid insert) wherein the nucleic acid insert encodes an amino acid sequence desired to be expressed in a transfected cell. In one embodiment, the nucleic acid insert may be cloned in frame with a nucleotide sequence encoding a signal peptide. Translation may start with the signal peptide and continue through the nucleic acid insert, thereby producing an expressed amino acid sequence having the desired amino acid sequence including a signal sequence.

The nucleic acid of the present invention may include an untranslated 3′ region which may include a polyadenylation coding sequence allowing the transcript directed by the ovomucoid gene expression controlling region of the invention to include, in addition to a certain heterologous amino acid sequence (i.e., not the ovomucoid protein that is expressed from the endogenous gene containing the ovomucoid gene expression controlling region), a 3′ untranslated region that may include a polyadenylated tail. Any functional polyadenylation signal sequence may be linked to the 3′ end of the nucleic acid insert including the SV40 polyadenylation signal sequence, bovine growth hormone adenylation sequence or the like. There are many know useful signal sequences including those disclosed in U.S. Pat. No. 5,856,187, the disclosure of which is incorporated in its entirety herein by reference.

The nucleic acid of the invention may include certain gene expression controlling elements, such as promoters, enhancers, IRES's from a source other than an ovomucoid gene, for example, from a non-avian gene.

The sequence of the expressed nucleic acid insert may be optimized for codon usage by the host cell or host organism. Codon usage can be determined by methods well known in the art. For example, codon usage may be determined for an avian by methods known in the art, for example, by examining nucleotide sequences which encode proteins such as ovalbumin, ovomucoid, ovomucin and ovotransferrin produced by a chicken and comparing the encoded amino acids to the corresponding codons.

Yet another aspect of the invention relates to expression vectors suitable for expressing the nucleic acid coding sequences as disclosed herein. Expression vectors of the present invention may include an avian ovomucoid gene expression controlling region operably linked to a nucleic acid insert encoding a non-ovomucoid amino acid sequence, and optionally, a non-coding sequence such as a polyadenylation signal sequence. The expression vector may also include a bacterial plasmid sequence, a viral nucleic acid sequence, or fragments or variants thereof or other sequences that will allow for maintaining the vector in a suitable host. As contemplated in the present invention, the vector may be a YAC, BAC, HAC, MAC, bacteriophage-derived artificial chromosome (BBPAC), cosmid or P1 derived artificial chromosome (PAC).

The present invention further relates to nucleic acid vectors and transgenes inserted therein that incorporate multiple amino acid sequence-encoding regions, wherein a first amino acid sequence-encoding region is operatively linked to a transcription promoter and a second amino acid sequence-encoding region is operatively linked to an Internal Ribosome Entry Sequence (IRES). For example, the vector may contain coding sequences for two different heterologous proteins (e.g., the heavy and light chains of an immunoglobulin), both sequences under the control of the same promoter. In one useful embodiment, the promoter is an ovomucoid gene expression controlling region as disclosed herein.

Nucleic acid constructs of the invention, when inserted into the genome of a bird and expressed therein, will produce amino acid sequences that may be post-translationally modified, for example, glycosylated or, in certain embodiments, be present as complexes, such as dimmers, (e.g., heterodimers).

Another aspect of the present invention is a method of expressing an amino acid sequence in a eukaryotic cell by transfecting the cell with a recombinant DNA comprising a gene expression controlling region of the invention operably linked to a nucleic acid insert encoding the amino acid sequence and, optionally, a non-coding sequence such as a polyadenylation signal sequence, and culturing the transfected cell in a medium suitable for expression of the amino acid sequence under the control of the gene expression controlling region. In certain embodiments, the amino acid sequence is a therapeutic protein such as a cytokine, growth factor, enzyme, structural protein, an immunoglobulin, or other therapeutic protein including, but not limited to, those disclosed elsewhere herein, or subunit or fragment thereof. In other embodiments, the amino acid sequence is a mammalian, such as a human, amino acid sequence or is substantially similar to a human or mammalian amino acid sequence.

Also within the scope of the present invention are recombinant cells, tissues and animals, for example, avians such as chickens, containing recombinant nucleic acid molecules of the present invention. In certain embodiments, the level of expression of a heterologous protein is greater than 1 μg, 5 μg, 10 μg, 50 μg, 100 μg, 250 μg, 500 μg, 750 μg, 1 mg, 2 mg, 5 mg, 10 mg, 20 mg, 50 mg, 100 mg, 200 mg, 500 mg, 700 mg, 1 gram, 2 grams, 3 grams, 4 grams or 5 grams in an egg produced by the transgenic avian of the invention. In one embodiment, the heterologous protein is present mostly or exclusively in the egg white.

In one embodiment of the invention, the cell is a chicken oviduct cell and the nucleic acid comprises a chicken ovomucoid gene expression controlling region, a nucleic acid insert encoding a heterologous amino acid sequence of interest, which optionally is codon optimized for expression in an avian cell, and a non-coding sequence such as a polyadenylation sequence, for example, an SV40 polyadenylation sequence. In one particularly useful embodiment, the oviduct cell is present in a live avian, such as a chicken.

The present invention includes nucleic acid molecules, for example, DNA, which comprise an artificial chromosome comprising an ovomucoid gene expression controlling region and methods of using the nucleic acid molecules, such as for the production of transgenic avians comprising an artificial chromosome.

In one embodiment, the gene expression controlling region of the present invention is a nucleotide sequence that hybridizes to the nucleotide sequence of SEQ ID NO: 36 or portions thereof such as Fragment A, Fragment B or Fragment C as disclosed in FIG. 14. In another embodiment, the gene expression controlling region of the present invention is a nucleotide sequence that hybridizes to the complement of the nucleotide sequence of SEQ ID NO: 36 or portions thereof such as Fragment A, Fragment B or Fragment C as disclosed in FIG. 14. In one embodiment, the hybridizations are under stringent conditions. High stringency conditions, when used in reference to nucleic acid hybridization, may comprise conditions equivalent to binding or hybridization at 65° C. in a solution consisting of 6×SSPE, 1% SDS, 5×Denhardt's reagent and 100 μg/ml denatured salmon sperm DNA followed by washing in a solution comprising 0.1×SSPE, and 0.1% SDS at 65° C. for about 15 to about 20 minutes. In certain embodiments, the wash conditions may include 50% formamide at 42° C. instead of 65° C. High stringency washes may include 0.1×SSC to 0.2×SSC and 1% SDS at 65° C. for about 15 to about 20 min. (see, Sambrook et al., Molecular Cloning—A Laboratory Manual (2nd ed.) Vol. 1-3, Cold Spring Harbor Laboratory, Cold Spring Harbor Press, N.Y., 1989, the disclosure of which is incorporated herein in its entirety by reference). Exemplary medium stringency conditions are as described above for high stringency except that the washes are carried out at 55° C. or at 37° C. when in the presence of 50% formamide. In a most useful aspect of the invention, a nucleotide sequence that hybridizes to an ovomucoid gene expression controlling region and its complement, such as a nucleotide sequence that hybridizes to the nucleotide sequence of SEQ ID NO: 36 or portions thereof such as Fragment A, Fragment B or Fragment C as disclosed in FIG. 14 and their complement, which serves as a functional gene expression controlling region, is operably linked or attached to a heterologous coding sequence such as a nucleotide sequence encoding a therapeutic protein. In one embodiment of the invention, fragments or portions of the ovomucoid gene expression controlling region as disclosed herein are useful as hybridization probes as is understood in the field of molecular biology.

In one embodiment, the ovomucoid gene expression controlling region is that of SEQ ID NO: 36 or portions thereof such as Fragment A, Fragment B or Fragment C as disclosed in FIG. 14. In another embodiment, the ovomucoid gene expression controlling region comprises a functional portion of SEQ ID NO: 36 or portions thereof such as Fragment A, Fragment B or Fragment C as disclosed in FIG. 14. The ovomucoid gene expression controlling region may also include the complement of SEQ ID NO: 36 or the complement of portions thereof such as the complement of Fragment A, the complement of Fragment B or the complement of Fragment C as disclosed in FIG. 14. In a particularly useful embodiment of the invention, a functional portion of SEQ ID NO: 26 or a functional portion of the avian nucleic acid contained in SEQ ID NO: 36 is operably linked or attached to a heterologous coding sequence such as a nucleotide sequence encoding a therapeutic protein.

In one embodiment, functional portions of the nucleotide sequence of the avian ovomucoid gene expression controlling region contained in SEQ ID NO: 36 are shown in FIG. 14. For example, Fragment A is an approximately 10 kb fragment which spans from about nucleotide 26,416 to about nucleotide 36,390 of FIG. 14 and of SEQ ID NO 36. Fragment B is an approximately 3.9 kb fragment which spans from about nucleotide 32,364 to about nucleotide 36,299 of FIG. 14 and of SEQ ID NO 36. Fragment C is an approximately 1.8 kb fragment which spans from about nucleotide 34,473 to about nucleotide 36,248 of FIG. 14 and of SEQ ID NO 36.

In another example, a potentially useful functional portion of the ovomucoid gene expression controlling region is the portion of SEQ ID NO: 36 which extends from the SbfI site at about nucleotide 14,727 to the EcoRI site at about nucleotide 48,185. Another example of a potentially useful functional portion of the ovomucoid gene expression controlling region is the portion of SEQ ID NO: 36 which extends from the HindIII site at about nucleotide 24,742 to the EcoRI site at about nucleotide 48,185. Another example of a potentially useful functional portion of the ovomucoid gene expression controlling region is the portion of SEQ ID NO: 36 which extends from the EcoRI site at about nucleotide 27,028 to the EcoRI site at about nucleotide 48,185. Another example of a potentially useful functional portion of the ovomucoid gene expression controlling region is the portion of SEQ ID NO: 36 which extends from the HindIII site at about nucleotide 28,381 to the EcoRI site at about nucleotide 48,185. Another example of a potentially useful functional portion of the ovomucoid gene expression controlling region is the portion of SEQ ID NO: 36 which extends from the EcoRI site at about nucleotide 27,028 to the EcoRI site at about nucleotide 54,424. In addition, a useful ovomucoid gene expression controlling region may extend from about nucleotide 35,861 to about nucleotide 36,252.

Methodologies are well known in the field that are useful to identify gene expression controlling regions within specified nucleic acid sequences (see, for example, Reese, M. G. and Eeckman, F. H. (1995) “Novel Neural Network Algorithms for improved Eukaryotic Promoter Site Recognition” The seventh international Genome sequencing and analysis conference, Hyatt Regency, Hilton Head Island, South Carolina Sep. 16-20, 1995 and Reese, M. G., Ph.D. Thesis (2000) UC Berkeley/University Hohenheim). Numerous computer programs are known in the art which can be used to identify gene expression controlling sequences such as promoter sequences within a certain nucleotide sequence. Using such sequence analysis programs, potential gene expression controlling regions can be identified and thereafter tested for gene expression controlling activity by methods known in the field of molecular biology such as those disclosed herein. For example, a 50 nucleotide sequence spanning from nucleotide 36,209 to nucleotide 36,258 was shown to be a potential promoter site with a relatively high degree (match score of 1.0) of certainty using the computer program available at http://www.fruitfly.org/seq_tools/nnppAbst.html.

In one embodiment, the gene expression controlling region comprises a nucleotide sequence that is at least 50% homologous or identical to the ovomucoid gene expression controlling region of the nucleotide sequence of SEQ ID NO: 36 or portions thereof such as Fragment A, Fragment B or Fragment C as disclosed in FIG. 14 or is at least 50% homologous to the complement of the ovomucoid gene expression controlling region of the nucleotide sequence of SEQ ID NO: 36 or portions thereof such as Fragment A, Fragment B or Fragment C as disclosed in FIG. 14. For example, the gene expression controlling region may comprise a nucleotide sequence that is at least 60% homologous or identical to the ovomucoid gene expression controlling region of the nucleotide sequence of SEQ ID NO: 36 or portions thereof such as Fragment A, Fragment B or Fragment C as disclosed in FIG. 14 or is at least 60% homologous to a complement thereof. In another example, the gene expression controlling region comprises a nucleotide sequence that is at least 70% homologous or identical to the ovomucoid gene expression controlling region of the nucleotide sequence of SEQ ID NO: 36 or portions thereof such as Fragment A, Fragment B or Fragment C as disclosed in FIG. 14 or is at least 70% homologous to a complement thereof. In another example, the gene expression controlling region comprises a nucleotide sequence that is at least 75% homologous or identical to the ovomucoid gene expression controlling region of the nucleotide sequence of SEQ ID NO: 36 or portions thereof such as Fragment A, Fragment B or Fragment C as disclosed in FIG. 14 or is at least 75% homologous or identical to a complement thereof. In another example, the gene expression controlling region comprises a nucleotide sequence that is at least 80% homologous or identical to the ovomucoid gene expression controlling region of the nucleotide sequence of SEQ ID NO: 36 or portions thereof such as Fragment A, Fragment B or Fragment C as disclosed in FIG. 14 or is at least 80% homologous or identical to a complement thereof. In another example, the gene expression controlling region comprises a nucleotide sequence that is at least 85% homologous or identical to the ovomucoid gene expression controlling region of the nucleotide sequence of SEQ ID NO: 36 or is at least 85% homologous or identical to a complement thereof. In another example, the gene expression controlling region comprises a nucleotide sequence that is at least 90% homologous or identical to the ovomucoid gene expression controlling region of the nucleotide sequence of SEQ ID NO: 36 or portions thereof such as Fragment A, Fragment B or Fragment C as disclosed in FIG. 14 or is at least 90% homologous or identical to a complement thereof. In another example, the gene expression controlling region comprises a nucleotide sequence that is at least 95% homologous or identical to the ovomucoid gene expression controlling region of the nucleotide sequence of SEQ ID NO: 36 or portions thereof such as Fragment A, Fragment B or Fragment C as disclosed in FIG. 14 or is at least 95% homologous or identical to a complement thereof. In another example, the gene expression controlling region comprises a nucleotide sequence that is at least 99% homologous or identical to the ovomucoid gene expression controlling region of the nucleotide sequence of SEQ ID NO: 36 or portions thereof such as Fragment A, Fragment B or Fragment C as disclosed in FIG. 14 or is at least 99% homologous or identical to a complement thereof.

Nucleotide sequences which are 80%, 85%, 90%, 91%, 92%, 93%, 94%, 95%, 96%, 97%, 98% and 99% identical or homologous to each of the nucleotide sequences disclosed herein are contemplated for use in accordance with the invention.

In one embodiment, nucleic acid molecules of the invention include an attB site. The use of attB is disclosed in, for example, U.S. patent application Ser. No. 10/790,455, filed Mar. 1, 2004, now abandoned, the disclosure of which is incorporated in its entirety herein by reference.

The nucleic acid molecules of the present invention may also include a signal sequence coding region which may be useful for secretion of an amino acid sequence product from a cell. In one embodiment, the signal sequence is cleaved from the amino acid sequence product during the secretion process. For the purposes of the present invention, “signal sequence peptide” refers to amino acid sequences of about 15 to about 25 amino acids in length which are known in the art to be generally located at the amino terminus of proteins and which are capable of facilitating secretion of a peptide or amino acid sequence from a cell.

In one particularly useful embodiment, the nucleic acid molecules of the present invention include an artificial chromosome. Any useful artificial chromosomes are contemplated for use in the present invention. In one embodiment, an artificial chromosome is a DNA molecule which includes a telomere and is capable of self replication in a cell, for example, in an avian cell. In another embodiment, an artificial chromosome includes a telomere and a centromere. Artificial chromosomes include, without limitation, BACs (bacterial artificial chromosomes), YACs (yeast artificial chromosomes), HACs (human artificial chromosomes) MACs (mammalian artificial chromosomes), BBPACs (bacteriophage derived artificial chromosomes) or PACs (P1 derived artificial chromosomes) or combinations thereof. Artificial chromosomes may include a gene expression controlling region as disclosed herein and may be present in cells of a transgenic avian such as a chicken or may be present in cells in culture.

The present invention also relates to compositions and methods for expressing certain peptides and amino acid sequences (e.g., peptides or proteins). The compositions can include a nucleic acid molecule comprising an artificial chromosome and an ovomucoid gene expression controlling region, as disclosed herein, which may be operably linked to a nucleotide sequence encoding an amino acid sequence. The nucleic acid may be inserted into a cell, for example, into a cell of an avian, where the amino acid sequence is expressed. In one embodiment, the nucleic acid molecule is present in cells of a transgenic avian including oviduct cells, for example, tubular gland cells of a transgenic avian. The coding region may encode any useful polynucleotide including pharmaceutical or therapeutic proteins which comprise an amino acid sequence.

The nucleic acid molecules of the present invention may be introduced into a cell, for example, into the cell of an avian, by any useful method. Such methods include, without limitation, microinjecting, transfection, electroporation and lipofection. The nucleic acid molecules may be introduced into a germinal disc or an avian embryo cell such as an early stage avian embryo. In one embodiment, the nucleic acid molecules of the present invention are introduced into an avian embryo cell such as a stage I avian embryo, stage II avian embryo, stage III avian embryo, stage IV avian embryo, stage V avian embryo, stage VI avian embryo, stage VII avian embryo, stage VIII avian embryo, stage IX avian embryo, stage X avian embryo, stage XI avian embryo or stage XII avian embryo.

Certain specific examples of pharmaceutical or therapeutic proteins which are contemplated for production as disclosed herein include, with out limitation, Factor VIII (e.g., Recombinate®, Bioclate®, Kogenate®, Helixate® (Centeon), B-domain deleted Factor VIII (e.g., ReFacto®), Factor VIIa (e.g., NovoSeven®), Factor IX (e.g., Benefix®), anticoagulant; recombinant hirudin (e.g., Revasc®, Refludan®) Alteplase, tPA (e.g., Activase®), Reteplase, tPA, tPA—3 of 5 domains deleted, Ecokinase®, Retavase®, Rapilysin®, insulin (e.g., Humulin®, Novolin®, Insuman®) insulin lispro (e.g., Humalog®), Bio Lysprol, Liprolog®), insulin Aspart, iNovoRapid®, insulin glargine, long-acting insulin analog (e.g., Lantus®), rhGH (e.g., Protropin®, Humatrope®, Nutropin®, BioTropin®, Genotropin®, Norditropin®, Saizen®, Serostim®), glucagons (e.g., Glucagen®), TSH (e.g., Thyrogen®, Gonal F®, Puregon®), follitropin-beta FSH (e.g., Follistim®), EPO (e.g., Epogen®, Procrit®, Neorecormon®), GM-CSF (e.g., Leukine®, Neupogen®), PDGH (e.g., Regranex®), hormones such as cytokines, IFN alpa2a (e.g., Roferon A®), INF-apha (e.g., Infergen®), IFN alpa2b (e.g., Intron A®, Alfatronol®, Virtron®), ribavirin & INF-alpha 2b (e.g., Robetron®) INF-beta 1b, (e.g., Betaferon®), IFN-beta 1a (e.g., Avonex®, Rebif®), IFN-gamma 1b (e.g., Actimmune®), IL-2 (e.g., Proleukin®) rIL-11 (e.g., Neumega®), rHBsAg (e.g., Recombivax®), Combination vaccine containing HBsAgn as one component (e.g., Comvax®, Tritarix®, Twinrix®, Primavax®, Procomax®), OspA, a lipoprotein found on the surface of B burgoeri (e.g., Lymerix®), murine MAb directed against t-lymphocyte antigen CD3 (e.g., Orthoclone OKT3®), murine MAb directed against TAG-72, tumor-associated glycoprotein (e.g., OncoScint CR/OV®), FAb fragments derived from chimeric MAb, directed against platelet surface receptor GPII(b)/III(a) (e.g., ReoPro®), murine MAb fragment directed against tumor-associated antigen CA125 (e.g., Indimacis®), murine MAb fragment directed against human carcinoembryonic antigen, CEA (e.g., CEA-scan®), murine MAb fragment directed against human cardiac myosin (e.g., MyoScint®), murine MAb fragment directed against tumor surface antigen PSMA (e.g., ProstaScint®), murine MAb fragments (FAb/FAb2 mix) directed against HMW-MAA (e.g., Tacnemab®), murine MAb fragment (FAb) directed against carcinoma-associated antigen (e.g., Verluma®), MAb fragments (FAb) directed against NCA 90, a surface granulocyte nonspecific cross reacting antigen (e.g., LeukoScan®), chimeric MAb directed against CD20 antigen found on surface of B lymphocytes (e.g., Rituxan®), humanized MAb directed against the alpha chain of the IL2 receptor (e.g., Zenapax®), chimeric MAb directed against the alpha chain of the IL2 receptor (e.g., Simulect®), chimeric MAb directed against TNF-alpha (e.g., Remicade®), humanized MAb directed against an epitope on the surface of respiratory synctial virus (e.g., Synagis®), humanized MAb directed against HER 2, i.e., human epidermal growth factor receptor 2 (e.g., Herceptin®), human MAb directed against cytokeratin tumor-associated antigen (e.g., Humaspect®), anti-CTLA4, chimeric MAb directed against CD 20 surface antigen of B lymphocytes (e.g., Mabthera®), dornase-alpha DNAse (e.g., Pulmozyme®), lysosomal acid lipase (LAL), beta glucocerebrosidase (e.g., Cerezyme®), TNF-alpha (e.g., Beromun®), IL-2-diptheria toxin fusion protein that targets cells displaying a surface IL-2 receptor (e.g., Ontak®), TNFR-IgG fragment fusion protein (e.g., Enbrel®), Laronidase, Recombinant DNA enzyme, (e.g., Aldurazyme®), Alefacept, Amevive®, Darbepoetin alfa (Colony stimulating factor) (e.g., Aranesp®), Tositumomab and iodine 1 131 tositumomab, murine MAb, Bexxar®, Alemtuzumab, Campath®, Rasburicase, Elitek®), Agalsidase beta, Fabrazyme®, FluMist®, Teriparatide, Parathyroid hormone derivative (e.g., Forteo®), Enfuvirtide Fuzeon®, Adalimumab (lgG1) (e.g., Humira®), Anakinra, Biological modifier (e.g., Kineret®), nesiritide, Human B-type natriuretic peptide (hBNP) (e.g., Natrecor®), Pegfilgrastim, Colony stimulating factor (e.g., Neulasta®), ribavarin and peg Intron A (e.g., Rebetron®), Pegvisomant, PEGylated human growth hormone receptor antagonist, (e.g., Somavert®), recombinant activated protein C (e.g., Xigris®), Omalizumab, Immunoglobulin E (lgE) blocker (e.g., Xolair®) and lbritumomab tiuxetan (murine MAb) (e.g., Zevalin®).

In one particularly useful embodiment, the amino acid sequence such as a pharmaceutical or therapeutic protein encoded by the nucleotide sequence operably linked to the ovomucoid gene expression controlling region is present in egg white produced by a transgenic avian of the present invention (i.e., an avian comprising a cell which includes a nucleic acid molecule of the present invention)

In one aspect of the invention, the nucleic acid molecule includes a nucleotide sequence encoding a light chain and/or a heavy chain of an antibody or a portion of a light chain and/or a heavy chain of an antibody which is operably linked to the ovomucoid gene expression controlling region. The antibody may be IgG (e.g., IgG1, IgG2, IgG3 or IgG4), IgA (e.g., IgA1 or IgA2), IgD, IgM or IgE. In addition, the light chain of the antibody may be a kappa light chain or a lambda light chain.

The present invention also contemplates the production of useful fusion proteins. For example, an antibody or a portion of an antibody may be produced as a fusion protein with another useful amino acid sequence.

The techniques used to isolate and characterize the nucleic acids and proteins of the present invention are well known to those of skill in the art and standard molecular biology and biochemical manuals may be consulted to select suitable protocols without undue experimentation. See, for example, Sambrook et al. (2001) Molecular Cloning: A Laboratory Manual, 3rd ed., Cold Spring Harbor Press, the content of which is herein incorporated by reference in its entirety.

Any combination of features described herein is included within the scope of the present invention provided that the features included in any such combination are not mutually inconsistent. Such combinations will be apparent based on this specification and on the knowledge of one of ordinary skill in the art.

### DEFINITIONS

Definitions of certain terms used in the present application are set forth below.

As used herein the terms “amino acid sequence” and “protein” refer to a polymer of amino acids of three or more amino acids in a serial array, linked through peptide bonds. The term “amino acid sequence” includes proteins, protein fragments, protein analogues, oligopeptides and the like. The term amino acid sequence as used herein can also refer to a peptide. The term “amino acid sequences” contemplates amino acid sequences as defined above that are encoded by nucleic acids, produced through recombinant technology (isolated from an appropriate source such as a bird), or synthesized. The term “amino acid sequences” further contemplates amino acid sequences as defined above that include chemically modified amino acids or amino acids covalently or noncovalently linked to labeling ligands.

The term “animal” is used herein to include all vertebrate animals, including humans. It also includes an individual animal in all stages of development, including embryonic and fetal stages.

The term “antisense DNA” as used herein refers to a gene sequence DNA that has a nucleotide sequence complementary to the “sense strand” of a gene when read in reverse orientation, i.e., DNA read into RNA in a 3′ to 5′ direction rather than in the 5′ to 3′ direction. The term “antisense RNA” is used to mean an RNA nucleotide sequence (for example that encoded by an antisense DNA or synthesized complementary with the antisense DNA). Antisense RNA is capable of hybridizing under stringent conditions with an antisense DNA. The antisense RNA of the invention is useful for regulating expression of a “target gene” either at the transcriptional or translational level. For example, transcription of the subject nucleic acids may produce antisense transcripts that are capable of inhibiting transcription by inhibiting initiation of transcription or by competing for limiting transcription factors; the antisense transcripts may inhibit transport of the “target RNA”, or, the antisense transcripts may inhibit translation of “target RNA”.

The term “avian” as used herein refers to any species, subspecies or race of organism of the taxonomic class ava, such as, but not limited to, such organisms as chicken, turkey, duck, goose, quail, pheasants, parrots, finches, hawks, crows and ratites including ostrich, emu and cassowary. The term includes the various known strains of Gallus gallus, or chickens, (for example, White Leghorn, Brown Leghorn, Barred-Rock, Sussex, New Hampshire, Rhode Island, Ausstralorp, Minorca, Amrox, California Gray, Italian Partidge-colored), as well as strains of turkeys, pheasants, quails, duck, ostriches and other poultry commonly bred in commercial quantities.

The term “antibody” as used herein refers to polyclonal and monoclonal antibodies and fragments thereof, and immunologic binding equivalents thereof. The term “antibody” refers to a homogeneous molecular entity, or a mixture such as a polyclonal serum product made up of a plurality of different molecular entities, and may further comprise any modified or derivatised variant thereof that retains the ability to specifically bind an epitope. A monoclonal antibody is capable of selectively binding to a target antigen or epitope. Antibodies may include, but are not limited to polyclonal antibodies, monoclonal antibodies (mAbs), humanized or chimeric antibodies, camelized antibodies, single chain antibodies (scFvs), Fab fragments, F(ab′)₂ fragments, disulfide-linked Fvs (sdFv) fragments, e.g., as produced by a Fab expression library, anti-idiotypic (anti-Id) antibodies, intrabodies, synthetic antibodies, and epitope-binding fragments of any of the above.

The term “cytokine” as used herein refers to any secreted amino acid sequence that affects the functions of cells and is a molecule that modulates interactions between cells in the immune, inflammatory or hematopoietic responses. A cytokine includes, but is not limited to, monokines and lymphokines regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epideral keratinocytes and B-lymphocytes. Lymphokines are generally referred to as being produced by lymphocyte cells. Examples of cytokines include, but are not limited to, Interleukin-1 (IL-1), Interleukin-6 (IL-6), Interleukin-8 (IL-8), Tumor Necrosis Factor-alpha (TNF-alpha) and Tumor Necrosis Factor beta (TNF-beta).

The term “capable of hybridizing under stringent conditions” as used herein refers to annealing a first nucleic acid to a second nucleic acid under stringent conditions as defined below. Stringent hybridization conditions typically permit the hybridization of nucleic acid molecules having at least 70% nucleic acid sequence identity with the nucleic acid molecule being used as a probe in the hybridization reaction. For example, the first nucleic acid may be a test sample or probe, and the second nucleic acid may be the sense or antisense strand of an ovomucoid gene expression controlling region or a fragment thereof. Hybridization of the first and second nucleic acids may be conducted under stringent conditions, e.g., high temperature and/or low salt content that tend to disfavor hybridization of dissimilar nucleotide sequences. Alternatively, hybridization of the first and second nucleic acid may be conducted under reduced stringency conditions, e.g. low temperature and/or high salt content that tend to favor hybridization of dissimilar nucleotide sequences. Low stringency hybridization conditions may be followed by high stringency conditions or intermediate medium stringency conditions to increase the selectivity of the binding of the first and second nucleic acids. The hybridization conditions may further include reagents such as, but not limited to, dimethyl sulfoxide (DMSO) or formamide to disfavor still further the hybridization of dissimilar nucleotide sequences. A suitable hybridization protocol may, for example, involve hybridization in 6×SSC (wherein 1×SSC comprises 0.015 M sodium citrate and 0.15 M sodium chloride), at 65° C. in an aqueous solution, followed by washing with 1×SSC at 65° C. Formulae to calculate appropriate hybridization and wash conditions to achieve hybridization permitting 30% or less mismatch between two nucleic acid molecules are disclosed, for example, in Meinkoth et al. (1984) Anal. Biochem. 138: 267-284; the content of which is herein incorporated by reference in its entirety. Protocols for hybridization techniques are well known to those of skill in the art and standard molecular biology manuals may be consulted to select a suitable hybridization protocol without undue experimentation. See, for example, Sambrook et al. (2001) Molecular Cloning: A Laboratory Manual, 3rd ed., Cold Spring Harbor Press, the contents of which are herein incorporated by reference in their entirety.

1 to 1.0 M Na ion concentration (or other salts) from about pH 7.0 to about pH 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° Celsius, and a wash in 1× to 2×SSC at 50 to 55° Celsius. Exemplary moderate stringency conditions include hybridization in 40 to 45% formamide, 1 M NaCl, 1% SDS at 37° Celsius, and a wash in 0.5× to 1×SSC at 55 to 60° Celsius. Exemplary high stringency conditions include hybridization in 50% formamide, 1 M NaCl, 1% SDS at 37° Celsius, and a wash in 0.1×SSC at 60 to 65° Celsius.

The term “coding region” as used herein refers to a continuous linear arrangement of nucleotides which may be translated into a protein. A full length coding region is translated into a full length protein; that is, a complete protein as would be translated in its natural state absent any post-translational modifications. A full length coding region may also include any leader protein sequence or any other region of the protein that may be excised naturally from the translated protein.

The term “complementary” as used herein refers to two nucleic acid molecules that can form specific interactions with one another. In the specific interactions, an adenine base within one strand of a nucleic acid can form two hydrogen bonds with thymine within a second nucleic acid strand when the two nucleic acid strands are in opposing polarities. Also in the specific interactions, a guanine base within one strand of a nucleic acid can form three hydrogen bonds with cytosine within a second nucleic acid strand when the two nucleic acid strands are in opposing polarities. Complementary nucleic acids as referred to herein, may further comprise modified bases wherein a modified adenine may form hydrogen bonds with a thymine or modified thymine, and a modified cytosine may form hydrogen bonds with a guanine or a modified guanine.

By the use of the term “enriched” in reference to nucleic acid it is meant that the specific DNA or RNA sequence constitutes a significantly higher fraction of the total DNA or RNA present in the cells or solution of interest than in normal or diseased cells or in the cells from which the sequence was taken. Enriched does not imply that there are no other DNA or RNA sequences present, just that the relative amount of the sequence of interest has been significantly increased, for example, by 1 fold, 2 fold, 5 fold, 10 fold, 50 fold, 100 fold, 500 fold, 1000 fold, 10,000 fold, 100,000 fold, or 1,000,000 fold. The other DNA may, for example, be derived from a yeast or bacterial genome, or a cloning vector, such as a plasmid or a viral vector.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein, an amino acid sequence or a portion thereof.

The term “expression vector” as used herein refers to a nucleic acid vector that comprises the ovomucoid gene expression controlling region operably linked to a nucleotide sequence coding at least one amino acid sequence. As used herein, the term “regulatory sequences” includes promoters, enhancers, and other elements that may control gene expression. Standard molecular biology textbooks such as Sambrook et al. eds “Molecular Cloning: A Laboratory Manual” 3rd ed., Cold Spring Harbor Press (2001) may be consulted to design suitable expression vectors that may further include an origin of replication and selectable gene markers. It should be recognized, however, that the choice of a suitable expression vector and the combination of functional elements therein depends upon multiple factors including the choice of the host cell to be transformed and/or the type of protein to be expressed.

The term “fragment” as used herein can refer to, for example, an at least about 10, 20, 50, 75, 100, 150, 200, 250, 300, 500, 1000, 2000, 5000, 6,000, 8,000, 10,000, 20,000, 30,000, 40,000, 50,000 or 60,000 nucleotide long portion of a nucleic acid (e.g., cDNA) that has been constructed artificially (e.g., by chemical synthesis) or by cleaving a natural product into multiple pieces, using restriction endonucleases or mechanical shearing, or enzymatically, for example, by PCR or any other polymerizing technique known in the art, or expressed in a host cell by recombinant nucleic acid technology known to one of skill in the art. The term “fragment” as used herein may also refer to, for example, an at least about 5, 10, 20, 30, 40, 50, 75, 100, 150, 200, 250, 300, 400, 500, 1000, 2000, 5000, 6,000, 8,000 or 10,000 amino acid portion of an amino acid sequence, which portion is cleaved from a naturally occurring amino acid sequence by proteolytic cleavage by at least one protease, or is a portion of the naturally occurring amino acid sequence synthesized by chemical methods or using recombinant DNA technology (e.g., expressed from a portion of the nucleotide sequence encoding the naturally occurring amino acid sequence) known to one of skill in the art. “Fragment” may also refer to a portion, for example, of about 5%, about 10%, about 20%, about 30%, about 40%, about 50%, about 60%, about 70%, about 80% about 90% about 95% or about 99% of a particular nucleotide or amino acid sequence.

“Functional portion” or “functional fragment” as used herein means a portion or fragment of a whole capable of performing, in whole or in part, a function of the whole. For example, a biologically functional portion of a molecule means a portion of the molecule that performs a biological function of the whole or intact molecule. For example, a functional portion of a gene expression controlling region is a fragment or portion of the specified gene expression controlling region that, in whole or in part, regulates or controls gene expression (e.g., facilitates either in whole or in part) in a biological system (e.g., a promoter). Functional portions may be of any useful size. For example, a functional fragment may range in size from about 20 bases in length to a length equal to the entire length of the specified sequence minus one nucleotide. In another example, a functional fragment may range in size from about 50 bases in length to a length equal to the entire length of the specified sequence minus one nucleotide. In another example, a functional fragment may range in size from about 50 bases in length to about 70 kb in length. In another example, a functional fragment may range in size from about 500 bases in length to about 70 kb in length. In another example, a functional fragment may range in size from about 1 kb in length to about 70 kb in length. In another example, a functional fragment may range in size from about 1 kb in length to about 20 kb in length. In another example, a functional fragment may range in size from about 1 kb in length to about 10 kb in length. Functional portions may include, for example, and without limitation, one or more of a matrix attachment region, a transcription enhancer, a hormone responsive element or a CR1 repeat element.

The term “gene” or “genes” as used herein refers to nucleic acid sequences (including both RNA or DNA) that encode genetic information for the synthesis of a whole RNA, a whole protein, or any portion of such whole RNA or whole protein. Genes that are not naturally part of a particular organism's genome are referred to as “foreign genes,” “heterologous genes” or “exogenous genes” and genes that are naturally a part of a particular organism's genome are referred to as “endogenous genes”. The term “gene product” refers to RNAs or proteins that are encoded by the gene. “Foreign gene products” are RNA or proteins encoded by “foreign genes” and “endogenous gene products” are RNA or proteins encoded by endogenous genes. “Heterologous gene products” are RNAs or proteins encoded by foreign, heterologous or foreign exogenous genes and are, therefore, not naturally expressed in the cell.

The term “gene expression controlling region” as used herein refers to a nucleotide sequence which regulate, in whole or in part, the expression of the nucleotide sequence, for example, regulate, in whole or in part, the transcription of a nucleotide sequence. Exemplary transcription regulatory sequences include enhancer elements, hormone response elements, steroid response elements, negative regulatory elements, and the like. The “transcription regulatory sequences” may be isolated and incorporated into a nucleic acid vector to enable regulated transcription in appropriate cells of portions of the vector DNA. The “transcription regulatory sequence” may precede, but is not limited to, the region of a nucleic acid sequence that is in the region 5′ of the end of a protein coding sequence that may be transcribed into mRNA. Transcriptional regulatory sequences may also be located within a protein coding region, in regions of a gene that are identified as “intron” regions, or may be in other regions of nucleic acid sequence. In addition, to “control gene expression,” or “controlling gene expression”, refers to regulation, in whole or in part, of the expression of a nucleotide sequence, for example, regulation, in whole or in part, of the transcription of a nucleotide sequence.

The term “immunoglobulin amino acid sequence” as used herein refers to an amino acid sequence derived from a constituent amino acid sequence of an immunoglobulin. An “immunoglobulin amino acid sequence” may be, but is not limited to, an immunoglobulin (preferably an antibody) heavy or light chain and may include a variable region, a diversity region, a joining region and/or a constant region or any combination, variant or truncated form thereof. The term “immunoglobulin amino acid sequences” further includes single-chain antibodies comprised of, but not limited to, an immunoglobulin heavy chain variable region, an immunoglobulin light chain variable region and optionally a peptide linker.

The term “isolated nucleic acid” as used herein refers to a nucleic acid that has been substantially removed from other components of the cell containing the nucleic acid or from other components of chemical/synthetic reaction used to generate the nucleic acid. In specific embodiments, the nucleic acid is 50%, 60%, 70%, 80%, 90%, 95%, 99% or 100% pure. The “isolated nucleic acid” does not include nucleic acids that are members of a library, e.g. cDNA or genomic library, unless identified and separated from the other members of the library. The techniques used to isolate and characterize the nucleic acids and proteins of the present invention are well known to those of skill in the art and standard molecular biology and biochemical manuals may be consulted to select suitable protocols without undue experimentation. See, for example, Sambrook et al, 2001, Molecular Cloning: A Laboratory Manual, 3rd ed., Cold Spring Harbor Press; the content of which is herein incorporated by reference in its entirety.

As used herein, the term “locus” or “loci” refers to the site of a gene on a chromosome. Pairs of genes control hereditary traits, each in the same position on a pair of chromosomes. These gene pairs, or alleles, may both be dominant or may both be recessive in expression of that trait. In either case, the individual is said to be homozygous for the trait controlled by that gene pair. If the gene pair (alleles) consists of one dominant and one recessive trait, the individual is heterozygous for the trait controlled by the gene pair. Natural variation in genes or nucleic acid molecules caused by, for example, recombination events or resulting from mutation, gives rise to allelic variants with similar, but not identical, nucleotide sequences. Such allelic variants typically encode proteins with similar activity to that of the protein encoded by the gene to which they are compared, because natural selection typically selects against variations that alter function. Allelic variants can also comprise alterations in the untranslated regions of the gene as, for example, in the 3′ or 5′ untranslated regions or can involve alternate splicing of a nascent transcript, resulting in alternative exons being positioned adjacently.

The term “nucleic acid” as used herein refers to any natural and synthetic linear and sequential arrays of nucleotides and nucleosides, for example cDNA, genomic DNA, mRNA, tRNA, oligonucleotides, oligonucleosides and derivatives thereof. Representative examples of the nucleic acids of the present invention include bacterial plasmid vectors including expression, cloning, cosmid and transformation vectors such as, but not limited to, plasmid vectors, animal viral vectors such as, but not limited to, modified adenovirus, influenza virus, polio virus, pox virus, retrovirus, and the like, vectors derived from bacteriophage nucleic acid, e.g., plasmids and cosmids, artificial chromosomes, such as but not limited to, Yeast Artificial Chromosomes (YACs) and Bacterial Artificial Chromosomes (BACs), and synthetic oligonucleotides like chemically synthesized DNA or RNA. The term “nucleic acid” further includes modified or derivatised nucleotides and nucleosides such as, but not limited to, halogenated nucleotides such as, but not only, 5-bromouracil, and derivatised nucleotides such as biotin-labeled nucleotides.

The term “nucleic acid vector” or “vector” as used herein refers to a natural or synthetic single or double stranded plasmid or viral nucleic acid molecule, or any other nucleic acid molecule, such as but not limited to YACs, BACs, bacteriophage-derived artificial chromosome (BBPAC), cosmid or P1 derived artificial chromosome (PAC), that can be transfected or transformed into cells and replicate independently of, or within, the host cell genome. A circular double stranded vector can be linearized by treatment with an appropriate restriction enzyme based on the nucleotide sequence of the vector. A nucleic acid can be inserted into a vector by cutting the vector with restriction enzymes and ligating the pieces together. The nucleic acid molecule can be RNA or DNA.

The terms “operably linked” or “operatively linked” can refer to a gene expression controlling sequence linked to a coding sequence. Control sequences operably linked to a coding sequence are capable of effecting the expression of the coding sequence and/or regulating in which tissues, at what developmental time points, or in response to which signals a gene is expressed. For example, a coding sequence is operably linked to or under the control of transcriptional regulatory regions in a cell when DNA polymerase will bind the promoter sequence and transcribe the coding sequence into mRNA that can be translated into the encoded protein. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence. Such intervening sequences include but are not limited to enhancer sequences which are not transcribed or are not bound by polymerase. “Operatively linked” in the context of gene expression controlling sequences being operatively linked to each other refers to one gene expression controlling sequence having an effect on another gene expression controlling sequence. For example, operably linked can refer to an OM Intron being in proximity to a promoter such that activity of the promoter is effected, for example, the activity of the promoter is increased resulting in an expression level greater than the expression level that the promoter would have without the OM Intron being operably linked to the promoter.

The terms “percent sequence identity” or “percent sequence homology” or “percent sequence similarity” as used herein refer to the degree of sequence identity between two nucleic acid sequences or two amino acid sequences as determined using the algorithm of Karlin & Attschul (1990) Proc. Natl. Acad. Sci. 87: 2264-2268, modified as in Karlin & Attschul (1993) Proc. Natl. Acad. Sci. 90: 5873-5877. Such an algorithm is incorporated into the NBLAST and XBLAST programs of Attschul et al. (1990) T. Mol. Biol. Q15: 403-410. BLAST nucleotide searches are performed with the NBLAST program, score=100, wordlength=12, to obtain nucleotide sequences homologous to a nucleic acid molecule of the invention. BLAST protein searches are performed with the XBLAST program, score=50, wordlength=3, to obtain amino acid sequences homologous to a reference amino acid sequence. To obtain gapped alignments for comparison purposes, Gapped BLAST is utilized as described in Attschul et al. (1997) Nucl. Acids Res. 25: 3389-3402. When utilizing BLAST and Gapped BLAST programs, the default parameters of the respective programs (e.g. XBLAST and NBLAST) are used. Other algorithms, programs and default settings may also be suitable such as, but not only, the GCG-Sequence Analysis Package of the U.K. Human Genome Mapping Project Resource Centre that includes programs for nucleotide or amino acid sequence comparisons.

A “pharmaceutical composition” is a substance that, in whole or in part, makes up a drug. “Therapeutic proteins” or “pharmaceutical proteins” include an amino acid sequence which in whole or in part makes up a drug. In one embodiment, a pharmaceutical composition includes one or more pharmaceutical proteins or therapeutic proteins.

The terms “polynucleotide” and “nucleic acid sequence” are used interchangeably herein and include, but are not limited to, coding sequences (polynucleotide(s) or nucleic acid sequence(s) which are transcribed and translated into amino acid sequence in vitro or in vivo when placed under the control of appropriate regulatory or control sequences); control sequences (e.g., translational start and stop codons, promoter sequences, ribosome binding sites, polyadenylation signals, transcription factor binding sites, transcription termination sequences, upstream and downstream regulatory domains, enhancers, silencers, and the like); and regulatory sequences (DNA sequences to which a transcription factor(s) binds and alters the activity of a gene's promoter either positively (induction) or negatively (repression)). No limitation as to length or to synthetic origin is suggested by the terms described herein.

The term “probe” as used herein, when referring to a nucleic acid, refers to a nucleotide sequence that can be used to hybridize with and thereby identify the presence of a complementary sequence, or a complementary sequence differing from the probe sequence but not to a degree that prevents hybridization under the hybridization stringency conditions used. The probe may be modified with labels such as, but not only, radioactive groups, biotin, and/the like that are well known in the art.

The term “promoter’ as used herein refers to the DNA sequence that determines the site of transcription initiation by an RNA polymerase. A “promoter-proximal element” may be a regulatory sequence within about 200 base pairs of the transcription start site. A “magnum-specific” promoter, as used herein, is a promoter that is primarily or exclusively active in the tubular gland cells of the avian magnum. Useful promoters also include exogenously inducible promoters. These are promoters that can be “turned on” in response to an exogenously supplied agent or stimulus, which is generally not an endogenous metabolite or cytokine. Examples include an antibiotic-inducible promoter, such as a tetracycline-inducible promoter, a heat-inducible promoter, a light-inducible promoter, or a laser inducible promoter. (e.g., Halloran et al. (2000) Development 127: 1953-1960; Gemer et al. (2000) Int. J. Hyperthermia 16: 171-81; Rang and Will, 2000, Nucleic Acids Res. 28: 1120-5; Hagihara et al. (1999) Cell Transplant 8: 4314; Huang et al. (1999) Mol. Med. 5: 129-37; Forster et al. (1999) Nucleic Acids Res. 27: 708-10; Liu et al. (1998) Biotechniques 24: 624-8, 630-2; the contents of which have been incorporated herein by reference in their entireties).

The term “recombinant cell” refers to a cell that has a new combination of nucleic acid segments that are not covalently linked to each other in nature in that particular configuration. A new configuration of nucleic acid segments can be introduced into an organism using a wide array of nucleic acid manipulation techniques available to those skilled in the art. A recombinant cell can be a single eukaryotic cell, such as a mammalian or avian cell (including within a transgenic mammal or avian) or a single prokaryotic cell. The recombinant cell may harbor a vector that is extragenomic. An extragenomic nucleic acid vector does not insert into the cell's genome. A recombinant cell may further harbor a vector or a portion thereof (e.g., the portion containing the regulatory sequences and the coding sequence) that is intragenomic. The term intragenomic defines a nucleic acid construct incorporated within the recombinant cell's genome.

The terms “recombinant nucleic acid” and “recombinant DNA” as used herein refer a combination of at least two nucleic acids that is not naturally found in a eukaryotic or prokaryotic cell in that particular configuration. The nucleic acids may include, but are not limited to, nucleic acid vectors, gene expression regulatory elements, origins of replication, suitable gene sequences that when expressed confer antibiotic resistance, protein-encoding sequences and the like. The term “recombinant amino acid sequence” is meant to include an amino acid sequence produced by recombinant DNA techniques such that it is distinct from a naturally occurring amino acid sequence either in its location, purity or structure. Generally, such a recombinant amino acid sequence will be present in a cell in an amount different from that normally observed in nature.

The term “sense strand” as used herein refers to a single stranded DNA molecule from a genomic DNA that may be transcribed into RNA and translated into the natural amino acid sequence product of the gene. The term “antisense strand” as used herein refers to the single strand DNA molecule of a genomic DNA that is complementary with the sense strand of the gene.

The terms “transformation” and “transfection” as used herein refer to the process of inserting a nucleic acid into a host. Many techniques are well known to those skilled in the art to facilitate transformation or transfection of a nucleic acid into a prokaryotic or eukaryotic organism. These methods involve a variety of techniques, such as treating the cells with high concentrations of salt such as, but not only, a calcium or magnesium salt, an electric field, detergent, or liposome mediated transfection, to render the host cell competent for the uptake of the nucleic acid molecules, and by such methods as sperm-mediated and restriction-mediated integration.

The term “transfecting agent” as used herein refers to a composition of matter added to the genetic material for enhancing the uptake of heterologous DNA segment(s) into a eukaryotic cell, preferably an avian cell. The enhancement is measured relative to the uptake in the absence of the transfecting agent. Examples of transfecting agents include adenovirus-transferrin-polylysine-DNA complexes. These complexes generally augment the uptake of DNA into the cell and reduce its breakdown during its passage through the cytoplasm to the nucleus of the cell. These complexes can be targeted to, e.g., the male germ cells using specific ligands that are recognized by receptors on the cell surface of the germ cell, such as the c-kit ligand or modifications thereof.

Other transfecting agents include but are not limited to lipofectin, Iipfectamine, DIMRIE C, Supeffect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecytammonium bromide), DHDEAB (N,N-di-n-hexadecyl-N,N-dihydroxyethyl ammonium bromide), HDEAB (N-n-hexadecylN,N-dihydroxyethylammonium bromide), polybrene, or poly(ethylenimine) (PEI). These non-viral agents have the advantage that they can facilitate stable integration of xenogeneic DNA sequences into the vertebrate genome, without size restrictions commonly associated with virus-derived transfecting agents.

As used herein, a “transgenic animal” is any non-human animal, such as an avian species, including the chicken, in which one or more of the cells of the animal contain a heterologous nucleic acid introduced by way of human intervention, such as by transgenic techniques well known in the art. The nucleic acid is introduced into a cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animal, the transgene causes cells to express a recombinant form of the subject amino acid sequence, e.g. either agonistic or antagonistic forms, or in which the gene has been disrupted. In certain embodiments, the genome of the animal has been modified such that a heterologous gene expression element is inserted so as to be operably linked to an endogenous coding sequence. The terms “chimeric animal” or “mosaic animal” are used herein to refer to animals in which the recombinant gene is found, or in which the recombinant gene is expressed in some but not all cells of the animal. The term “tissue-specific chimeric animal” indicates that the recombinant gene is present and/or expressed in some tissues but not others.

As used herein, the term “transgene” means a nucleic acid sequence (encoding, for example, a human interferon amino acid sequence) that is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location that differs from that of the natural gene or its insertion results in a knockout). A trangene also includes a regulatory sequence designed to be inserted into the genome such that it regulates the expression of an endogenous coding sequence, e.g., to increase expression and or to change the timing and or tissue specificity of expression, etc. (e.g., to effect “gene activation”).

The terms “unique nucleic acid region” and “unique protein (amino acid sequence) region” as used herein refer to sequences present in a nucleic acid or protein (amino acid sequence) respectively that is not present in any other nucleic acid or protein sequence. The terms “conserved nucleic acid region” as referred to herein is a nucleotide sequence present in two or more nucleic acid sequences, to which a particular nucleic acid sequence can hybridize under low, medium or high stringency conditions. The greater the degree of conservation between the conserved regions of two or more nucleic acid sequences, the higher the hybridization stringency that will allow hybridization between the conserved region and a particular nucleic acid sequence.

This description uses gene nomenclature accepted by the Cucurbit Genetics Cooperative as it appears in the Cucurbit Genetics Cooperative Report 18:85 (1995), herein incorporated by reference in its entirety. Using this gene nomenclature, genes are symbolized by italicized Roman letters. If a mutant gene is recessive to the normal type, then the symbol and name of the mutant gene appear in italicized lower case letters.

### Abbreviations

Abbreviations used in the present specification include the following: aa, amino acid(s); bp, base pair(s); cDNA, DNA complementary to RNA; ml, milliliter; min, minute(s); nt, nucleotide(s); SSC, sodium chloride-sodium citrate; ug, microgram(s); ul, microliter(s); uM, micromolar; UTR, untranslated region; DMSO, dimethyl sulfoxide.

Additional objects and aspects of the present invention will become more apparent upon review of the detailed description set forth below when taken in conjunction with the accompanying figures, which are briefly described as follows.

### BRIEF DESCRIPTION OF THE FIGURES

FIG. 1 illustrates an agarose gel analysis of PCR products from PCR amplification of chicken genomic DNA using the primers OVINs2 (SEQ ID NO: 1) and OVMUa2 (SEQ ID NO: 2).

FIG. 2 illustrates the approximately 10 kb nucleic acid region that is 5′ upstream of the chicken ovomucoid transcription start site, and the positions and orientations of primers used to sequence this region.

FIG. 3 shows the PCR primers SEQ ID NOS: 1-25 used to PCR amplify and/or sequence the approximately 10 kb nucleic acid region that is 5′ upstream of the chicken ovomucoid transcription start site.

FIG. 4A-4D shows the nucleic acid sequence SEQ ID NO: 26 of the approximately 10 kb nucleic acid region that is 5′ upstream of the chicken ovomucoid transcription start site.

FIG. 5 illustrates the approximately 10 kb ovomucoid promoter linked to the luciferase or human IFNα-2b coding sequences.

FIG. 6A shows the results of transfections of plasmids containing the ovomucoid promoter or CMV promoter linked to a luciferase gene into HD11 cells, a chicken myeloid cell line. FIG. 6B shows the results of transfections of plasmids containing the ovomucoid promoter or CMV promoter linked to a luciferase gene into primary quail tubular gland cells isolated from the magnum portion of the oviduct of a laying quail hen. FIG. 6C shows the results of transfection into primary quail tubular gland cells isolated from the magnum of a laying quail hen for the approximately 10 kb ovomucoid promoters and the ovomucoid BAC-IRES construct each comprising an operably linked luciferase coding sequence.

FIG. 7 shows the results of transfections of plasmids containing the ovomucoid promoter or CMV promoter linked to an interferon gene into primary quail tubular gland cells isolated from the magnum portion of the oviduct of a laying quail hen.

FIG. 8 shows an ovomucoid gene and bacterial artificial chromosome. FIG. 8 A. The ovoinhibitor (OI) and adjacent ovomucoid (OM) regions are shown with transcriptional start sites indicated with bent arrows. The left and right sides of the BAC, relative to an EcoR1 site found in the 3′ UTR, are shown with their approximate sizes in kilobase pairs (kb). FIG. 8 B. The coding region of ovomucoid is shown with exons as white boxes and introns as black boxes. C. The IRES and polynucleotide coding sequence for the light chain and heavy chain of the IgG1 inserted at the EcoR1 site.

FIG. 9 shows an SDS-PAGE analysis of partially purified hMab derived from a single transgenic hen. (M) Multi-mark standard, lane 1) 1 mg purified hMab (produced by mammalian cells), lane 2) 5 mg pre-column (transgenic avian egg white), lane 3) 5 mg column flow thru from transgenic avian egg white, lane 4) partially purified hMab from transgenic avian egg white.

FIG. 10 shows plots of the binding ability of an IgG1 monoclonal antibody produced by a transgenic chicken and the binding ability of the same IgG1 monoclonal antibody produced by mammalian cells.

FIG. 11A-11F shows the ability of avian derived hMab to bind target antigen expressed on a cell surface relative to the ability of the mammalian cell derived hMab.

FIG. 12 shows the stability of hMab expression in transgenic hen. Eggs from transgenic hens #4992 and #1251 were collected over several weeks. The amount of hMab in egg white material was quantitated over time via sandwich ELISA for the specific human IgG1 (H+L).

FIG. 13 shows ADCC (antibody dependent cellular cytotoxicity) and CDCC (complement-dependent cellular cytotoxicity) for an IgG1 produced in transgenic avians.

FIG. 14 shows the nucleotide sequence of the approximately 70 kb ovomucoid gene expression controlling region which is included in SEQ ID NO: 36. Also indicated in the figure is the approximately 10 kb ovomucoid gene expression controlling region which is designated Fragment A and shown in bold, the approximately 3.9 kb ovomucoid gene expression controlling region which is designated Fragment B and is shown underlined and the approximately 1.8 kb ovomucoid gene expression controlling region which is designated Fragment C and is shown in lower case.

FIG. 15 shows construction of the pOM-3.9-CTLA4 expression vector which includes the approximately 3.9 kb ovomucoid gene expression controlling region (Fragment B of FIG. 14) operably linked to a CTLA4 coding sequence and the construction of pNLB-OM-1.8-CTLA4 which includes the approximately 1.8 kb ovomucoid gene expression controlling region (Fragment C of FIG. 14) operably linked to a CTLA4 coding sequence. In the figure, “A” represents the transcription start site; “B” represents the ovomucoid CDS; “C” represents the approximately 3.9 kb ovomucoid gene expression controlling region; “D” represents the translation start site; and “E” represents the approximately 1.8 kb ovomucoid gene expression controlling region. pNLB is a replication deficient avian leukosis viral vector (ALV). See, for example, U.S. Pat. No. 6,730,822, issued May 4, 2004, the disclosure of which is incorporated in its entirety herein by reference.

FIG. 16 shows the pOM-3.9-luc construct, the pOM-3.9-intron-lucpA construct and the pOM-3.9-lucpA construct.

FIG. 17 shows relative measurements in a quail TGC assay for six vectors. LRLU stands for luciferase relative light units.

FIG. 18. Map of pSIN-1.8-OM-I-GCSF-3′UTR vector (SEQ ID NO: 47).

| | |
|---|---|
| OM Intron A | 1886-2824 |
| OM 3′ UTR | 3520-4117 |
| Exon 1 with ATGs removed | 1775-1885 |
| Start of Exon 2 | 2825-2826 |
| G-CSF | 2836-3447 |
| LTR | 8024-8196 |
| LTR | 4576-4921 |
| 1.8 kb OM promoter | 1-1774 |

FIG. 19. Map of the integrated SIN-1.8-OM-I-GCSF-3′UTR vector.

FIG. 20. Map of pOM-5′Shuttle (SEQ ID NO: 48).

### DETAILED DESCRIPTION OF THE INVENTION

The present invention relates to avian gene expression controlling regions and to methods of their use. In one embodiment, the invention relates to avian (e.g., chicken) ovomucoid promoters and to methods of using such promoters in the production of useful amino acid sequences such as peptides and proteins.

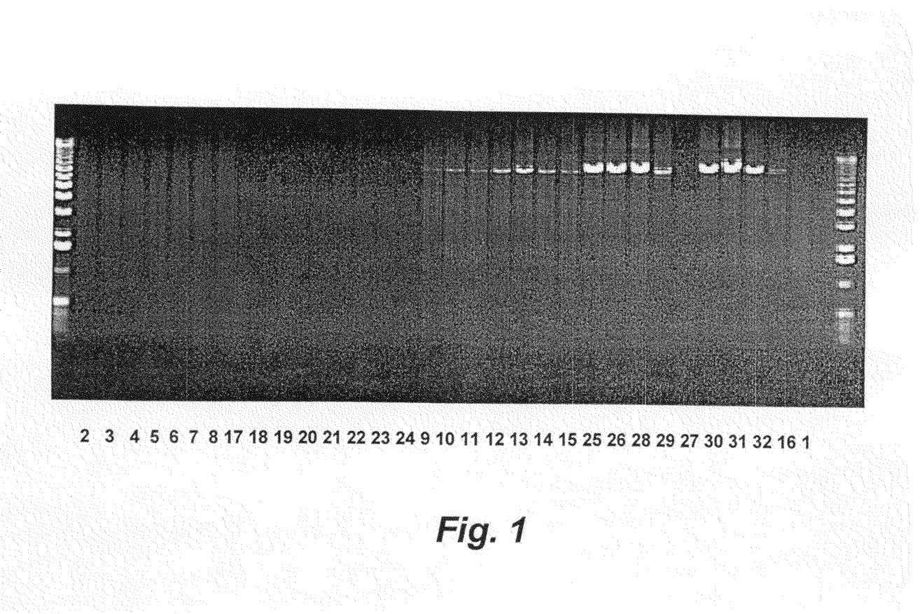

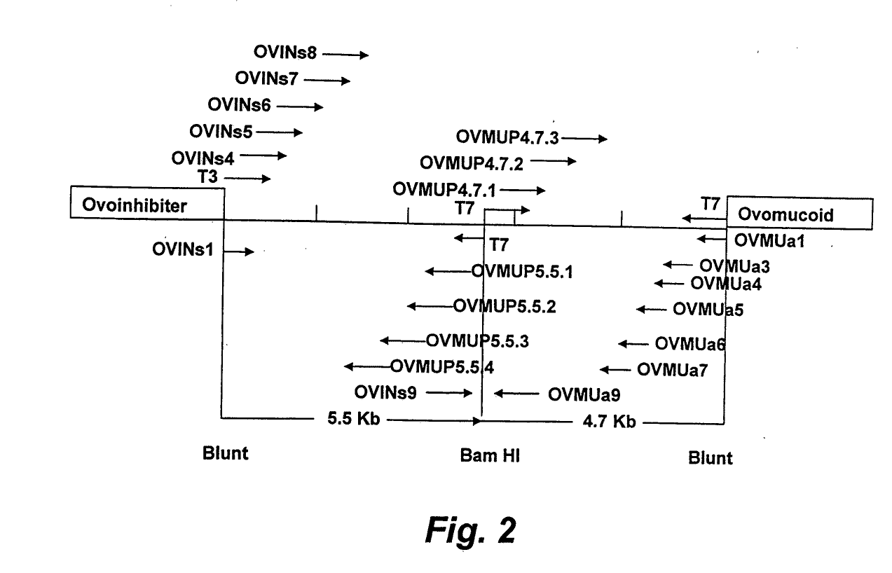

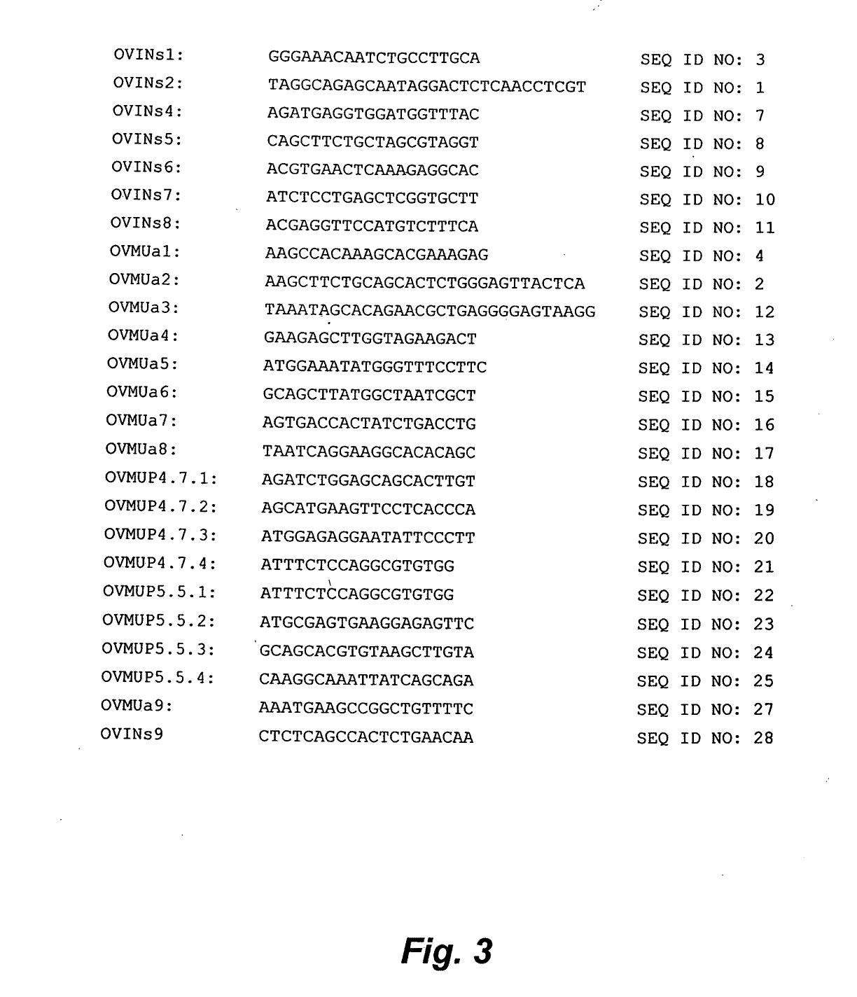

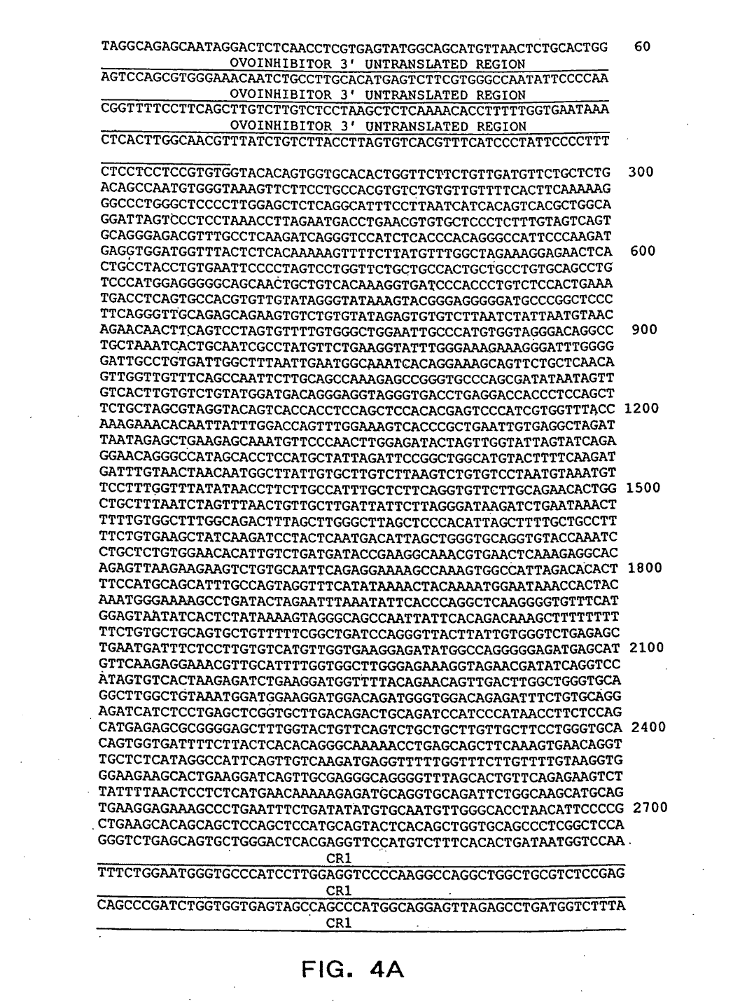

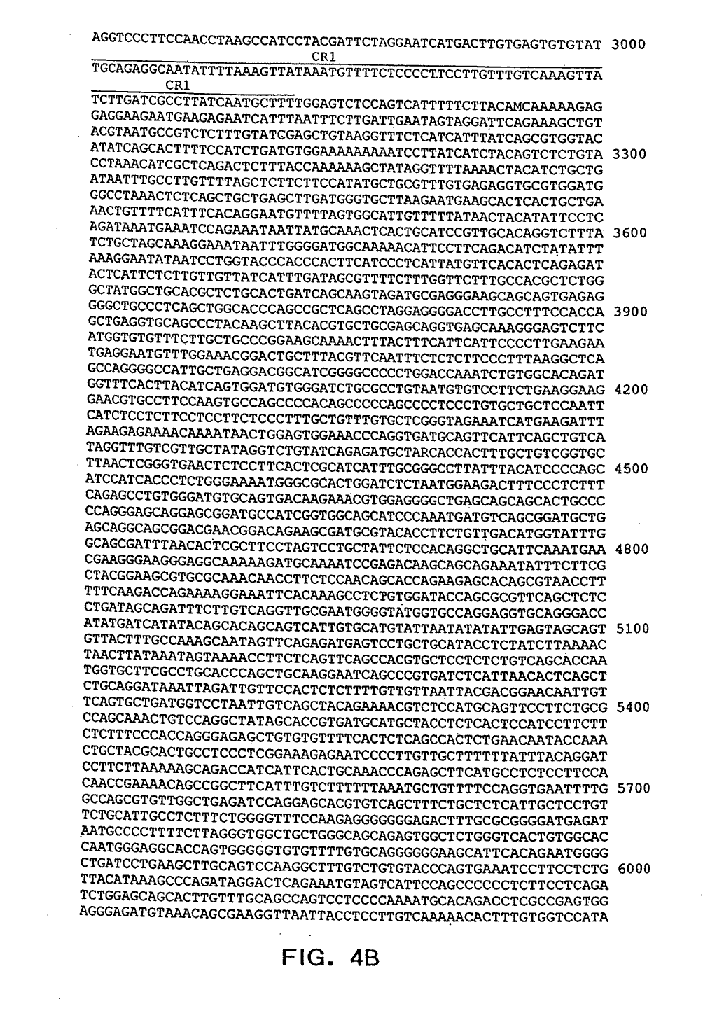

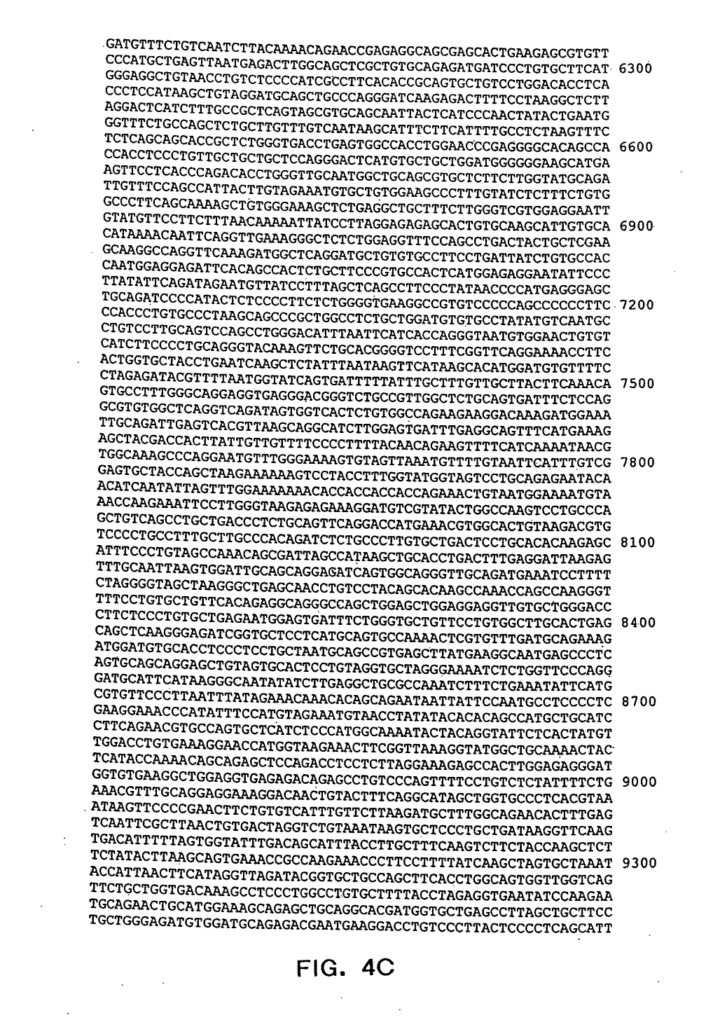

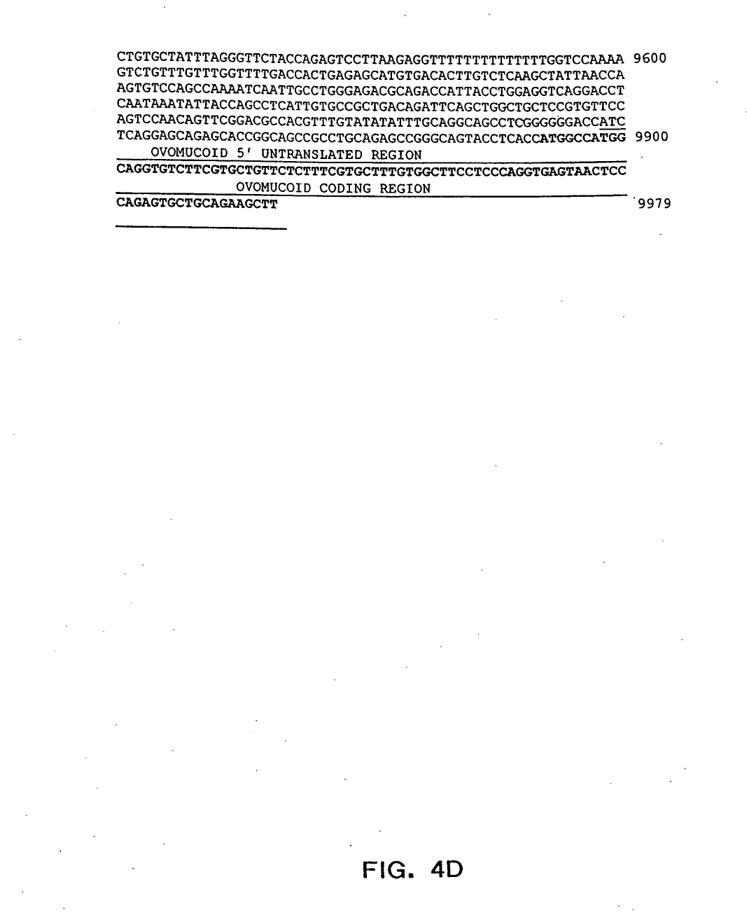

A series of PCR amplifications of template chicken genomic DNA were used to isolate the gene expression controlling region of the chicken ovomucoid locus. For example, the region of the chicken genome lying between the 3′ end of the ovoinhibitor gene and the 5′ transcription start site of the ovomucoid gene was PCR amplified using the primers OVINs 2,5′-TAGGCAGAGCAATAGGACTCTCAACCTCGT-3′ (SEQ ID NO: 1) and OVMUa2,5′-AAGCTTCTGCAGCACTCTGGGAGTTACTCA-3′ (SEQ ID NO: 2) as described in detail in Example 1 below and FIG. 1. The approximately 10 kb fragment was blunt-ended and cleaved with the restriction endonuclease Bam HI. The resulting fragments of about 4.7 kb and 5.5 kb were subcloned into the linearized plasmid vector pBluescript KS II (+/−) (Stratagene, La Jolla, Calif.). Each insert was sequenced using the primers SEQ ID NOS: 5 to 25 shown in FIGS. 2 and 3 and as described in Example 3 below. The compiled nucleic acid sequence (SEQ ID NO: 26) of the approximately 10 kb nucleic acid region that is 5′ upstream of the chicken ovomucoid transcription start site is shown in FIG. 4.

SEQ ID NO: 26 includes the ovoinhibitor gene 3′ untranslated region described by Scott et al. (1987) J. Biol. Chem. 262: 5899-5909, from base positions 1-255 as shown in FIG. 4. A CR1-like element (Scott et al., Biochemistry (1987) 26: 6831-6840; Genbank Accession No: M17966) is located at base positions 2761-3024 as shown in FIG. 4. The region of SEQ ID NO: 26 from base positions 9403-9920, as shown in FIG. 4, has been described in Genbank Accession No: J00897 and in Lai et al., Cell (1979) 18: 829-842 and includes a portion of the 5′ untranslated region of the ovomucoid gene.

An avian ovomucoid gene region has been identified in a chicken artificial chromosome library. The library was constructed with HindIII chicken DNA inserts ligated into a BAC vector (see, Crooijmans et al. (2000) Mammalian Genome 11: 360-363, the disclosure of which is incorporated in its entirety by reference). However, the present invention contemplates the employment of any useful artificial chromosome library including, but not limited to, libraries constructed from YACs, HACs, MACs, BBPACs or PACs.

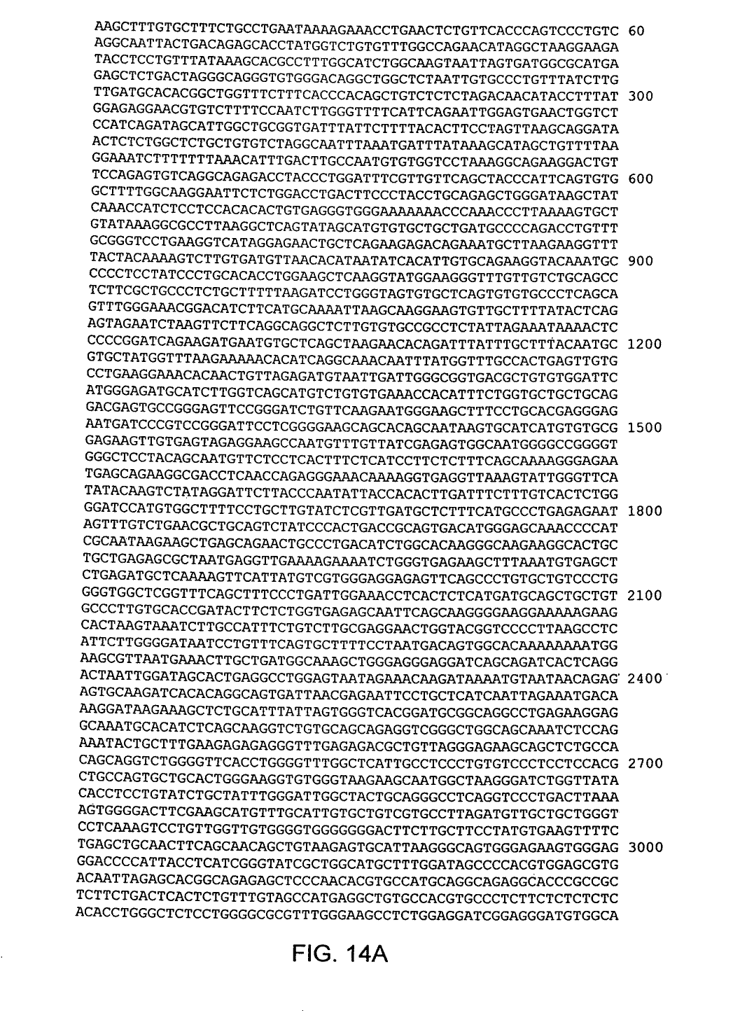

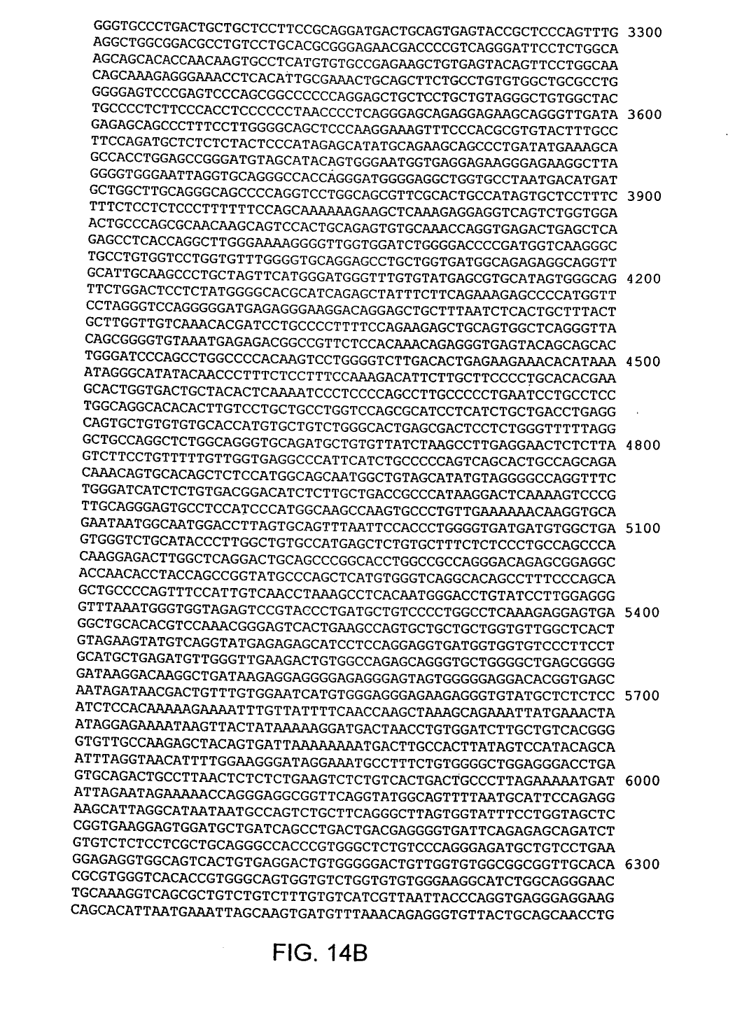

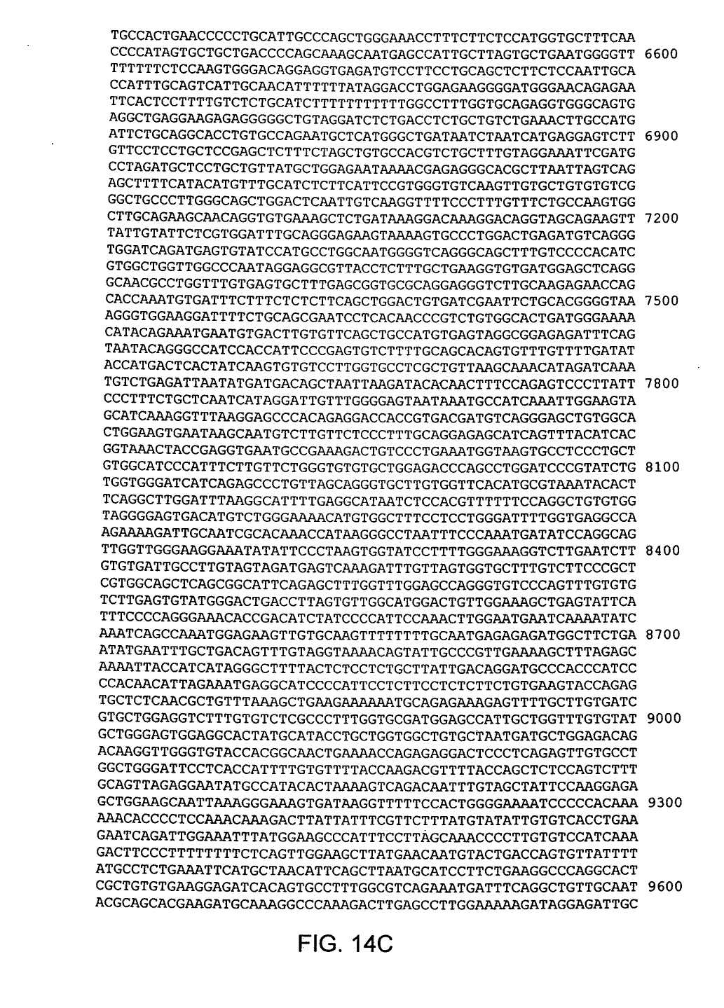

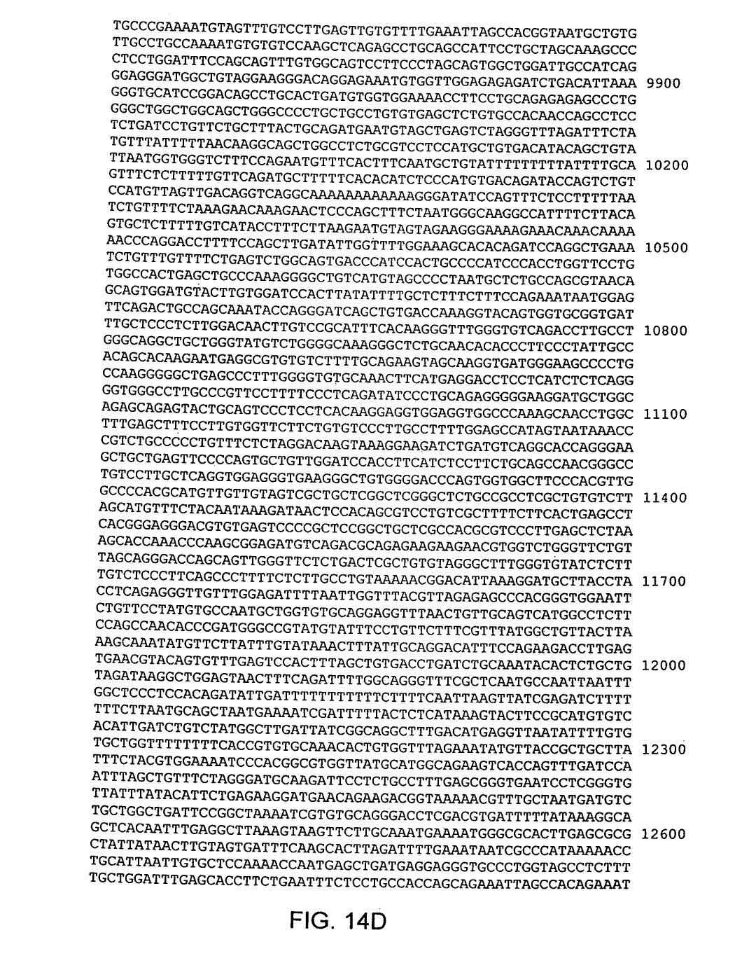

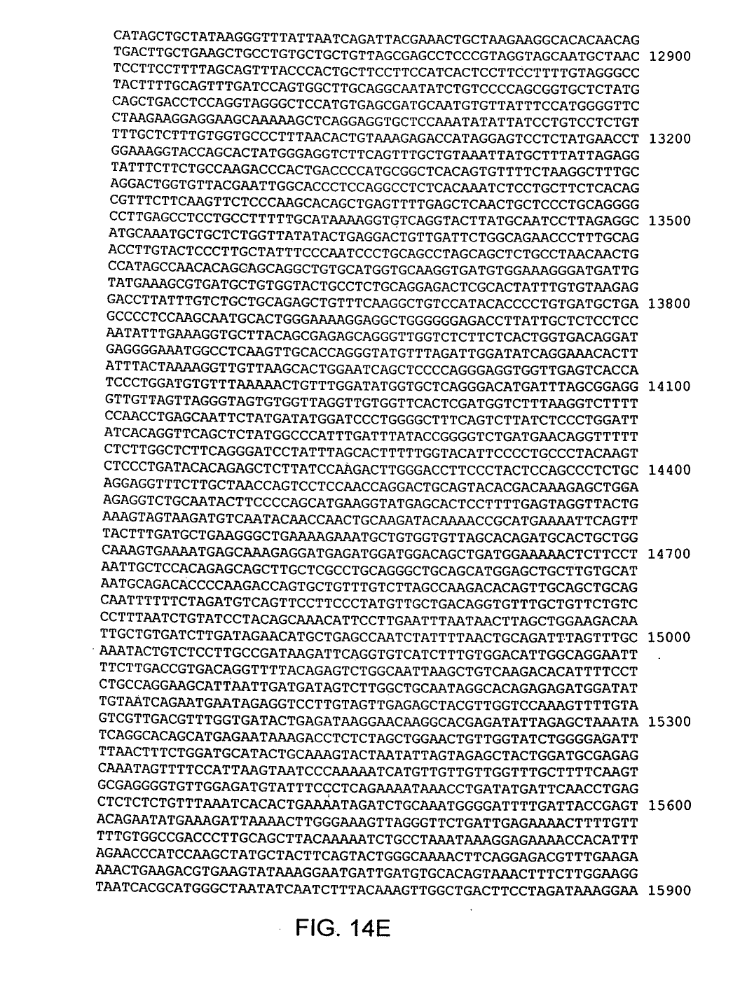

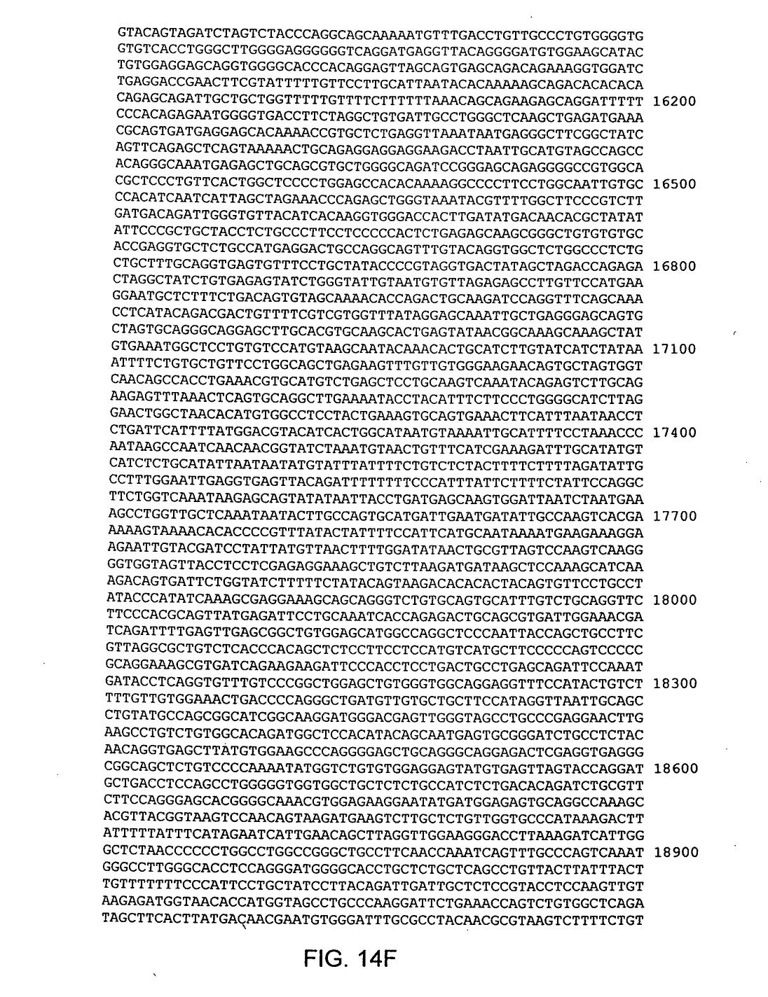

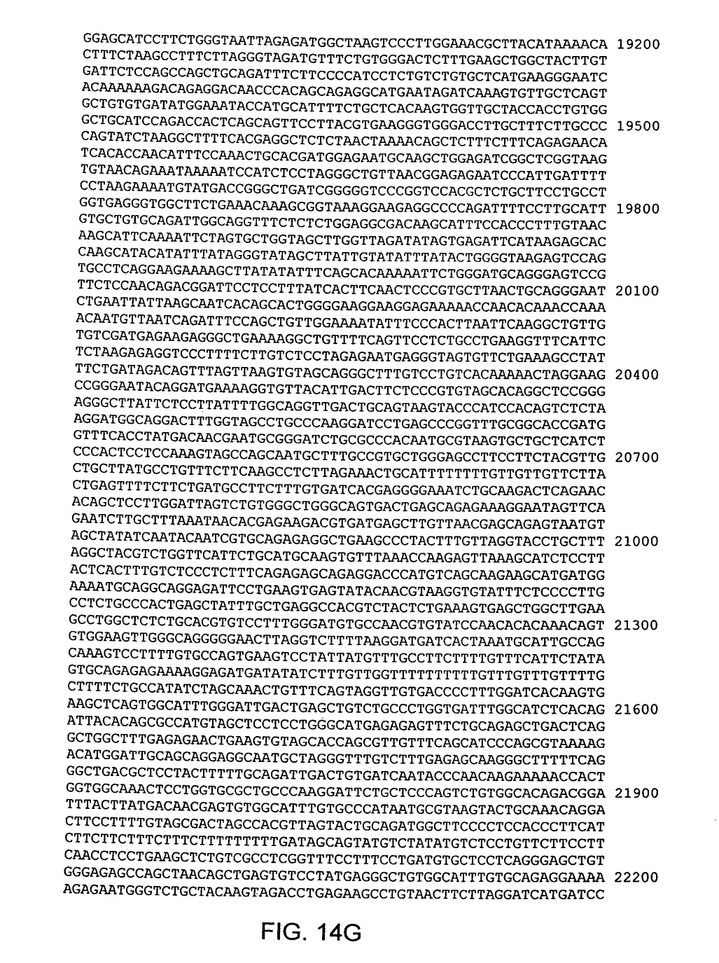

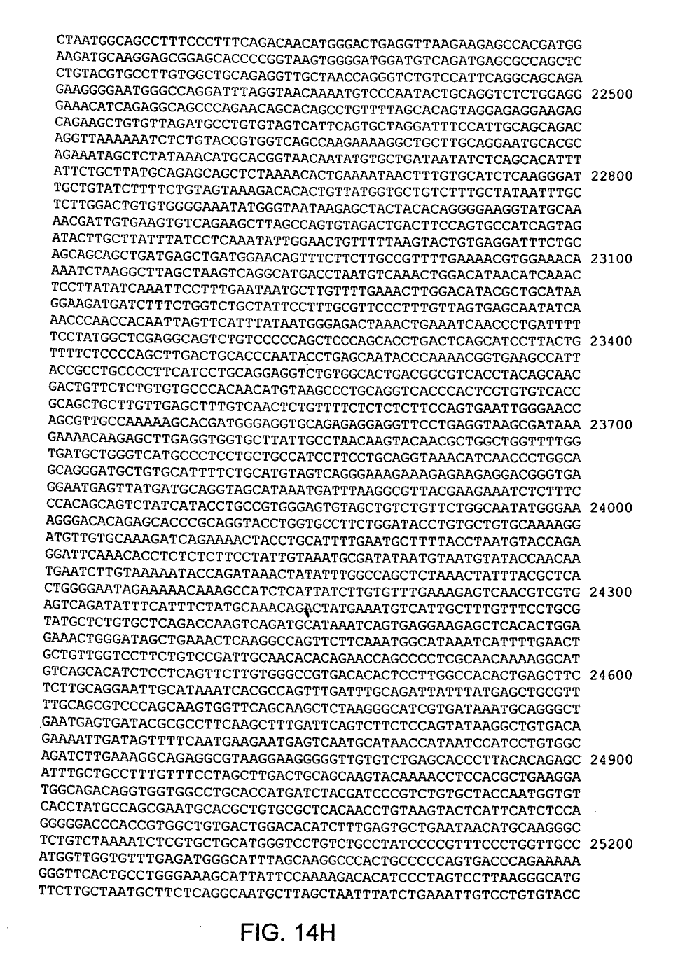

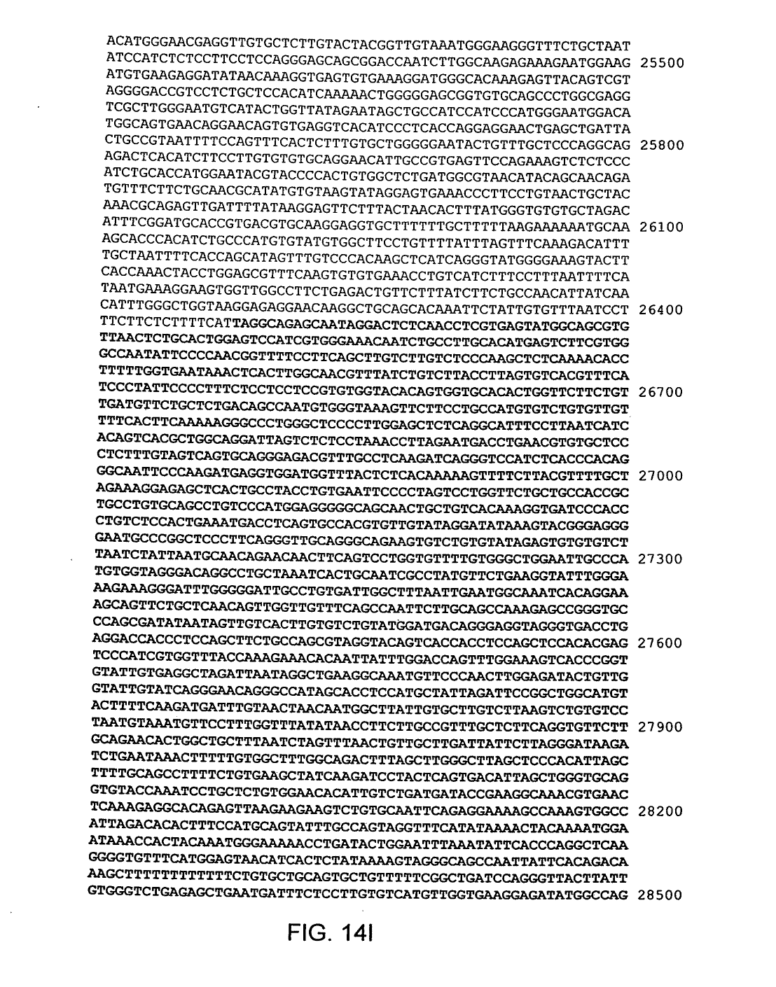

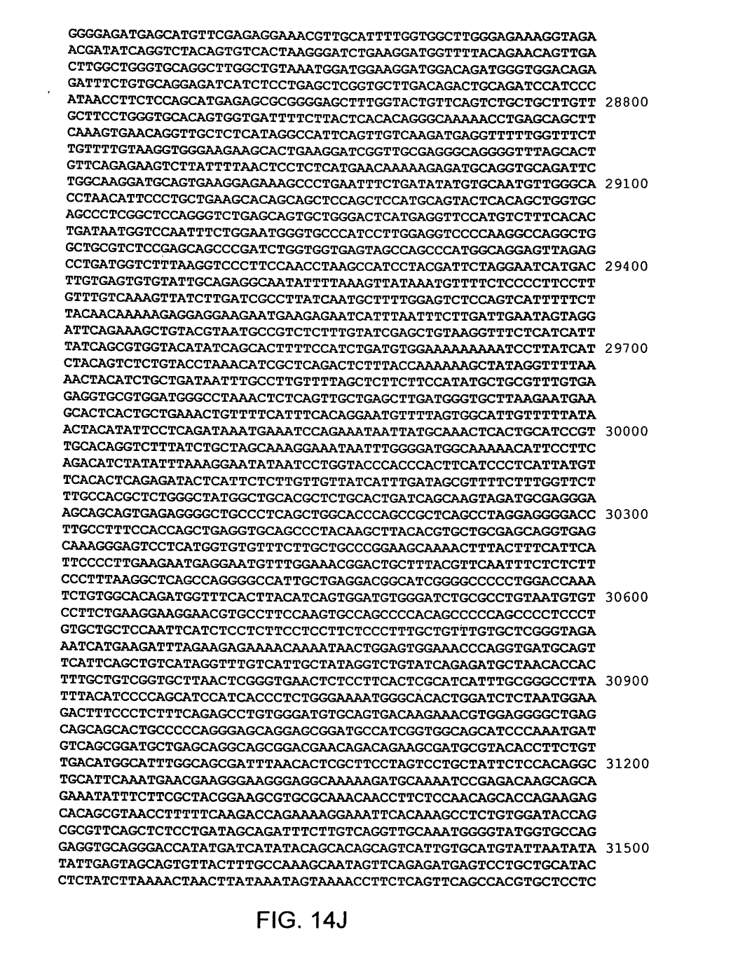

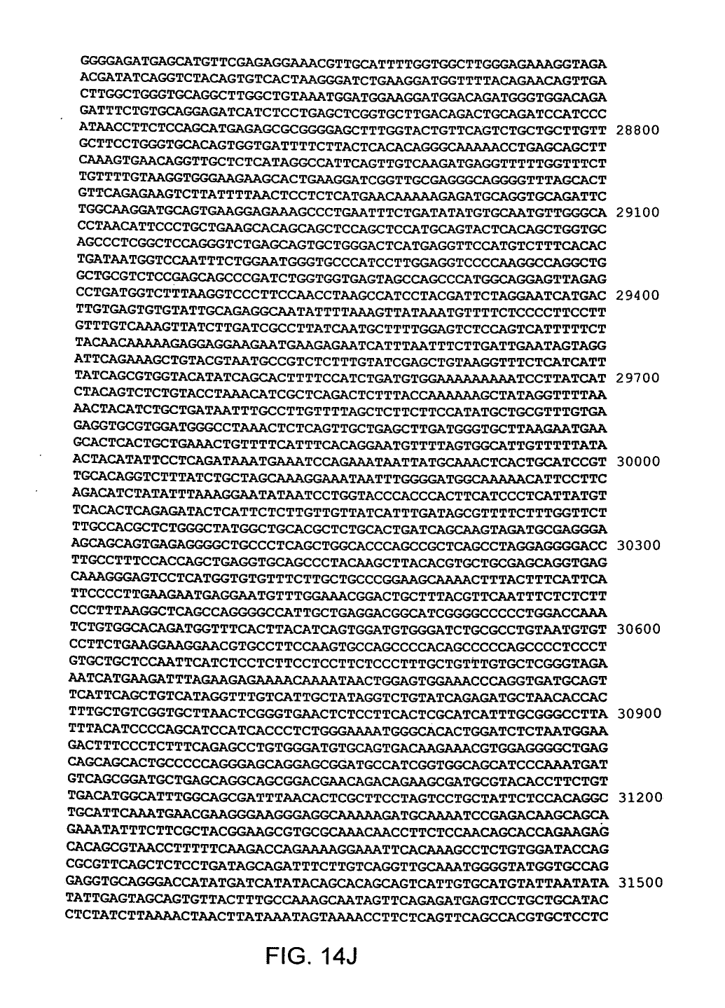

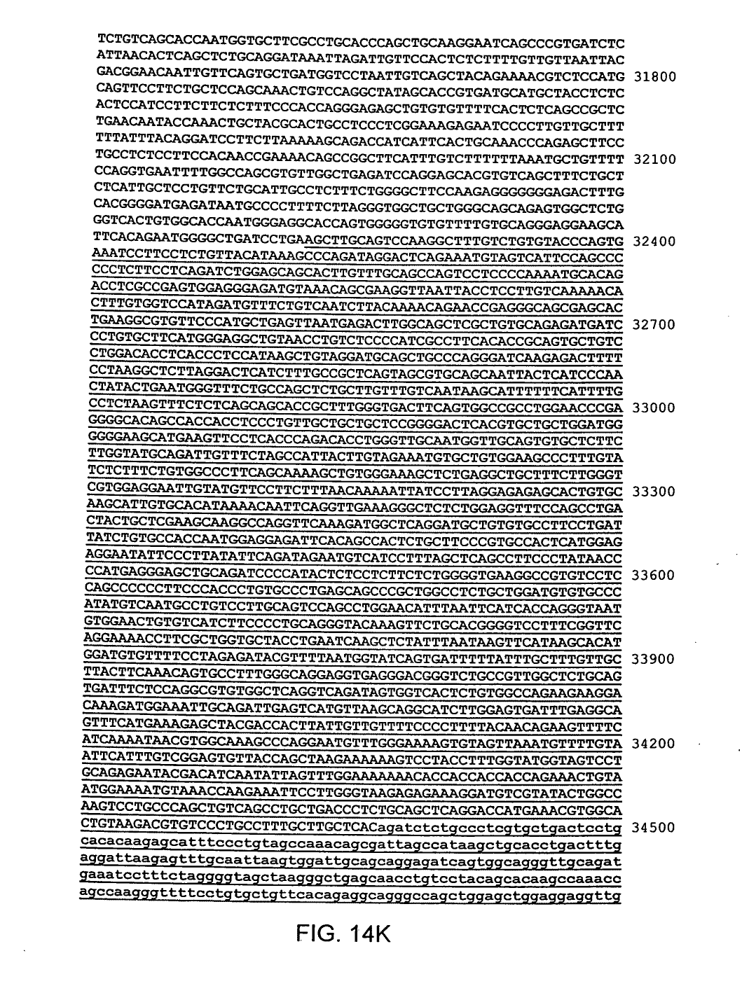

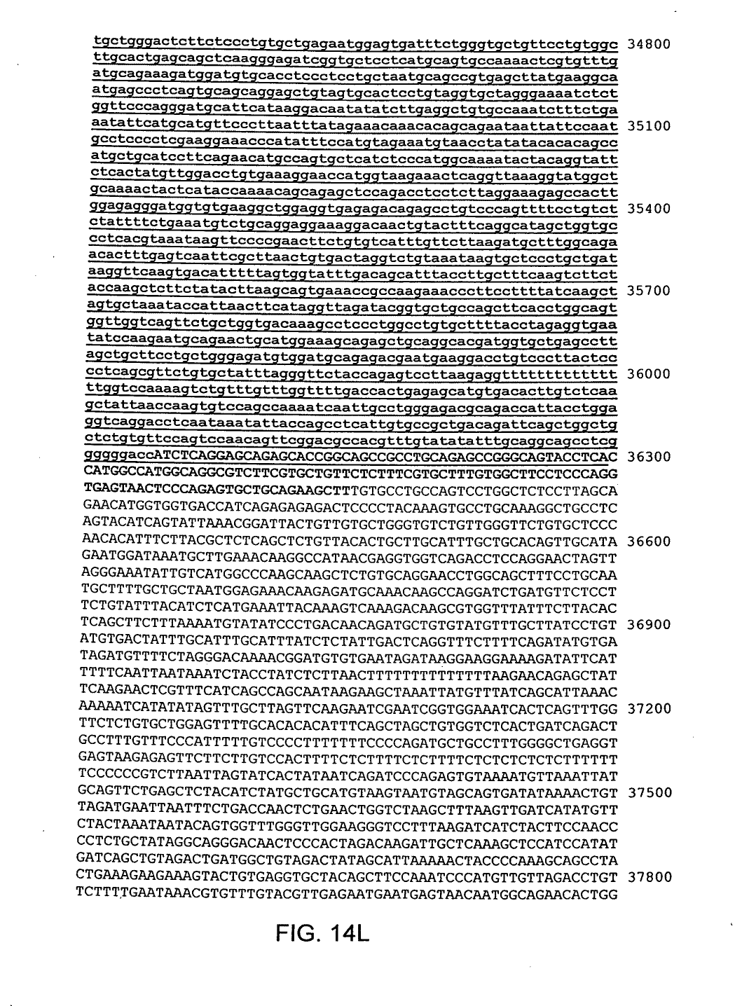

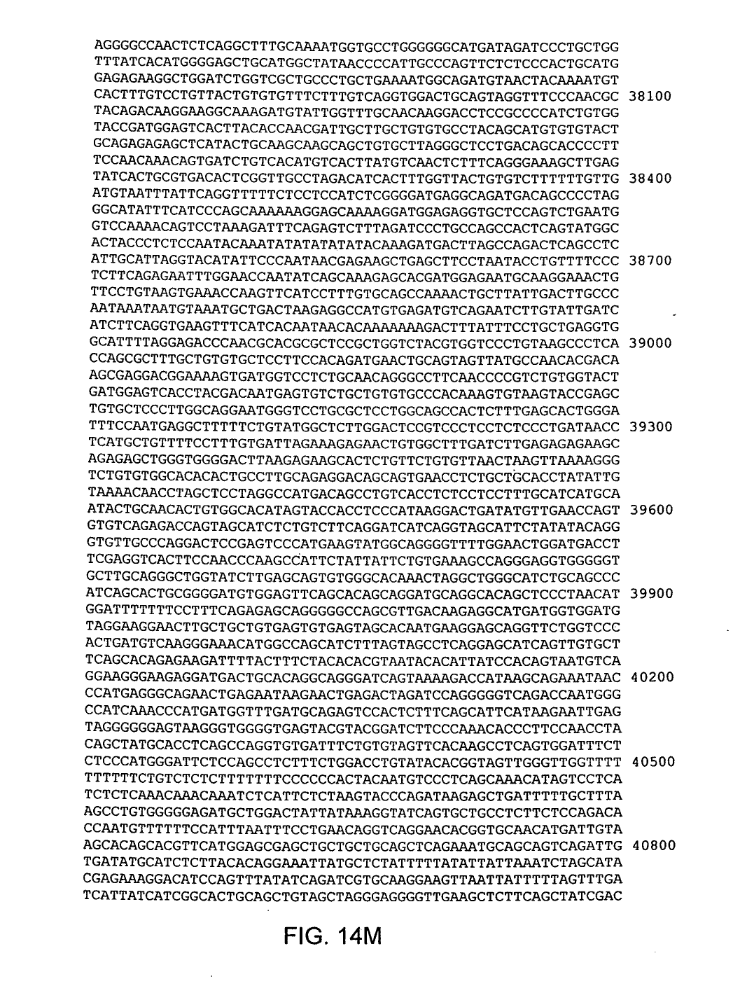

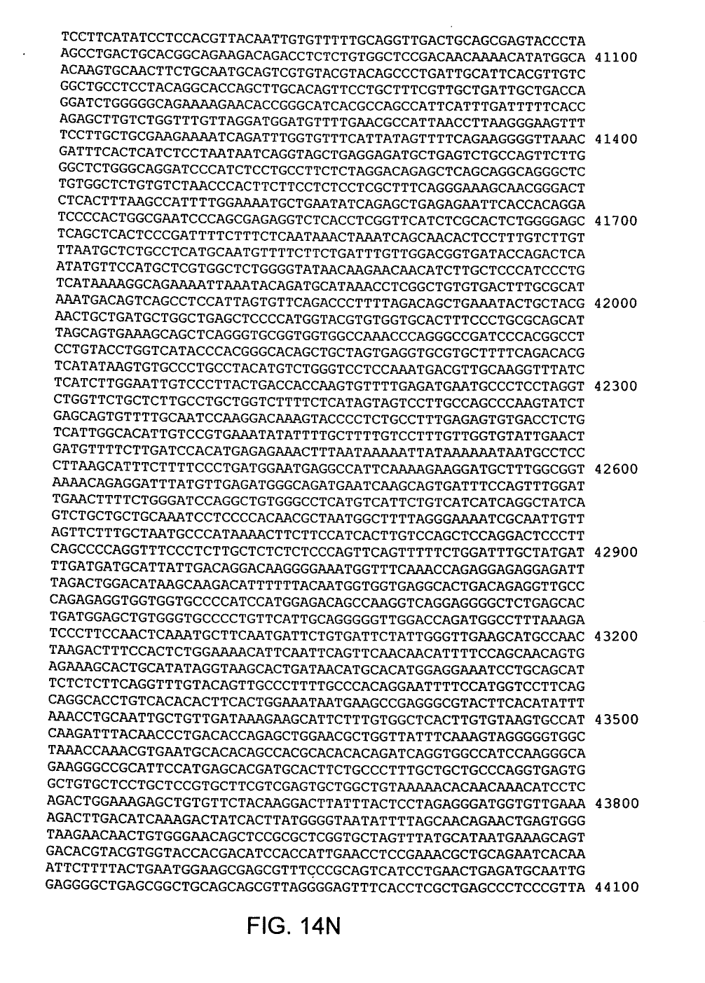

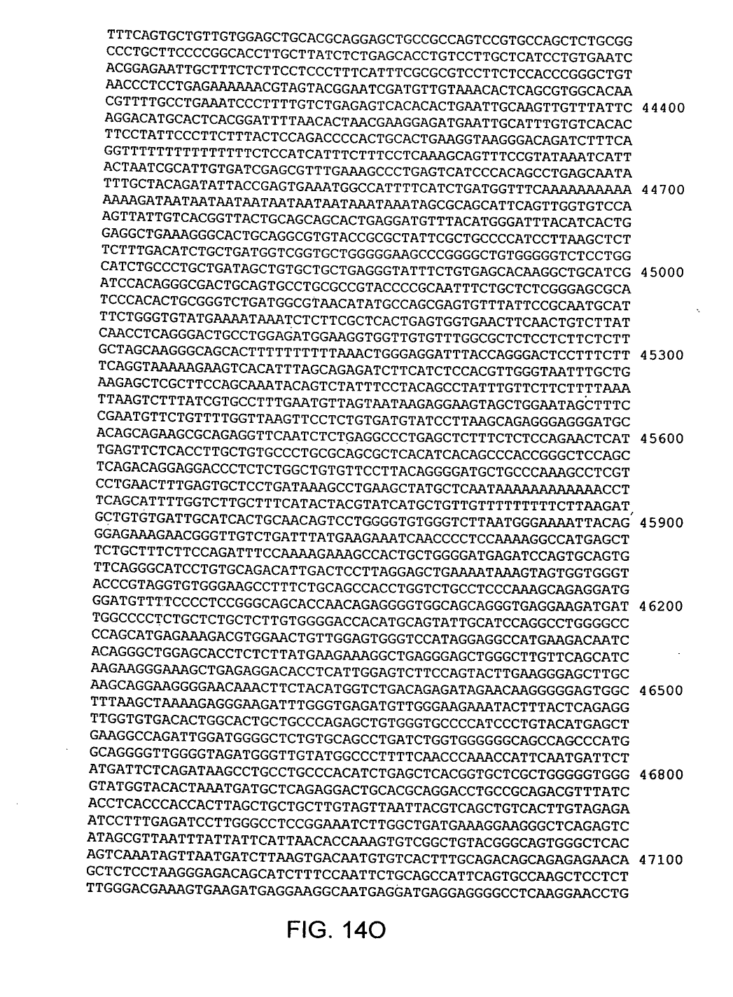

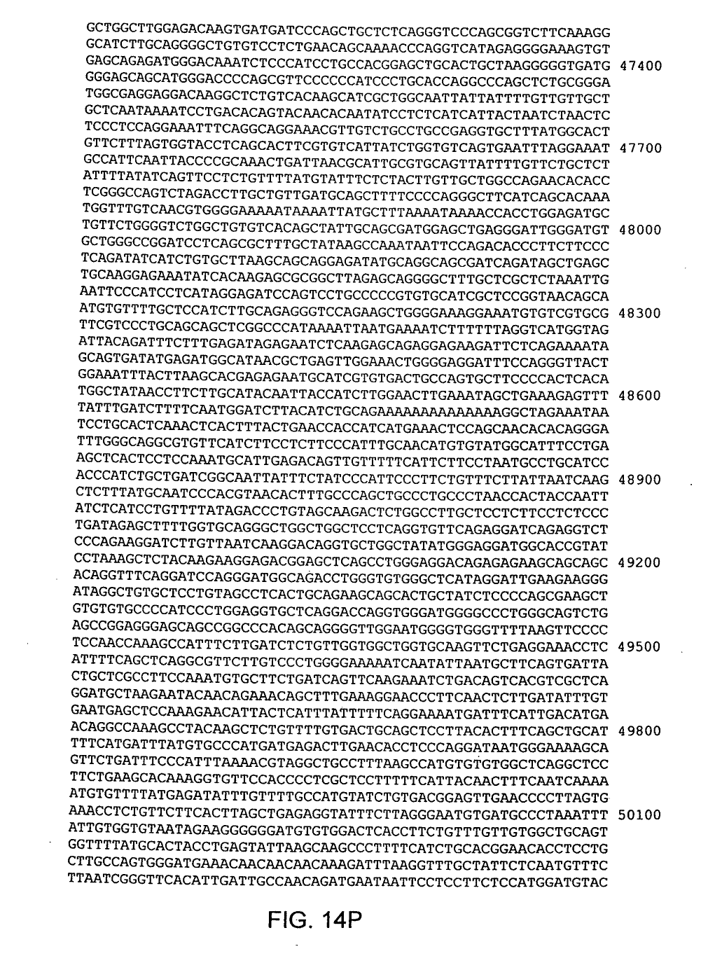

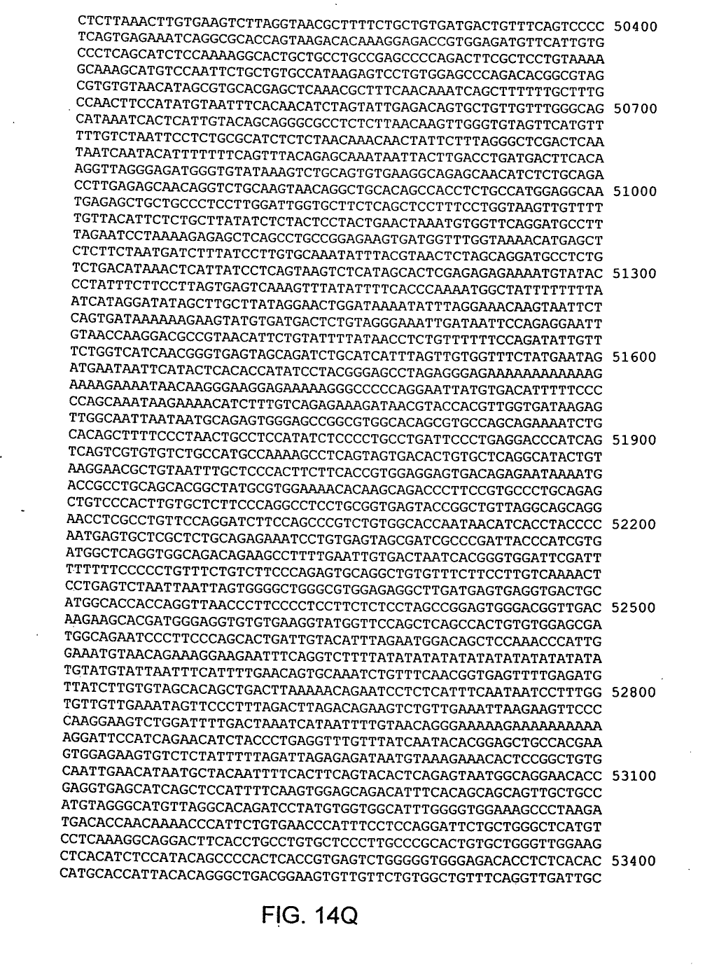

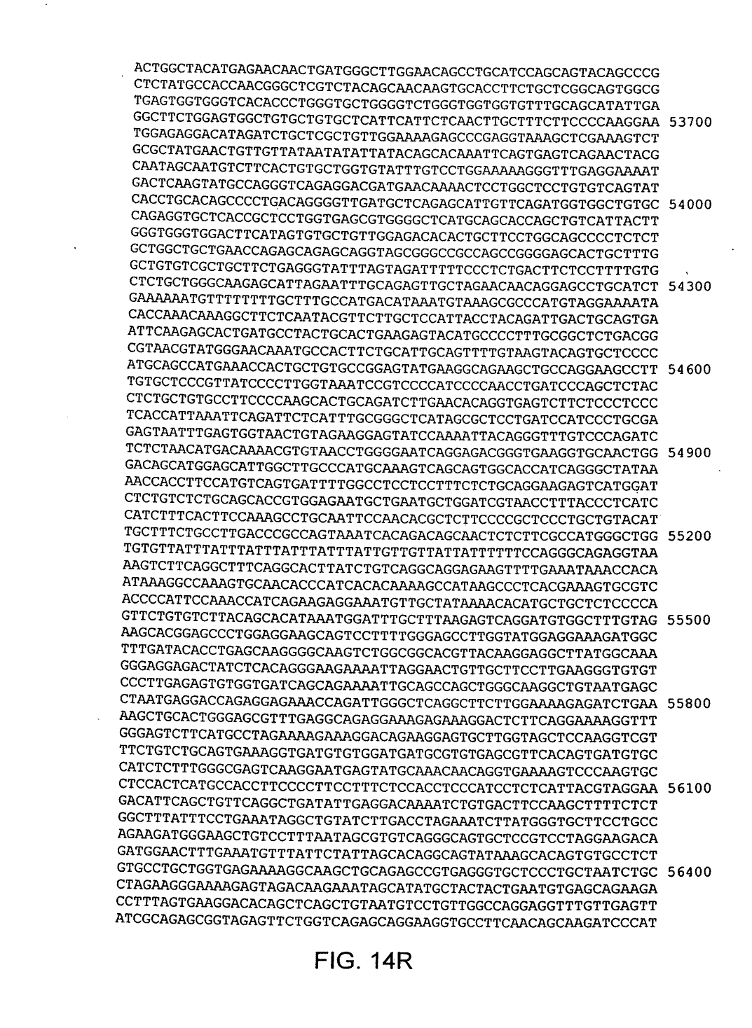

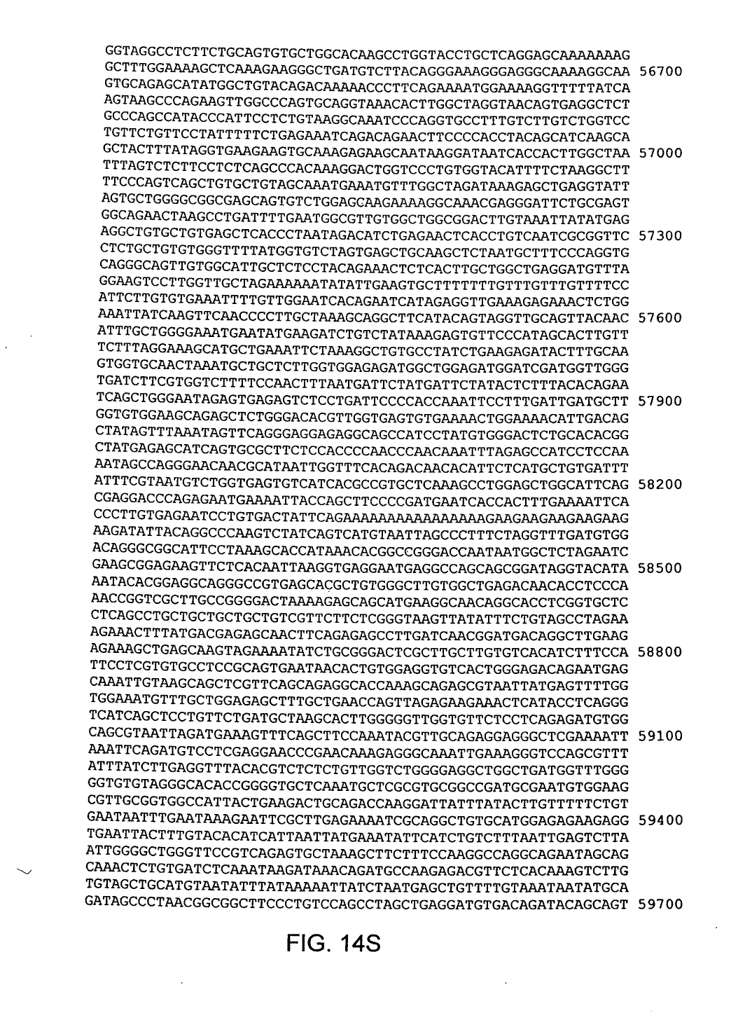

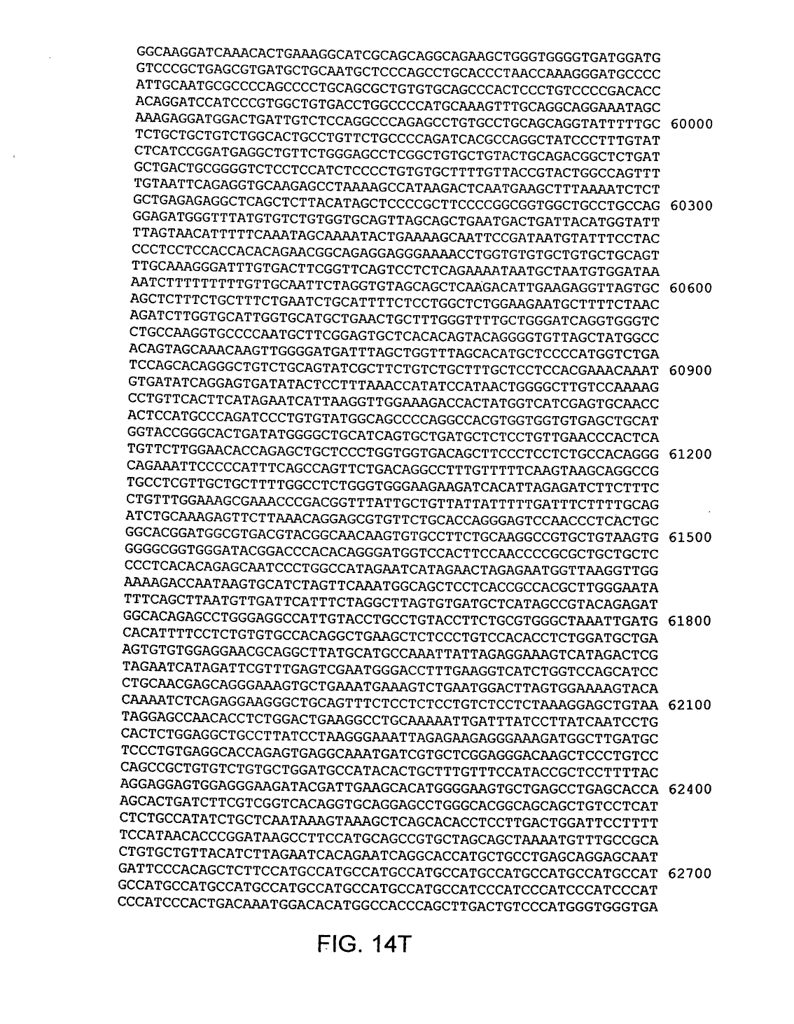

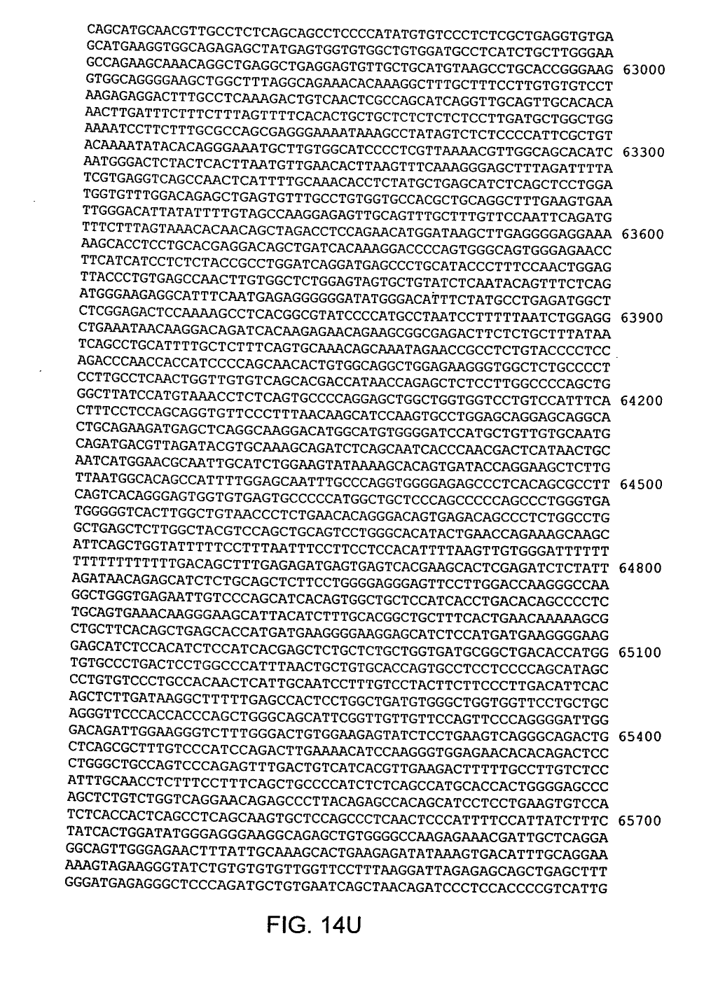

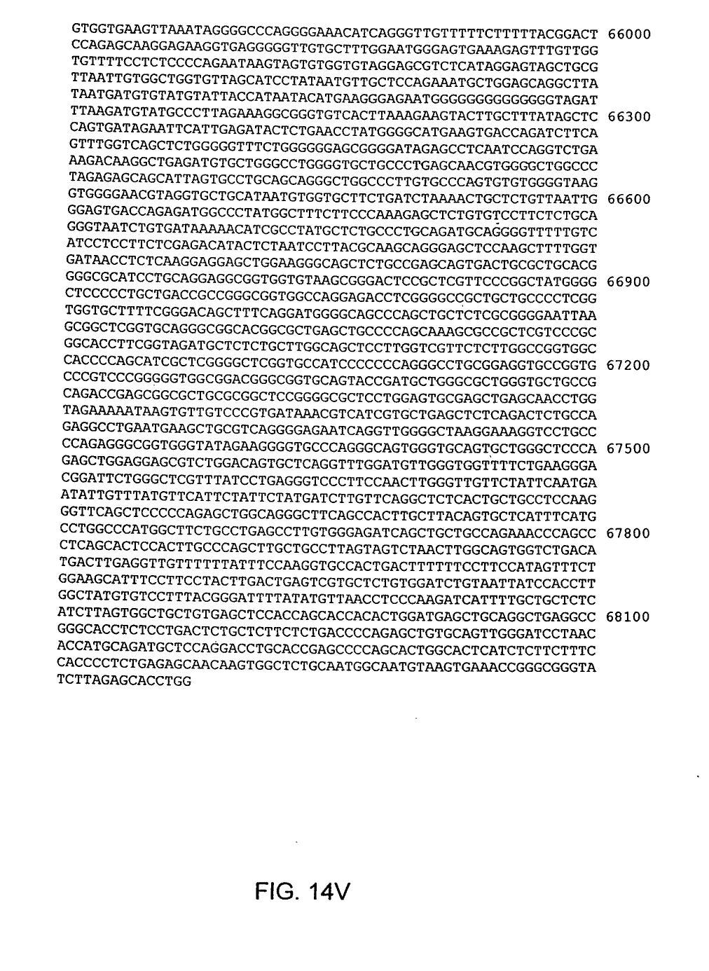

The library was screened by PCR identifying a BAC clone which included a single chicken DNA segment which extends into both the 5′ untranslated region of the ovomucoid gene and the 3′ ovoinhibitor gene. The nucleotide sequence of the clone, designated OMC24, is shown in SEQ ID NO: 36. The nucleotide region spanning from about nucleotide 68,296 to about nucleotide 75,815 of SEQ ID NO: 36 represents the BAC vector. The ovomucoid region spans from about nucleotide 1 to about nucleotide 68,295 of SEQ ID NO: 36 and is shown in FIG. 14.

The nucleotide sequence of the gene expression controlling region disclosed in SEQ ID NO: 26 is essentially encompassed in SEQ ID NO: 36 from about nucleotide 26,416 to about nucleotide 36,390. Nucleotide sequence alignment between SEQ ID NO: 26 and nucleotides 26,416 to 36,390 of SEQ ID NO: 36 show a 99.0% sequence homology. The chicken genomic DNAs which yielded SEQ ID NO: 26 and SEQ ID NO: 36 were isolated from different strains of white leghorn chickens (SEQ ID NO: 26—American Strain, SEQ ID NO: 36: Dutch Strain) thus showing the sequence diversity of the ovomucoid gene expression controlling region of the present invention. Other useful fragments or functional portions of SEQ ID NO: 36 can be easily obtained by standard techniques well known in the art.

Fragments or portions of certain DNA sequences which function to control gene expression can be identified by techniques that are well know to practitioners of ordinary skill in the art. For example, promoter analysis by saturation mutagenesis has been describe in Biol. Proced. Online (2001) Vol 1, No. 3, pp 64-69, the disclosure of which is incorporated by reference herein in its entirety. Also, for example, fragments or functional portions of the chicken ovomucoid gene region effective to control gene expression, for example, control transcription in a cell, can be identified by techniques disclosed in the Examples of the present specification. For example, functional fragments of SEQ ID NO: 36 can be identified by methods as disclosed in the present specification and by any useful method known in the field of molecular biology.

In one embodiment, the gene expression controlling region comprises a nucleotide or portion of a nucleotide sequence that is at least 50% homologous to the avian nucleic acid contained in SEQ ID NO: 36 or to the complement of the avian nucleic acid contained in SEQ ID NO: 36. For example, the gene expression controlling region may comprise a nucleotide sequence or portion of a nulceotide sequence that is at least 60% homologous to the avian nucleic acid contained in SEQ ID NO: 36 or its complement. In another example, the gene expression controlling region comprises a nucleotide sequence or portion of a nulceotide sequence that is at least 70% homologous to the avian nucleic acid contained in SEQ ID NO: 36 or its complement. In another example, the gene expression controlling region comprises a nucleotide sequence or portion of a nulceotide sequence that is at least 75% homologous to the avian nucleic acid contained in SEQ ID NO: 36 or its complement. In another example, the gene expression controlling region comprises a nucleotide sequence or portion of a nulceotide sequence that is at least 80% homologous to the avian nucleic acid contained in SEQ ID NO: 36 or its complement. In another example, the gene expression controlling region comprises a nucleotide sequence or portion of a nulceotide sequence that is at least 85% homologous to the avian nucleic acid contained in SEQ ID NO: 36 or its complement. In another example, the gene expression controlling region comprises a nucleotide sequence or portion of a nulceotide sequence that is at least 90% homologous to the avian nucleic acid contained in SEQ ID NO: 36 or its complement. In another example, the gene expression controlling region comprises a nucleotide sequence or portion of a nulceotide sequence that is at least 95% homologous to the avian nucleic acid contained in SEQ ID NO: 36 or its complement. In another example, the gene expression controlling region comprises a nucleotide sequence or portion of a nulceotide sequence that is at least 99% homologous to the avian nucleic acid contained in SEQ ID NO: 36 or its complement.

Nucleotide sequences encoding the heavy chain and light chain of an IgG1 monoclonal antibody were inserted into the 3′ UTR of the ovomucoid transcript encoding region in two separate ovomucoid BAC clones of SEQ ID NO: 36. The heavy chain and light chain coding sequences each included a signal sequence located at their 5′ ends; however, use of a signal sequence may not be required in the present invention. The resulting mRNA transcript produced by the ovomucoid gene expression controlling region for each clone contains two coding sequences; one for the ovomucoid protein and another for the antibody light chain or heavy chain downstream of the ovomucoid coding sequence. To facilitate translation of the downstream heavy chain or light chain coding sequence, an internal ribosome entry site (IRES) was inserted immediately upstream of the heavy chain or light chain coding sequence in each clone.

In another example, a CTLA4-Fc fusion coding sequence comprising a nucleotide coding sequence for the extracellular domains of the CTLA4 (cytotoxic T lymphocyte antigen 4) receptor protein linked to a nucleotide coding sequence for an immunoglobulin constant region (IgG1 Fc) was cloned into an ovomucoid BAC clone of SEQ ID NO: 36. In addition, an attB site was included in the construct. To produce this clone, the IRES-LC portion of the ovomucoid-IRES-antibody light chain clone was deleted and was replaced with an IRES-CTLA4-Fc cassette.

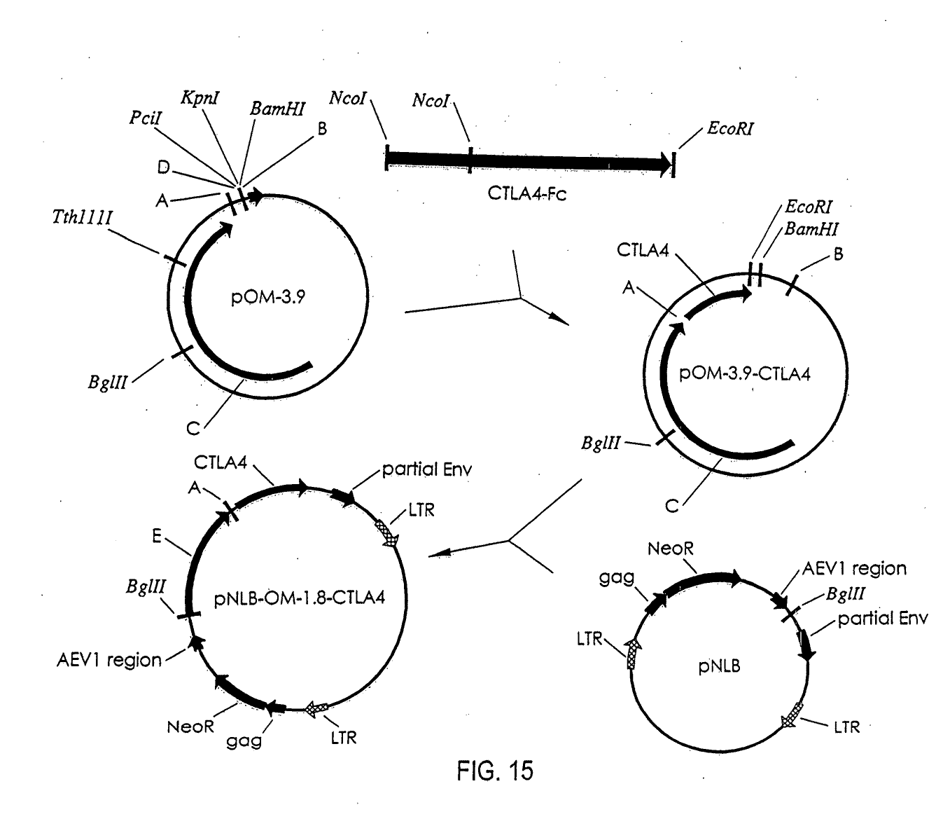

The present invention contemplates the introduction of an ovomucoid gene expression controlling region, for example, operably linked to a coding sequence of interest, which is present on a retrovirus vector, such as an ALV vector (e.g., replication deficient ALV vector), into an avian to produce a transgenic avian. One example of an ALV based vector contemplated for use herein is a pNLB vector described in for example, Cosset et al., 1991, J. Virology 65: 3388-3394, the disclosure of which is incorporated in its entirety herein by reference and U.S. patent application Ser. No. 10/463,980, filed Jun. 17, 2003, the disclosure of which is incorporated in its entirety herein by reference. In one example, a CTLA4-Fc fusion coding sequence was operably linked to an approximately 3.9 kb ovomucoid gene expression controlling region (Fragment B of FIG. 14). In yet another example, a CTLA4-Fc fusion coding sequence was operably linked to an approximately 1.8 kb ovomucoid gene expression controlling region (Fragment C of FIG. 14). The Promoter-coding sequence cassette was inserted into a replication deficient avian leucosis virus (ALV) based vector as shown in FIG. 15.

Disclosed above are examples of expression constructs that can be produced in accordance with the present invention. However, these are merely examples and it is contemplated that any nucleic acid sequence encoding a useful amino acid sequence can be operably linked to an avian ovomucoid gene expression controlling region of the present invention so as to be expressed in an avian cell, for example, in cells of a transgenic avian such as a chicken, turkey, duck, goose, quail, pheasant, parrot, finch, ratites including ostrich, emu or cassowary.

The present invention can be used to express, in large yields and at low cost, a wide range of desired proteins including those used as human and animal pharmaceuticals, diagnostics, and livestock feed additives. Proteins such as growth hormones, cytokines, structural proteins and enzymes, including human growth hormone, interferon, lysozyme, and β-casein, are examples of proteins that are desirably expressed in the oviduct and deposited in eggs according to the invention. Other possible proteins to be produced include, but are not limited to, albumin, α-1 antitrypsin, antithrombin III, collagen, factors VIII, IX, X (and the like), fibrinogen, hyaluronic acid, insulin, lactoferrin, protein C, erythropoietin (EPO), granulocyte colony-stimulating factor (G-CSF), granulocyte macrophage colony-stimulating factor (GM-CSF), tissue-type plasminogen activator (tPA), feed additive enzymes, somatotropin, and chymotrypsin Immunoglobulins and genetically engineered antibodies, including immunotoxins that bind to surface antigens on human tumor cells and destroy them, can also be expressed for use as pharmaceuticals or diagnostics. It is contemplated that immunoglobulin amino acid sequences expressed in avian cells following transfection by the methods of the present invention may include monomeric heavy and light chains, single-chain antibodies or multimeric immunoglobulins comprising variable heavy and light chain regions, i.e., antigen-binding domains, or intact heavy and light immunoglobulin chains.

The chicken ovomucoid gene expression controlling region of the present invention may include the nucleotide elements that are positioned 5′ upstream of the transcription start site of the native chicken ovomucoid locus and which are necessary for the regulated expression of a downstream amino acid sequence-encoding nucleic acid. It is contemplated that this region may include transcription controlling regions which are regulated by certain hormones including, for example, steroid hormones and the like.

One aspect of the present invention, therefore, provides a novel isolated nucleic acid that comprises the nucleotide sequence SEQ ID NO: 26, shown in FIG. 4, (Genbank Accession No: AF 453747) and derivatives and variants thereof, that is located immediately 5′ upstream of the transcription start site of the chicken ovomucoid gene locus.

In one embodiment of the present invention, the isolated nucleic acid may be isolated from an avian selected from the group consisting of a chicken, a turkey, a duck, a goose, a quail, a pheasant, a ratite, an ornamental bird or a feral bird.

In another embodiment of the present invention, the isolated nucleic acid is obtained from a chicken. In this embodiment, the isolated nucleic acid has the sequence of SEQ ID NO: 26, as shown in FIG. 4, or a variant thereof. SEQ ID NO: 26 was cloned into pBluescript KS II (+/−) vector, as described in Example 2, and named pBS-OVMUP-10. pBS-OVMUP-10 was deposited with American Type Culture Collection (ATCC), 10801 University Blvd., Manassas, Va. 20110, as ATCC No. PTA-4821 on Nov. 26, 2002 under the conditions set forth in the Budapest Treaty.

Another aspect of the invention provides nucleic acids that can hybridize under high, medium or low stringency conditions to an isolated nucleic acid comprising a chicken ovomucoid gene expression controlling region having all, a derivative of, or a portion of the nucleic acid sequence SEQ ID NO: 26 shown in FIG. 4 and direct expression of an amino acid sequence coding sequence in an avian oviduct cell. The nucleotide sequence determined from the isolation of the ovomucoid gene expression controlling region from a chicken (SEQ ID NO: 26) will allow for the generation of probes designed for use in identifying ovomucoid gene expression controlling regions, or homologs thereof in other avian species.

Fragments of a nucleic acid comprising a portion of the subject ovomucoid gene expression controlling region are also within the scope of the invention. As used herein, a fragment of the nucleic acid comprising an active portion of a ovomucoid gene expression controlling region refers to a nucleotide sequence having fewer nucleotides than the nucleotide sequence comprising the entire nucleic acid sequence of the ovomucoid gene expression controlling region.

A fragment of the ovomucoid gene expression controlling region may contain one or more of the following elements: the ovoinhibitor gene 3′ untranslated region from bases positions 1-255 as shown in FIG. 4, a CR1-like element located at base positions 2761-3024 as shown in FIG. 4, the region from base positions 9403-9920, as shown in FIG. 4 which includes a portion of the 5′ untranslated region of the ovomucoid gene. Alternatively, the fragment may be about 10 or about 20 or about 50 or about 75 or about 100 or about 150 or about 200 or about 250 or about 300 or about 500 or about 1000 or about 2000 or about 4000 or about 5000 or about 6000 or about 7000 or about 8000 or about 9000 or about 10,000 or about 20,000 or about 30,000 or about 40,000 or about 50,000 or about 60,000 nucleotides in length and be capable of directing expression of an operably linked heterologous gene sequence, particularly in an avian cell, for example, in an avian oviduct cell of a transgenic avian or in an avian cell in culture.

In one embodiment of the present invention, the nucleotide sequence of the isolated DNA molecule of the present invention may be used as a probe in nucleic acid hybridization assays for the detection of the ovomucoid gene expression controlling region. The nucleotide sequence of the present invention may be used in any nucleic acid hybridization assay system known in the art, including, but not limited to, Southern blots (Southern, E. M. J. Mol. Biol. 98: 508 (1975)), Northern blots (Thomas et al. (1980) Proc. Natl. Acad. Sci. 77: 5201-05), and Colony blots (Grunstein et al. (1975) Proc. Natl. Acad. Sci. 72: 3961-65), which are hereby incorporated by reference in their entireties. Alternatively, the isolated DNA molecules of the present invention can be used in a gene amplification detection procedure such as a polymerase chain reaction (Erlich et al. (1991) Science 252: 1643-51, which is hereby incorporated by reference in its entirety) or in restriction fragment length polymorphism (RFLP) diagnostic techniques, as described in Watson et al., (2d ed. 1992), Recombinant DNA, Scientific American Books, 519-522, 545-547, which is hereby incorporated by reference.

Nucleic acids constructed in accordance with the present invention can be labeled to provide a signal as a means of detection. For example, radioactive elements such as ³²P, ³H, and ³⁵S or the like provide sufficient half-life to be useful as radioactive labels. Other materials useful for labeling synthetic nucleotides include fluorescent compounds, enzymes and chemiluminescent moieties. Methods useful in selecting appropriate labels and binding protocols for binding the labels to the synthetic nucleotides are well known to those of skill in the art. Standard immunology manuals such as Promega: Protocol and Applications Guide, 2nd Edition, 1991 (Promega Corp., Madison, Wis., the disclosure of which is incorporated herein in its entirety) may be consulted to select an appropriate labeling protocol without undue experimentation.

In another embodiment of the present invention, an isolated nucleic acid molecule of the present invention includes a nucleic acid that hybridizes to SEQ ID NO: 26 or the complement thereof, or the insert in pBS-OVMUP-10, under high, moderate or low stringency hybridization conditions.

In another embodiment of the present invention, an avian ovomucoid gene expression controlling region gene or nucleic acid molecule can be an allelic variant of SEQ ID NO: 26 or SEQ ID NO: 36 or a homolog from a different avian, e.g., quail, duck, etc.

The present invention also contemplates the use of antisense nucleic acid molecules that are designed to be complementary to a coding strand of a nucleic acid (i.e., complementary to an mRNA sequence) or, alternatively, complimentary to a 5′ or 3′ untranslated region of the mRNA. Another use of synthetic nucleotides is as primers (DNA or RNA) for a polymerase chain reaction (PCR), ligase chain reaction (LCR), or the like.

Synthesized oligonucleotides can be produced in variable lengths. The number of bases synthesized will depend upon a variety of factors, including the desired use for the probes or primers. Additionally, sense or anti-sense nucleic acids or oligonucleotides can be chemically synthesized using modified nucleotides to increase the biological stability of the molecule or of the binding complex formed between the anti-sense and sense nucleic acids. For example, acridine substituted nucleotides can be synthesized. Protocols for designing isolated nucleotides, nucleotide probes, and/or nucleotide primers are well-known to those of ordinary skill, and can be purchased commercially from a variety of sources (e.g., Sigma Genosys, The Woodlands, Tex. or The Great American Gene Co., Ramona, Calif.).

The nucleic acid sequence of a chicken ovomucoid gene expression controlling region nucleic acid molecule of the present invention allows one skilled in the art to, for example, (a) make copies of those nucleic acid molecules by procedures such as, but not limited to, insertion into a cell for replication by the cell, by chemical synthesis or by procedures such as PCR or LCR, (b) obtain nucleic acid molecules which include at least a portion of such nucleic acid molecules, including full-length genes, full-length coding regions, regulatory control sequences, truncated coding regions and the like, (c) obtain ovomucoid gene expression controlling region nucleic acid homologs in other avian species such as, but not limited to, turkey, duck, goose, quail, pheasant, parrot, finch, ratites including ostrich, emu and cassowary and, (d) to obtain isolated nucleic acids capable of hybridizing to an avian ovomucoid gene expression controlling region nucleic acid and be used to detect the presence of nucleic acid-related sequences by complementation between the probe and the target nucleic acid.

Such nucleic acid homologs can be obtained in a variety of ways including by screening appropriate expression libraries with antibodies of the present invention, using traditional cloning techniques to screen appropriate libraries, amplifying appropriate libraries or DNA using oligonucleotide primers of the present invention in a polymerase chain reaction or other amplification method, and screening public and/or private databases containing genetic sequences using nucleic acid molecules of the present invention to identify targets. Examples of libraries to screen, or from which to amplify nucleic acid molecules, include but are not limited to mammalian BAC libraries, genomic DNA libraries, and cDNA libraries. Similarly, sequence databases useful for screening to identify sequences in other species homologous to chicken ovomucoid gene expression controlling region include, but are not limited to, GenBank and the mammalian Gene Index database of The Institute of Genomics Research (TIGR).

Another aspect of the present invention is a recombinant DNA molecule comprising the novel isolated avian ovomucoid gene expression controlling region of the present invention operably linked to a selected amino acid sequence-encoding nucleic acid insert, and which may express the nucleic acid insert when transfected to a suitable host cell, preferably an avian cell. The nucleic acid insert may be placed in frame with a signal peptide sequence, whereby translation initiation from the transcript may start with the signal peptide and continue through the nucleic acid insert, thereby producing an expressed amino acid sequence having the desired amino acid sequence.

It is anticipated that the recombinant DNA may further comprise a polyadenylation signal sequence that will allow the transcript directed by the novel ovomucoid gene expression controlling region to proceed beyond the nucleic acid insert encoding an amino acid sequence and allow the transcript to further comprise a 3′ untranslated region and a polyadenylated tail. Any functional polyadenylation signal sequence may be linked to the 3′ end of the nucleic acid insert including the SV40 polyadenylation signal sequence, bovine growth hormone adenylation sequence or the like, or derivatives thereof. One embodiment of the present invention is a recombinant DNA molecule comprising the isolated avian ovomucoid gene expression controlling region of the present invention, operably linked to a nucleic acid insert encoding an amino acid sequence which may include a polyadenylation signal sequence. In certain embodiments, the recombinant DNA molecule which includes include a polyadenylation signal sequence is an artificial chromosome.

Another aspect of the present invention is to provide nucleic acid sequences of a protein optimized for expression in avian cells, and derivatives and fragments thereof. For example, it is contemplated that when the recombinant DNA is to be delivered to a recipient cell for expression therein, the sequence of the nucleic acid sequence may be modified so that the codons are optimized for the codon usage of the recipient species. When a heterologous nucleic acid is to be delivered to a recipient cell for expression therein, the sequence of the nucleic acid sequence may be modified so that the codons are optimized for the codon usage of the recipient species. For example, if the heterologous nucleic acid is transfected into a recipient chicken cell, the sequence of the expressed nucleic acid insert is optimized for chicken codon usage. This may be determined from the codon usage of at least one, and preferably more than one, protein expressed in a chicken cell. For example, the codon usage may be determined from the nucleic acid sequences encoding the proteins ovalbumin, lysozyme, ovomucin and ovotransferrin of chicken. Briefly, the DNA sequence for the target protein may be optimized using the BACKTRANSLATE® program of the Wisconsin Package, version 9.1 (Genetics Computer Group, Inc., Madison, Wis.) with a codon usage table compiled from the chicken (Gallus gallus) ovalbumin, lysozyme, ovomucoid, and ovotransferrin proteins. The template and primer oligonucleotides are then amplified, by any means known in the art, including but not limited to PCR with Pfu polymerase (STRATAGENE®, La Jolla Calif.).

In one exemplary embodiment of a heterologous nucleic acid for use by the methods of the present invention, a nucleic acid insert encoding the human interferon α2b amino acid sequence optimized for codon-usage by the chicken is used. Optimization of the sequence for codon usage is useful in elevating the level of translation in avian eggs.

It is contemplated to be within the scope of the present invention for any nucleic acid encoding an amino acid sequence to be optimized for expression in avian cells. It is further contemplated that the codon usage may be optimized for a particular avian species used as a source of the host cells. In one embodiment of the present invention, the heterologous amino acid sequence is encoded using the codon-usage of a chicken.

In yet another embodiment of the present invention, the recombinant DNA comprises the isolated avian ovomucoid gene expression controlling region operably linked to a nucleic acid encoding a human interferon a2b and the SV40 polyadenylation sequence.

Proteins produced in accordance with methods of the present invention may be purified by any known conventional technique. In a one embodiment, the protein is purified from chicken eggs, preferably egg whites. For example, chicken cells may be homogenized and centrifuged. The supernatant is then subjected to sequential ammonium sulfate precipitation and heat treatment. The fraction containing the protein of the present invention is subjected to gel filtration in an appropriately sized dextran or polyacrylamide column to separate the proteins. If necessary, the protein fraction may be further purified by HPLC.

The invention provides methods for producing multimeric proteins, preferably immunoglobulins, such as antibodies, and antigen binding fragments thereof.

In one embodiment of the present invention, the multimeric protein is an immunoglobulin, wherein the first and second heterologous amino acid sequences are an immunoglobulin heavy and light chain respectively. Illustrative examples of this and other aspects and embodiments of the present invention for the production of heterologous multimeric amino acid sequences in avian cells are fully disclosed in U.S. patent application Ser. No. 09/877,374, filed Jun. 8, 2001, published as US-2002-0108132-A1 on Aug. 8, 2002, and U.S. patent application Ser. No. 10/251,364, filed Sep. 18, 2002, now U.S. Pat. No. 7,312,374, issued Dec. 25, 2007, the disclosures of which are incorporated herein by reference in their entirety. In one embodiment of the present invention, therefore, the multimeric protein is an immunoglobulin wherein the first and second heterologous amino acid sequences are an immunoglobulin heavy and light chain respectively. Accordingly, the invention provides immunoglobulin and other multimeric proteins that have been produced by transgenic avians of the invention.

In the various embodiments of this aspect of the present invention, an immunoglobulin amino acid sequence encoded by the transcriptional unit of at least one expression vector may be an immunoglobulin heavy chain amino acid sequence comprising a variable region or a variant thereof, and may further comprise a D region, a J region, a C region, or a combination thereof. An immunoglobulin amino acid sequence encoded by the transcriptional unit of an expression vector comprising an ovomucoid gene expression controlling region may also be an immunoglobulin light chain amino acid sequence comprising a variable region or a variant thereof, and may further comprise a J region and a C region. It is also contemplated to be within the scope of the present invention for the immunoglobulin regions to be derived from the same animal species, or a mixture of species including, but not only, human, mouse, rat, rabbit and chicken. In certain embodiments, the antibodies are human or humanized.

In other embodiments of the present invention, the immunoglobulin amino acid sequence encoded by the transcriptional unit of at least one expression vector comprises an immunoglobulin heavy chain variable region, an immunoglobulin light chain variable region, and a linker peptide thereby forming a single-chain antibody capable of selectively binding an antigen.

Another aspect of the present invention provides a method for the production in an avian of a heterologous protein capable of forming an antibody suitable for selectively binding an antigen comprising the step of producing a transgenic avian incorporating at least one transgene, wherein the transgene encodes at least one heterologous amino acid sequence selected from an immunoglobulin heavy chain variable region, an immunoglobulin heavy chain comprising a variable region and a constant region, an immunoglobulin light chain variable region, an immunoglobulin light chain comprising a variable region and a constant region, and a single-chain antibody comprising two peptide-linked immunoglobulin variable regions.

In an embodiment of this method of the present invention, the isolated heterologous protein is an antibody capable of selectively binding to an antigen. In one embodiment, the antibody may be generated by combining at least one immunoglobulin heavy chain variable region and at least one immunoglobulin light chain variable region, preferably cross-linked by at least one di-sulfide bridge. The combination of the two variable regions will generate a binding site capable of binding an antigen using methods for antibody reconstitution that are well known in the art.

It is, however, contemplated to be within the scope of the present invention for immunoglobulin heavy and light chains, or variants or derivatives thereof, to be expressed in separate transgenic avians, and therefore isolated from separate media including serum or eggs, each isolate comprising a single species of immunoglobulin amino acid sequence. The method may include combining certain isolated heterologous immunoglobulin amino acid sequences, thereby producing an antibody capable of selectively binding to an antigen. In this embodiment, two individual transgenic avians may be generated wherein one transgenic produces serum or eggs having an immunoglobulin heavy chain variable region, or an amino acid sequence comprising such, expressed therein. A second transgenic avian, having a second transgene, produces serum or eggs having an immunoglobulin light chain variable region, or an amino acid sequence comprising such, expressed therein. The amino acid sequences may be isolated from their respective sera and eggs and combined in vitro to generate a binding site capable of binding an antigen.

The present invention is useful for the production of many biological products such as, pharmaceutical or therapeutic proteins. For example, the present invention can be useful for the production of biological molecules such as hormones including cytokines (i.e., secreted amino acid sequences that affect a function of cells and modulates an interaction between cells in an immune, inflammatory or hematopoietic response), antibodies and other useful pharmaceutical molecules which include amino acid sequences. Cytokines includes, but are not limited to, monokines and lymphokines. Examples of cytokines include, but are not limited to, interferon α2b, Interleukin-1 (IL-1), Interleukin-6 (IL-6), Interleukin-8 (IL-8), Tumor Necrosis Factor-α (TNF-α.) and Tumor Necrosis Factor β (TNF-β), antibodies such as polyclonal and monoclonal antibodies and fragments thereof, and immunologic binding equivalents thereof. Antibodies may include, but are not limited to polyclonal antibodies, monoclonal antibodies (MAbs), humanized or chimeric antibodies, single chain antibodies, FAb fragments, F(Ab′)₂ fragments, fragments produced by a FAb expression library, anti-idiotypic (anti-Id) antibodies, and epitope-binding fragments thereof. Also contemplated is the production of antibody fusion proteins, for example, Fc fusion proteins in accordance with the present methods. The methods of the present invention can also be useful for producing immunoglobulin amino acid sequences which are constituent amino acid sequences of an antibody or an amino acid sequence derived therefrom. An “immunological amino acid sequence” may be, but is not limited to, an immunological heavy or light chain and may include a variable region, a diversity region, joining region and a constant region or any combination, variant or truncated form thereof. Immunological amino acid sequences also include single-chain antibodies comprised of, but not limited to, an immunoglobulin heavy chain variable region, an immunoglobulin light chain variable region and optionally a peptide linker.

Examples of certain antibodies that can be produced in methods of the invention may include but are not limited to HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein Hb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-a antibody (CAT/BASF); CDP870 is a humanized anti-TNF-a Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-a IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

Another potentially useful application of the novel isolated ovomucoid gene expression controlling region of the present invention is the possibility of increasing the amount of a heterologous protein present in a bird, (especially the chicken) by gene transfer. In most instances, a heterologous amino acid sequence-encoding nucleic acid insert transferred into the recipient animal host will be operably linked with the ovomucoid gene expression controlling region to allow the cell to initiate and continue production of the genetic product protein. A recombinant DNA molecule of the present invention can be transferred into the extra-chromosomal or genomic DNA of the host.

The recombinant ovomucoid gene expression controlling region of the present invention and amino acid sequence coding sequence, which may include an artificial chromosome and/or a polyadenylation coding sequence, may be introduced into cells by any useful method. The recombinant molecules may be inserted into a cell to which the amino acid sequence-encoding nucleic acid is heterologous (i.e. not normally present). Alternatively, as described more fully below, the recombinant DNA molecule may be introduced into cells which normally contain the amino acid sequence-encoding nucleic acid insert of the recombinant DNA molecule, for example, to correct a deficiency in the expression of an amino acid sequence, or where over-expression of the amino acid sequence is desired.

For expression in heterologous systems, the heterologous DNA molecule is inserted into the expression system or vector of the present invention in proper sense orientation and correct reading frame. The vector contains the necessary elements for the transcription and translation of the inserted protein-coding sequences, including the novel isolated ovomucoid gene expression controlling region.

U.S. Pat. No. 4,237,224 to Cohen & Boyer, which is hereby incorporated by reference in its entirety, describes the production of expression systems in the form of recombinant plasmids using restriction enzyme cleavage and ligation with DNA ligase. These recombinant plasmids are then introduced to a cell by means of transformation and replicated in cultures, including eukaryotic cells grown in tissue culture.

One aspect of the present invention, therefore, is an ovomucoid gene expression controlling region expression vector suitable for delivery to a recipient cell for replication or expression of an amino acid sequence-encoding nucleic acid of the vector therein. It is contemplated to be within the scope of the present invention for the expression vector to comprise an isolated avian ovomucoid gene expression controlling region operably linked to a nucleic acid insert encoding an amino acid sequence, and optionally a polyadenylation signal sequence. The expression vector of the present invention may further comprise a bacterial plasmid sequence, a viral nucleic acid sequence, or fragments or variants thereof that may allow for replication of the vector in a suitable host.

The recombinant nucleic acid molecules of the present invention can be delivered to cells using viruses such as vaccinia virus. Methods for making a viral recombinant vector useful for expressing a protein under the control of the ovomucoid promoter are analogous to the methods disclosed in U.S. Pat. Nos. 4,603,112; 4,769,330; 5,174,993; 5,505,941; 5,338,683; 5,494,807; 4,722,848; Paoletti, E. Proc. Natl. Acad. Sci. 93: 11349-11353 (1996); Moss Proc. Natl. Acad. Sci. 93: 11341-11348 (1996); Roizman Proc. Natl. Acad. Sci. 93: 11307-11302 (1996); Frolov et al. Proc. Natl. Acad. Sci. 93: 11371-11377 (1996); Grunhaus et al. Seminars in Virology 3: 237-252 (1993) and U.S. Pat. Nos. 5,591,639; 5,589,466; and 5,580,859 relating to DNA expression vectors, inter alia; the disclosure of each of these patents and publications is incorporated herein by reference in their entireties.

Recombinant viruses can also be generated by transfection of plasmids into cells infected with virus. Suitable vectors include, but are not limited to, viral vectors such as lambda vector system λgt11, λgt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif., which is hereby incorporated by reference), pQE, pIH821, pGEX, pET series (see Studier, F. W. et. al. (1990) “Use of T7 RNA Polymerase to Direct Expression of Cloned Genes” Gene Expression Technology, vol. 185, which is hereby incorporated by reference in its entirety) and any derivatives thereof, cosmid vectors and, in certain embodiments, artificial chromosomes, such as, but not limited to, YACs, BACs, BBPACs or PACs. Such artificial chromosomes are useful in that a large nucleic acid insert can be propagated and introduced into the avian cell. Recombinant molecules can be introduced into cells via transformation, particularly transduction, conjugation, mobilization, or electroporation. The introduction of recombinant virus to embryonic cells such as blastodermal cells may be accomplished by employing replication defective or replication competent retroviral particles as disclosed in, for example, U.S. Pat. No. 6,730,822, issued May 4, 2004 and U.S. patent application Ser. No. 10/463,980, filed Jun. 17, 2003, the disclosures of which are incorporated in their entirety herein by reference. The DNA sequences are cloned into the vector using standard cloning procedures in the art, as described by Sambrook et al. Molecular Cloning: A Laboratory Manual, 3^{rd} ed., Cold Spring Harbor Laboratory, Cold Springs Harbor, N.Y. (2001), which is hereby incorporated by reference in its entirety.

The vectors of the invention comprise one or more nucleotide sequences encoding a heterologous protein desired to be expressed in the transgenic avian, as well as regulatory elements such as promoters, enhancers, Matrix Attachment Regions, IRES's and other translation control elements, transcriptional termination elements, polyadenylation sequences, etc. In particular embodiments, the vector of the invention contains at least two nucleotide sequences coding for heterologous proteins, for example, but not limited to, the heavy and light chains of an immunoglobulin.

The present invention further relates to nucleic acid vectors and transgenes inserted therein, having the avian ovomucoid gene expression controlling region of the invention, that incorporate multiple amino acid sequence-encoding regions, wherein a first amino acid sequence-encoding region is operatively linked to a transcription promoter and a second amino acid sequence-encoding region is operatively linked to an IRES. For example, the vector may contain coding sequences for two different heterologous proteins (e.g., the heavy and light chains of an immunoglobulin).

Such nucleic acid constructs, when inserted into the genome of a bird and expressed therein, will generate individual amino acid sequences that may be post-translationally modified, for example, glycosylated or, in certain embodiments, form complexes, such as heterodimers with each other in the white of the avian egg. Alternatively, the expressed amino acid sequences may be isolated from an avian egg and combined in vitro, or expressed in a non-reproductive tissue such as serum. In other embodiments, for example, but not limited to, when expression of both heavy and light chains of an antibody is desired, two separate constructs, each containing a coding sequence for one of the heterologous proteins operably linked to the ovomucoid gene expression controlling region of the invention are introduced into the avian cell. Alternatively, two transgenic avians each containing one of the two heterologous proteins (e.g., one transgenic avian having a transgene encoding the light chain of an antibody and a second transgenic avian having a transgene encoding the heavy chain of the antibody) can be bred to obtain an avian containing both transgenes in its germline and expressing both transgene encoded proteins, preferably in eggs.

Once the ovomucoid gene expression controlling region of the present invention has been cloned into a vector system, it is ready to be incorporated into a host cell. Such incorporation can be carried out by the various forms of transformation noted above, depending upon the vector/host cell system. Suitable host cells include, but are not limited to, bacteria, virus, yeast, mammalian or avian cells, and the like. Alternatively, it is contemplated that the incorporation of the DNA of the present invention into a recipient cell may be by any suitable method such as, but not limited to, viral transfer, electroporation, gene gun insertion, sperm mediated transfer to an ovum, microinjection, cytoplasmic injection, pronuclear injection and the like.

Another aspect of the present invention, therefore, is a method of expressing a heterologous amino acid sequence in a eukaryotic cell by transfecting the cell with a recombinant DNA comprising an avian ovomucoid gene expression controlling region operably linked to a nucleic acid insert encoding an amino acid sequence and, optionally, a polyadenylation signal sequence, and culturing the transfected cell in a medium suitable for expression of the heterologous amino acid sequence under the control of the avian ovomucoid gene expression controlling region.

In certain embodiments, the ovomucoid gene expression controlling region directs a level of expression of the heterologous protein in avian eggs that is greater than 5 μg, 10 μg, 50 μg, 100 μg, 250 μg, 500 μg, or 750 μg, more preferably greater than 1 mg, 2 mg, 5 mg, 10 mg, 20 mg, 50 mg, 100 mg, 200 mg, 500 mg, 700 mg, 1 gram, 2 grams, 3 grams, 4 grams or 5 grams per egg. Such levels of expression can be obtained using the expression controlling regions of the invention.

In one embodiment of the method of the present invention, the recipient eukaryotic cell is derived from an avian. In one embodiment, the avian is a chicken.

Yet another aspect of the present invention is a eukaryotic cell transformed with an expression vector according to the present invention and described above. In one embodiment of the present invention, the transformed cell is a chicken oviduct cell and the nucleic acid insert comprises the chicken ovomucoid gene expression controlling region, a nucleic acid insert encoding a human interferon a2d with codons optimized for expression in an avian cell, and an SV40 polyadenylation sequence.

It is contemplated that the transfected cell according to the present invention may be transiently transfected, whereby the transfected recombinant DNA or expression vector may not be integrated into the genomic nucleic acid. It is further contemplated that the transfected recombinant DNA or expression vector may be stably integrated into the genomic DNA of the recipient cell, thereby replicating with the cell so that each daughter cell receives a copy of the transfected nucleic acid. It is still further contemplated for the scope of the present invention to include a transgenic animal (e.g., a transgenic avian) producing a heterologous protein expressed from a transfected nucleic acid according to the present invention.

One certain aspect of the present invention relates to transgenic animals including avians and methods of producing them. Transgenic animals of the present invention contain a transgene which includes an isolated ovomucoid gene expression controlling region of the present invention and which preferably, though optionally, expresses a heterologous gene in one or more cells in the animal. Transgenic avians can be produced by introduction of nucleic acid molecules disclosed herein into the cells of avians including, but not limited to chicken, turkey, duck, goose, quail, pheasants, parrots, finches, hawks, crows and ratites including ostrich, emu and cassowary. Any useful method for introducing nucleic acid into the cells of an animal may be employed in the present invention.

In one embodiment of the present invention, the transgenic animal is an avian selected from a turkey, duck, goose, quail, pheasant, ratite, an ornamental bird or a feral bird. In another embodiment, the avian is a chicken and the heterologous protein produced under the transcriptional control of the isolated avian ovomucoid gene expression controlling region according to the present invention is primarily localized to the white of an egg.

An exemplary approach for the in vivo introduction of an amino acid sequence-encoding nucleic acid operably linked to the subject novel isolated ovomucoid gene expression controlling region into a cell is by use of a viral vector containing nucleic acid, e.g. a cDNA, encoding the gene product. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors provide efficient delivery of genes into cells, and the transferred nucleic acids are stably integrated into the chromosomal DNA of the host. Recombinant retrovirus can be constructed in the part of the retroviral coding sequence (gag, pol, env) that has been replaced by nucleic acid comprising a ovomucoid gene expression controlling region, thereby rendering the retrovirus replication defective. Protocols for producing recombinant retroviruses and for infecting cells in vitro or in vivo with such viruses may be found in Current Protocols in Molecular Biology, Ausubel et al. (1989) (eds.) Greene Publishing Associates, Sections 9.10-9.14 and other standard laboratory manuals. Examples of suitable retroviruses include pLJ, pZIP, pWE and pEM which are all well known to those skilled in the art. Examples of suitable packaging virus lines for preparing both ecotropic and amphotropic retroviral systems include psiCrip, psiCre, psi2 and psiAm.

Furthermore, it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include coupling antibodies specific for cell surface antigens to the viral env protein (Roux et al., Proc. Natl. Acad. Sci. 86: 9079-9083 (1989); Julan et al., J. Gen. Virol. 73: 3251-3255 (1992); and Goud et al., Virology 163: 251-254 (1983)) or coupling cell surface ligands to the viral env proteins (Neda et al., J. Biol. Chem. 266: 14143-14146 (1991)), all of which are incorporated herein by reference in their entireties. Coupling can be in the form of the chemical cross-linking with a protein or other moiety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector into an amphotropic vector.

Another viral gene delivery system useful in the present invention utilizes adenovirus-derived vectors. The genome of an adenovirus can be manipulated such that it encodes a gene product of interest, but is inactivated in terms of its ability to replicate in a normal lytic viral life cycle (see, for example, Berkner et al., BioTechniques 6: 616 (1988); Rosenfeld et al., Science 252: 43 1434 (1991); and Rosenfeld et al., Cell 68: 143-155 (1992)), all of which are incorporated herein by reference in their entireties. Suitable adenoviral vectors derived from the adenovirus strain Ad type 5 dl324 or other strains of adenovirus (e.g., Ad2, Ad3, Ad7 etc.) are well known to those skilled in the art. The virus particle is relatively stable and amenable to purification and concentration, and as above, can be modified so as to affect the spectrum of infectivity. Additionally, introduced adenoviral DNA (and foreign DNA contained therein) may not be integrated into the genome of a host cell but remains episomal, thereby avoiding potential problems that can occur as a result of insertional mutagenesis in situations where introduced DNA becomes integrated into the host genome (e.g., retroviral DNA). Most replication-defective adenoviral vectors currently in use and therefore favored by the present invention are deleted for all or parts of the viral E1 and E3 genes but retain as much as 80% of the adenoviral genetic material (see, e.g., Jones et al., Cell 16:683 (1979); Berkner et al., supra; and Graham et al., in Methods in Molecular Biology, E. J. Murray, (1991) Ed. (Humana, Clifton, N.J.) vol. 7. pp. 109-127), all of which are incorporated herein by reference in their entireties. Expression of an inserted gene such as, for example, encoding the human interferon α2b, can be under control of the exogenously added ovomucoid gene expression controlling region sequences.

Yet another viral vector system useful for delivery of, for example, the subject avian ovomucoid gene expression controlling region operably linked to a nucleic acid encoding an amino acid sequence, is the adeno-associated virus (AAV). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993)), all of which are incorporated herein by reference in their entireties.

Most non-viral methods of gene transfer rely on normal mechanisms used by eukaryotic cells for the uptake and intracellular transport of macromolecules. In one embodiment, non-viral gene delivery systems of the present invention rely on endocytic pathways for the uptake of the subject ovomucoid gene expression controlling region and operably linked amino acid sequence-encoding nucleic acid by the targeted cell. Exemplary gene delivery systems of this type include liposomal derived systems, poly-lysine conjugates, and artificial viral envelopes.

In a representative embodiment, a nucleic acid comprising the novel isolated ovomucoid gene expression controlling region of the present invention can be entrapped in liposomes bearing positive charges on their surface (e.g., lipofectins) and (optionally) which are tagged with antibodies against cell surface antigens of the target tissue (Mizuno et al., NO Shinkei Geka 20:547-551 (1992); PCT publication WO91/06309; Japanese patent application 1047381; and European patent publication EP-A-43075), all of which are incorporated herein by reference in their entireties.

In similar fashion, the gene delivery system comprises an antibody or cell surface ligand that is cross-linked with a gene binding agent such as polylysine (see, for example, PCT publications WO93/04701, WO92/22635, WO92/20316, WO92/19749, and WO92/06180), all of which are incorporated herein by reference in their entireties. It will also be appreciated that effective delivery of the subject nucleic acid constructs via receptor-mediated endocytosis can be improved using agents which enhance escape of gene from the endosomal structures. For instance, whole adenovirus or fusogenic peptides of the influenza HA gene product can be used as part of the delivery system to induce efficient disruption of DNA-containing endosomes (Mulligan et al., Science 260: 926 (1993); Wagner et al., Proc. Natl. Acad. Sci. 89:7934 (1992); and Christiano et al., Proc. Natl. Acad. Sci. 90:2122 (1993)), all of which are incorporated herein by reference in their entireties. It is further contemplated that a recombinant DNA molecule comprising the novel isolated ovomucoid gene expression controlling region of the present invention may be delivered to a recipient host cell by other non-viral methods including by gene gun, microinjection, sperm-mediated transfer as described in PCT/US02/30156, filed Sep. 23, 2002 and incorporated herein by reference in its entirety, nuclear transfer, or the like.

Suitable methods for the generation of transgenic avians having heterologous DNA incorporated therein, for example, cytoplasmic injection and pronuclear injection, are described, for example, in U.S. patent application Ser. No. 10/251,364 filed Sep. 18, 2002, now U.S. Pat. No. 7,312,374, issued Dec. 25, 2007, and U.S. patent application Ser. No. 10/679,034, filed Oct. 2, 2003, the disclosure of both of these patent applications is incorporated herein by reference in its entirety. Other methods for the introduction of nucleic acids of the present invention include those disclosed in U.S. patent application Ser. No. 10/842,606 filed May 10, 2004, the disclosure of which is incorporated herein by reference in its entirety, and other methods disclosed herein.

In various embodiments of the present invention, the expression of the transgene may be restricted to specific subsets of cells, tissues or developmental stages utilizing, for example, cis-acting sequences acting on the ovomucoid gene expression controlling region of the present invention and which control gene expression in the desired pattern. Tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

One embodiment of the present invention, therefore, is a transgenic avian having a heterologous polynucleotide sequence comprising a nucleic acid insert encoding the heterologous amino acid sequence and operably linked to the novel isolated avian ovomucoid gene expression controlling region. In an embodiment of the present invention, the transgenic avian is selected from a chicken, a turkey, a duck, a goose, a quail, a pheasant, a ratite, an ornamental bird or a feral bird. In another embodiment of the present invention, the transgenic avian is a chicken.

In still another embodiment of the transgenic avian of the present invention, the transgenic avian includes an avian ovomucoid gene expression controlling region included in SEQ ID NO: 36 or a functional portion thereof.

In yet another embodiment of the transgenic avian of the present invention, the transgenic avian further comprises a polyadenylation signal sequence.

In still yet another embodiment of the transgenic avian of the present invention, the polyadenylation signal sequence is derived from the SV40 virus.

In another embodiment of the transgenic avian of the present invention, the nucleic acid insert encoding an amino acid sequence has a codon complement optimized for protein expression in an avian.

In another embodiment of the transgenic avian of the present invention, the transgenic avian produces the heterologous amino acid sequence in the serum or an egg white. In another embodiment of the transgenic avian of the present invention, the transgenic avian produces the heterologous amino acid sequence in an egg white.

In one embodiment, certain pharmaceutical comprising agents that can modulate the regulation of the expression of an amino acid sequence-encoding nucleic acid operably linked to a ovomucoid gene expression controlling region can be administered in dosages and by techniques well known to those skilled in the medical or veterinary arts, taking into consideration such factors as the age, sex, weight, species and condition of the recipient animal, and the route of administration. Standard pharmaceutical texts, such as Remmington's Pharmaceutical Science, 17th edition, 1985 may be consulted to prepare suitable preparations, without undue experimentation. Dosages can generally range from a few hundred milligrams to a few grams.

The present invention is further illustrated by the following examples, which are provided by way of illustration and should not be construed as limiting. The contents of all references, published patents and patents cited throughout the present application are hereby incorporated by reference in their entireties.

### Example 1

### PCR Amplification of Ovomucoid Promoter

Sense primer OVINs2, 5′-TAGGCAGAGCAATAGGACTCTCAACCTCGT-3′(SEQ ID NO: 1) and the antisense primer, OVMUa2, 5′-AAGCTTCTGCAGCACTCTGGGAGTTACTCA-3′ (SEQ ID NO: 2) were designed according to the sequences of chick ovoinhibitor exon 16 (Genbank Accession No: M16141) and a fragment of the chick ovomucoid promoter region (Genbank Accession No: J00897) respectively. The template DNA for PCR amplification of the ovomucoid promoter region was prepared from white Leghorn chick blood.

A series of different PCR conditions were carried out to optimize synthesis of the approximately 10.0 kb product. In these tests, the template DNA concentrations were 500 ng, 100 ng, 50 ng, or 10 ng. Two sets of primers, OVINs1 (SEQ ID NO: 3) and OVMUa1 (SEQ ID NO: 4), or OVINs2 (SEQ ID NO: 1) and OVMUa2 (SEQ ID NO: 2) shown in FIG. 3, three Mg⁺⁺ concentrations (1.0 mM, 1.5 mM and 2.0 mM) and annealing temperatures from 50° C. to 70° C. were used.

The results of the tests were as shown in FIG. 1. As shown in lanes 1 through 8, test reactions having 500 ng DNA template, the OVINs1 (SEQ ID NO: 3) and OVMUa1 (SEQ ID NO: 4) primers, 60 mM Tris-SO₄, pH 9.1, 18 mM (NH₄)₂SO₄, 1.0 mM Mg²⁺, and annealing temperatures between 50° C. to 58° C. gave no specific DNA product. Also, as shown in lanes 17 through 24 of FIG. 1, in test reactions having 100 ng DNA template, the OVINs1 and OVMUa1 primers, 60 mM Tris-SO₄, pH 9.1, 18 mM (NH₄)₂SO₄, 1.0 mM Mg²⁺, and annealing temperatures between 50° C. to 58° C., no specific bands were seen. However, as shown in lanes 9 through 16 of FIG. 1, test reactions having 500 ng DNA template, the OVINs2 (SEQ ID NO: 1) and OVMUa2 (SEQ ID NO: 2) primers, 60 mM Tris-SO₄, pH 9.1, 18 mM (NH₄)₂SO₄, 2 mM Mg²⁺ and annealing temperatures between 60° C. to 68° C. have the band of the desired length of approximately 10 kb. As shown in lanes 25 through 32, reaction conditions containing 100 ng DNA template, the OVINs2 (SEQ ID NO: 1) and OVMUa2 (SEQ ID NO: 2) primers, 60 mM Tris-SO₄, pH 9.1, 18 mM (NH₄)₂SO₄, 2 mM Mg²⁺ and annealing temperatures between about 60° C. to about 68° C. gave an increased yield of the desired product.

An approximately 10 kb product was, therefore, detected when the following conditions were used: the optimum DNA template concentration was between about 50 ng to 500 ng; the primers were OVINs2 (SEQ ID NO: 1) and OVMUa2 (SEQ ID NO: 2); the Mg²⁺ concentration was 2 mM; the annealing temperature was at or between about 60° C. to about 68° C. Each 50 μl PCR reaction consisted of 50 ng or 100 ng of template DNA, 0.1 μg each primer, 5 μl buffer B (from Elongase Enzyme Mix kit, Invitrogen Corp., Carlsbad, Calif.), 1 ml of 10 μM dNTP solution, and distilled deionized water. The PCR protocol was one cycle at 94° C. for 30 secs; thirty cycles at 94° C. for 30 secs, 60° C. for 30 secs and 68° C. for 10 mins. One cycle was performed at 68° C. for 10 mins, 35° C. for 30 mins with a final hold at 4° C. The PCR products were examined by 0.65% agarose gel analysis.

### Example 2

### Cloning of PCR Products

The PCR products were purified by standard methods. Briefly, PCI (phenol: chloroform: isoamyl alcohol, 24:25:1) and chloroform extraction were performed once. The DNA was precipitated by adding 3M sodium acetate pH 5.2 to a final concentration of 0.3M together with 2.5 volumes of 100% ethanol. The DNA pellet was dried and dissolved in distilled deionized water and then sequenced on a ABI3700 automatic sequencer (Applied Biosystems, Foster City, Calif.) using the primers OVINs2 (SEQ ID NO: 1) and OVMUa2 (SEQ ID NO: 2) to confirm the identity of each PCR product. After confirmation of the identities, the approximately 10 kb PCR product was treated with T4 polynucleotide kinase to add a phosphate to the 5′ end. Mung bean nuclease removed any overhanging adenines from the ends of the PCR products, thereby producing a blunt end. The PCR product was purified by PCI and chloroform extraction and precipitated by standard methods. This approximately 10 kb product was then cleaved with Bam HI to give two fragments, of about 4.7 and about 5.5 kb respectively.

The vector plasmid pBluescript II KS (+/−) was cut by Bam HI and Eco RV and treated with calf intestinal alkaline phosphatase. DNA fragments to be ligated into the vector were analyzed by agarose gel electrophoresis and purified from agarose gel slices using a NucleoTrap Nucleic Acid Purification Kit (BD Biosciences Clontech, Palo Alto, Calif.). Fragments of 4.7 kb and 5.5 kb were inserted into the Bam HI/Eco RV-treated pBluescript to give the constructs pBS-OVMUP4.7 and pBS-OVMUP5.5 respectively.

Positive clones were screened by Xba I/Xho I digestion. Clone pBS-OVMUP4.7, gave fragments of about 4.7 kb and 2.96 kb. Clone pBS-OVMUP5.5 gave fragments of about 5.5 kb and 2.96 kb. Apparent positive clones having the 4.7 kb insert were further confirmed by Xba I/Hind III digestion that gave three fragments of 0.5 kb, 4.2 kb and 2.9 kb. The apparent positive clones with an insert of about 5.5 kb insert were further confirmed by Xba I/Kpn I digestion that gave three fragments of 2 kb, 3.5 kb and 2.96 kb.

A construct, pBS-OVMUP-10, containing the entire approximately 10 kb PCR product cloned into the pBluescript KS II (+/−) vector was made by taking a 4.7 kb Bam HI/Xho I fragment from the pBS-OVMUP4.7 plasmid and inserting it into the Bam HI/Xba I cleaved sites of pBS-OVMUP5.5. The Xho I and Xba I cut ends were blunt-ended by treating the digested fragments with Klenow enzyme and dNTPs at 25° C. for 15 mins before the digestion with Bam HI.

### Example 3

### Sequencing

The plasmids pBS-OVMUP4.7 and pBS-OVMUP5.5 were sequenced from both ends of each insert as shown in FIG. 2. The initial primers were T7 and T3 having the nucleic acid sequences 5′-TAATACGACTCACTATAGGG-3′ (SEQ ID NO: 5) and 5′-ATTAACCCTCACTAAAGGGA-3′ (SEQ ID NO: 6) respectively. Subsequent primers (SEQ ID NOS: 7-25), as shown in FIG. 3, were designed according to the sequence results as they became available. The approximately 10 kb sequence was edited and assembled by the ContigExpress software of the Vector NTI Suite, version 6.0 (InforMax, Inc.). The region of the approximately 10 kb PCR product described in Example 1 above that encompassed the Bam HI junction was sequenced using the primers OVMUa9 (SEQ ID NO 27) and OVINs9 (SEQ ID NO 28) (shown in FIG. 3).

Each sequence chromatogram was visually checked for sequence accuracy and to locate base ambiguities. Regions containing ambiguous bases were re-sequenced with the same primer or, if still ambiguous, with a new primer designed to sequence the complementary strand. Sequencing of the original approximately 10 kb PCR fragment using the primers OVMUa9 (SEQ ID NO 27) and OVINs9 (SEQ ID NO 28) showed that the subcloned inserts of the plasmids pBS-OVMUP4.7 and pBS-OVMUP5.5 included all of the nucleic acid sequence of the parent fragment and no intervening Bam HI-Bam HI fragments were included in the final sequence SEQ ID NO: 26. The sequence (SEQ ID NO: 26) of the region lying between the 3′ end of the ovoinhibitor gene and the transcription start site of the ovomucoid-encoding region is shown in FIG. 4.

### Example 4

### Expression in Transfected Cultured Avian Myeloid and Oviduct Cells of Luciferase Regulated by the approximately 10 kb Ovomucoid Promoter Construction of p10-OM-luc

To facilitate insertion of coding sequences behind the ovomucoid promoter and in frame with the second ATG of the ovomucoid coding sequence, the Nco I site which overlaps the second ATG was changed to a Pci I site as depicted below. On the top is the wild type ovomucoid sequence at the start site of translation. On the bottom, the second Nco I site was changed to a Pci I site.

| | |
|---|---|
| Nco I Nco I | |
| MetAlaMet | |
| CTCACCATGGCCATGGC | (SEQ ID NO: 32) |
| GAGTGGTACCGGTACCG | (SEQ ID NO: 33) |
| Nco I Pci I | |
| MetAspMet | |
| CTCACCATGGACATGGA | (SEQ ID NO: 34) |
| GAGTGGTACCGGTACCG | (SEQ ID NO: 35) |

The Pci I site in the Bluescript backbone of pBS-OVMUP-10 was destroyed by cutting with Pci I, filling in the ends with Klenow polymerase and religating, creating pOM-10-alpha. The proximal promoter region was PCR amplified with primers OM-5 (SEQ ID NO.:29) and OM-6 (SEQ ID NO.:30) and template pBS-OVMUP-10. The resulting PCR product (SEQ ID NO.:31) was cut with Not I and Tth111 I and cloned into the 12059 by Not I-Tth111 I fragment of pOM-10-alpha, thereby creating pOM-10-Pci. The 1964 Nco I-S1-treated Kpn I fragment of gWiz-luciferase (Gene Therapy Systems, Inc., San Diego, Calif.) was cloned into the 12824 Pci I-Sma I fragment of pOM-10-Pci, creating p10-OM-luc.

### Primer Sequences

CGGGCAGTACCTCACCATGGACATGT (NOTE: sequence of OM5 may not be 100% complementary to the target ovomucoid sequence)

OM-5
(SEQ ID NO: 29)
5′-GCGCGGCCGCCCGGGACATGTCCATGGTGAGAGTACTGCCC-3′
OM-6
(SEQ ID NO: 30)
5′-GGCCCGGGATTCGCTTAACTGTGACTAGG-3′

### PCR Product

(SEQ ID NO: 31)
GCGCGGCCGCCCGGGACATGTCCATGGTGAGAGTACTGCC
CGGCTCTGCAGGCGGCTGCCGGTGCTCTGCTCCTGAGATG
GTCCCCCCGAGGCTGCCTGCAAATATATACAAACGTGGCG
TCCGAACTGTTGGACTGGAACACGGAGCAGCCAGCTGAAT
CTGTCAGCGGCACAATGAGGCTGGTAATATTTATTGAGGT
CCTGACCTCCAGGTAATGGTCTGCGTCTCCCAGGCAATTG
ATTTTGGCTGGACACTTGGTTAATAGCTTGAGACAAGTGT
CACATGCTCTCAGTGGTCAAAACCAAACAAACAGACTTTT
GGACCAAAAAAAAAAAAAACCTCTTAAGGACTCTGGTAGA
ACCCTAAATAGCACAGAATGCTGAGGGGAGTAAGGGACAG
GTCCTTCATTCGTCTCTGCATCCACATCTCCCAGCAGGAA
GCAGCTAAGGCTCAGCACCATCGTGCCTGCAGCTCTGCTT
TCCATGCAGTTCTGCATTCTTGGATATTCACCTCTAGGTA
AAAGCACAGGCCAGGGAGGCTTTGTCACCAGCAGAACTGA
CCAACCACTGCCAGGTGAAGCTGGCAGCACCGTATCTAAC
CTATGAAGTTAATGGTATTTAGCACTAGCTTGATAAAAGG
AAGGGTTTCTTGGCGGTTTCACTGCTTAAGTATAGAAGAG
CTTGGTAGAAGACTTGAAAGCAAGGTAAATGCTGTCAAAT
ACCACTAAAAATGTCACTTGAACCTTATCAGCAGGGAGCA
CTTATTTACAGACCTAGTCACAGTTAAGCGAATTCCCGGG
CC

The 1^{st} and 2^{nd} ATGs of the ovomucoid sequence are shown underlined. Note that the ovomucoid coding sequence is in reverse. The underlined, bold A is not in the wildtype sequence but was incorporated into pOM-10-Pci due to a error in the oligo OM-5.

### Expression of Luciferase

For expression in avian cells of non-magnum origin, HD11 cells, a chicken myeloid cell line was used. Cells were cultured as described in Beug, H., et al. (Chicken hematopoietic cells transformed by seven strains of defective avian leukemia viruses display three distinct phenotypes of differentiation. (1979) Cell, 18: 375-90, in which these cells were referred to as HBCI cells), herein incorporated by reference in its entirety. Plasmid DNA was transfected into HD11 cells with Lipofectamine 2000 (Invitrogen Corporation, Carlsbad, Calif.) according to the manufacturer's instructions.

48 hours post-transfection, the cells were harvested and pelleted. The supernatant was removed and 20 ml of 10 mM Tris, pH 7.8, 1 mM EDTA (TE) was added. The cells were frozen at −80° C. and thawed. 5 ml of the cell suspension was mixed with 25 ml of Bright-Glo™ reagent (Bright-Glo™ Luciferase Assay System, Promega, Madison, Wis.) and relative light units per second measured on a Berthold Detection Systems (Oak Ridge, Tenn.) FB12 luminometer.

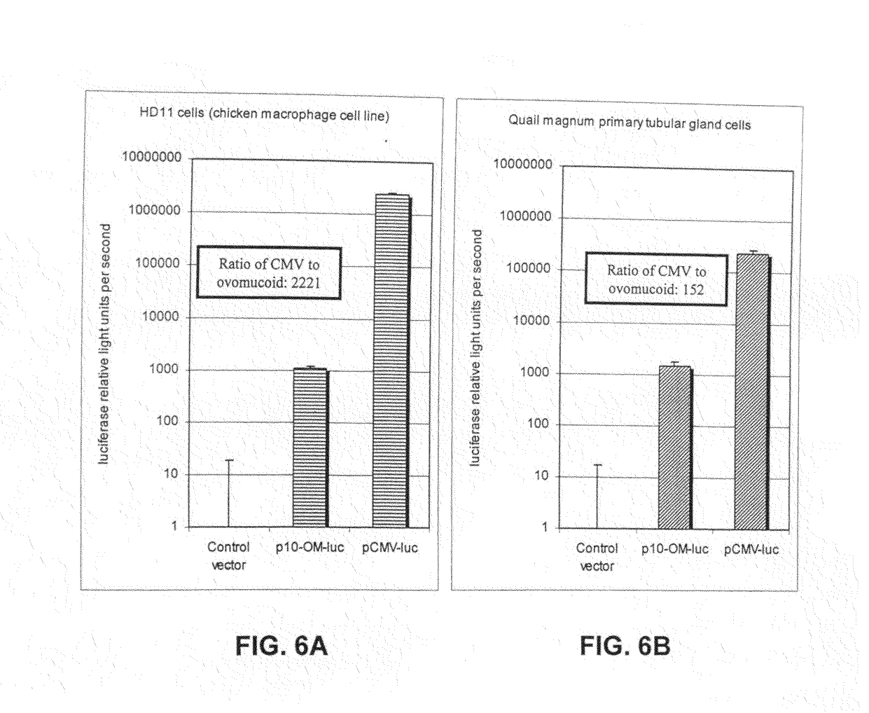

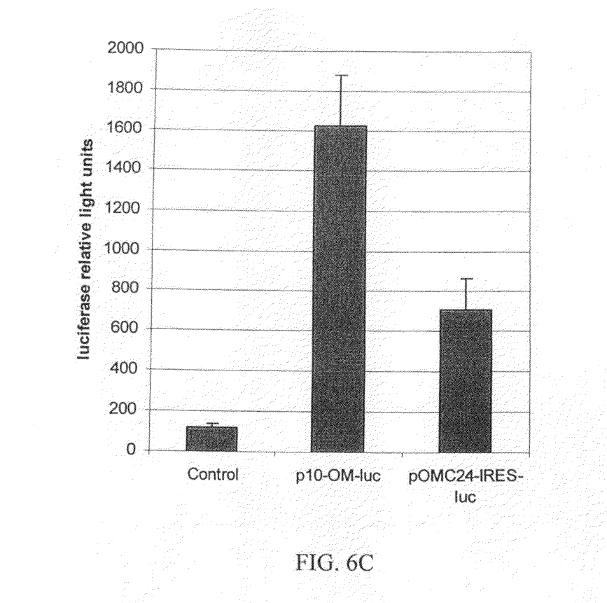

Results are depicted in FIG. 6A. HD11 cells are permissive for the CMV promoter and is able to weakly activate the ovomucoid promoter. Some expression of the luciferase gene linked to the approximately 10 kb ovomucoid is evident.

For expression in avian oviduct cells, primary tubular gland cells were isolated as follows. The oviduct of a Japanese quail (Coturnix coturnix japonica) was removed and the magnum portion minced and enzymatically dissociated with 0.8 mg/ml collagenase (Sigma Chemical Co., St. Louis, Mo.) and 1.0 mg/ml dispase (Roche Molecular Biochemicals, Indianapolis, Ind.) by shaking and titurating for 30 minutes at 37° C. The cell suspension was then filtered through sterile surgical gauze, washed three times with F-12 medium (Life Technologies, Grand Island, N.Y.) by centrifugation at 200×g, and resuspended in OPTIMEM™ (Life Technologies) such that the OD₆₀₀ was approximately 2. 800 μl of the cell suspension was plated in each well of a 6-well dish. For each transfection, 4.0 μl of DMRIE-C liposomes (Life Technologies) and 2.0 μg of plasmid DNA was preincubated for 15 minutes at room temperature in 200 μl of OPTIMEM™, and then added to the oviduct cells. Cells with DNA/liposomes were incubated for about 5 hours at 37° C. in 5% CO₂. Next, 2.0 ml of DMEM (Life Technologies), supplemented with 15% fetal bovine serum (FBS) (Atlanta Biologicals, Atlanta, Ga.), 2× penicillin/streptomycin (Life Technologies), 50 ng/ml insulin (Sigma), 10⁻⁷ M α-estradiol (Sigma), and 10⁻⁶ M corticosterone (Sigma) were added to each well, and incubation continued for about 40 hours. Medium was then harvested and centrifuged at 110×g for 5 minutes.

For quantitation, the cells were scraped into the media with a rubber policeman. One milliliter was transferred to an eppendorf tube and the cells pelleted. The supernatant was removed and 20 ml of 10 mM Tris, ph 7.8, 1 mM EDTA (TE) was added. The cells were frozen at −80° C. and thawed. 5 ml of the cell suspension was mixed with 25 ml of Bright-Glo™ reagent (Bright-Glo™ Luciferase Assay System, Promega, Madison, Wis.) and relative light units per second measured on a Berthold Detection Systems (Oak Ridge, Tenn.) FB12 luminometer.

The results are depicted in FIG. 6B. Expression of luciferase is evident from the CMV and approximately 10 kb ovomucoid promoters. The ovomucoid promoter has more activity relative to the CMV promoter in the tubular gland cells (ratio of CMV to ovomucoid is 152) than in the HD11 cells (ratio of CMV to ovomucoid is 2221). FIG. 6C shows the expression of luciferase from a OMC24-IRES-luc vector. This vector is the OMC24-IRES clone described in Example 6 with a luciferase coding sequence inserted 3′ to the IRES.

### Example 5

### Expression in Transfected Cultured Avian Oviduct Cells of Human Interferon α2b Regulated by the Approximately 10 kb Ovomucoid Promoter Construction of p10-OM-IFN

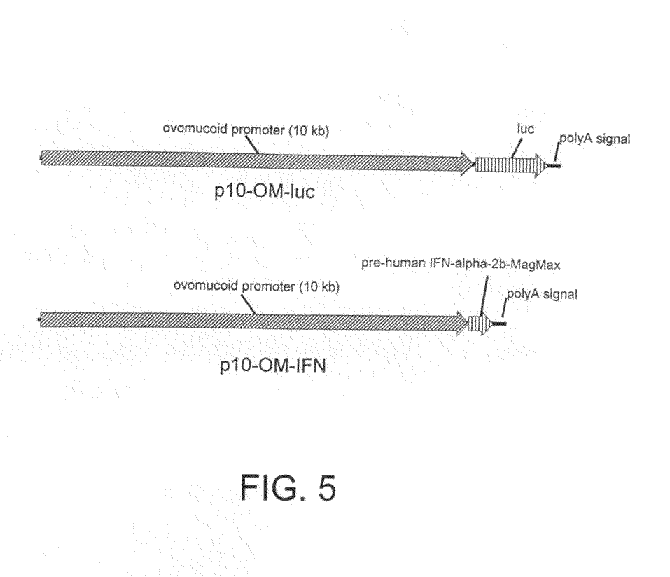

The approximately 10 kb ovomucoid promoter fragment of Example 5 was placed in front of a MagMax IFN coding sequence creating p10-OM-IFN as seen in FIG. 5 (MagMag=codon optimized for expression in the magnum of a chicken based on the frequency of codon usage of proteins such as ovalbumin, ovomucoid, lysozyme and ovomucin).

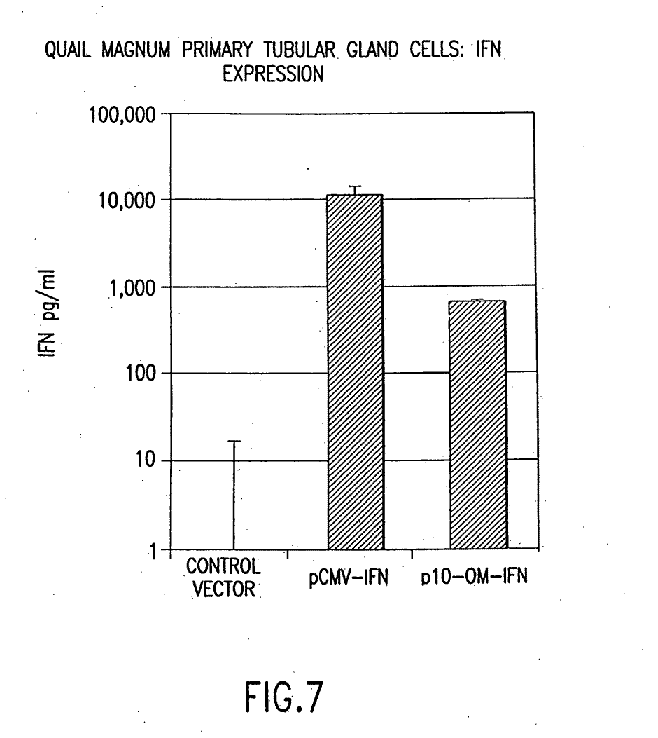

Quail primary tubular gland cells were isolated and treated as described in Example 4. 100 ml of supernatants were analyzed by ELISA (PBL Biomedical Laboratories, Flanders, N.J.) for human interferon α2b content. The results are depicted in FIG. 7. Expression of interferon is evident from the CMV and approximately 10 kb ovomucoid promoters.

### Example 6

### Construction of an Ovomucoid Promoter-Bacterial Artificial Chromosome Expression Vector with an Antibody Heavy Chain or Antibody Light Chain Coding Sequence

A chicken BAC library constructed with HindIII inserts ligated into pECBAC1 (see, Crooijmans et al., Mammalian Genome 11: 360-363, 2000, the disclosure of which is incorporated herein in its entirety by reference) was screened by PCR with two sets of primers using methods well known in the art. One primer set, OM7 and OM8, was designed to anneal in the 5′ untranslated region of the ovomucoid gene. The other primer set, Ovoinhibitor 1 and Ovoinhibitor 2, was designed to anneal in exon 3 and exon 4 of the ovoinhibitor gene.

A BAC clone was identified which yielded the expected size PCR fragment for each primer set. The BAC clone which included an insert encompassing the ovoinhibitor and ovomucoid gene was sequenced by standard techniques and designated OMC24 The sequence for OMC24 is shown in SEQ ID NO: 36.

### Primer Sequences

| (SEQ ID NO: 37) | |
|---|---|
| OM7: | CGGGCAGTACCTCACCATGGACATGT |
| (SEQ ID NO: 38) | |
| OM8: | ATTCGCTTAACTGTGACTAGG |
| (SEQ ID NO: 39) | |
| OVOINHIBITOR-1: | CGAGGAACTTGAAGCCTGTC |
| (SEQ ID NO: 40) | |
| OVOINHIBITOR-2: | GGCCTGCACTCTCCATCATA |

Polynucleotide sequences encoding the heavy chain and light chain of an IgG1 (IgG1K) monoclonal antibody were inserted into the 3′ UTR of the ovomucoid transcript coding region in two separate OMC24 clones. The heavy chain and light chain coding sequences each included a signal sequence located at their 5′ ends. For each clone, the coding sequence of each antibody chain and signal sequence was inserted into the OMC24 vector as an IRES-LC or IRES-HC cassette with the light chain and heavy chain inserts each positioned in the sense orientation

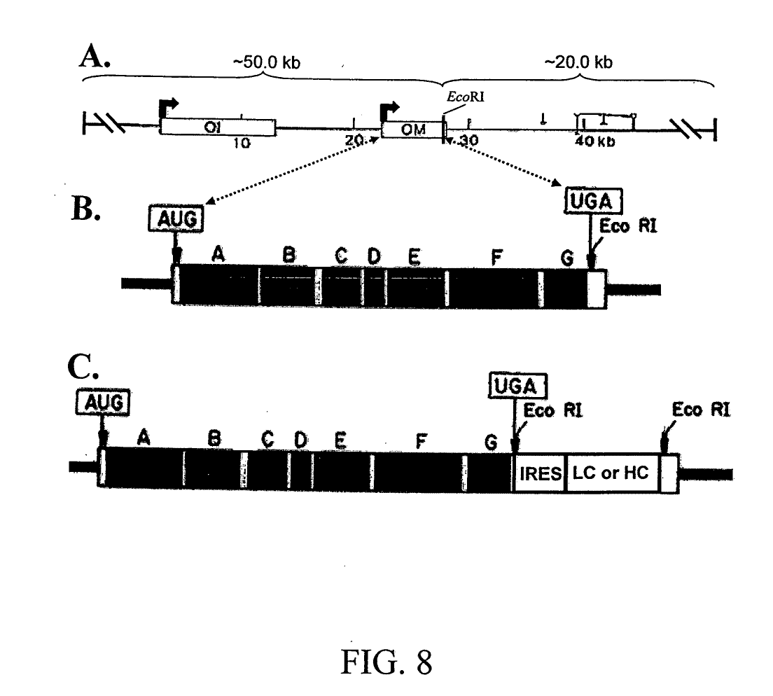

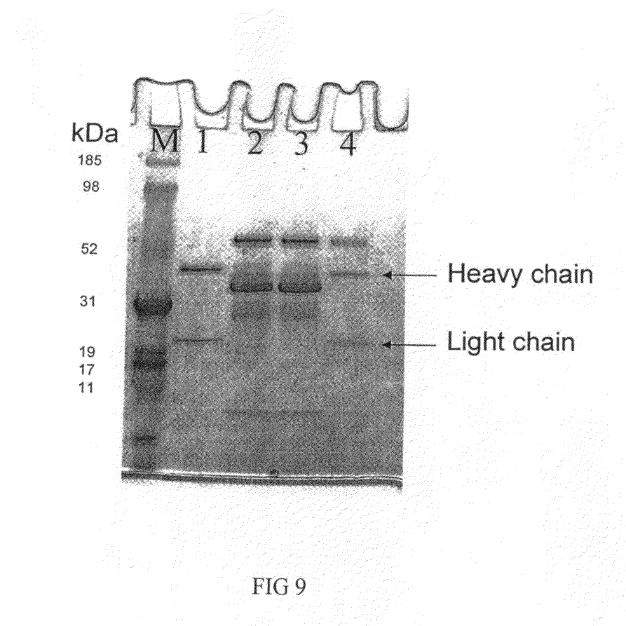

SEQ ID NO: 41 shows the IRES-LC cassette inserted in the OMC24 clone. SEQ ID NO: 42 shows the IRES-HC cassette inserted in the OMC24 clone. The IRES sequence is shown in bold. The conserved regions of the IgG1 antibody light chain and heavy chain coding sequence are underlined. The nucleotides for the coding sequences of the variable regions for the IgG1 light chain and heavy chains are represented by N's. The nucleotides encoding the signal sequences in each clone are represented by italicized N's with the start codon indicated as ATG. OMC24 nucleotide sequence flanking the IRES and the antibody coding sequence is also shown for each of the two sequences. These constructs are shown in FIG. 8.

The IRES-antibody light chain and heavy chain cassettes were each inserted into an OMC24 clone at a natural EcoRI site that resides in the 3′ UTR of ovomucoid at about position 41,627 of SEQ ID NO: 36. Because there are many EcoRI sites in OMC24, RecA-assisted restriction endonuclease cleavage (RARE) was used to cut only at the desired site. RecA assisted restriction endonuclease cleavage is described in Molecular Biotechnology (2001) Vol 18, pp 233 to 241, the disclosure of which is incorporated herein in its entirety by reference. A portion of the vector from which the cassettes were obtained of about 26 nucleotides in length can be seen 3′ of the coding sequence of the light chain and heavy chain in SEQ ID NO: 41 and SEQ ID NO: 42.

OMC24-IRES-LC
(SEQ ID NO: 41)
gatttcactc atctcctaat aatcaggtag ctgaggagat
gctgagtctg ccagttcttg ggctctgggc aggatcccat
ctcctgcctt ctctaggaca gagctcagca ggcagggctc
tgtggctctg tgtctaaccc acttcttcct ctcctcgctt
tcagggaaag caacgggact ctcactttaa gccattttgg
aaaatgctga atatcagagc tgagagaatt ccgcccctct
ccctcccccc cccctaacgt tactggccga agccgcttgg
aataaggccg gtgtgcgttt gtctatatgt tattttccac
catattgccg tcttttggca atgtgagggc ccggaaacct
ggccctgtct tcttgacgag cattcctagg ggtctttccc
ctctcgccaa aggaatgcaa ggtctgttga atgtcgtgaa
ggaagcagtt cctctggaag cttcttgaag acaaacaacg
tctgtagcga ccctttgcag gcagcggaac cccccacctg
gcgacaggtg cctctgcggc caaaagccac gtgtataaga
tacacctgca aaggcggcac aaccccagtg ccacgttgtg
agttggatag ttgtggaaag agtcaaatgg ctctcctcaa
gcgtattcaa caaggggctg aaggatgccc agaaggtacc
ccattgtatg ggatctgatc tggggcctcg gtgcacatgc
tttacgtgtg tttagtcgag gttaaaaaac gtctaggccc
cccgaaccac ggggacgtgg ttttcctttg aaaaacacga
tgataagctt gccacaacca tgnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnacggtgg cggcgccatc tgtcttcatc ttcccgccat
ctgatgagca gttgaaatct tggaaggtgg ataacgccct
ccaatcgggt aactcccagg agagtgtcac agagcaggac
agcaaggaca gcacctacag cctcagcagc accctgacgc
tgagcaaagc agactacgag aaacacaaag tctacgcctg
cgaagtcacc catcagggcc tgagctcgcc cgtcacaaag
agcttcaaca ggggagagtg ttagggatcc actagtccag
tgtggtggaa ttcaccacag gatccccact ggcgaatccc
agcgagaggt ctcacctcgg ttcatctcgc actctgggga
gctcagctca ctcccgattt tctttctcaa taaactaaat
cagcaacact cctttgtctt
OMC24-IRES-HC
(SEQ ID NO: 42)
gatttcactc atctcctaat aatcaggtag ctgaggagat
gctgagtctg ccagttcttg ggctctgggc aggatcccat
ctcctgcctt ctctaggaca gagctcagca ggcagggctc
tgtggctctg tgtctaaccc acttcttcct ctcctcgctt
tcagggaaag caacgggact ctcactttaa gccattttgg
aaaatgctga atatcagagc tgagagaatt ccgcccctct
ccctcccccc cccctaacgt tactggccga agccgcttgg
aataaggccg gtgtgcgttt gtctatatgt tattttccac
catattgccg tcttttggca atgtgagggc ccggaaacct
ggccctgtct tcttgacgag cattcctagg ggtctttccc
ctctcgccaa aggaatgcaa ggtctgttga atgtcgtgaa
ggaagcagtt cctctggaag cttcttgaag acaaacaacg
tctgtagcga ccctttgcag gcagcggaac cccccacctg
gcgacaggtg cctctgcggc caaaagccac gtgtataaga
tacacctgca aaggcggcac aaccccagtg ccacgttgtg
agttggatag ttgtggaaag agtcaaatgg ctctcctcaa
gcgtattcaa caaggggctg aaggatgccc agaaggtacc
ccattgtatg ggatctgatc tggggcctcg gtgcacatgc
tttacgtgtg tttagtcgag gttaaaaaac gtctaggccc
cccgaaccac ggggacgtgg ttttcctttg aaaaacacga
tgataagctt gccacaacca tgnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnnnnnnnn nnnnnnnnnn
nnnnnnnnnn nnnnnnnnnn nnnntcagct agcaccaagg
gcccatcggt cttccccctg gcaccctcct ccaagagcac
ctctgggggc acagcggccc tgggctgcct ggtcaaggac
tacttccccg aaccggtgac ggtgtcgtgg aactcaggcg
ccctgaccag cggcgtgcac accttcccgg ccgtcctaca
gtcctcagga ctctactccc tcagcagcgt ggtgaccgtg
ccctccagca gcttgggcac ccagacctac atctgcaacg
tgaatcacaa gcccagcaac accaaggtgg acaagagagt
tgagcccaaa tcttgtgaca aaactcacac atgcccaccg
tgcccagcac ctgaactcct ggggggaccg tcagtcttcc
tcttcccccc aaaacccaag gacaccctca tgatctcccg
gacccctgag gtcacatgcg tggtggtgga cgtgagccac
acaaagccgc gggaggagca gtacaacagc acgtaccgtg
tggtcagcgt cctcaccgtc ctgcaccagg actggctgaa
tggcaaggag tacaagtgca aggtctccaa caaagccctc
ccagccccca tcgagaaaac catctccaaa gccaaagggc
agccccgaga accacaggtg tacaccctgc ccccatcccg
ggatgaqctg accaagaacc aggtcagcct gacctgcctg
gtcaaaggct tctatcccag cgacatcgcc gtggagtggg
agagcaatgg gcagccggag aacaactaca agaccacgcc
tcccgtgctg gactccgacg gctccttctt cctctacagc
aagctcaccg tggacaagag caggtggcag caggggaacg
tcttctcatg ctccgtgatg catgaggctc tgcacaacca
ctacacgcag aagagcctct ccctgtctcc gggtaaatag
ggatccacta gtccagtgtg gtggaattca ccacaggatc
cccactggcg aatcccagcg agaggtctca cctcggttca
tctcgcactc tggggagctc agctcactcc cgattttctt

The resulting mRNA transcript from the ovomucoid promoter for each clone contains two coding sequences; one for the ovomucoid protein and another for the downstream light chain or heavy chain coding sequence. The internal ribosome entry site (IRES) engineered into the vectors is useful to facilitate translation of the downstream heavy chain or light chain coding sequence.

### Example 7

### Production of Transgenic Hens with an Ovomucoid Promoter-Bacterial Artificial Chromosome Expression Vector Transgene

100 μg each of BAC clone OMC24-IRES-LC and OCM24-IRES-HC were linearized by enzymatic restriction digest. The digested DNA was phenol/CHCl₃ extracted, ethanol precipitated, suspended in 0.25 M KCl and diluted to a working concentration of approximately 60 μg/ml. The DNA was mixed with SV40 T antigen nuclear localization signal peptide (NLS peptide, amino acid sequence CGGPKKKRKVG (SEQ ID NO: 43) with a peptide DNA molar ratio of 100:1 (Collas and Alestrom, 1996, Mol. Reprod. Develop. 45: 431-438, the disclosure of which is incorporated by reference in its entirety). The DNA samples were allowed to associate with the SV40 T antigen NLS peptide by incubation at room temperature for 15 minutes.

Introduction of the DNA-NLS complex into an avian egg was accomplished essentially as described in U.S. patent application Ser. No. 10/251,364, filed Sep. 18, 2002, now U.S. Pat. No. 7,312,374, issued Dec. 25, 2007, the disclosure of which is incorporated in its entirety herein by reference. Briefly, the germinal disc of an avian egg was illuminated by an incident light beam and visualized by an oblique macromonitering system. A micropipette injection needle was positioned by micromanipulation such that the tip of the needle was pressed into the vitelline membrane of the avian egg to a depth of about 20 μM. The injection needle was inserted through the membrane into the germinal disc to a point where only the end of the beveled opening of the needle was visible above the membrane, while the remaining of the opening was present inside the germinal disk. The DNA-NLS was then injected into the germinal disc. Approximately 100 nanoliters of DNA were injected into a germinal disc of stage I White Leghorn embryos obtained two hours after oviposition of the previous egg.

Injected embryos were surgically transferred to recipient hens via ovum transfer according to the method of Christmann et al. (PCT Publication WO 02/20752, the disclosure of which is incorporated herein in its entirety by reference) and hard shell eggs were incubated and hatched. See, Olsen and Neher, 1948, J. Exp. Zoo. 109: 355-366, the disclosure of which is incorporated in its entirety herein by reference.

Genomic DNA samples from one-week old chicks were analyzed for the presence of OMC24-IRES-LC or HC by PCR using methods well known in the field of avian transgenics. Briefly, three hundred nanograms of genomic DNA and 1.25 units of Taq DNA polymerase (Promega) were added to a 500 reaction mixture of 1× Promega PCR Buffer with 1.5 mM MgCl₂, 200 μM of each dNTP, 5 μM primers. The reaction mixtures were heated for 4 minutes at 94° C., and then amplified for 34 cycles each consisting of: 94° C. for 1 min, 60° C. for 1 min and 72° C. for 1 min. A final cycle of 4 minutes at 72° C. was performed. PCR products were detected by visualization on a 0.8% agarose gel stained with ethidium bromide.

### Example 8

### Production of Antibody by Transgenic Hens

Transgenic chicks produced as described in Example 7 were grown to maturity. Eggs were collected from the hens and egg white material was assayed for the IgG1 using sandwich ELISA.

The eggs were cracked and opened and the whole yolk portion was discarded. Both the thick and thin egg white portions were kept. 1 ml of egg white was measured and added to a plastic Stomacher 80 bag. A volume of egg white buffer (5% 1M Tris-HCl pH 9 and 2.4% NaCl) equal to two times the volume of egg white was added to the egg white. The egg white-buffer mixture was paddle homogenized in the Stomacher 80 at normal speed for one minute. The sample was allowed to stand overnight and homogenation was repeated. A 1 ml sample of the mixture was used for testing.

A Costar flat 96-well plate was coated with 100 ul of C Goat-anti-Human kappa at a concentration of 5 μg/ml in PBS. The plate was incubated at 37° C. for two hours and then washed. 200 μA of 5% PBA was added to the wells followed by an incubation at 37° C. for about 60-90 minutes followed by a wash. 100 ul of egg white samples (diluted in 1% PBA:LBP) was added to each well and the plate was incubated at 37° C. for about 60-90 min followed by a wash. 100 ul of a 1:2000 dilution of F′2 Goat anti-Human IgG Fc-AP in 1% PBA was added to the wells and the plate was incubated at 37° C. for 60-90 min followed by a wash.

The transgenic antibody was detected by placing 75 ul of 1 mg/ml PNPP (p-nitrophenyl phosphate) in 5× developing buffer in each well and incubating for about 10-30 mins at room temperature. The detection reaction was stopped using 75 ul of 1N NaOH. The OD405-650 nm was then determined for each sample well. Each OD405-650 nm value was compared to a standard curve to determine the amount of recombinant antibody present in each sample Approximately 0.3% of hens analyzed expressed antibody in their eggs. Two hens which expressed antibody are Hen 1251 which was found to produce an average of 19 ng of IgG per ml of egg white and Hen 4992 which was found to produce an average of 150 ng of IgG per ml of egg white.

FIG. 9 shows the results of an SDS-PAGE analysis of the transgenic avian derived hMab compared to the same antibody produced in mammalian cells. The antibody was first purified from egg white proteins by protein A affinity chromatography. The transgenic protein (lane 4) heavy chain and light chain had virtually an identical mobility compared to heavy and light chains of the same antibody produced by standard mammalian cell culture (lane 1). Also shown are pre-chromatography transgenic egg white (lane 2) and affinity chromatography transgenic egg white flow through (lane 3).

### Example 9

### Human Antibody Produced by Transgenic Hens Demonstrates Target Antigen Binding

The human monoclonal antibody produced and identified as described in Examples 7 and 8 was assayed for target antigen binding.

Antibody was captured from the egg white in microplate wells coated with the antibodies target antigen. Antigen-antibody complexes were quantitated using isotype-specific secondary antibody conjugated with alkaline phosphatase. The ability of the transgenic avian produced hMab to bind its target antigen was compared with the binding ability of the same hMab produced in mammalian cells.

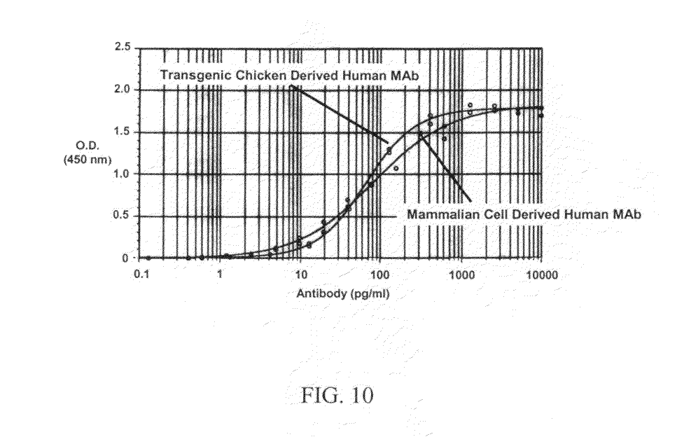

Plots showing the binding ability of each antibody are shown in FIG. 10. The plots show the level of antigen binding per picogram of antibody tested for both the antibody from transgenic chicken egg white and the antibody from a mammalian cell line. The similarity of the binding curves produced by these two antibodies indicate that the transgenic human antibody has an affinity that is substantially similar to the affinity of the antibody produced by standard methods (i.e., produced in mammalian cells).

A CHO cell line stably transfected with a plasmid that expressed the corresponding cell-surface antigen for the antibody produced by the transgenic avian was used in FACS analysis of the antibody.

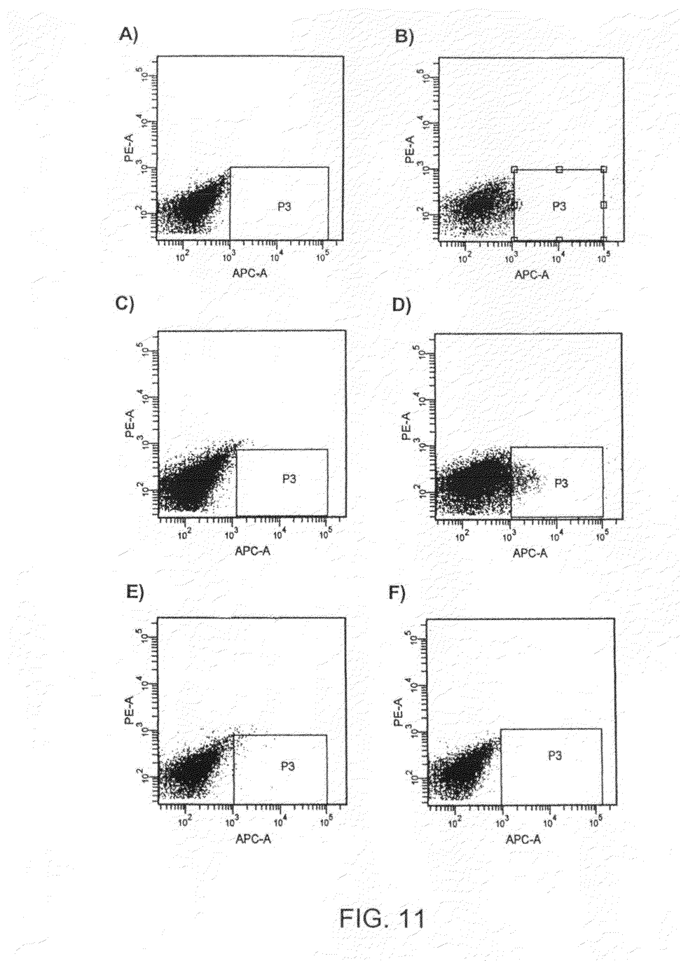

FIG. 11 shows the ability of the transgenic avian derived hMab to bind target antigen expressed on the cell surface of CHO cells relative to the ability of the antibody produced in mammalian cells. CHO cells were transfected with either a luciferase expression plasmid (11 A, 11 C, and 11 E) or an expression plasmid carrying cDNA of the hMab's target antigen (11 B, 11 D, and 11 F). Cells were collected and treated with one of three primary antibodies: 1) the antigen specific hMab produced by mammalian cells (11 A and 11 B), the antigen specific hMab produced by a transgenic hen (11 C and 11 D), or 3) human antibody of the same isotype as the antibody produced by the transgenic hen but with different antigen specificity (11 E and 11 F). An isotype specific antibody conjugated with APC (Allophycocyanin) was used to detect primary antibodies bound to the cells. Cells were sorted by FACS, counted and signal generated by the APC of the secondary antibody was quantitated. Cells that exhibited APC-associated fluorescence are delineated with a box within each graph.

Together the ELISA and FACS data show that a human antibody molecule produced by transgenic hens can bind efficiently to its target antigen.

### Example 10

### Human Antibody Produced by Transgenic Hens Demonstrates Stability

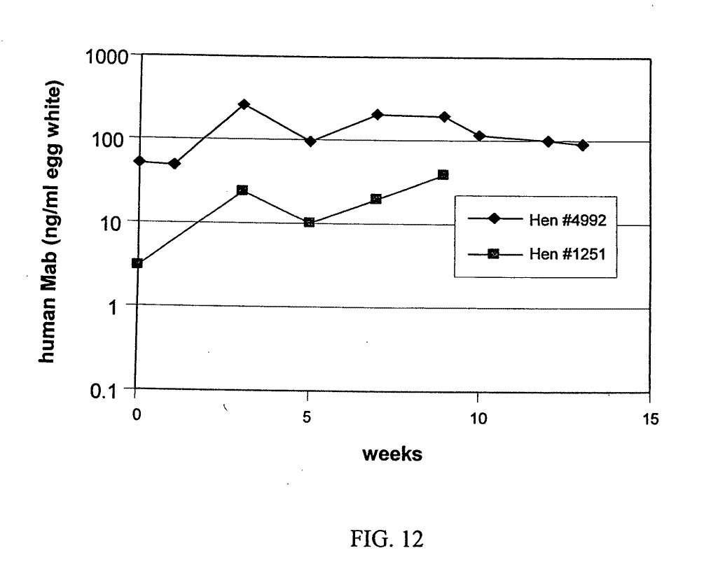

FIG. 12 shows the stability of hMab expression in transgenic hen. Eggs from transgenic hens #4992 and #1251 of Example 8 were collected over several weeks. The amount of hMab in egg white material was quantitated via sandwich ELISA for the specific human IgG1. The results indicate that the antibody produced by an avian and collected in the egg white are stable over a significant period of time.

### Example 11

### Human Antibody Produced by Transgenic Hens Demonstrates Target Cell Killing

The primary mechanism of action of many antibody therapeutics is the cytolysis of target antigen expressing cells via serum complement. This activity may require secondary modifications of the antibody in the form of proper glycosylation of the Fc portion of the antibody. Proper glycosylation has been shown to be essential for the antibody interaction with the C1q molecule of complement and with the Fcγ-family of receptors on effector cells.

The activity of the transgenic IgG1 antibody produced in Example 8 was assessed in antibody-dependent cellular cytotoxicity (ADCC) and complement-dependent cellular cytotoxicity (CDCC) assays using the antigen-expressing CHO cell line described in Example 9 as target cells.

ADCC assay: Surface antigen expressing CHO cells were incubated with purified transgenic MAb at 0.5 μg/ml or no MAb in serum free media. Human PBMCs (peripheral blood mononuclear cells) were added at an effector:target cell ratio of 20:1. The mixture was incubated at 37° C. for 4 hours. Cell lysis was assayed by LDH release and maximal release accomplished by addition of 1% Triton.

CDCC assay: Surface antigen expressing CHO cells were incubated overnight 37° C. with 0.5 μg/ml purified transgenic MAb or no MAb in the presence of 20% normal human serum. Plates were then washed and cell viability was assayed by LDH assay release and maximal release accomplished by addition of 1% Triton.

Activity was calculated for both the ADCC assay and the CDCC assay by methods well known in the art.

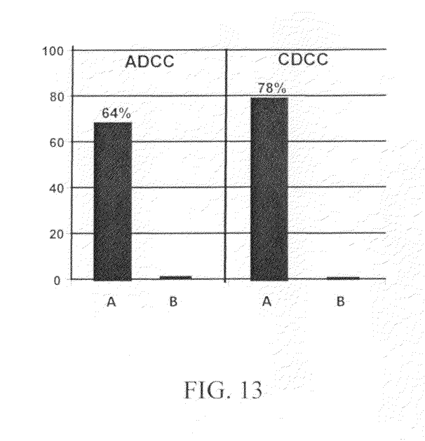

FIG. 13 shows the percent cytotoxicity for incubations with the transgenic antibody (columns A) and incubations with no antibody in serum free medium (columns B). As can be seen in FIG. 13, the transgenic human antibody efficiently mediated both ADCC and CDCC activities indicating that the antibody is appropriately glycosylated during production in avians and is effective in cytolysis of target cells.

### Example 12

### Construction of an Ovomucoid Promoter-Bacterial Artificial Chromosome Expression Vector with a CTLA4-Fc Fusion Coding Sequence and an attB Site

An ovomucoid gene expression controlling region-bacterial artificial chromosome expression vector with a CTLA4-Fc fusion coding sequence and attB site was constructed using nucleotide coding sequences for the extracellular domains of the CTLA4 (cytotoxic T lymphocyte antigen 4) receptor protein linked to nucleotide coding sequences for an immunoglobulin constant region (IgG1 Fc). The nucleotide sequence for the vector is shown in SEQ ID NO: 44

To produce this construct, an attB fragment was inserted into an EcoR1 site of the OMC24-IRES-LC clone described in Example 6. RecA-assisted restriction endonuclease cleavage (RARE) was used to cut only at the desired EcoRI site in the OMC24-IRES-LC clone. The attB fragment is shown inserted approximately at nucleotide number 26,722 to 27,029 of SEQ ID NO: 44. The attB site is shown in bold below in SEQ ID NO: 45 as it appears in the OMC24-attB-IRES-LC construct.

SEQ ID NO: 45
CCCAGAGCTG TGCAGTTGGG ATCCTAACAC CATGCAGATG
CTCCAGGACC TGCACCGAGC CCCAGCACTG GCACTCATCT
CTTCTTTCCA CCCCTCTGAG AGCAACAAGT GGCTCTGCAA
TGGCAATGTA AGTGAAACCG GGCGGGTATC TTAGAGCACC
TGGAAGCTTG CATGCCTGCA GGTCGACTCT AGAGGATCCC
CGGGTACCGA GCTCGAATTC CAGGTACCGT CGACGATGTA
GGTCACGGTC TCGAAGCCGC GGTGCGGGTG CCAGGGCGTG
CCCTTGGGCT CCCCGGGCGC GTACTCCACC TCACCCATCT
GGTCCATCAT GATGAACGGG TCGAGGTGGC GGTAGTTGAT
CCCGGCGAAC GCGCGGCGCA CCGGGAAGCC CTCGCCCTCG
AAACCGCTGG GCGCGGTGGT CACGGTGAGC ACGGGACGTG
CGACGGCGTC GGCGGGTGCG GATACGCGGG GCAGCGTCAG
CGGGTTCTCG ACGGTCACGG CGGGCATGTC GACAGCCAAG
CCGAATTCGC CCTATAGTGA GTCGTATTAC AATTCACTGG
CCGTCGTTTT ACAACGTCGT GACTGGGAAA ACCCTGGCGT
TACCCAACTT AATCGCCTTG CAGCACATCC CCCTTTCGCC
AGCTGGCGTA ATAGCGAAGA GGCCCGCACC GATCGCCCTT
CCCAACAGTT GCGCAGCCTG AATGGCGAAT GGCGCCTGAT
GCGGTATTTT CTCCTTACGC ATCTGTGCGG TATTTCACAC
CGCATATGGT GCACTCTCAG

To produce the OMC24-attB-IRES-CTLA4 clone shown in SEQ ID NO: 44, the IRES-LC portion of the OMC24-attB-IRES-LC clone was deleted using RARE and was replaced with an IRES-CTLA4-Fc coding sequence (spanning approximately from nucleotides 76,124 to 77,872 of SEQ ID NO: 44). The portion of the OMC24-attB-IRES-CTLA4-Fc clone comprising the IRES and CTLA4-Fc portions is shown below in SEQ ID NO: 46. The IRES is shown in bold and the CTLA4-Fc coding region is underlined.

SEQ ID NO: 46
ATAATCAGGT AGCTGAGGAG ATGCTGAGTC TGCCAGTTCT
TGGGCTCTGG GCAGGATCCC ATCTCCTGCC TTCTCTAGGA
CAGAGCTCAG CAGGCAGGGC TCTGTGGCTC TGTGTCTAAC
CCACTTCTTC CTCTCCTCGC TTTCAGGGAA AGCAACGGGA
CTCTCACTTT AAGCCATTTT GGAAAATGCT GAATATCAGA
GCTGAGAGAA TTCCGCCCCT CTCCCTCCCC CCCCCCTAAC
GTTACTGGCC GAAGCCGCTT GGAATAAGGC CGGTGTGCGT
TTGTCTATAT GTTATTTTCC ACCATATTGC CGTCTTTTGG
CAATGTGAGG GCCCGGAAAC CTGGCCCTGT CTTCTTGACG
AGCATTCCTA GGGGTCTTTC CCCTCTCGCC AAAGGAATGC
AAGGTCTGTT GAATGTCGTG AAGGAAGCAG TTCCTCTGGA
AGCTTCTTGA AGACAAACAA CGTCTGTAGC GACCCTTTGC
AGGCAGCGGA ACCCCCCACC TGGCGACAGG TGCCTCTGCG
GCCAGCAGCC ACGTGTATAA GATACACCTG CAAAGGCGGC
ACAACCCCAG TGCCACGTTG TGAGTTGGAT AGTTGTGGAA
AGAGTCAAAT GGCTCTCCTC AAGCGTATTC AACAAGGGGC
TGAAGGATGC CCAGAAGGTA CCCCATTGTA TGGGATCTGA
TCTGGGGCCT CGGTGCACAT GCTTTACATG TGTTTAGTCG
AGGTTAAAAA AACGTCTAGG CCCCCCGAAC CACGGGGACG
TGGTTTTCCT TTGAAAAACA CGATGATAAG CTTGCCACAA
CCATGGGTGT ACTGCTCACA CAGAGGACGC TGCTCAGTCT
GGTCCTTGCA CTCCTGTTTC CAAGCATGGC GAGCATGGCA
ATGCACGTGG CCCAGCCTGC TGTGGTACTG GCCAGCAGCC
GAGGCATCGC CAGCTTTGTG TGTGAGTATG CATCTCCAGG
CAAAGCCACT GAGGTCCGGG TGACAGTGCT TCGGCAGGCT
GACAGCCAGG TGACTGAAGT CTGTGCGGCA ACCTACATGA
TGGGGAATGA GTTGACCTTC CTAGATGATT CCATCTGCAC
GGGCACCTCC AGTGGAAATC AAGTGAACCT CACTATCCAA
GGACTGAGGG CCATGGACAC GGGACTCTAC ATCTGCAAGG
TGGAGCTCAT GTACCCACCG CCATACTACC TGGGCATAGG
CAACGGAACC CAGATTTATG TAATTGATCC AGATACCGTG
CCCAGATTCT GATCAGGAGC CCAAATCTTC TGACAAAACT
CACACATCCC CACCGTCCCC AGCACCTGAA CTCCTGGGTG
GATCGTCAGT CTTCCTCTTC CCCCCAAAAC CCAAGGACAC
CCTCATGATC TCCCGGACCC CTGAGGTCAC ATGCGTGGTG
GTGGACGTGA GCCACGAAGA CCCTGAGGTC AAGTTCAACT
GGTACGTGGA CGGCGTGGAG GTGCATAATG CCAAGACAAA
GCCGCGGGAG GAGCAGTACA ACAGCACGTA CCGGGTGGTC
AGCGTCCTCA CCGTCCTGCA CCAGGACTGG CTGAATGGCA
AGGAGTACAA GTGCAAGGTC TCCAACAAAG CCCTCCCAGC
CCCCATCGAG AAAACCATCT CCAAAGCCAA AGGGCAGCCC
CGAGAACCAC AGGTGTACAC CCTGCCCCCA TCCCGGGATG
AGCTGACCAA GAACCAGGTC AGCCTGACCT GCCTGGTCAA
AGGCTTCTAT CCCAGCGACA TCGCCGTGGA GTGGGAGAGC
AATGGGCAGC CGGAGAACAA CTACAAGACC ACGCCTCCCG
TGCTGGACTC CGACGGCTCC TTCTTCCTCT ACAGCAAGCT
CACCGTGGAC AAGAGCAGGT GGCAGCAGGG GAACGTCTTC
TCATGCTCCG TGATGCATGA GGCTCTGCAC AACCACTACA
CGCAGAAGAG CCTCTCCCTG TCTCCGGGTA AATGAGGAAT
TCACCACAGG ATCCCCACTG GCGAATCCCA GCGAGAGGTC
TCACCTCGGT TCATCTCGCA CTCTGGGGAG CTCAGCTCAC

### Example 13

### Production of Transgenic Hens with an OMC24-IRES-attB-CTLA4-Fc Fusion Coding Sequence

Twenty-five μg of OMC24-attB-IRES-CTLA4-Fc and 2.5 μg of SV40 integrase mRNA was placed in 200 μl of 28 mM Hepes (pH 7.4). The DNA/Hepes was mixed with an equal volume of PEI was diluted 10-fold with water and the mixture was incubated at room temperature for 15 mins. About 5 μl of the mixture was injected into chicken eggs essentially as described in Example 7.

Birds that produce egg white which includes CTLA4-Fc were identified using a procedure essentially as described in Example 8 but tailored specifically for CTLA4-Fc as is understood by a practitioner of ordinary skill in the art. Approximately 20% of the birds analyzed produced eggs positive for CTLA4-Fc.

### Example 14

### Construction of an Ovomucoid Promoter-Bacterial Artificial Chromosome Expression Vector Encoding an Antibody which binds to CD3

A single vector is constructed to include a cassette comprising an IRES attached to the coding sequence of the light chain of an IgG antibody which binds to CD3 and a cassette comprising an IRES attached to the coding sequence of the heavy chain of an IgG antibody which binds to CD3. The coding sequences for each of the antibody chains are produced by assembling synthetic oligonucleotides to form double stranded DNA segments which encode either the amino acid sequence for the antibody light chain (LC) or heavy chain (HC). Sequences for this particular antibody have been described in, for example, U.S. Pat. No. 6,706,265, the disclosure of which is incorporated in its entirety herein by reference. The IRES-LC cassette and IRES-HC cassette are each inserted into the ovomucoid UTR of a single OMC24 clone described in Example 6.

Transgenic hens which produce egg white which includes IgG antibody that binds to CD3 are produced essentially as described in Example 7.

### Example 15

### Construction of an Ovomucoid Promoter-Human Artificial Chromosome Expression Vector Encoding an Antibody which binds to CD3

A chicken HAC library constructed with genomic chicken DNA restriction digest inserts ligated into a HAC vector is screened by PCR with two sets of primers using methods well known in the art. One primer set is designed to anneal in the 5′ untranslated region of the ovomucoid gene. The other primer set is designed to anneal in exon 3 and exon 4 of the ovoinhibitor gene. A single HAC-chicken DNA clone is identified that includes both the UTR and the ovoinhibitor sequences and is designated HAC-O.

Two vectors are constructed to include a cassette comprising an IRES attached to the coding sequence of either the light chain or the heavy chain of an IgG antibody which binds to CD3. The coding sequences are produced by assembling synthetic oligonucleotides to form two double stranded DNA segments which encode either the amino acid sequence of the antibody light chain (LC) or heavy chain (HC). The IRES-LC cassette and IRES-HC cassette are each inserted into the ovomucoid UTR of a HAC-O clone to produce HAC-O-IRES-LC and HAC-O-IRES-HC.

Transgenic hens which produce egg white which includes IgG antibody that binds to CD3 are produced essentially as described in Example 7.

### Example 16

### Construction of an Ovomucoid Promoter P1 Derived Artificial Chromosome Expression Vector Encoding EPO

A chicken PAC library constructed with chicken genomic DNA restriction digest inserts ligated into PAC vector is screened by PCR with two sets of primers using methods well known in the art. One primer set is designed to anneal in the 5′ untranslated region of the ovomucoid gene. The other primer set is designed to anneal in exon 3 and exon 4 of the ovoinhibitor gene. A single PAC-chicken DNA clone is identified that includes both the UTR and the ovoinhibitor sequences and is designated PAC-O.

A vector is constructed which includes a cassette comprising an IRES attached to the coding sequence of human erythropoietin. Sequences for erythropoietin have been described in, for example, U.S. Pat. No. 4,703,008, the disclosure of which is incorporated in its entirety herein by reference. The IRES-EPO cassette is inserted into the ovomucoid UTR of the PAC-O clone.

Transgenic hens which produce egg white which includes EPO are produced essentially as described in Example 7.

### Example 17

### Construction of an Ovomucoid Promoter-Bacterial Artificial Chromosome Expression Vector Encoding Human Gamma-Interferon

A vector is constructed which includes a cassette coding sequence of an IRES and human gamma-interferon. Sequences for gamma-interferon have been previously described in, for example, U.S. Pat. No. 4,970,161, the disclosure of which is incorporated in its entirety herein by reference. The interferon coding sequence is inserted into the ovomucoid UTR in an OMC24 clone of Example 6.

Transgenic hens which produce egg white which includes gamma-interferon are produced essentially as described in Example 7.

### Example 18

### Construction of an Ovomucoid Promoter-Yeast Artificial Chromosome Expression Vector Encoding the Fc portion of an Antibody which binds to CD3

A chicken YAC library constructed with restriction digest inserts ligated into YAC vector is screened by PCR with two sets of primers using methods well known in the art. One primer set is designed to anneal in the 5′ untranslated region of the ovomucoid gene. The other primer set is designed to anneal in exon 3 and exon 4 of the ovoinhibitor gene. A single YAC-chicken DNA clone is identified that includes both the UTR and the ovoinhibitor sequences and is designated YAC-O.

One vector is constructed to include a cassette comprising an IRES attached to the coding sequence of the Lc portion of an IgG antibody which binds to CD3. The coding sequences are produced by assembling synthetic oligonucleotides to form two double stranded DNA segments which encode the Lc portion of an IgG antibody which binds to CD3. The IRES-Lc cassette is inserted into the ovomucoid UTR of a YAC-O clone to produce YAC-O-IRES-Lc.

Transgenic hens which produce egg white which includes the Lc portion of an IgG antibody that binds to CD3 are produced essentially as described in Example 7.

### Example 19

### Construction of an Ovomucoid Promoter-Bacterial Artificial Chromosome Expression Vector Encoding a Monoclonal Antibody that Specifically Recognizes Phosphatidylinositol-3,4-Bisphosphate

Two vectors are constructed to include a cassette comprising an IRES attached to the coding sequence of either the light chain or the heavy chain of a monoclonal antibody that specifically recognizes phosphatidylinositol-3,4-bisphosphate. The coding sequences are produced by assembling synthetic oligonucleotides to form two double stranded DNA segments which encode the amino acid sequence of either the antibody light chain (LC) or heavy chain (HC). Sequences for this particular antibody are disclosed in, for example, U.S. Pat. No. 6,709,833, the disclosure of which is incorporated in its entirety herein by reference. The IRES-LC cassette and IRES-HC cassette are each inserted into an OMC24 clone essentially as described in Example 6.

Transgenic hens which produce egg white that includes a monoclonal antibody that specifically recognizes phosphatidylinositol-3,4-bisphosphate are produced essentially as described in Example 7.

### Example 20

### Construction of pNLB-3.9-OM-CTLA4-Fc and CTLA4 Expression, Vector

The approximately 3.9 kb ovomucoid gene expression controlling region shown underlined in FIG. 14 (Fragment B) was cloned into a pBluescript vector using methodologies well know in the art to create the pOM-3.9 vector shown in FIG. 15. In order to facilitate the cloning of a coding sequence to be under the control of the approximately 3.9 kb ovomucoid gene expression controlling region, the first NcoI site that overlaps the start codon of the ovomucoid CDS (and is followed immediately by a second NcoI site) was converted into a PciI site. A NcoI 1155 by coding sequence fragment for the extracellular domains of the CTLA4 (cytotoxic T lymphocyte antigen 4) receptor protein linked to nucleotide coding sequences for an immunoglobulin constant region (IgG1 Fc) was cloned into the PciI site of the pOM-3.9 vector to produce the pOM-3.9-CTLA4 vector as shown in FIG. 15.

### Example 21

### Construction of pNLB-1.8-OM-CTLA4-Fc Expression Vector

The 2993 by Bgl II/BamHI fragment of pOM-3.9-CTLA4 (FIG. 15) bearing a 1776 by fragment of the ovomucoid promoter and the CTLA4-Fc coding region was inserted into the BglII site of the pNLB vector shown in FIG. 15 using standard recombinant DNA methodologies, creating pNLB-OM-1.8-CTLA4.

### Example 22

### Production and Concentration of VSV-G Typed pNLB-1.8-OM-CTLA4-Fc Particles

Sentas and Isoldes are cultured in F10 (Gibco), 5% newborn calf serum (Gibco), 1% chicken serum (Gibco), 50 μg/ml phleomycin (Cayla Laboratories) and 50 μg/ml hygromycin (Sigma). Transduction particles are produced essentially as described in Cosset et al., 1991, J. Virology 65: 3388-3394, herein incorporated by reference, with the following exceptions. Two days after transfection of the retroviral vector pNLB-OM-1,8-CTLA4 (from Example 21, above) into 3×10⁵ Sentas, virus is harvested in fresh media for 6-16 hours and filtered. All of the media is used to transduce 3×10⁶ Isoldes in 3 100 mm plates with polybrene added to a final concentration of 4 μg/ml. The following day the media is replaced with media containing 50 μg/ml phleomycin (Cayla Laboratories), 50 μg/ml hygromycin (Gibco) and 200 μg/ml G4 18 (Gibco).

After 10-12 days, single G418^{R} colonies are isolated and transferred to 24-well plates. After 7-10 days, the titer from each colony is determined by transduction of Sentas followed by G418 selection. Typically, 2 out of 60 colonies give titers at 1-3×10⁵. Those colonies are expanded and virus concentrated to 2-7×10⁷ as described in Allioli et al., (1994) Dev. Biol. 165:30-7, herein incorporated by reference. The virus particles are stored at −70 degrees C.

### Example 23

### Direct Oviduct Transgenesis (DOT) of pNLB-1.8-OM-CTLA4-Fc Particles and Promoter Assay

White Leghorn pullets which are between 10 and 20 weeks old are used in this procedure. One to ten days prior to treatment, the pullets are given daily dosages of diethylstilbestrol (DES, a potent form of estrogen) and progesterone to stimulate proliferation of magnum cells. Typically, doses for a 1 kg hen are 1 mg of DES and 0.8 mg of progesterone, injected intramuscularly in a volume of 0.1 ml of 95% ethanol or sesame oil. Testosterone may be substituted for progesterone.

Additional hormone injections may be given the day of surgery and for several days after. The day before treatment, the pullets are taken off of their diet and 1 mg of DES and 0.8 mg of progesterone per kg of pullet is injected daily for three days.

On the morning of the fourth day, the magnum of the oviduct is accessed by surgical procedures. Pullets are anesthetized with a standard dose of isoflurane. Aliquots of the concentrated pNLB-1,8-OM-CTLA4-Fc particles of Example 22 are thawed on ice. The magnum region of the oviduct is approached through a left lateral abdominal incision. Laparoscopic grasping forceps are used to secure the oviduct during the injection. Typically a volume of 0.5-0.6 ml of particles (1-5×10⁵ VSV-G typed particles from Example 22) is injected into three locations into the lumen of the magnum using a 1 ml syringe and 22G needle. The incision is sutured and the birds allowed to wake. The pullets are returned to their cages and given one final injection of DES and progesterone. Particle solutions remaining after injection are retitered on Isoldes and Sentas to confirm the viral titer. Six days later the same pullets are taken off their diet.

One week later the magnum is accessed through the same incision used for the injections. 0.5 ml of phosphate-buffered saline (PBS) is injected into the lumen. The lumen is gently massaged to mix the PBS with the lumen fluid. 0.1 ml PBS samples are removed from the lumen of DOT-treated hens which is assayed with a CTLA4 ELISA kit using a high sensitivity protocol reveals the presence of CTLA4 in the lumen fluid.

### Example 24

### Expression in Transfected Cultured Avian Myeloid and Oviduct Cells of Luciferase Regulated by the Approximately 3.9 kb ovomucoid Promoter

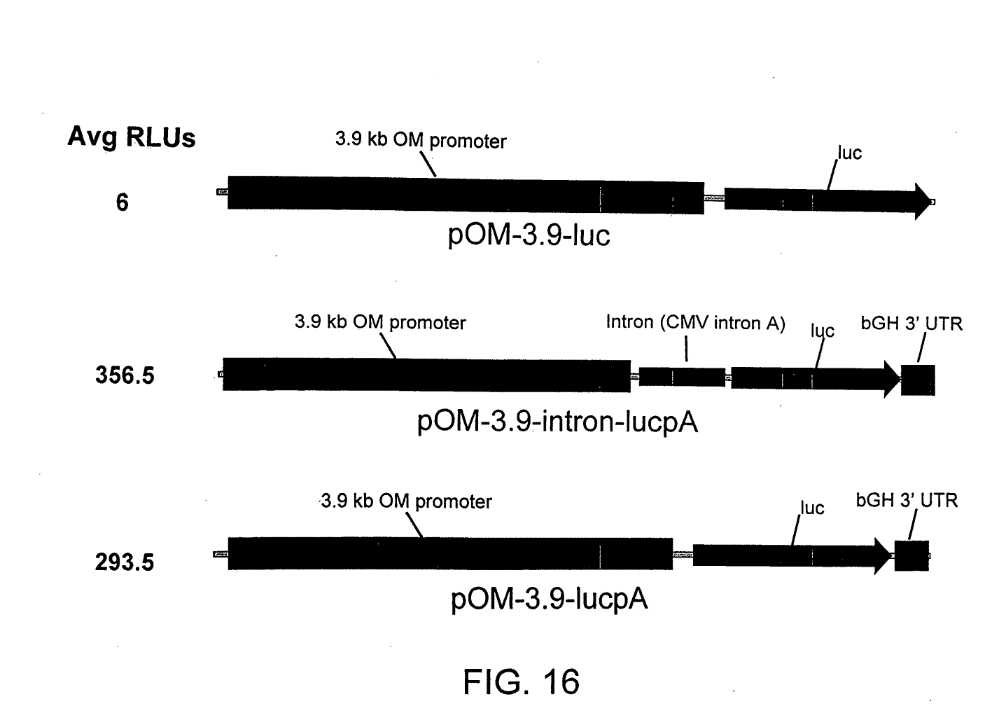

pOM-3.9-lucpA was constructed by cloning the 1972 by NcoI-KpnI fragment of pCMV-luciferase (gWiz™ Expression Vector, Gene Therapy Systems, inc.) into the 7297 by PciI-KpnI fragment of pOM-3.9. pOM-3.9-luc was constructed by cloning the 1672 by NcoI-BamHI fragment of pCMV-luciferase (gWiz™ Expression Vector, Gene Therapy Systems, inc.) into the 7295 by PciI-BamHI fragment of pOM-3.9. pOM-3.9-intron-lucpA was constructed by cloning the 2899 by SacII (mung bean nuclease treated)-KpnI fragment of pCMV-luciferase (gWiz™ Expression Vector, Gene Therapy Systems, inc.) into the 7297 by PciI (mung bean nuclease treated)-KpnI fragment of pOM-3.9. These constructs are shown in FIG. 16.

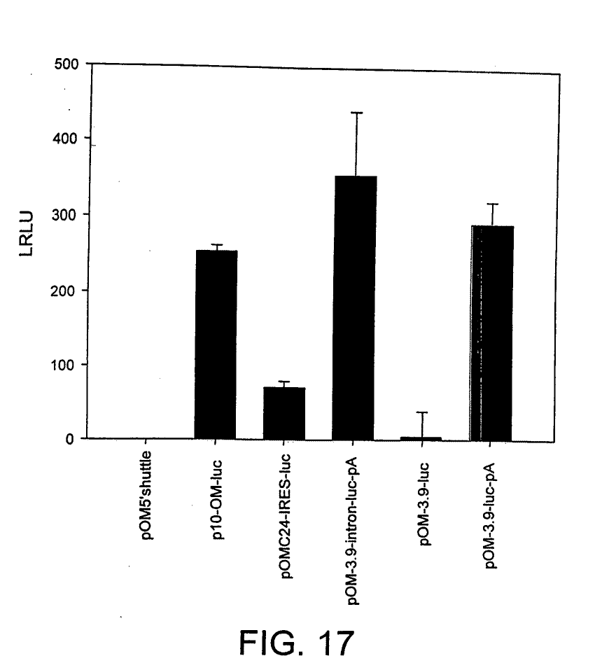

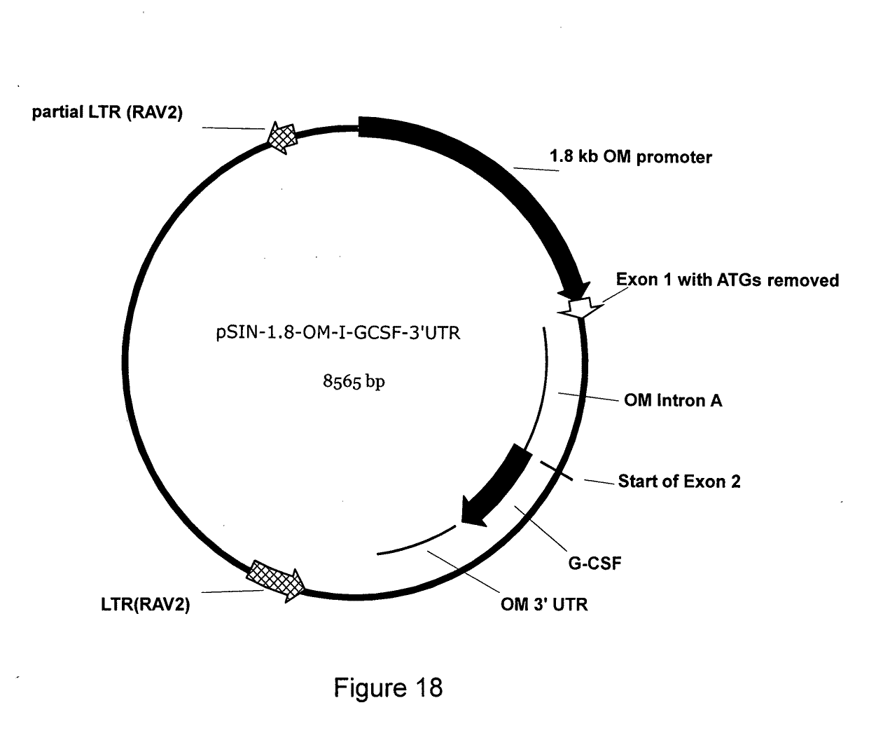

Primary tubular gland cells were isolated as described in Example 4. Transfection was performed for each of the six plasmids indicated in FIG. 17. 4.0 μl of DMRIE-C liposomes (Life Technologies) and 2.0 μg of DNA was preincubated for 15 minutes at room temperature each in a 200 μl aliquot of OPTIMEM™, which was then added to a well containing 800 ul of oviduct cells. Cells with DNA/liposomes were incubated for about 5 hours at 37° C. in 5% CO₂. 2.0 ml of DMEM (Life Technologies), supplemented with 15% fetal bovine serum (FBS) (Atlanta Biologicals, Atlanta, Ga.), 2× penicillin/streptomycin (Life Technologies), 50 ng/ml insulin (Sigma), 10⁻⁷ M α-estradiol (Sigma), and 10⁻⁶ M corticosterone (Sigma) were added to each well, and incubation continued for about 40 hours.

For each plasmid to be tested, the cells were scraped into the media with a rubber policeman. One milliliter of the resuspended cells was transferred to an eppendorf tube and the cells pelleted. The supernatant was removed and 20 ml of 10 mM Tris, ph 7.8, 1 mM EDTA (TE) was added to the cell pellet. The cells were frozen at −80° C. and thawed. 5 ml of the cell suspension was mixed with 25 ml of Bright-Glo™ reagent (Bright-Glo™ Luciferase Assay System, Promega, Madison, Wis.) and relative light units per second measured on a Berthold Detection Systems (Oak Ridge, Tenn.) FB12 luminometer.

The results are depicted in FIG. 17. Expression of luciferase is evident from the approximately 3.9 kb OM fragment. The approximately 3.9 kb OM fragment which includes the CMV intron A appears to have more activity relative to the approximately 3.9 kb OM fragment without the CMV intron. Therefore, including an intron in an expression construct may provide for a greater level of expression by an ovomucoid gene expression controlling region, or a functional fragment, relative to the expression level provided by an identical construct without the intron.

### Example 25

### Use of OM Intron to Increase Activity of Ovomucoid Promoter

Vector pSIN-1.8-OM-1-GCSF-3′UTR, the sequence of which is disclosed in SEQ ID NO: 47, can be constructed by any useful process. An example of such construction process follows: A region of avian ovomucoid gene intron A was isolated by PCR from the nucleotide sequence of SEQ ID NO: 36 with the primers 5′-gcgcacctgcgtattgccttggcaggcgtatcgtgctg-3′ (SEQ ID NO: 49) and 5′-ggcgcggccgcgaattcgacatgttggcagaatctggggaaaaaaaggggacaaaaatgggaaacaaaggcagtctg-3′(SEQ ID NO: 50). A 668 by SpeI/NotI fragment of the PCR product was ligated to a 7230 by NotI/SpeI fragment of pOM-5′ shuttle to create pShuttle-3.9-OM-parintron-082807A. A synthetic fragment that encompasses part of exon 1 and intron A was designed to remove the first and second methionine codons (ATG) of the ovomucoid coding sequence (i.e., syn OM Exon1/intron1 junction sequence; SEQ ID NO: 51). A 566 by MfeI/SpeI fragment of syn OM Exon1/intron1 junction was ligated to the 7304 by SpeI/MfeI fragment of pShuttle-3.9-OM-parintron-082807A, creating pShuttle-3.9-OM-Intron-082807A. A region of the 3′ untranslated region of the ovomucoid gene was isolated by PCR of the nucleotide sequence of SEQ ID NO: 36 with the primers 5′-ggcccaattgcccgggtctagaatatcagagctgagagaatt-3′ (SEQ ID NO: 52) and 5′-ggagcggccgccatgtaggcagggcacactt-3′-SEQ ID NO: 53). A 615 by MfeI/NotI fragment of the PCR product was ligated to a 7863 by fragment of pShuttle-3.9-OM-Intron-082807A, producing pShuttle-3.9-OM-Intron-3′UTR-082807A. A 676 by NcoI/EcoRI fragment from pNLB-CMV-G-CSF (disclosed in U.S. Pat. No. 7,511,120, issued Mar. 31, 2009, the disclosure of which is incorporated in its entirety herein by reference) containing the G-CSF coding sequence with the EcoRI site filled in with Klenow was ligated to the 8463 by SmaI/PciI fragment from pShuttle-3.9-OM-Intron-3′UTR-082807A, creating p3.9-OM-I-GCSF-3′UTR-082807B. p3.9-OM-I-GCSF-3′UTR-082807B was cut with BglII and Not I, both ends filled in with Klenow fragment, and the 4121 by fragment ligated with the 4444 by BamHI/NruI sites of pALV-SIN after the BamHI site was filled in, creating pSIN-1,8-OM-I-GCSF-3′UTR. pALV-SIN is disclosed in US patent publication No. 2009/0307786, Dec. 10, 2009, the disclosure of which is incorporated in its entirety herein by reference.

The resultant pSIN-1.8-OM-I-GCSF-3′UTR vector contains sequence of Exon 1 of the ovomucoid promoter, which is 5′ of the OM Intron, in which the two existing translation start codons have been eliminated. A start codon has been inserted into nucleotide sequence which is 3′ of the OM Intron in order to take advantage of the increased expression facilitated by the OM Intron sequence.

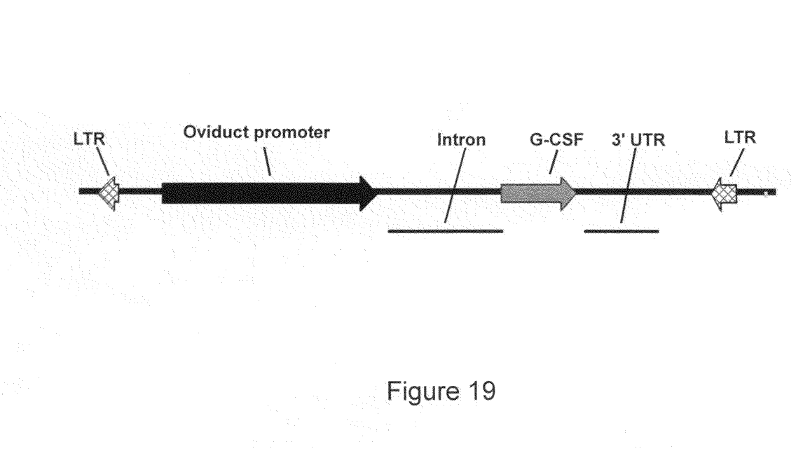

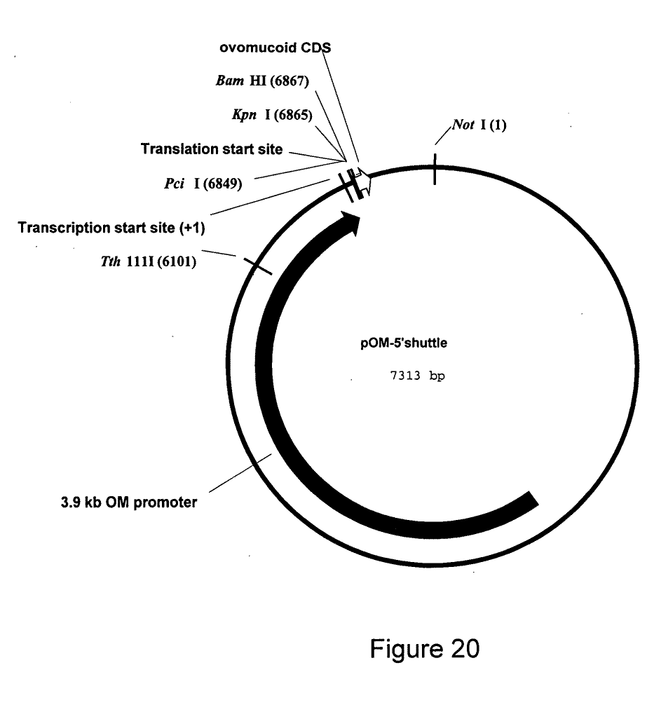

The pSIN-1.8-OM-I-GCSF-3′UTR vector was introduced into chicken embryos essentially as disclosed in U.S. Pat. No. 7,524,626, issued Apr. 8, 2009, the disclosure of which is incorporated in its entirety herein by reference, to produce transgenic birds. Two lines obtained from two G0 males were analyzed (OM-A and OM-B) which express human G-CSF in egg white of G1 hens. A diagram of the integrated pSIN-1.8-OM-I-GCSF-3′UTR vector is shown in FIG. 19.

The effect of OM Intron on the expression of the heterologous protein (G-CSF) was evaluated by construction of vector, pSIN-1.8-OM-GCSF-3′UTR-beta, in which the intron is absent. G1 hens having the pSIN-1,8-OM-GCSF-3′UTR-beta integrated vector expressed G-CSF in egg white but at a reduced level relative to G1 hens having the pSIN-1,8-OM-I-GCSF-3′UTR integrated vector. G1 hens bearing pSIN-1,8-OM-I-GCSF-3′UTR expressed G-CSF at levels of about three to five fold greater than G1 hens without the OM Intron (vector pSIN-1,8-OM-GCSF-3′UTR-beta).

All references cited herein are incorporated by reference herein in their entirety and for all purposes to the same extent as if each individual publication, patent or patent application is specifically and individually indicated to be incorporated by reference in its entirety for all purposes.

The citation of any publication is for its disclosure prior to the filing date and should not be construed as an admission that the present invention is not entitled to antedate such publication by virtue of prior invention.

While this invention has been described with respect to various specific examples and embodiments, it is to be understood that the invention is not limited thereto and that it can be variously practiced with the scope of the following claims.

What is claimed is:

1. A method comprising producing a heterologous protein from an egg laid by a transgenic avian wherein the avian contains in its genome a recombinant nucleic acid comprising a nucleotide sequence at least 85% identical to the sequence of about nucleotide 1886 to about nucleotide 2824 of SEQ ID NO: 47 operably linked to a coding sequence encoding the heterologous protein.

2. The method of claim 1 wherein the nucleotide sequence comprises a sequence at least 90% identical to the sequence of about 1886 to about nucleotide 2824 of SEQ ID NO: 47.

3. The method of claim 1 wherein the nucleotide sequence comprises a sequence at least 99% identical to the sequence of about 1886 to about nucleotide 2824 of SEQ ID NO: 47.

4. The method of claim 1 wherein the heterologous protein is an enzyme.

5. The method of claim 1 wherein the nucleic acid comprises a signal sequence coding region.

6. The method of claim 1 comprising isolating the protein.

7. The method of claim 1 wherein the nucleic acid comprises a vector.

8. The method of claim 7 wherein the vector is selected from the group consisting of a plasmid, a viral vector or an artificial chromosome.

9. The method of claim 1 wherein the nucleic acid comprises an artificial chromosome.

10. The method of claim 1 wherein the transgenic avian is selected from the group consisting of chicken and quail.

11. The method of claim 1 wherein the heterologous protein is a therapeutic protein.

12. The method of claim 1 wherein the heterologous protein is an antibody.

13. The method of claim 1 wherein the heterologous protein is a fusion protein.

14. The method of claim 1 wherein the heterologous protein is selected from the group consisting of erythropoietin (EPO), granulocyte colony-stimulating factor (G-CSF), granulocyte macrophage colony-stimulating factor (GM-CSF), CTLA4-Fc, interferon, interferon alpha, interferon beta, FSH, beta glucocerebrosidase and lysosomal acid lypase.

15. The method of claim 1 wherein the heterologous protein is selected from the group consisting of MAb directed against t-lymphocyte antigen CD3, MAb directed against TAG-72, tumor-associated glycoprotein, MAb or MAb fragments directed against platelet surface receptor GPII(b) and/or III(a), MAb fragment or MAb directed against tumor-associated antigen CA125, MAb or MAb fragment directed against human carcinoembryonic antigen, CEA, MAb fragment or MAb directed against human cardiac myosin, MAb fragment or MAb directed against tumor surface antigen PSMA, MAb fragments or MAb directed against HMW-MAA, MAb fragment or MAb directed against carcinoma-associated antigen, MAb fragments or MAb directed against NCA 90, MAb directed against CD20 antigen found on surface of B lymphocytes, MAb directed against the alpha chain of the IL2 receptor, MAb directed against the alpha chain of the IL2 receptor, MAb directed against TNF-alpha, MAb directed against an epitope on the surface of respiratory syncytial virus, MAb directed against HER 2 (human epidermal growth factor receptor 2), MAb directed against cytokeratin tumor-associated antigen, anti-CTLA4, dornase-alpha DNAse, TNF-alpha, IL-2-diptheria toxin fusion protein that targets cells displaying a surface IL-2 receptor, TNFR-IgG fragment fusion protein, enbrel, laronidase, teriparatide and parathyroid hormone derivatives.

16. A method comprising isolating a heterologous protein from egg white produced by a transgenic avian wherein the genome of the avian contains a recombinant nucleic acid comprising a nucleotide sequence at least 85% identical to the sequence of about 1886 to about nucleotide 2824 of SEQ ID NO: 47 operably linked to a heterologous coding sequence and the amino acid sequence encoded by the heterologous coding sequence is produced in an oviduct cell and is isolated from the egg white.

17. The method of claim 16 wherein the nucleotide sequence comprises a sequence at least 90% identical to the sequence of about 1886 to about nucleotide 2824 of SEQ ID NO: 47.

18. The method of claim 16 wherein the nucleotide sequence comprises a sequence at least 99% identical to the sequence of about 1886 to about nucleotide 2824 of SEQ ID NO: 47.

19. The method of claim 16 wherein the oviduct cell is a tubular gland cell.

20. The method of claim 16 wherein the heterologous coding sequence encodes at least one of a light chain and a heavy chain of an antibody.

21. The method of claim 16 wherein the heterologous coding sequence, encodes an enzyme.

22. The method of claim 16 wherein the transgenic avian is selected from the group consisting of chicken and quail.

23. A method comprising isolating a heterologous protein from an egg of a transgenic chicken wherein the genome of the transgenic chicken contains a heterologous nucleic acid comprising a nucleotide sequence at least 85% identical to the sequence of about 1886 to about nucleotide 2824 of SEQ ID NO: 47 operably linked to a heterologous coding sequence, the amino acid sequence encoded by the heterologous coding sequence being produced in an oviduct cell and packaged into the egg laid by the chicken.

24. The method of claim 23 wherein the nucleotide sequence comprises a sequence at least 90% identical to the sequence of about 1886 to about nucleotide 2824 of SEQ ID NO: 47.

25. The method of claim 23 wherein the nucleotide sequence comprises a sequence at least 99% identical to the sequence of about 1886 to about nucleotide 2824 of SEQ ID NO: 47.

26. The method of claim 23 wherein the heterologous nucleic acid comprises a sequence 95% identical to a nucleic acid sequence corresponding to the sequence of about 34,473 to about 36,248 of SEQ ID NO: 36.

27. The method of claim 23 wherein the heterologous protein is a therapeutic protein.

28. The method of claim 27 wherein the therapeutic protein is an enzyme.

29. The method of claim 23 wherein the heterologous protein is selected from the group consisting of erythropoietin (EPO), granulocyte colony-stimulating factor (G-CSF), granulocyte macrophage colony-stimulating factor (GM-CSF), CTLA4-Fc, interferon, interferon alpha, interferon beta, FSH, an antibody, beta glucocerebrosidase and lysosomal acid lypase.

30. The method of claim 23 wherein the heterologous protein is selected from the group consisting of MAb directed against t-lymphocyte antigen CD3, MAb directed against TAG-72, tumor-associated glycoprotein, MAb or MAb fragments directed against platelet surface receptor GPII(b) and/or III(a), MAb fragment or MAb directed against tumor-associated antigen CA125, MAb or MAb fragment directed against human carcinoembryonic antigen, CEA, MAb fragment or MAb directed against human cardiac myosin, MAb fragment or MAb directed against tumor surface antigen PSMA, MAb fragments or MAb directed against HMW-MAA, MAb fragment or MAb directed against carcinoma-associated antigen, MAb fragments or MAb directed against NCA 90, MAb directed against CD20 antigen found on surface of B lymphocytes, MAb directed against the alpha chain of the IL2 receptor, MAb directed against the alpha chain of the IL2 receptor, MAb directed against TNF-alpha, MAb directed against an epitope on the surface of respiratory syncytial virus, MAb directed against HER 2 (human epidermal growth factor receptor 2), MAb directed against cytokeratin tumor-associated antigen, anti-CTLA4, dornase-alpha DNAse, TNF-alpha, IL-2-diptheria toxin fusion protein that targets cells displaying a surface IL-2 receptor, TNFR-IgG fragment fusion protein, enbrel, laronidase, teriparatide and parathyroid hormone derivatives.
